(19) United States
(12) Patent Application Publication
(10) Pub. No.: US 2012/0258873 A1
(43) Pub. Date: Oct. 11, 2012

(54) REDUCTION OF 2,3-DIHYDROXY-2-METHYL BUTYRATE (DHMB) IN BUTANOL PRODUCTION

(75) Inventors: KATHARINE J. GIBSON, Wilmington, DE (US); Arthur Leo Kruckeberg, Wilmington, DE (US); Lori Ann Maggio-Hall, Wilmington, DE (US); Mark J. Nelson, Newark, DE (US); Ranjan Patnaik, Newark, DE (US)

(73) Assignee: Butamax(TM) Advanced Biofuels LLC, Wilmington, DE (US)

(21) Appl. No.: 13/153,866

(22) Filed: Jun. 6, 2011

Related U.S. Application Data
(60) Provisional application No. 61/472,487, filed on Apr. 6, 2011.

(51) Int. Cl. C40B 30/00; C12P 7/16; C07C 31/12; C12N 1/19

(52) U.S. Cl. 506/7; 435/254.2; 435/160; 568/840

(57) ABSTRACT

The invention relates generally to the field of industrial microbiology and butanol production. More specifically, the invention relates methods of reducing 2,3-dihydroxy-2-methyl butyrate (DHMB) in butanol production. DHMB can be reduced by inhibiting the reduction of acetolactate to DHMB, for example, by knocking out enzymes that catalyze the reduction or by removing DHMB during or after fermentation. Yeast strains, compositions, and methods for reducing DHMB and increasing butanol yield are provided.

### REFERENCE TO SEQUENCE LISTING SUBMITTED ELECTRONICALLY

The content of the electronically submitted sequence listing (Size: 410,154 bytes; and Date of Creation: Oct. 12, 2011) is incorporated herein by reference in its entirety.

### BACKGROUND OF THE INVENTION

1. Field of the Invention

The invention relates generally to the field of industrial microbiology and butanol production. More specifically, the invention relates to methods of reducing 2,3-dihydroxy-2-methylbutyrate (DHMB) in butanol production.

2. Background Art

Butanol is an important industrial chemical with a variety of applications, including use as a fuel additive, as a feedstock chemical in the plastics industry, and as a food-grade extractant in the food and flavor industry. Accordingly, there is a high demand for butanol, as well as for efficient and environmentally friendly production methods.

Production of butanol utilizing fermentation by microorganisms is one such environmentally friendly production method, and genetically engineered yeast strains that are capable of producing butanol have been produced. However, there is a need to improve the efficacy and reduce the cost of butanol production.

The biosynthesis pathway for the production of butanol in genetically engineered yeast includes the conversion of acetolactate to 2,3-dihydroxy-3-isovalerate (DHIV), which is subsequently converted to butanol. See FIG. 1. However, a side reaction in this pathway, which decreases the overall production of butanol, is the conversion of acetolactate to 2,3-dihydroxy-2-methylbutyrate (DHMB). For an efficient biosynthetic process, there is a need to prevent the conversion of acetolactate to DHMB and/or to remove DHMB from the fermentation broth.

The present invention satisfies this current need by providing methods to reduce DHMB by preventing conversion of acetolactate to DHMB or by removing DHMB from a fermentation broth. For example, DHMB can be reduced by providing recombinant yeast that comprise reduced or eliminated ability to convert acetolactate to DHMB (e.g., by modification of a polynucleotide encoding a polypeptide having acetolactate reductase activity or by modification of a polypeptide having acetolactate reductase activity). In addition, DHMB concentrations can be reduced by removal of DHMB from butanol-producing fermentations in order to provide a more pure product.

### BRIEF SUMMARY OF THE INVENTION

Methods of reducing DHMB during fermentation are provided. For example, in some embodiments, a recombinant yeast comprises a biosynthetic pathway capable of converting pyruvate to acetolactate, and the yeast produces less than 0.01 moles 2,3-dihydroxy-2-methylbutyrate (DHMB) per mole of sugar consumed.

In other embodiments, a recombinant yeast comprises a biosynthetic pathway capable of converting pyruvate to acetolactate, and the yeast produces DHMB at a rate of less than about 1.0 mM/hour.

In other embodiments, a recombinant yeast comprises a biosynthetic pathway capable of converting pyruvate to acetolactate, and the yeast produces an amount of 2,3-dihydroxy-3-isovalerate (DHIV) that is at least about 1.5 times the amount of DHMB produced.

In other embodiments, a recombinant yeast comprises a heterologous biosynthetic pathway capable of converting pyruvate to acetolactate, and the yeast comprises reduced or eliminated acetolactate reductase activity.

The biosynthetic pathway can be a butanol producing pathway. The yeast can also comprise a recombinant ketol-acid reductoisomerase (KARI) enzyme. In some embodiments, the KARI enzyme is capable of utilizing NADH. In some embodiments, the yeast is capable of producing a butanol product under anaerobic conditions.

Recombinant yeast described herein can comprise at least one deletion, mutation, and/or substitution in an endogenous polynucleotide encoding a polypeptide having acetolactate reductase activity. In some embodiments, the yeast is free of an enzyme having acetolactate reductase activity.

A polypeptide having acetolactate reductase activity can comprise a polypeptide encoded by a polynucleotide selected from the group consisting of SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:134, and SEQ ID NO:136. In some embodiments, a polypeptide having acetolactate reductase activity is YMR226C.

In some embodiments, a recombinant yeast comprises polynucleotides encoding polypeptides that catalyze the conversion of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the recombinant yeast comprises polynucleotides encoding polypeptides having acetolactate synthase, keto acid reductoisomerase, dihydroxyacid dehydratase, ketoisovalerate decarboxylase, and alcohol dehydrogenase activities.

Recombinant yeast described herein can comprise at least one deletion, mutation, and/or substitution in an endogenous polynucleotide encoding a polypeptide having pyruvate decarboxylase activity. The polypeptide having pyruvate decarboxylate activity can be PDC1, PDC5, PDC6, and combinations thereof. In some embodiments, the yeast is free of an enzyme having pyruvate decarboxylase activity.

In some embodiments, the butanol-producing pathway produces isobutanol.

Methods for the production of butanol are also described herein. The methods can comprise growing the recombinant yeast described above under conditions whereby butanol is produced. The butanol can be isobutanol.

The methods can also comprise growing a recombinant yeast comprising a biosynthetic pathway capable of converting pyruvate to acetolactate under conditions whereby butanol is produced and removing DHMB from the culture. The DHMB can be removed by extraction into an organic phase. The DHMB can also be removed by reactive extraction.

In some embodiments, the recombinant yeast in the method for producing butanol comprises a recombinant ketol-acid reductoisomerase (KARI) enzyme. The KARI enzyme can be an enzyme that is capable of utilizing NADH.

In some embodiments, the recombinant yeast used in the methods of producing butanol comprises at least one deletion, mutation, and/or substitution in an endogenous polynucleotide encoding a polypeptide having pyruvate decarboxylase activity. In some embodiments, the recombinant yeast is free of an enzyme having pyruvate decarboxylase activity.

In some embodiments, the recombinant yeast used in the methods of producing butanol comprises at least one deletion, mutation, and/or substitution in an endogenous polynucleotide encoding a polypeptide having acetolactate reductase activity. In some embodiments, the recombinant yeast is free of an enzyme having acetolactate reductase activity. The enzyme having acetolactate reductase activity can comprise a polypeptide encoded by a polynucleotide selected from the group consisting of SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:134, and SEQ ID NO:136. The polypeptide having acetolactate reductase activity can be YMR226C.

In some embodiments, the butanol produced in the methods is isobutanol.

In some embodiments of the methods described herein, the growing occurs in anaerobic conditions.

Compositions comprising butanol and no more than about 0.5 mM DHMB are also described herein.

In addition, methods of identifying a gene involved in DHMB production are described. The methods can comprise i) providing a collection of yeast strains comprising at least two or more gene deletions; ii) measuring the amount of DHMB produced by individual yeast strains; iii) selecting a yeast strain that produces no more than about 1.0 mM DHMB/hour; and iv) identifying the gene that is deleted in the selected yeast strain.

In other embodiments, the method can comprise i) providing a collection of yeast strains that over-express at least two or more genes; ii) measuring the amount of DHMB produced by individual yeast strains; iii) selecting a yeast strain that produces at least about 1.0 mM DHMB; and iv) identifying the gene that is over-expressed in the selected yeast strain.

The methods can further comprise creating a deletion, mutation, and/or substitution in the identified gene in a recombinant yeast comprising a biosynthetic pathway capable of converting pyruvate to acetolactate.

Recombinant yeast produced by such methods are also encompassed. Such recombinant yeast can further comprise a recombinant ketol-acid reductoisomerase (KARI) enzyme, which can be capable of utilizing NADH.

The recombinant yeast can comprise a biosynthetic pathway that is a butanol producing pathway. In some embodiments, the recombinant yeast comprises at least one deletion, mutation, and/or substitution in an endogenous polynucleotide encoding a polypeptide having pyruvate decarboxylase activity. In some embodiments, the recombinant yeast is free of an enzyme having pyruvate decarboxylase activity. In some embodiments, the recombinant yeast is free of an enzyme having acetolactate reductase activity.

Methods of producing butanol using recombinant yeast produced by methods of identifying a gene involved in DHMB production are also described herein. In some embodiments, the methods comprise growing the recombinant yeast identified under conditions whereby butanol is produced. In some embodiments, the butanol is isobutanol. In some embodiments, the growing occurs in anaerobic conditions.

Compositions comprising a recombinant yeast capable of producing butanol, butanol, and no more than about 0.5 mM DHMB are also provided. In some embodiments, the recombinant yeast comprises a butanol biosynthetic pathway. In some embodiments, the recombinant yeast comprises at least one deletion, mutation, and/or substitution in an endogenous polynucleotide encoding a polypeptide having pyruvate decarboxylase activity. In some embodiments, the recombinant yeast comprises at least one deletion, mutation, and/or substitution in an endogenous polynucleotide encoding a polypeptide having acetolactate reductase activity. In some embodiments, the polypeptide having acetolactate reductase activity comprises a polypeptide encoded by a polynucleotide selected from the group consisting of SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:134, and SEQ ID NO:136. In some embodiments, the polypeptide having acetolactate reductase activity is YMR226C. In some embodiments, the butanol is isobutanol.

Methods for the production of butanol comprising a) growing a recombinant yeast comprising a biosynthetic pathway capable of converting pyruvate to acetolactate under conditions whereby butanol is produced; and b) measuring DHIV concentration are also described herein. Steps a) and b) can be performed simultaneously or sequentially and in any order. In some embodiments, the measuring comprises liquid chromatography-mass spectrometry.

Methods for the production of butanol comprising a) growing a recombinant yeast comprising a biosynthetic pathway capable of converting pyruvate to acetolactate under conditions whereby butanol is produced; and b) measuring DHMB concentration are also described herein. Steps a) and b) can be performed simultaneously or sequentially and in any order. In some embodiments, the measuring comprises liquid chromatography-mass spectrometry.

Methods for increasing ketol-acid reductoisomerase (KARI) activity comprising a) providing a composition comprising acetolactate, a KARI enzyme, and an acetolactate reductase enzyme and b) decreasing DHMB levels are also provided. In some embodiments, decreasing DHMB levels is achieved by decreasing acetolactate reductase enzyme activity. In some embodiments, decreasing DHMB levels is achieved by removing DHMB from the composition. In some embodiments, the acetolactate, the KARI enzyme, and/or the acetolactate reductase enzyme are present in a recombinant yeast. In some embodiments, the recombinant yeast comprises a biosynthetic pathway capable of converting pyruvate to acetolactate.

Methods for increasing dihydroxyacid dehydratase (DHAD) activity comprising a) providing a composition comprising dihydroxyisovalerate (DHIV) and a DHAD enzyme and b) decreasing DHMB levels. In some embodiments, decreasing DHMB levels is achieved by removing DHMB from the composition. In some embodiments, the DHIV and/or the DHAD enzyme are present in a recombinant yeast. In some embodiments, the recombinant yeast comprises a biosynthetic pathway capable of converting pyruvate to acetolactate.

Methods of measuring DHMB in a composition comprising are also provided. In some embodiments, the composition comprises isobutanol. In some embodiments, the composition comprises yeast.

Methods of measuring DHIV in a composition comprising are also provided. In some embodiments, the composition comprises isobutanol. In some embodiments, the composition comprises yeast.

### BRIEF DESCRIPTION OF THE DRAWINGS/FIGURES

The various embodiments of the invention can be more fully understood from the following detailed description, the figures, and the accompanying sequence descriptions, which form a part of this application.

FIG. 1 shows an isobutanol biosynthetic pathway. Step “a” represents the conversion of pyruvate to acetolactate. Step “b” represents the conversion of acetolactate to DHIV. Step “c” represents the conversion of DHIV to KIV. Step “d” represents the conversion of KIV to isobutyraldehyde. Step “e” represents the conversion of isobutyraldehyde to isobutanol. Step “f” represents the conversion of acetolactate to DHMB.

FIG. 2 shows a phyolgenetic tree of YMR226c homologs from species of ascomycete yeast. A filamentous fungi (Neurospora crassa) sequence is included as an outgroup.

FIG. 3 shows a multiple sequence alignment (MSF Format) of nucleotide sequences of ORFs with homology to YMR226C. The gene names shown correspond to the accession numbers given in Table 6. The alignment was produced by AlignX (Vector NTI).

FIG. 4 shows a graph of the molar yield of DHMB over time.

FIG. 5 shows the specific rate of isobutanol production, Qp, of the two strains, PNY1910 and PNY2242.

FIG. 6 shows the accumulation of DHIV+DHMB in the culture supernatant during the fermentation time course with PNY1910 (triangles) and PNY2242 (diamonds). (DHMB and DHIV are not distinguished by the HPLC method used.)

FIG. 7 shows the yield of glycerol, pyruvic acid, butanediol (BDO), DHIV/DHMB, α-ketoisovalerate (aKIV), and isobutyric acid (iBuAc). DHIV and DHMB are shown together as these are not distinguished by the HPLC method used.

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All publications, patents and other references mentioned herein are incorporated by reference in their entireties for all purposes.

Although methods and materials similar or equivalent to those disclosed herein can be used in practice or testing of the present invention, suitable methods and materials are disclosed below. The materials, methods, and examples are illustrative only and are not intended to be limiting. Other features and advantages of the invention will be apparent from the detailed description and from the claims.

In order to further define this invention, the following terms, abbreviations and definitions are provided.

As used herein, the terms “comprises,” “comprising,” “includes,” “including,” “has,” “having,” “contains,” or “containing,” or any other variation thereof, are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances, i.e., occurrences, of the element or component. Therefore “a” or “an” should be read to include one or at least one, and the singular word form of the element or component also includes the plural unless the number is obviously meant to be singular.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether or not modified by the term “about,” the claims include equivalents to the quantities. In one embodiment, the term “about” means within 10% of the reported numerical value, preferably within 5% of the reported numerical value.

The term “invention” or “present invention” as used herein is a non-limiting term and is not intended to refer to any single embodiment of the particular invention but encompasses all possible embodiments as disclosed in the application.

The term “butanol” as used herein refers to 2-butanol, 1-butanol, isobutanol or mixtures thereof. Isobutanol is also known as 2-methyl-1-propanol.

The term “butanol biosynthetic pathway” as used herein refers to an enzyme pathway to produce 1-butanol, 2-butanol, or isobutanol. For example, isobutanol biosynthetic pathways are disclosed in U.S. Patent Application Publication No. 2007/0092957, which incorporated by reference herein.

A recombinant host cell comprising an “engineered alcohol production pathway” (such as an engineered butanol or isobutanol production pathway) refers to a host cell containing a modified pathway that produces alcohol in a manner different than that normally present in the host cell. Such differences include production of an alcohol not typically produced by the host cell, or increased or more efficient production. The term “heterologous biosynthetic pathway” as used herein refers to an enzyme pathway to produce a product in which at least one of the enzymes is not endogenous to the host cell containing the biosynthetic pathway.

The term “extractant” as used herein refers to one or more organic solvents which can be used to extract butanol from a fermentation broth.

“Fermentable carbon source” as used herein means a carbon source capable of being metabolized by the microorganisms disclosed herein. Suitable fermentable carbon sources include, but are not limited to, monosaccharides, such as glucose or fructose; disaccharides, such as lactose or sucrose; oligosaccharides; polysaccharides, such as starch or cellulose; one carbon substrates; and mixtures thereof.

“Fermentation broth” as used herein means the mixture of water, sugars (fermentable carbon sources), dissolved solids, microorganisms producing alcohol, product alcohol and all other constituents of the material held in the fermentation vessel in which product alcohol is being made by the reaction of sugars to alcohol, water and carbon dioxide (CO₂) by the microorganisms present. From time to time, as used herein the term “fermentation medium” and “fermented mixture” can be used synonymously with “fermentation broth”.

The term “aerobic conditions” as used herein means growth conditions in the presence of oxygen.

The term “microaerobic conditions” as used herein means growth conditions with low levels of oxygen (i.e., below normal atmospheric oxygen levels).

The term “anaerobic conditions” as used herein means growth conditions in the absence of oxygen.

The terms “PDC-,” “PDC knockout,” or “PDC-KO” as used herein refer to a cell that has a genetic modification to inactivate or reduce expression of a gene encoding pyruvate decarboxylase (PDC) so that the cell substantially or completely lacks pyruvate decarboxylase enzyme activity. If the cell has more than one expressed (active) PDC gene, then each of the active PDC genes may be inactivated or have minimal expression thereby producing a PDC-cell.

The term “carbon substrate” refers to a carbon source capable of being metabolized by the recombinant host cells disclosed herein. Non-limiting examples of carbon substrates are provided herein and include, but are not limited to, monosaccharides, oligosaccharides, polysaccharides, ethanol, lactate, succinate, glycerol, carbon dioxide, methanol, glucose, fructose, sucrose, xylose, arabinose, dextrose, or mixtures thereof.

“Biomass” as used herein refers to a natural product containing a hydrolysable starch that provides a fermentable sugar, including any cellulosic or lignocellulosic material and materials comprising cellulose, and optionally further comprising hemicellulose, lignin, starch, oligosaccharides, disaccharides, and/or monosaccharides. Biomass can also comprise additional components, such as protein and/or lipids. Biomass can be derived from a single source, or biomass can comprise a mixture derived from more than one source. For example, biomass can comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood, and forestry waste. Examples of biomass include, but are not limited to, corn grain, corn cobs, crop residues such as corn husks, corn stover, grasses, wheat, rye, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof.

“Feedstock” as used herein means a product containing a fermentable carbon source. Suitable feedstock include, but are not limited to, rye, wheat, corn, cane, and mixtures thereof.

The term “carbon substrate” refers to a carbon source capable of being metabolized by the microorganisms and cells disclosed herein. Non-limiting examples of carbon substrates are provided herein and include, but are not limited to, monosaccharides, oligosaccharides, polysaccharides, ethanol, lactate, succinate, glycerol, carbon dioxide, methanol, glucose, fructose, sucrose, xylose, arabinose, dextrose, or mixtures thereof.

The term “effective titer” as used herein, refers to the total amount of a particular alcohol (e.g., butanol) produced by fermentation per liter of fermentation medium.

The term “separation” as used herein is synonymous with “recovery” and refers to removing a chemical compound from an initial mixture to obtain the compound in greater purity or at a higher concentration than the purity or concentration of the compound in the initial mixture.

The term “aqueous phase,” as used herein, refers to the aqueous phase of a biphasic mixture obtained by contacting a fermentation broth with a water-immiscible organic extractant. In an embodiment of a process described herein that includes fermentative extraction, the term “fermentation broth” then specifically refers to the aqueous phase in biphasic fermentative extraction.

The term “organic phase,” as used herein, refers to the non-aqueous phase of a biphasic mixture obtained by contacting a fermentation broth with a water-immiscible organic extractant.

The term “polynucleotide” is intended to encompass a singular nucleic acid as well as plural nucleic acids, and refers to a nucleic acid molecule or construct, e.g., messenger RNA (mRNA) or plasmid DNA (pDNA). A polynucleotide can contain the nucleotide sequence of the full-length cDNA sequence, or a fragment thereof, including the untranslated 5′ and 3′ sequences and the coding sequences. The polynucleotide can be composed of any polyribonucleotide or polydeoxyribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. “Polynucleotide” embraces chemically, enzymatically, or metabolically modified forms.

A polynucleotide sequence can be referred to as “isolated,” in which it has been removed from its native environment. For example, a heterologous polynucleotide encoding a polypeptide or polypeptide fragment having enzymatic activity (e.g., the ability to convert a substrate to xylulose) contained in a vector is considered isolated for the purposes of the present invention. Further examples of an isolated polynucleotide include recombinant polynucleotides maintained in heterologous host cells or purified (partially or substantially) polynucleotides in solution. Isolated polynucleotides or nucleic acids according to the present invention further include such molecules produced synthetically. An isolated polynucleotide fragment in the form of a polymer of DNA can be comprised of one or more segments of cDNA, genomic DNA, or synthetic DNA.

The term “gene” refers to a nucleic acid fragment that is capable of being expressed as a specific protein, optionally including regulatory sequences preceding (5′ non-coding sequences) and following (3′ non-coding sequences) the coding sequence.

As used herein the term “coding region” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence that influence the transcription, RNA processing or stability, or translation of the associated coding sequence. Regulatory sequences can include promoters, translation leader sequences, introns, polyadenylation recognition sequences, RNA processing sites, effector binding sites and stem-loop structures.

As used herein, the term “polypeptide” is intended to encompass a singular “polypeptide” as well as plural “polypeptides” and refers to a molecule composed of monomers (amino acids) linearly linked by amide bonds (also known as peptide bonds). The term “polypeptide” refers to any chain or chains of two or more amino acids, and does not refer to a specific length of the product. Thus, “peptides,” “dipeptides,” “tripeptides,” “oligopeptides,” “protein,” “amino acid chain,” or any other term used to refer to a chain or chains of two or more amino acids, are included within the definition of “polypeptide,” and the term “polypeptide” can be used instead of, or interchangeably with any of these terms. A polypeptide can be derived from a natural biological source or produced by recombinant technology, but is not necessarily translated from a designated nucleic acid sequence. It can be generated in any manner, including by chemical synthesis.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

As used herein, “pyruvate decarboxylase activity” refers to the activity of any polypeptide having a biological function of a pyruvate decarboxylase enzyme, including the examples provided herein. Such polypeptides include a polypeptide that catalyzes the conversion of pyruvate to acetaldehyde. Such polypeptides also include a polypeptide that corresponds to Enzyme Commission Number 4.1.1.1. Such polypeptides can be determined by methods well known in the art and disclosed herein. A polypeptide having pyruvate decarboxylate activity can be, by way of example, PDC1, PDC5, PDC6, or any combination thereof.

As used herein, “acetolactate reductase activity” refers to the activity of any polypeptide having the ability to catalyze the conversion of acetolactate to DHMB. Such polypeptides can be determined by methods well known in the art and disclosed herein.

As used herein, “DHMB” refers to 2,3-dihydroxy-2-methyl butyrate. DHMB includes “fast DHMB,” which has the 2S, 3S configuration, and “slow DHMB,” which has the 2S, 3R configurate. See Kaneko et al., Phytochemistry 39: 115-120 (1995), which is herein incorporated by reference in its entirety and refers to fast DHMB as anglyceric acid and slow DHMB as tiglyceric acid.

As used herein, the term “KARI” is the abbreviation for the enzyme Ketol-acid reductoisomerase. Ketol-acid reductoisomerase catalyzes the reaction of (S)-acetolactate to 2,3-dihydroxyisovalerate. KARI enzymes include enzymes having the EC number, EC 1.1.1.86 (Enzyme Nomenclature 1992, Academic Press, San Diego). These enzymes are available from a number of sources, including, but not limited to E. coli GenBank Accession Number NC-000913 REGION: 3955993.3957468, Vibrio cholerae GenBank Accession Number NC-002505 REGION: 157441.158925, Pseudomonas aeruginosa, GenBank Accession Number NC-002516, REGION: 5272455.5273471, and Pseudomonas fluorescens GenBank Accession Number NC-004129 REGION: 6017379.6018395. KARI enzymes are described for example, in U.S. Published Application Nos. 2008/0261230, 2009/0163376 and 2010/0197519, which are herein incorporated by reference in their entireties.

KARI is found in a variety of organisms and amino acid sequence comparisons across species have revealed that there are 2 types of this enzyme: a short form (class I) found in fungi and most bacteria, and a long form (class II) typical of plants. Class I KARIs typically have between 330-340 amino acid residues. The long form KARI enzymes have about 490 amino acid residues. However, some bacteria such as Escherichia coli possess a long form, where the amino acid sequence differs appreciably from that found in plants. KARI is encoded by the ilvC gene and is an essential enzyme for growth of E. coli and other bacteria in a minimal medium. Class II KARIs generally consist of a 225-residue N-terminal domain and a 287-residue C-terminal domain. The N-terminal domain, which contains the NADPH-binding site, has an αβstructure and resembles domains found in other pyridine nucleotide-dependent oxidoreductases. The C-terminal domain consists almost entirely of α-helices.

As used herein, the term “NADPH consumption assay” refers to an enzyme assay for the determination of the specific activity of the KARI enzyme involving measuring the disappearance of the KARI cofactor, NADPH, from the enzyme reaction. Such assays are described in Aulabaugh and Schloss, Biochemistry 29: 2824-2830, 1990, which is herein incorporated by reference in its entirety.

As used herein, “specific activity” refers to enzyme units/mg protein where an enzyme unit is defined as moles of product formed/minute.

As used herein, “reduced activity” refers to any measurable decrease in a known biological activity of a polypeptide when compared to the same biological activity of the polypeptide prior to the change resulting in the reduced activity. Such a change can include a modification of a polypeptide or a polynucleotide encoding a polypeptide as described herein. A reduced activity of a polypeptide disclosed herein can be determined by methods well known in the art and disclosed herein.

As used herein, “eliminated activity” refers to the complete abolishment of a known biological activity of a polypeptide when compared to the same biological activity of the polypeptide prior to the change resulting in the eliminated activity. Such a change can include a modification of a polypeptide or a polynucleotide encoding a polypeptide as described herein. An eliminated activity includes a biological activity of a polypeptide that is not measurable when compared to the same biological activity of the polypeptide prior to the change resulting in the eliminated activity. An eliminated activity of a polypeptide disclosed herein can be determined by methods well known in the art and disclosed herein.

As used herein, “native” refers to the form of a polynucleotide, gene, or polypeptide as found in nature with its own regulatory sequences, if present.

As used herein, “endogenous” refers to the native form of a polynucleotide, gene or polypeptide in its natural location in the organism or in the genome of an organism. “Endogenous polynucleotide” includes a native polynucleotide in its natural location in the genome of an organism. “Endogenous gene” includes a native gene in its natural location in the genome of an organism. “Endogenous polypeptide” includes a native polypeptide in its natural location in the organism.

As used herein, “heterologous” refers to a polynucleotide, gene, or polypeptide not normally found in the host organism but that is introduced into the host organism. “Heterologous polynucleotide” includes a native coding region, or portion thereof, that is reintroduced into the source organism in a form that is different from the corresponding native polynucleotide. “Heterologous gene” includes a native coding region, or portion thereof, that is reintroduced into the source organism in a form that is different from the corresponding native gene. For example, a heterologous gene can include a native coding region that is a portion of a chimeric gene including non-native regulatory regions that is reintroduced into the native host. “Heterologous polypeptide” includes a native polypeptide that is reintroduced into the source organism in a form that is different from the corresponding native polypeptide.

As used herein, the term “modification” refers to a change in a polynucleotide disclosed herein that results in altered activity of a polypeptide encoded by the polynucleotide, as well as a change in a polypeptide disclosed herein that results in altered activity of the polypeptide. Such changes can be made by methods well known in the art, including, but not limited to, deleting, mutating (e.g., spontaneous mutagenesis, random mutagenesis, mutagenesis caused by mutator genes, or transposon mutagenesis), substituting, inserting, altering the cellular location, altering the state of the polynucleotide or polypeptide (e.g., methylation, phosphorylation or ubiquitination), removing a cofactor, chemical modification, covalent modification, irradiation with UV or X-rays, homologous recombination, mitotic recombination, promoter replacement methods, and/or combinations thereof. Guidance in determining which nucleotides or amino acid residues can be modified, can be found by comparing the sequence of the particular polynucleotide or polypeptide with that of homologous polynucleotides or polypeptides, e.g., yeast or bacterial, and maximizing the number of modifications made in regions of high homology (conserved regions) or consensus sequences.

As used herein, the term “variant” refers to a polypeptide differing from a specifically recited polypeptide of the invention by amino acid insertions, deletions, mutations, and substitutions, created using, e.g., recombinant DNA techniques, such as mutagenesis. Guidance in determining which amino acid residues can be replaced, added, or deleted without abolishing activities of interest, can be found by comparing the sequence of the particular polypeptide with that of homologous polypeptides, e.g., yeast or bacterial, and minimizing the number of amino acid sequence changes made in regions of high homology (conserved regions) or by replacing amino acids with consensus sequences.

Alternatively, recombinant polynucleotide variants encoding these same or similar polypeptides can be synthesized or selected by making use of the “redundancy” in the genetic code. Various codon substitutions, such as silent changes which produce various restriction sites, can be introduced to optimize cloning into a plasmid or viral vector for expression. Mutations in the polynucleotide sequence can be reflected in the polypeptide or domains of other peptides added to the polypeptide to modify the properties of any part of the polypeptide.

Amino acid “substitutions” can be the result of replacing one amino acid with another amino acid having similar structural and/or chemical properties, i.e., conservative amino acid replacements, or they can be the result of replacing one amino acid with an amino acid having different structural and/or chemical properties, i.e., non-conservative amino acid replacements. “Conservative” amino acid substitutions can be made on the basis of similarity in polarity, charge, solubility, hydrophobicity, hydrophilicity, or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Alternatively, “non-conservative” amino acid substitutions can be made by selecting the differences in polarity, charge, solubility, hydrophobicity, hydrophilicity, or the amphipathic nature of any of these amino acids. “Insertions” or “deletions” can be within the range of variation as structurally or functionally tolerated by the recombinant proteins. The variation allowed can be experimentally determined by systematically making insertions, deletions, or substitutions of amino acids in a polypeptide molecule using recombinant DNA techniques and assaying the resulting recombinant variants for activity.

The term “promoter” refers to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters which cause a gene to be expressed in most cell types at most times are commonly referred to as “constitutive promoters.” It is further recognized that since in most cases the exact boundaries of regulatory sequences have not been completely defined, DNA fragments of different lengths can have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “expression,” as used herein, refers to the transcription and stable accumulation of sense (mRNA) or antisense RNA derived from the nucleic acid fragment of the invention. Expression can also refer to translation of mRNA into a polypeptide.

The term “overexpression,” as used herein, refers to an increase in the level of nucleic acid or protein in a host cell. Thus, overexpression can result from increasing the level of transcription or translation of an endogenous sequence in a host cell or can result from the introduction of a heterologous sequence into a host cell. Overexpression can also result from increasing the stability of a nucleic acid or protein sequence.

As used herein the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

The terms “plasmid” and “vector” as used herein, refer to an extra chromosomal element often carrying genes which are not part of the central metabolism of the cell, and usually in the form of circular double-stranded DNA molecules. Such elements can be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein the term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism.

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 1. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

TABLE 1
The Standard Genetic Code

| | T | C | A | G |
|---|---|---|---|---|
| T | TTT Phe (F) | TCT Ser (S) | TAT Tyr (Y) | TGT Cys (C) |
| | TTC Phe (F) | TCC Ser (S) | TAC Tyr (Y) | TGC |
| | TTA Leu (L) | TCA Ser (S) | TAA Stop | TGA Stop |
| | TTG Leu (L) | TCG Ser (S) | TAG Stop | TGG Trp (W) |
| C | CTT Leu (L) | CCT Pro (P) | CAT His (H) | CGT Arg (R) |
| | CTC Leu (L) | CCC Pro (P) | CAC His (H) | CGC Arg (R) |
| | CTA Leu (L) | CCA Pro (P) | CAA Gln (Q) | CGA Arg (R) |
| | CTG Leu (L) | CCG Pro (P) | CAG Gln (Q) | CGG Arg (R) |
| A | ATT Ile (I) | ACT Thr (T) | AAT Asn (N) | AGT Ser (S) |
| | ATC Ile (I) | ACC Thr (T) | AAC Asn (N) | AGC Ser (S) |
| | ATA Ile (I) | ACA Thr (T) | AAA Lys (K) | AGA Arg (R) |
| | ATG Met | ACG Thr (T) | AAG Lys (K) | AGG Arg (R) |
| | (M) | | | |
| G | GTT Val (V) | GCT Ala (A) | GAT Asp (D) | GGT Gly (G) |
| | GTC Val (V) | GCC Ala (A) | GAC Asp (D) | GGC Gly (G) |
| | GTA Val (V) | GCA Ala (A) | GAA Glu (E) | GGA Gly (G) |
| | GTG Val (V) | GCG Ala (A) | GAG Glu (E) | GGG Gly (G) |

Many organisms display a bias for use of particular codons to code for insertion of a particular amino acid in a growing peptide chain. Codon preference, or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage. Codon usage tables are readily available, for example, at the “Codon Usage Database” available at http://www.kazusa.or.jp/codon/ (visited Mar. 20, 2008), and these tables can be adapted in a number of ways. See Nakamura, Y., et al. Nucl. Acids Res. 28:292 (2000). Codon usage tables for yeast, calculated from GenBank Release 128.0 [15 Feb. 2002], are reproduced below as Table 2. This table uses mRNA nomenclature, and so instead of thymine (T) which is found in DNA, the tables use uracil (U) which is found in RNA. Table 2 has been adapted so that frequencies are calculated for each amino acid, rather than for all 64 codons.

TABLE 2
Codon Usage Table for
Saccharomyces cerevisiae Genes

| Amino | | | Frequency per |
|---|---|---|---|
| Acid | Codon | Number | thousand |
| Phe | UUU | 170666 | 26.1 |
| Phe | UUC | 120510 | 18.4 |
| Leu | UUA | 170884 | 26.2 |
| Leu | UUG | 177573 | 27.2 |
| Leu | CUU | 80076 | 12.3 |
| Leu | CUC | 35545 | 5.4 |
| Leu | CUA | 87619 | 13.4 |
| Leu | CUG | 68494 | 10.5 |
| Ile | AUU | 196893 | 30.1 |
| Ile | AUC | 112176 | 17.2 |
| Ile | AUA | 116254 | 17.8 |
| Met | AUG | 136805 | 20.9 |
| Val | GUU | 144243 | 22.1 |
| Val | GUC | 76947 | 11.8 |
| Val | GUA | 76927 | 11.8 |
| Val | GUG | 70337 | 10.8 |
| Ser | UCU | 153557 | 23.5 |
| Ser | UCC | 92923 | 14.2 |
| Ser | UCA | 122028 | 18.7 |
| Ser | UCG | 55951 | 8.6 |
| Ser | AGU | 92466 | 14.2 |
| Ser | AGC | 63726 | 9.8 |
| Pro | CCU | 88263 | 13.5 |
| Pro | CCC | 44309 | 6.8 |
| Pro | CCA | 119641 | 18.3 |
| Pro | CCG | 34597 | 5.3 |
| Thr | ACU | 132522 | 20.3 |
| Thr | ACC | 83207 | 12.7 |
| Thr | ACA | 116084 | 17.8 |
| Thr | ACG | 52045 | 8.0 |
| Ala | GCU | 138358 | 21.2 |
| Ala | GCC | 82357 | 12.6 |
| Ala | GCA | 105910 | 16.2 |
| Ala | GCG | 40358 | 6.2 |
| Tyr | UAU | 122728 | 18.8 |
| Tyr | UAC | 96596 | 14.8 |
| His | CAU | 89007 | 13.6 |
| His | CAC | 50785 | 7.8 |
| Gln | CAA | 178251 | 27.3 |
| Gln | CAG | 79121 | 12.1 |
| Asn | AAU | 233124 | 35.7 |
| Asn | AAC | 162199 | 24.8 |
| Lys | AAA | 273618 | 41.9 |
| Lys | AAG | 201361 | 30.8 |
| Asp | GAU | 245641 | 37.6 |
| Asp | GAC | 132048 | 20.2 |
| Glu | GAA | 297944 | 45.6 |
| Glu | GAG | 125717 | 19.2 |
| Cys | UGU | 52903 | 8.1 |
| Cys | UGC | 31095 | 4.8 |
| Trp | UGG | 67789 | 10.4 |
| Arg | CGU | 41791 | 6.4 |
| Arg | CGC | 16993 | 2.6 |
| Arg | CGA | 19562 | 3.0 |
| Arg | CGG | 11351 | 1.7 |
| Arg | AGA | 139081 | 21.3 |
| Arg | AGG | 60289 | 9.2 |
| Gly | GGU | 156109 | 23.9 |
| Gly | GGC | 63903 | 9.8 |
| Gly | GGA | 71216 | 10.9 |
| Gly | GGG | 39359 | 6.0 |
| Stop | UAA | 6913 | 1.1 |
| Stop | UAG | 3312 | 0.5 |
| Stop | UGA | 4447 | 0.7 |

By utilizing this or similar tables, one of ordinary skill in the art can apply the frequencies to any given polypeptide sequence, and produce a nucleic acid fragment of a codon-optimized coding region which encodes the polypeptide, but which uses codons optimal for a given species.

Randomly assigning codons at an optimized frequency to encode a given polypeptide sequence, can be done manually by calculating codon frequencies for each amino acid, and then assigning the codons to the polypeptide sequence randomly. Additionally, various algorithms and computer software programs are readily available to those of ordinary skill in the art. For example, the “EditSeq” function in the Lasergene Package, available from DNAstar, Inc., Madison, Wis., the backtranslation function in the Vector NTI Suite, available from InforMax, Inc., Bethesda, Md., and the “backtranslate” function in the GCG-Wisconsin Package, available from Accelrys, Inc., San Diego, Calif. In addition, various resources are publicly available to codon-optimize coding region sequences, e.g., the “backtranslation” function at http://www.entelechon.com/bioinformatics/backtranslation.php?lang=eng (visited Apr. 15, 2008) and the “backtranseq” function available at http://bioinfo.pbi.nrc.ca:8090/EMBOSS/index.html (visited Jul. 9, 2002). Constructing a rudimentary algorithm to assign codons based on a given frequency can also easily be accomplished with basic mathematical functions by one of ordinary skill in the art.

Codon-optimized coding regions can be designed by various methods known to those skilled in the art including software packages such as “synthetic gene designer” (http://phenotype.biosci.umbc.edu/codon/sgd/index.php).

A polynucleotide or nucleic acid fragment is “hybridizable” to another nucleic acid fragment, such as a cDNA, genomic DNA, or RNA molecule, when a single-stranded form of the nucleic acid fragment can anneal to the other nucleic acid fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor, N.Y. (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washes with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least about 30 nucleotides. Furthermore, the skilled artisan will recognize that the temperature and wash solution salt concentration can be adjusted as necessary according to factors such as length of the probe.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Altschul, S. F., et al., J. Mol. Biol., 215:403-410 (1993)). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides can be used in sequence-dependent methods of gene identification (e.g., Southern hybridization) and isolation (e.g., in situ hybridization of bacterial colonies or bacteriophage plaques). In addition, short oligonucleotides of 12-15 bases can be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The instant specification teaches the complete amino acid and nucleotide sequence encoding particular proteins. The skilled artisan, having the benefit of the sequences as reported herein, can now use all or a substantial portion of the disclosed sequences for purposes known to those skilled in this art. Accordingly, the instant invention comprises the complete sequences as provided herein, as well as substantial portions of those sequences as defined above.

The term “complementary” is used to describe the relationship between nucleotide bases that are capable of hybridizing to one another. For example, with respect to DNA, adenosine is complementary to thymine and cytosine is complementary to guanine.

The term “percent identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those disclosed in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humania: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991).

Preferred methods to determine identity are designed to give the best match between the sequences tested. Methods to determine identity and similarity are codified in publicly available computer programs. Sequence alignments and percent identity calculations can be performed using the MegAlign™ program of the LASERGENE bioinformatics computing suite (DNASTAR Inc., Madison, Wis.). Multiple alignment of the sequences is performed using the “Clustal method of alignment” which encompasses several varieties of the algorithm including the “Clustal V method of alignment” corresponding to the alignment method labeled Clustal V (disclosed by Higgins and Sharp, CABIOS. 5:151-153 (1989); Higgins, D. G. et al., Comput. Appl. Biosci., 8:189-191 (1992)) and found in the MegAlign™ program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. Additionally the “Clustal W method of alignment” is available and corresponds to the alignment method labeled Clustal W (described by Higgins and Sharp, CABIOS. 5:151-153 (1989); Higgins, D. G. et al., Comput. Appl. Biosci. 8:189-191 (1992)) and found in the MegAlign™ v6.1 program of the LASERGENE bioinformatics computing suite (DNASTAR Inc.). Default parameters for multiple alignment (GAP PENALTY=10, GAP LENGTH PENALTY=0.2, Delay Divergen Seqs(%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

It is well understood by one skilled in the art that many levels of sequence identity are useful in identifying polypeptides, from other species, wherein such polypeptides have the same or similar function or activity. Useful examples of percent identities include, but are not limited to: 55%, 60%, 65%, 70%, 75%, 80%, 85%, 90%, or 95%, or any integer percentage from 55% to 100% can be useful in describing the present invention, such as 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

The term “sequence analysis software” refers to any computer algorithm or software program that is useful for the analysis of nucleotide or amino acid sequences. “Sequence analysis software” can be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol., 215:403-410 (1990)); 3.) DNASTAR (DNASTAR, Inc. Madison, Wis.); 4.) Sequencher (Gene Codes Corporation, Ann Arbor, Mich.); and 5.) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et al., Current Protocols in Molecular Biology, published by Greene Publishing Assoc. and Wiley-Interscience (1987). Additional methods used here are in Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.).

The genetic manipulations of cells disclosed herein can be performed using standard genetic techniques and screening and can be made in any cell that is suitable to genetic manipulation (Methods in Yeast Genetics, 2005, Cold Spring Harbor Laboratory Press, Cold Spring Harbor, N.Y., pp. 201-202). Suitable strains of S. cerevisiae are known in the art and include BY4741 and CEN.PK 113-7D as well as those used for ethanol fermentations, including, but not limited to, those available from LeSaffre, Gert Strand AB, Ferm Solutions, North American Bioproducts, Martrex, and Lallemand, and including, but not limited to Ethanol Red, Prestige Turbo, Ferm Pro, Bio-Ferm XR, Distillers Yeast, FerMax Green, FerMax Gold, Thermosacc, BG-1, PE-2, CAT-1, CBS7959, CBS7960, and CBS7961.

Reduction of DHMB

DHMB can be produced as a result of a side-reaction that occurs when yeast are genetically manipulated to include a biosynthetic pathway, e.g., a biosynthetic pathway that involves the production of acetolactate. The presence of DHMB indicates that not all of the pathway substrates are being converted to the desired product. Thus, yield is lowered. In addition, DHMB present in the fermentation media can have inhibitory effects on product production. For example, DHMB can decrease the activity of enzymes in the biosynthetic pathway or have other inhibitory effects on yeast growth and/or productivity during fermentation. Thus, the methods described herein provide ways of reducing DHMB during fermentation. The methods include both methods of decreasing the production of DHMB and methods of removing DHMB from fermenting compositions.

Decreasing DHMB Production

In some embodiments described herein, a recombinant host cell can comprise reduced or eliminated ability to convert acetolactate to DHMB. The ability of a host cell to convert acetolactate to DHMB can be reduced or eliminated, for example, by a modification or disruption of a polynucleotide or gene encoding a polypeptide having acetolactate reductase activity or a modification or disruption of a polypeptide having acetolactate reductase activity. In other embodiments, the recombinant host cell can comprise a deletion, mutation, and/or substitution in an endogenous polynucleotide or gene encoding a polypeptide having acetolactate reductase activity or in an endogenous polypeptide having acetolactate reductase. Such modifications, disruptions, deletions, mutations, and/or substitutions can result in acetolactate reductase activity that is reduced or eliminated.

In some embodiments, the host cell comprises at least one deletion, mutation, and/or substitution in at least one endogenous polynucleotide encoding a polypeptide having acetolactate reductase activity. In some embodiments, the host cell comprises at least one deletion, mutation, and/or substitution in each of at least two endogenous polynucleotides encoding polypeptides having acetolactate reductase activity.

In some embodiments, a polypeptide having acetolactate reductase activity can catalyze the conversion of acetolactate to DHMB. In some embodiments, a polypeptide having acetolactate reductase activity is capable of catalyzing the reduction of acetolactate to 2S,3S-DHMB (fast DHMB) and/or 2S,3R-DHMB (slow DHMB).

In some embodiments, the conversion of acetolactate to DHMB in a recombinant host cell is reduced or eliminated. In still other embodiments, a polynucleotide, gene or polypeptide having acetolactate reductase activity can correspond to Enzyme Commission Number. In some embodiments, the polypeptide having acetolactate reducatase activity is selected from the group consisting of: YMR226c, YER081W, YIL074C, YBR006W, YPL275W, YOL059W, YIR036c, YPL061W, YPL088W, YCR105W, and YDR541C. In some embodiments, the polypeptide having acetolactate reductase activity is a polypeptide comprising a sequence listed in Table 4 or a sequence that is at least about 70%, at least about 75%, at least about 80%, at least about 85%, at least about 90%, at least about 95%, or at least about 99% identical to a polypeptide sequence listed in Table 4. In some embodiments, the polypeptide having acetolactate reducatase activity is a polypeptide encoded by a polynucleotide sequence listed in Table 4 or a sequence that is at least about 70%, at least about 75%, at least about 80%, at least about 85%, at least about 90%, at least about 95%, or at least about 99% identical to a polynucleotide sequence listed in Table 4.

In some embodiments, a polypeptide having acetolactate reductase activity is YMR226C or a homolog of YMR226C. Thus, in some embodiments, the polypeptide having acetolactate reducatase activity is a polypeptide comprising a sequence listed in Table 6 or a sequence that is at least about 70%, at least about 75%, at least about 80%, at least about 85%, at least about 90%, at least about 95%, or at least about 99% identical to a polypeptide sequence listed in Table 6. In some embodiments, the polypeptide having acetolactate reducatase activity is a polypeptide encoded by a polynucleotide sequence listed in Table 6 or a sequence that is at least about 70%, at least about 75%, at least about 80%, at least about 85%, at least about 90%, at least about 95%, or at least about 99% identical to a polynucleotide sequence listed in Table 6.

Acetolactate reductases capable of converting acetolactate to DHMB can be identified, for example, by screening genetically altered yeast for changes in acetolactate consumption, changes in DHMB production, changes in DHIV production, or changes in other downstream product (e.g., butanol) production.

DHMB can be measured using any technique known to those of skill in the art. For example, DHMB can be separated and quantified by methods known to those of skill in the art and techniques described in the Examples provided herein. For example, DHMB can be separated and quantified using liquid chromatography-mass spectrometry, liquid chromatography-nuclear magnetic resonance (NMR), thin-layer chromatography, and/or HPLC with UV/Vis detection.

Thus, one way of identifying a gene involved in DHMB production comprises measuring the amount of DHMB produced by individual yeast strains in a yeast knock-out library. Knock-out libraries are available, for example, from Open Biosystems® (a division of Thermo Fisher Scientific, Waltham, Mass.). In this method, a decrease in DHMB production indicates that the gene that has been knocked-out functions to increase DHMB production, and an increase in DHMB production indicates that the gene that has been knocked-out functions to decrease DHMB production.

Two ways that a knockout (“KO”) library can be used to identify candidate genes for involvement in DHMB synthesis include: (1) DHMB and DHIV accumulated in the culture during growth from endogenous substrates (acetolactate and NADPH or NADH) can be analyzed in samples from cultures. These samples can be placed in a hot (80-100° C.) water bath for 10-20 min, or diluted into a solution such as 2% formic acid that will kill and permeabilize the cells. After either treatment, small molecules will be found in the supernatant after centrifugation (5 min, 1100×g). The DHMB/DHIV ratio of a control strain (e.g., BY4743) can be compared to that of the different KO derivatives, and the gene(s) missing from any strain(s) with exceptionally low DHMB/DHIV ratios can encode acetolactate reductase (ALR). (2) DHMB and/or DHIV formation rates in vitro from exogenous substrates (acetolactate and NADH and/or NADPH) can be measured in timed samples taken from a suspension of permeabilized cells, and inactivated in either of the ways described above. Since the substrates for DHMB and DHIV synthesis are the same, this allows one to measure the relative levels of ALR and KARI activity in the sample.

Another way of identifying a gene involved in DHMB production comprises measuring the amount of DHMB produced by individual yeast strains in a yeast overexpression library. Overexpression libraries are available, for example, from Open Biosystems® (a division of Thermo Fisher Scientific, Waltham, Mass.). In this method, a decrease in DHMB production indicates that the overexpressed gene functions to decrease DHMB production, and an increase in DHMB production indicates that the overexpressed gene functions to increase DHMB production.

Another way of identifying a gene involved in DHMB production is to biochemically analyze a DHMB-producing yeast strain. For example, DHMB-producing cells can be disrupted. This disruption can be performed at low pH and cold temperatures. The cell lysates can be separated into fractions, e.g., by adding ammonium sulfate or other techniques known to those of skill in the art, and the resulting fractions can be assayed for enzymatic activity. For example, the fractions can be assayed for the ability to convert acetolactate to DHMB. Fractions with enzymatic activity can be treated by methods known in the art to purify and concentrate the enzyme (e.g., dialysis and chromatographic separation). When a sufficient purity and concentration is achieved, the enzyme can be sequenced, and the corresponding gene encoding the acetolactate reductase capable of converting acetolactate to DHMB can be identified.

Furthermore, since the reduction of acetolactate to DHMB occurs in yeast, but does not occur in E. coli, acetolactate reductases that are expressed in yeast, but not expressed in E. coli, can be selected for screening. Selected enzymes can be expressed in yeast or other protein expression systems and screened for the capability to convert acetolactate to DHMB.

Enzymes capable of catalyzing the conversion of acetolactate to DHMB can be screened by assaying for acetolactate levels, by assaying for DHMB levels, by assaying for DHIV levels, or by assaying for any of the downstream products in the conversion of DHIV to butanol, including isobutanol.

In embodiments, selected acetolactate reductase polynucleotides, genes and/or polypeptides disclosed herein can be modified or disrupted. Many methods for genetic modification and disruption of target genes to reduce or eliminate expression are known to one of ordinary skill in the art and can be used to create a recombinant host cell disclosed herein. Modifications that can be used include, but are not limited to, deletion of the entire gene or a portion of the gene encoding an acetolactate reductase protein, inserting a DNA fragment into the encoding gene (in either the promoter or coding region) so that the protein is not expressed or expressed at lower levels, introducing a mutation into the coding region which adds a stop codon or frame shift such that a functional protein is not expressed, and introducing one or more mutations into the coding region to alter amino acids so that a non-functional or a less active protein is expressed. In other embodiments, expression of a target gene can be blocked by expression of an antisense RNA or an interfering RNA, and constructs can be introduced that result in cosuppression. In other embodiments, the synthesis or stability of the transcript can be lessened by mutation. In embodiments, the efficiency by which a protein is translated from mRNA can be modulated by mutation. All of these methods can be readily practiced by one skilled in the art making use of the known or identified sequences encoding target proteins.

In other embodiments, DNA sequences surrounding a target acetolactate reductase coding sequence are also useful in some modification procedures and are available, for example, for yeasts such as Saccharomyces cerevisiae in the complete genome sequence coordinated by Genome Project ID9518 of Genome Projects coordinated by NCBI (National Center for Biotechnology Information) with identifying GOPID #13838. An additional non-limiting example of yeast genomic sequences is that of Candida albicans, which is included in GPID #10771, #10701 and #16373. Other yeast genomic sequences can be readily found by one of skill in the art in publicly available databases.

In other embodiments, DNA sequences surrounding a target acetolactate reductase coding sequence can be useful for modification methods using homologous recombination. In a non-limiting example of this method, acetolactate reductase gene flanking sequences can be placed bounding a selectable marker gene to mediate homologous recombination whereby the marker gene replaces the acetolactate reductase gene. In another non-limiting example, partial acetolactate reductase gene sequences and acetolactate reductase gene flanking sequences bounding a selectable marker gene can be used to mediate homologous recombination whereby the marker gene replaces a portion of the target acetolactate reductase gene. In embodiments, the selectable marker can be bounded by site-specific recombination sites, so that following expression of the corresponding site-specific recombinase, the resistance gene is excised from the acetolactate reductase gene without reactivating the latter. In embodiments, the site-specific recombination leaves behind a recombination site which disrupts expression of the acetolactate reductase protein. In other embodiments, the homologous recombination vector can be constructed to also leave a deletion in the acetolactate reductase gene following excision of the selectable marker, as is well known to one skilled in the art.

In other embodiments, deletions can be made to an acetolactate reductase target gene using mitotic recombination as described by Wach et al. (Yeast, 10:1793-1808; 1994). Such a method can involve preparing a DNA fragment that contains a selectable marker between genomic regions that can be as short as 20 bp, and which bound a target DNA sequence. In other embodiments, this DNA fragment can be prepared by PCR amplification of the selectable marker gene using as primers oligonucleotides that hybridize to the ends of the marker gene and that include the genomic regions that can recombine with the yeast genome. In embodiments, the linear DNA fragment can be efficiently transformed into yeast and recombined into the genome resulting in gene replacement including with deletion of the target DNA sequence (as disclosed, for example, in Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.)).

Moreover, promoter replacement methods can be used to exchange the endogenous transcriptional control elements allowing another means to modulate expression such as described by Mnaimneh et al., ((2004) Cell 118(1):31-44).

In other embodiments, the acetolactate reductase target gene encoded activity can be disrupted using random mutagenesis, which can then be followed by screening to identify strains with reduced or eliminated activity. In this type of method, the DNA sequence of the target gene encoding region, or any other region of the genome affecting carbon substrate dependency for growth, need not be known. In embodiments, a screen for cells with reduced acetolactate reductase activity, or other mutants having reduced acetolactate reductase activity, can be useful for recombinant host cells of the invention.

Methods for creating genetic mutations are common and well known in the art and can be applied to the exercise of creating mutants. Commonly used random genetic modification methods (reviewed in Methods in Yeast Genetics, 2005, Cold Spring Harbor Laboratory Press, Cold Spring Harbor, N.Y.) include spontaneous mutagenesis, mutagenesis caused by mutator genes, chemical mutagenesis, irradiation with UV or X-rays, or transposon mutagenesis.

Chemical mutagenesis of host cells can involve, but is not limited to, treatment with one of the following DNA mutagens: ethyl methanesulfonate (EMS), nitrous acid, diethyl sulfate, or N-methyl-N′-nitro-N-nitroso-guanidine (MNNG). Such methods of mutagenesis have been reviewed in Spencer et al. (Mutagenesis in Yeast, 1996, Yeast Protocols: Methods in Cell and Molecular Biology. Humana Press, Totowa, N.J.). In embodiments, chemical mutagenesis with EMS can be performed as disclosed in Methods in Yeast Genetics, 2005, Cold Spring Harbor Laboratory Press, Cold Spring Harbor, N.Y. Irradiation with ultraviolet (UV) light or X-rays can also be used to produce random mutagenesis in yeast cells. The primary effect of mutagenesis by UV irradiation is the formation of pyrimidine dimers which disrupt the fidelity of DNA replication. Protocols for UV-mutagenesis of yeast can be found in Spencer et al. (Mutagenesis in Yeast, 1996, Yeast Protocols Methods in Cell and Molecular Biology. Humana Press, Totowa, N.J.). In embodiments, the introduction of a mutator phenotype can also be used to generate random chromosomal mutations in host cells. In embodiments, common mutator phenotypes can be obtained through disruption of one or more of the following genes: PMS1, MAG1, RAD18 or RAD51. In other embodiments, restoration of the non-mutator phenotype can be obtained by insertion of the wildtype allele. In other embodiments, collections of modified cells produced from any of these or other known random mutagenesis processes may be screened for reduced or eliminated acetolactate reductase activity.

Genomes have been completely sequenced and annotated and are publicly available for the following yeast strains: Ashbya gossypii ATCC 10895, Candida glabrata CBS 138, Kluyveromyces lactis NRRL Y-1140, Pichia stipitis CBS 6054, Saccharomyces cerevisiae S288c, Schizosaccharomyces pombe 972h-, and Yarrowia lipolytica CLIB122. Typically BLAST (described above) searching of publicly available databases with known acetolactate reductase polynucleotide or polypeptide sequences, such as those provided herein, is used to identify acetolactate reductase-encoding sequences of other host cells, such as yeast cells.

The modification of acetolactate reductase in a recombinant host cell disclosed herein to reduce or eliminate acetolactate reductase activity can be confirmed using methods known in the art. For example, the presence or absence of an acetolactate reductase-encoding polynucleotide sequence can be determined using PCR screening. A decrease in acetolactate reductase activity can also be determined based on a reduction in conversion of acetolactate to DHMB. A decrease in acetolactate reductase activity can also be determined based on a reduction in DHMB production. A decrease in acetolactate reductase activity can also be determined based on an increase in butanol production.

Thus, in some embodiments, a yeast that is capable of producing butanol produces no more than about 5 mM, about 4 mM, about 3 mM, about 2 mM, about 1 mM, about 0.9 mM, about 0.8 mM., about 0.7 mM, about 0.6 mM, about 0.5 mM, about 0.4 mM or about 0.3 mM DHMB. In some embodiments, a yeast producing butanol produces no more than about 5 mM, about 4 mM, about 3 mM, about 2 mM, about 1 mM, about 0.9 mM, about 0.8 mM., about 0.7 mM, about 0.6 mM, about 0.5 mM, about 0.4 mM or about 0.3 mM DHMB. In some embodiments, a yeast producing butanol produces no more than about 0.2 mM or 0.2 mM DHMB.

In some embodiments, a yeast capable of producing butanol produces no more than about 10 mM DHMB when cultured under fermentation conditions for at least about 50 hours. In some embodiments, a yeast capable of producing butanol produces no more than about 5 mM DHMB when cultured under fermentation conditions for at least about 20 hours, at least about 25 hours, at least about 30 hours, at least about 35 hours, at least about 40 hours, at least about 45 hours, or at least about 50 hours. In some embodiments, a yeast capable of producing butanol produced no more than about 3 mM DHMB when cultured under fermentation conditions for at least about 5 hours, at least about 10 hours, at least about 15 hours, at least about 20 hours, at least about 25 hours, at least about 30 hours, at least about 35 hours, at least about 40 hours, at least about 45 hours, or at least about 50 hours. In some embodiments, a yeast capable of producing butanol produced no more than about 1 mM DHMB when cultured under fermentation conditions for at least about 1 hour, at least about 5 hours, at least about 10 hours, at least about 15 hours, at least about 20 hours, at least about 25 hours, at least about 30 hours, at least about 35 hours, at least about 40 hours, at least about 45 hours, or at least about 50 hours. In some embodiments, a yeast capable of producing butanol produced no more than about 0.5 mM DHMB when cultured under fermentation conditions for at least about 1 hour, at least about 5 hours, at least about 10 hours, at least about 15 hours, at least about 20 hours, at least about 25 hours, at least about 30 hours, at least about 35 hours, at least about 40 hours, at least about 45 hours, or at least about 50 hours.

In some embodiments, a yeast comprising at least one deletion, mutation, and/or substitution in an endogenous polynucleotide encoding an acetolactate reductase produces no more than about 0.5 times, about 0.4 times, about 0.3 times, about 0.2 times, about 0.1 times, about 0.05 times the amount of DHMB produced by a yeast containing the endogenous polynucleotide encoding an acelotacatate reductase when cultured under fermentation conditions for the same amount of time.

In some embodiments, a yeast that is capable of producing butanol produces an amount of DHIV that is at least about 5 mM, at least about 6 mM, at least about 7 mM, at least about 8 mM, at least about 9 mM, or at least about 10 mM.

In some embodiments, a yeast that is capable of producing butanol produces an amount of DHIV that is at least about the amount of DHMB produced. In some embodiments, a yeast that is capable of producing butanol produces an amount of DHIV that is at least about twice, about three times, about five times, about ten times, about 15 times, about 20 times, about 25 times, about 30 times, about 35 times, about 40 times, about 45 times, or about 50 times the amount of DHMB produced.

In some embodiments, a yeast that is capable of producing butanol produces DHIV at a rate that is at least about equal to the rate of DHMB production. In some embodiments, a yeast that is capable of producing butanol produces DHIV at a rate that is at least about twice, about three times, about five times, about ten times, about 15 times, about 20 times, about 25 times, about 30 times, about 35 times, about 40 times, about 45 times, or about 50 times the rate of DHMB production.

In some embodiments, a yeast that is capable of producing butanol produces less than 0.010 moles of DHMB per mole of glucose consumed. In some embodiments, a yeast produces less than about 0.009, less than about 0.008, less than about 0.007, less than about 0.006, or less than about 0.005 moles of DHMB per mole of glucose consumed. In some embodiments, a yeast produces less than about 0.004, less than about 0.003, less than about 0.002, or less than about 0.001 moles of DHMB per mole of glucose consumed.

In some embodiments, acetolactate reductase activity is inhibited by chemical means. For example, acetolactate reductase could be inhibited using other known substrates such as those listed in Fujisawa et al. including L-serine, D-serine, 2-methyl-DL-serine, D-threonine, L-allo-threonine, L-3-hydroxyisobutyrate, D-3-hydroxyisobutyrate, 3-hydroxypropionate, L-3-hydroxybutyrate, and D-3-hydroxybutyrate. Biochimica et Biophysica Acta 1645:89-94 (2003), which is herein incorporated by reference in its entirety.

DHMB Removal

In other embodiments described herein, a reduction in DHMB can be achieved by removing DHMB from a fermentation. Thus, fermentations with reduced DHMB concentrations are also described herein. Removal of DHMB can result in a product of greater purity. Therefore, compositions comprising products of biosynthetic pathways such as ethanol or butanol with increased purity are also provided.

DHMB can be removed during or after a fermentation process and can be removed by any means known in the art. DHMB can be removed, for example, by extraction into an organic phase or reactive extraction.

In some embodiments, the fermentation broth comprises less than about 0.5 mM DHMB. In some embodiments, the fermentation broth comprises less than about 1.0 mM DHMB after about 5 hours, about 10 hours, about 15 hours, about 20 hours, about 25 hours, about 30 hours, about 35 hours, about 40 hours, about 45 hours, or about 50 hours of fermentation. In some embodiments, the fermentation broth comprises less than about 5.0 mM DHMB after about 20 hours, about 25 hours, about 30 hours, about 35 hours, about 40 hours, about 45 hours, or about 50 hours of fermentation.

Host Cells

In some embodiments, the recombinant host cell comprises a biosynthetic pathway. The biosynthetic pathway can be a pathway that is capable of converting pyruvate to acetolactate. In some embodiments, a host cell comprising a biosynthetic pathway capable of converting pyurvate to acetolacatate comprises a polynucleotide encoding a polypeptide having acetolactate synthase activity. For example, the biosynthetic pathway can be a butanol producing pathway or a butanediol producing pathway. The biosynthetic pathway can also be a branched-chain amino acid (e.g., leucine, isoleucine, valine) producing pathway.

In some embodiments, the recombinant host cell can comprise a butanol biosynthetic pathway as described further herein. In some embodiments, the butanol biosynthetic pathway is an isobutanol biosynthetic pathway. Production of isobutanol in a recombinant host cell disclosed herein benefits from a reduction, substantial elimination or elimination of an acetolactate reductase activity.

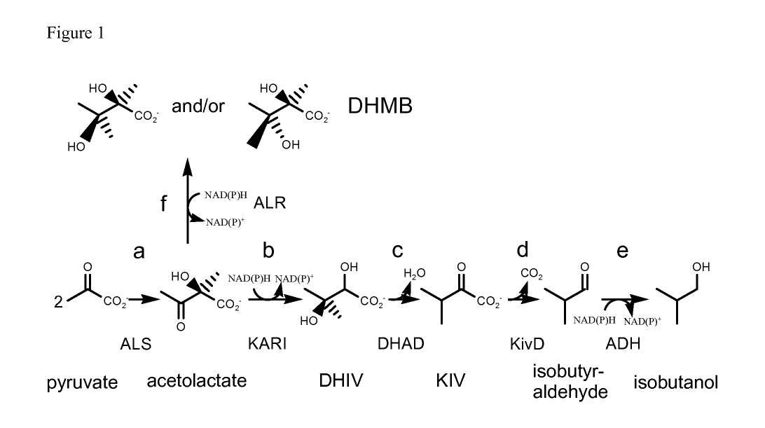

Isobutanol biosynthetic pathways are disclosed in U.S. Patent Application Publication No. US 2007/0092957, which is incorporated by reference herein. A diagram of an isobutanol biosynthetic pathways is provided in FIG. 1 therein. Steps in an isobutanol biosynthetic pathway can include conversion of:
pyruvate to acetolactate (see FIG. 1, pathway step a therein), as catalyzed for example by acetolactate synthase;
acetolactate to 2,3-dihydroxyisovalerate (see FIG. 1, pathway step b therein) as catalyzed for example by acetohydroxy acid isomeroreductase;
2,3-dihydroxyisovalerate to 2-ketoisovalerate (see FIG. 1, pathway step c therein) as catalyzed for example by acetohydroxy acid dehydratase, also called dihydroxy-acid dehydratase (DHAD);
2-ketoisovalerate to isobutyraldehyde (see FIG. 1, pathway step d therein) as catalyzed for example by branched-chain 2-keto acid decarboxylase; and
isobutyraldehyde to isobutanol (see FIG. 1, pathway step e therein) as catalyzed for example by branched-chain alcohol dehydrogenase.

The substrate to product conversions, and enzymes involved in these reactions are described in U.S. Patent Application Publication No. US 2007/0092957, which is incorporated by reference herein.

Genes and polypeptides that can be used for the substrate to product conversions described above as well as those for additional isobutanol pathways, are described in U.S. Patent Appl. Pub. No. 20070092957 and PCT Pub. No. WO 2011/019894. US Appl. Pub. Nos. 2011/019894, 2007/0092957, and 2010/0081154, describe dihydroxyacid dehydratases. Ketol-acid reductoisomerase (KARI) enzymes are described in U.S. Patent Appl. Pub. Nos. 2008/0261230, 2009/0163376, 2010/0197519, 2010/0143997, U.S. application Ser. No. 12/893,077. Examples of KARIs disclosed therein are those from Vibrio cholerae, Pseudomonas aeruginosa PAO1, and Pseudomonas fluorescens PFS. SEQ ID NOs: 259 (“K9G9”) and 258 (“K9D3”) and 257 (“K9”) are examples of suitable polypeptides for catalyzing the substrate to product conversion acetolactate to 2,3-dihydroxyisovalerate. Suitable polypeptides to catalyze the substrate to product conversion acetolactate to 2,3-dihydroxyisovalerate include those that that have a K_{M} for NADH less than about 300 μM, less than about 100 μM, less than about 50 μM, less than about 20 μM or less than about 10 μM. U.S. Patent Appl. Publ. No. 2009/0269823 and U.S. Prov. Patent Appl. No. 61/290,636, describe alcohol dehydrogenases. Suitable alcohol dehydrogenases include SadB from Achromobacter xylosoxidans. Additional alcohol dehydrogenases include horse liver ADH and Beijerinkia indica ADH, and those that utilize NADH as a cofactor. In one embodiment a butanol biosynthetic pathway comprises a) a ketol-acid reductoisomerase that has a K_{M} for NADH less than about 300 μM, less than about 100 μM, less than about 50 μM, less than about 20 μM or less than about 10 μM; b) an alcohol dehydrogenase that utilizes NADH as a cofactor; or c) both a) and b).

Additional genes that can be used can be identified by one skilled in the art through bioinformatics or using methods well-known in the art.

Additionally described in U.S. Patent Application Publication No. US 2007/0092957 A1, which is incorporated by reference herein, are construction of chimeric genes and genetic engineering of bacteria and yeast for isobutanol production using the disclosed biosynthetic pathways.

In some embodiments, the isobutanol biosynthetic pathway can comprise a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway can comprise polynucleotides encoding polypeptides having acetolactate synthase, keto acid reductoisomerase, dihydroxy acid dehydratase, ketoisovalerate decarboxylase, and alcohol dehydrogenase activity.

In addition, in some embodiments, the microorganism comprises a functional deletion of a hexokinase 2 gene. Deletion of hexokinase 2 has been used to reduce glucose repression and to increase the availability of pyruvate for utilization in biosynthetic pathways. For example, International Publication No. WO 2000/061722 A1, which is herein incorporated by reference in its entirety, discloses the production of yeast biomass by aerobically growing yeast having one or more functionally deleted hexokinase 2 genes or analogs.

In addition, in some embodiments, the microorganism comprises at least one deletion, mutation, and/or substitution in a polynucleotide encoding a polypeptide having pyruvate decarboxylase activity. The polypeptide having pyruvate decarboxylate activity can be, by way of example, PDC1, PDC5, PDC6, or any combination thereof. In some embodiments, the recombinant host cell has reduced or eliminated pyruvate decarboxylase activity. In some embodiments, the microorganism is free of an enzyme having pyruvate decarboxylase activity. In some embodiments, the microorganism is a PDC knockout. Examples of host cells comprising reduced pyruvate decarboxylase activity are described in U.S. Patent Application Publication No. 2009/0305363, which is herein incorporated by reference in its entirety. U.S. Patent Application Publication Nos. 2007/0031950 and 2005/0059136, each of which is herein incorporated by reference in its entirety, also disclose host cells with decrease pyruvate decarboxylase activity.

In some embodiments, the recombinant host cell comprises a recombinant ketol-acid reductoisomerase enzyme (KARI) enzyme. Highly active KARI enzymes are disclosed, for example, in U.S. Patent Application Publication No. 2008/0261230, which is incorporated by reference herein. Examples of high activity KARIs disclosed therein are those from Vibrio cholerae, Pseudomonas aeruginosa PAO1, and Pseudomonas fluorescens PFS. In some embodiments, the KARI enzyme has a specific activity of at least about 0.1 micromoles/min/mg, at least about 0.2 micromoles/min/mg, at least about 0.3 micromoles/min/mg, at least about 0.4 micromoles/min/mg, at least about 0.5 micromoles/min/mg, at least about 0.6 micromoles/min/mg, at least about 0.7 micromoles/min/mg, at least about 0.8 micromoles/min/mg, at least about 0.9 micromoles/min/mg, at least about 1.0 micromoles/min/mg, or at least about 1.1 micromoles/min/mg.

In some embodiments, the KARI utilizes NADPH. Methods of measuring NADPH consumption are known in the art. For example, US Published Application No. 2008/0261230, which is herein incorporated by reference in its entirety, provides methods of measuring NADPH consumption. In some embodiments, an NADPH consumption assay is a method that measures the disappearance of the cofactor, NADPH, during the enzymatic conversion of acetolactate to α-β-dihydroxy-isovalerate at 340 nm. The activity is calculated using the molar extinction coefficient of 6220 M⁻¹ cm⁻¹ for NADPH and is reported as μmole of NADPH consumed per min per mg of total protein in cell extracts (see Aulabaugh and Schloss, Biochemistry 29: 2824-2830, 1990). In some embodiments, the NADPH consumption assay is run under the following conditions: i) pH of about 7.5; ii) a temperature of about 22.5° C.; and iii) greater than about 10 mM potassium.

In some embodiments, the KARI is capable of utilizing NADH. In some embodiments, the KARI is capable of utilizing NADH under anaerobic conditions. KARI enzymes using NADH are described, for example, in U.S. Patent Application Publication No. 2009/0163376, which is herein incorporated by reference in its entirety.

In some embodiments, the recombinant host cell comprises increased dihydroxy-acid dehydratase (DHAD) activity compared to a wildtype. Methods of increasing DHAD activity are described, for example, in U.S. Patent Application Publication No. 2010/0081173 and U.S. patent application Ser. No. 13/029,558, filed Feb. 17, 2011, which are herein incorporated by reference in their entireties.

In some embodiments, the recombinant host cell comprises the alcohol dehydrogenase (ADH) sadB from Achromobacter xylosoxidans. Host cells comprising sadB are described, for example, in U.S. Patent Application Publication No. 2009/0269823, which is herein incorporated by reference in its entirety. In some embodiments, the recombinant host cell can comprise a biosynthetic pathway comprising the step of converting pyruvate to acetolactate. In some embodiments, the biosynthetic pathway is a butanediol (BDO) production pathway. BDO biosynthetic pathways are described, for example, in U.S. Patent Application Publication No. 2009/0305363, which is herein incorporated by reference in its entirety.

According to the methods described herein, any yeast containing a biosynthetic pathway involving the production of acetolactate as an intermediate can be cultured to produce a product. In some embodiments, the yeast cell is a member of a genus selected from the group consisting of: Saccharomyces, Schizosaccharomyces, Hansenula, Candida, Kluyveromyces, Yarrowia, Issatchenkia, and Pichia. In some embodiments, the yeast cell is Yarrowia lipolytica, Kluvyeromyces marxianus, or Saccharomyces cerevisiae. In still another aspect, the yeast cell is Saccharomyces cerevisiae.

Isobutanol and Other Products

In embodiments of the invention, methods for the production of a product of a biosynthetic pathway are provided which comprise (a) providing a recombinant host cell disclosed herein; and (b) growing the host cell under conditions whereby the product of the biosynthetic pathway is produced. In other embodiments, the product is produced as a co-product along with ethanol. In still other embodiments, the product of the biosynthetic pathway is butanol or isobutanol. In still other embodiments, the product of the biosynthetic pathway is butanediol (BDO).

In other embodiments of the invention, the product of the biosynthetic pathway is produced at a greater yield or amount compared to the production of the same product in a recombinant host cell that does not comprise reduced or eliminated ability to convert acetolactate to DHMB. In embodiments, this greater yield includes production at a yield of greater than about 10% of theoretical, at a yield of greater than about 20% of theoretical, at a yield of greater than about 25% of theoretical, at a yield of greater than about 30% of theoretical, at a yield of greater than about 40% of theoretical, at a yield of greater than about 50% of theoretical, at a yield of greater than about 60% of theoretical, at a yield of greater than about 70% of theoretical, at a yield of greater than about 75% of theoretical, at a yield of greater than about 80% of theoretical at a yield of greater than about 85% of theoretical, at a yield of greater than about 90% of theoretical, at a yield of greater than about 95% of theoretical, at a yield of greater than about 96% of theoretical, at a yield of greater than about 97% of theoretical, at a yield of greater than about 98% of theoretical, at a yield of greater than about 99% of theoretical, or at a yield of about 100% of theoretical. In some embodiments, the theoretical yield is the product yield of a recombinant host cell that does not comprise a reduced or eliminated ability to convert acetolactate to DHMB and that comprises a biosynthetic pathway for the product.

Thus, the product can be a composition comprising butanol that is substantially free of, or free of DHMB. In some embodiments, the composition comprising butanol contains no more than about 5 mM, about 4 mM, about 3 mM, about 2 mM, about 1 mM, about 0.5 mM, about 0.4 mM, about 0.3 mM DHMB, or about 0.2 mM DHMB.

The product can also be a composition comprising BDO that is substantially free of, or free of DHMB. In some embodiments, the composition comprising BDO contains no more than about 5 mM, about 4 mM, about 3 mM, about 2 mM, about 1 mM, about 0.5 mM, about 0.4 mM, about 0.3 mM DHMB, or about 0.2 mM DHMB.

Any product of a biosynthetic pathway that involves the conversion of acetolactate to a substrate other than DHMB can be produced with greater effectiveness in a recombinant host cell disclosed herein having the described modification of acetolactate reductase activity. Such products include, but are not limited to, butanol, e.g., isobutanol, 2-butanol, and BDO, and branched chain amino acids.

Growth for Production

Recombinant host cells disclosed herein are grown in fermentation media which contains suitable carbon substrates. Additional carbon substrates may include, but are not limited to, monosaccharides such as fructose, oligosaccharides such as lactose, maltose, galactose, or sucrose, polysaccharides such as starch or cellulose or mixtures thereof and unpurified mixtures from renewable feedstocks such as cheese whey permeate, cornsteep liquor, sugar beet molasses, and barley malt. Other carbon substrates may include ethanol, lactate, succinate, or glycerol.

Additionally the carbon substrate may also be one-carbon substrates such as carbon dioxide, or methanol for which metabolic conversion into key biochemical intermediates has been demonstrated. In addition to one and two carbon substrates, methylotrophic organisms are also known to utilize a number of other carbon containing compounds such as methylamine, glucosamine and a variety of amino acids for metabolic activity. For example, methylotrophic yeasts are known to utilize the carbon from methylamine to form trehalose or glycerol (Hellion et al., Microb. Growth C1-Compd., [Int. Symp.], 7th (1993), 415-32, Editor(s): Murrell, J. Collin; Kelly, Don P. Publisher: Intercept, Andover, UK). Similarly, various species of Candida will metabolize alanine or oleic acid (Sulter et al., Arch. Microbial. 153:485-489 (1990)). Hence it is contemplated that the source of carbon utilized in the present invention may encompass a wide variety of carbon containing substrates and will only be limited by the choice of organism.

Although it is contemplated that all of the above mentioned carbon substrates and mixtures thereof are suitable in the present invention, in some embodiments, the carbon substrates are glucose, fructose, and sucrose, or mixtures of these with C5 sugars such as xylose and/or arabinose for yeasts cells modified to use C5 sugars. Sucrose may be derived from renewable sugar sources such as sugar cane, sugar beets, cassava, sweet sorghum, and mixtures thereof. Glucose and dextrose may be derived from renewable grain sources through saccharification of starch based feedstocks including grains such as corn, wheat, rye, barley, oats, and mixtures thereof. In addition, fermentable sugars may be derived from renewable cellulosic or lignocellulosic biomass through processes of pretreatment and saccharification, as described, for example, in U.S. Patent Application Publication No. 2007/0031918 A1, which is herein incorporated by reference. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof.

In addition to an appropriate carbon source, fermentation media must contain suitable minerals, salts, cofactors, buffers and other components, known to those skilled in the art, suitable for the growth of the cultures and promotion of an enzymatic pathway described herein.

Culture Conditions

Typically cells are grown at a temperature in the range of about 20° C. to about 40° C. in an appropriate medium. Suitable growth media in the present invention are common commercially prepared media such as Luria Bertani (LB) broth, Sabouraud Dextrose (SD) broth, Yeast Medium (YM) broth, or broth that includes yeast nitrogen base, ammonium sulfate, and dextrose (as the carbon/energy source) or YPD Medium, a blend of peptone, yeast extract, and dextrose in optimal proportions for growing most Saccharomyces cerevisiae strains. Other defined or synthetic growth media may also be used, and the appropriate medium for growth of the particular microorganism will be known by one skilled in the art of microbiology or fermentation science. The use of agents known to modulate catabolite repression directly or indirectly, e.g., cyclic adenosine 2′:3′-monophosphate, may also be incorporated into the fermentation medium.

Suitable pH ranges for the fermentation are between about pH 5.0 to about pH 9.0. In one embodiment, about pH 6.0 to about pH 8.0 is used for the initial condition. Suitable pH ranges for the fermentation of yeast are typically between about pH 3.0 to about pH 9.0. In one embodiment, about pH 5.0 to about pH 8.0 is used for the initial condition. Suitable pH ranges for the fermentation of other microorganisms are between about pH 3.0 to about pH 7.5. In one embodiment, about pH 4.5 to about pH 6.5 is used for the initial condition.

Fermentations may be performed under aerobic or anaerobic conditions. In one embodiment, anaerobic or microaerobic conditions are used for fermentations.

Industrial Batch and Continuous Fermentations

Isobutanol, or other products, may be produced using a batch method of fermentation. A classical batch fermentation is a closed system where the composition of the medium is set at the beginning of the fermentation and not subject to artificial alterations during the fermentation. A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the media. Batch and fed-batch fermentations are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992), herein incorporated by reference.

Isobutanol, or other products, may also be produced using continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth. Continuous fermentation allows for the modulation of one factor or any number of factors that affect cell growth or end product concentration. Methods of modulating nutrients and growth factors for continuous fermentation processes as well as techniques for maximizing the rate of product formation are well known in the art of industrial microbiology and a variety of methods are detailed by Brock, supra.

It is contemplated that the production of isobutanol, or other products, may be practiced using batch, fed-batch or continuous processes and that any known mode of fermentation would be suitable. Additionally, it is contemplated that cells may be immobilized on a substrate as whole cell catalysts and subjected to fermentation conditions for isobutanol production.

### Methods for Isobutanol Isolation from the Fermentation Medium

Bioproduced isobutanol may be isolated from the fermentation medium using methods known in the art for ABE fermentations (see, e.g., Durre, Appl. Microbial. Biotechnol. 49:639-648 (1998), Groot et al., Process. Biochem. 27:61-75 (1992), and references therein). For example, solids may be removed from the fermentation medium by centrifugation, filtration, decantation, or the like. Then, the isobutanol may be isolated from the fermentation medium using methods such as distillation, azeotropic distillation, liquid-liquid extraction, adsorption, gas stripping, membrane evaporation, or pervaporation.

Because isobutanol forms a low boiling point, azeotropic mixture with water, distillation can be used to separate the mixture up to its azeotropic composition. Distillation may be used in combination with another separation method to obtain separation around the azeotrope. Methods that may be used in combination with distillation to isolate and purify butanol include, but are not limited to, decantation, liquid-liquid extraction, adsorption, and membrane-based techniques. Additionally, butanol may be isolated using azeotropic distillation using an entrainer (see, e.g., Doherty and Malone, Conceptual Design of Distillation Systems, McGraw Hill, New York, 2001).

The butanol-water mixture forms a heterogeneous azeotrope so that distillation may be used in combination with decantation to isolate and purify the isobutanol. In this method, the isobutanol containing fermentation broth is distilled to near the azeotropic composition. Then, the azeotropic mixture is condensed, and the isobutanol is separated from the fermentation medium by decantation. The decanted aqueous phase may be returned to the first distillation column as reflux. The isobutanol-rich decanted organic phase may be further purified by distillation in a second distillation column.

The isobutanol can also be isolated from the fermentation medium using liquid-liquid extraction in combination with distillation. In this method, the isobutanol is extracted from the fermentation broth using liquid-liquid extraction with a suitable solvent. The isobutanol-containing organic phase is then distilled to separate the butanol from the solvent.

Distillation in combination with adsorption can also be used to isolate isobutanol from the fermentation medium. In this method, the fermentation broth containing the isobutanol is distilled to near the azeotropic composition and then the remaining water is removed by use of an adsorbent, such as molecular sieves (Aden et al., Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation may be used to isolate and purify the isobutanol from the fermentation medium. In this method, the fermentation broth containing the isobutanol is distilled to near the azeotropic composition, and then the remaining water is removed by pervaporation through a hydrophilic membrane (Guo et al., J. Membr. Sci. 245, 199-210 (2004)).

In situ product removal (ISPR) (also referred to as extractive fermentation) can be used to remove butanol (or other fermentative alcohol) from the fermentation vessel as it is produced, thereby allowing the microorganism to produce butanol at high yields. One method for ISPR for removing fermentative alcohol that has been described in the art is liquid-liquid extraction. In general, with regard to butanol fermentation, for example, the fermentation medium, which includes the microorganism, is contacted with an organic extractant at a time before the butanol concentration reaches a toxic level. The organic extractant and the fermentation medium form a biphasic mixture. The butanol partitions into the organic extractant phase, decreasing the concentration in the aqueous phase containing the microorganism, thereby limiting the exposure of the microorganism to the inhibitory butanol.

Liquid-liquid extraction can be performed, for example, according to the processes described in U.S. Patent Appl. Pub. No. 2009/0305370, the disclosure of which is hereby incorporated in its entirety. U.S. Patent Appl. Pub. No. 2009/0305370 describes methods for producing and recovering butanol from a fermentation broth using liquid-liquid extraction, the methods comprising the step of contacting the fermentation broth with a water immiscible extractant to form a two-phase mixture comprising an aqueous phase and an organic phase. Typically, the extractant can be an organic extractant selected from the group consisting of saturated, mono-unsaturated, poly-unsaturated (and mixtures thereof) C₁₂ to C₂₂ fatty alcohols, C₁₂ to C₂₂ fatty acids, esters of C₁₂ to C₂₂ fatty acids, C₁₂ to C₂₂ fatty aldehydes, and mixtures thereof. The extractant(s) for ISPR can be non-alcohol extractants. The ISPR extractant can be an exogenous organic extractant such as oleyl alcohol, behenyl alcohol, cetyl alcohol, lauryl alcohol, myristyl alcohol, stearyl alcohol, 1-undecanol, oleic acid, lauric acid, myristic acid, stearic acid, methyl myristate, methyl oleate, undecanal, lauric aldehyde, 20-methylundecanal, and mixtures thereof.

In some embodiments, an alcohol ester can be formed by contacting the alcohol in a fermentation medium with an organic acid (e.g., fatty acids) and a catalyst capable of esterfiying the alcohol with the organic acid. In such embodiments, the organic acid can serve as an ISPR extractant into which the alcohol esters partition. The organic acid can be supplied to the fermentation vessel and/or derived from the biomass supplying fermentable carbon fed to the fermentation vessel. Lipids present in the feedstock can be catalytically hydrolyzed to organic acid, and the same catalyst (e.g., enzymes) can esterify the organic acid with the alcohol. The catalyst can be supplied to the feedstock prior to fermentation, or can be supplied to the fermentation vessel before or contemporaneously with the supplying of the feedstock. When the catalyst is supplied to the fermentation vessel, alcohol esters can be obtained by hydrolysis of the lipids into organic acid and substantially simultaneous esterification of the organic acid with butanol present in the fermentation vessel. Organic acid and/or native oil not derived from the feedstock can also be fed to the fermentation vessel, with the native oil being hydrolyzed into organic acid. Any organic acid not esterified with the alcohol can serve as part of the ISPR extractant. The extractant containing alcohol esters can be separated from the fermentation medium, and the alcohol can be recovered from the extractant. The extractant can be recycled to the fermentation vessel. Thus, in the case of butanol production, for example, the conversion of the butanol to an ester reduces the free butanol concentration in the fermentation medium, shielding the microorganism from the toxic effect of increasing butanol concentration. In addition, unfractionated grain can be used as feedstock without separation of lipids therein, since the lipids can be catalytically hydrolyzed to organic acid, thereby decreasing the rate of build-up of lipids in the ISPR extractant.

In situ product removal can be carried out in a batch mode or a continuous mode. In a continuous mode of in situ product removal, product is continually removed from the reactor. In a batchwise mode of in situ product removal, a volume of organic extractant is added to the fermentation vessel and the extractant is not removed during the process. For in situ product removal, the organic extractant can contact the fermentation medium at the start of the fermentation forming a biphasic fermentation medium. Alternatively, the organic extractant can contact the fermentation medium after the microorganism has achieved a desired amount of growth, which can be determined by measuring the optical density of the culture. Further, the organic extractant can contact the fermentation medium at a time at which the product alcohol level in the fermentation medium reaches a preselected level. In the case of butanol production according to some embodiments of the present invention, the organic acid extractant can contact the fermentation medium at a time before the butanol concentration reaches a toxic level, so as to esterify the butanol with the organic acid to produce butanol esters and consequently reduce the concentration of butanol in the fermentation vessel. The ester-containing organic phase can then be removed from the fermentation vessel (and separated from the fermentation broth which constitutes the aqueous phase) after a desired effective titer of the butanol esters is achieved. In some embodiments, the ester-containing organic phase is separated from the aqueous phase after fermentation of the available fermentable sugar in the fermentation vessel is substantially complete.

### EXAMPLES

The present invention is further defined in the following Examples. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

### Example 1

### Identification of Genes that Encode Acetolactate Reductase (ALR) Activity Enzymes Using Yeast Knockout Library

From a knockout (“KO”) collection of >6000 yeast strains derived from the strain BY4743, available from Open Biosystems® (a division of Thermo Fisher Scientific, Waltham, Mass.), 95 candidate dehydrogenase gene knockout strains were chosen. Starter cultures of knockout strains were grown in 96-well deepwell plates (Costar 3960, Corning Inc., Corning N.Y., or similar) on rich medium YPD, and subcultured at a starting OD 600 nm of ˜0.3 in medium containing 0.67% Yeast Nitrogen Base, 0.1% casamino acids, 2% glucose, and 0.1 M K⁺-MES, pH 5.5. Samples were taken over a 5-day period for DHMB and DHIV measurements. DHIV and the two isomers of DHMB were separated and quantified by liquid chromatography-mass spectrometry (“LC/MS”) on a Waters (Milford, Mass.) AcquityTQD system, using an Atlantis T3 (part #186003539) column. The column was maintained at 30° C., and the flow rate was 0.5 ml/min. The A mobile phase was 0.1% formic acid in water, and the B mobile phase was 0.1% formic acid in acetonitrile. Each run consisted of 1 min at 99% A, a linear gradient over 1 min to 25% B, followed by 1 min at 99% A. The column effluent was monitored for peaks at m/z=133 (negative ESI), with cone voltage 32.5V, by Waters ACQ TQD (s/n QBA688) mass spec detector. The so-called “fast DHMB” typically emerged at 1.10 min, followed by DHIV at 1.2 min, and “slow” DHMB emerged at 1.75 min. Baseline separation was obtained and peak areas for DHIV were converted to 1 μM DHIV concentrations by reference to analyses of standards solutions made from a 1M aqueous stock. These measurements showed that most of the changes in DHMB levels occurred in the first 48-60 hours, so a single sample was collected at about that time in subsequent experiments. In this experiment, fast DHMB was found at much higher levels than slow DHMB, which was not always detectable. The ratio of DHIV to fast DHMB in most cultures was ˜3, but a strain lacking the YMR226C gene consistently showed very low levels of fast DHMB, and normal DHIV, so that the DHIV/fast DHMB ratio was about 100. This suggested that YMR226Cp is the major ALR in this background. The gene is encoded by EMBL reference Z49939.

To confirm that YMR226Cp is the major ALR in this background, the in vitro levels of ALR and KARI were tested in the ymr226c deletion strain (American Type Culture Collection (ATCC), Manassas Va., ATCC #4020812) and its parent, BY4743 (ATCC #201390; American Type Culture Collection, Manassas Va.). Fifty ml tubes containing 6 ml YPD were inoculated from YPD agar plates and allowed to grow overnight (30° C., 250 rpm). The cells were pelleted, washed once in water, and resuspended in 1 ml yeast cytoplasm buffer (Van Eunen et al. FEBS Journal 277: 749-760 (2010)) containing a yeast protease inhibitor cocktail (Roche, Basel, Switzerland, Cat #11836170001, used as directed by the vendor, 1 tablet per 10 mls of buffer). Toluene (0.02 ml, Fisher Scientific, Fair Lawn N.J.) was added, and the tubes were shaken at top speed for 10 min on a Vortex Genie 2 shaker (Scientific Industries, Bohemia N.Y., Model G-560) for permeabilization. The tubes were placed in a water bath at 30° C., and substrates were added to the following final concentrations: (S)-acetolactate (made enzymatically as described below in Example 6) to 9.4 mM, NADPH (Sigma-Aldrich, St. Louis Mo.) 0.2 mM plus a NAD(P)H-regeneration system consisting of ˜10 mM glucose-6-phosphate and 2.5 U/ml Leuconostoc mesenteroides glucose-6-phosphate dehydrogenase (Sigma, St. Louis, Mo., Cat # G8404). At timed intervals, aliquots (0.15 ml) were added to 0.15 ml aliquots of 2% formic acid to stop the reaction. The samples were then analyzed for DHIV and both isomers of DHMB by LC/MS as described above; only fast DHMB and DHIV were observed. The specific activities of the two enzymes in the two strains are shown in Table 3.

TABLE 3
KARI and ALR Enzyme Activities

| Strain | KARI | ALR |
|---|---|---|
| BY4743 | 1.7 mU/mg protein | 20 mU/mg |
| ΔYMR226C KO | 2.2 mU/mg protein | 0.1 mU/mg |

The data suggests that the YMR226C gene product accounted for >99% of the ALR activity.

### Example 2

### Identification of Genes that Encode Acetolactase Reductase (ALR) Activity Enzymes Using Yeast Overeexpresion Library

From a “Yeast ORF” collection of >5000 transformants of Y258 each with a plasmid carrying a known yeast gene plus a C-terminal tag, under the control of an inducible promoter (Open Biosystems®, a division of Thermo Fisher Scientific, Waltham, Mass.), ninety-six strains with plasmids containing genes associated with dehydrogenase activity were grown in 96-well format by adaptation of the growth and induction protocol recommended by the vendor (Open Biosystems®). The cells were pelleted and permeabilized with toluene as described above, and a concentrated substrate mix was added to give final concentrations as in Example 1. Timed samples were taken and analyzed for DHIV and both isomers of DHMB. The ratios of the ALR/KARI were calculated and compared. Strains with elevated ratios were candidates for overproduction of ALR activities. When the data were displayed in a Minitab® (Microsoft Inc., Redmond, Wash.) boxplot, the typical ALR/KARI ratio was about 10, but a few strains showed higher ALR/KARI ratios, some of which were statistically significant. Among these were YMR226C and YER081W, which increased synthesis of both DHMBs. In addition, YIL074C and YBR006W increased fast DHMB synthesis, and YPL275W and YOL059W increased slow DHMB synthesis. The genomic DNA sequences (which may include introns) and ORF translation sequences of genes identified in overexpression are provided below in Table 4.

TABLE 4
ALR Genes Identified Using Overexpression

| | |
|---|---|
| Gene | Sequence |

| | |
|---|---|
| YIL074C | ATGTCTTATTCAGCTGCCGATAATTTACAAGATTCATTCCAACGTGCC |
| (Chr 9) | ATGAACTTTTCTGGCTCTCCTGGTGCAGTCTCAACCTCACCAACTCAG |
| | TCATTTATGAACACACTACCTCGTCGTGTAAGCATTACAAAGCAACC |
| | AAAGGCTTTAAAACCTTTTTCTACTGGTGACATGAATATTCTACTGTT |
| | GGAAAATGTCAATGCAACTGCAATCAAAATCTTCAAGGATCAGGGTT |
| | ACCAAGTAGAGTTCCACAAGTCTTCTCTACCTGAGGATGAATTGATTG |
| | AAAAAATCAAAGACGTACACGCTATCGGTATAAGATCCAAAACTAGA |
| | TTGACTGAAAAAATACTACAGCATGCCAGGAATCTAGTTTGTATTGG |
| | TTGTTTTTGCATAGGTACCAATCAAGTAGACCTAAAATATGCCGCTAG |
| | TAAAGGTATTGCTGTTTTCAATTCGCCATTCTCCAATTCAAGATCCGT |
| | AGCAGAATTGGTAATTGGTGAGATCATTAGTTTAGCAAGACAATTAG |
| | GTGATAGATCCATTGAACTGCATACAGGTACATGGAATAAAGTCGCT |
| | GCTAGGTGTTGGGAAGTAAGAGGAAAAACTCTCGGTATTATTGGGTA |
| | TGGTCACATTGGTTCGCAATTATCAGTTCTTGCAGAAGCTATGGGCCT |
| | GCATGTGCTATACTATGATATCGTGACAATTATGGCCTTAGGTACTGC |
| | CAGACAAGTTTCTACATTAGATGAATTGTTGAATAAATCTGATTTTGT |
| | AACACTACATGTACCAGCTACTCCAGAAACTGAAAAAATGTTATCTG |
| | CTCCACAATTCGCTGCTATGAAGGACGGGGCTTATGTTATTAATGCCT |
| | CAAGAGGTACTGTCGTGGACATTCCATCTCTGATCCAAGCCGTCAAG |
| | GCCAACAAAATTGCAGGTGCTGCTTTAGATGTTTATCCACATGAACC |
| | AGCTAAGAACGGTGAAGGTTCATTTAACGATGAACTTAACAGCTGGA |
| | CTTCTGAGTTGGTTTCATTACCAAATATAATCCTGACACCACATATTG |
| | GTGGCTCTACAGAAGAAGCTCAAAGTTCAATCGGTATTGAGGTGGCT |
| | ACTGCATTGTCCAAATACATCAATGAAGGTAACTCTGTCGGTTCTGTG |
| | AACTTCCCAGAAGTCAGTTTGAAGTCTTTGGACTACGATCAAGAGAA |
| | CACAGTACGTGTCTTGTATATTCATCGTAACGTTCCTGGTGTTTTGAA |
| | GACCGTTAATGATATCTTATCCGATCATAATATCGAGAAACAGTTTTC |
| | TGATTCTCACGGCGAGATCGCTTATCTAATGGCAGACATCTCTTCTGT |
| | TAATCAAAGTGAAATCAAGGATATATATGAAAAGTTGAACCAAACTT |
| | CTGCCAAAGTTTCCATCAGGTTATTATACTAA (SEQ ID NO: 25) |
| | MSYSAADNLQDSFQRAMNFSGSPGAVSTSPTQSFMNTLPRRVSITKQPK |
| | ALKPFSTGDMNILLLENVNATAIKIFKDQGYQVEFHKSSLPEDELIEKIKD |
| | VHAIGIRSKTRLTEKILQHARNLVCIGCFCIGTNQVDLKYAASKGIAVFNS |
| | PFSNSRSVAELVIGEIISLARQLGDRSIELHTGTWNKVAARCWEVRGKTL |
| | GIIGYGHIGSQLSVLAEAMGLHVLYYDIVTIMALGTARQVSTLDELLNKS |
| | DFVTLHVPATPETEKMLSAPQFAAMKDGAYVINASRGTVVDIPSLIQAV |
| | KANKIAGAALDVYPHEPAKNGEGSFNDELNSWTSELVSLPNIILTPHIGG |
| | STEEAQSSIGIEVATALSKYINEGNSVGSVNFPEVSLKSLDYDQENTVRVL |
| | YIHRNVPGVLKTVNDILSDHNIEKQFSDSHGEIAYLMADISSVNQSEIKDI |
| | YEKLNQTSAKVSIRLLY (SEQ ID NO: 26) |
| YIR036C | ATGGGCAAGGTTATTTTGATTACAGGTGCCTCCCGTGGGATTGGCCTG |
| (Chr 9) | CAATTGGTGAAAACTGTTATCGAAGAGGACGATGAATGCATCGTCTA |
| | CGGCGTAGCAAGAACGGAAGCTGGTCTGCAGTCTTTGCAAAGAGAAT |
| | ACGGTGCAGACAAATTTGTCTATCGTGTCCTCGACATCACGGACAGG |
| | TCTCGAATGGAAGCGTTGGTGGAGGAAATCCGGCAAAAGCATGGAA |
| | AACTGGACGGTATTGTCGCAAATGCGGGGATGCTAGAACCGGTGAAG |
| | TCCATCTCCCAGTCCAACTCCGAACACGACATCAAGCAGTGGGAACG |
| | GCTGTTCGATGTGAACTTTTTCAGCATTGTCTCTTTGGTGGCACTGTGT |
| | TTACCCCTCTTGAAGAGCTCGCCATTTGTAGGCAACATTGTCTTCGTC |
| | AGCTCTGGAGCCAGTGTGAAACCATATAACGGATGGTCGGCGTACGG |
| | CTGCTCGAAAGCCGCATTAAACCACTTTGCCATGGACATTGCCAGTG |
| | AAGAGCCCAGTGATAAAGTGCGTGCCGTGTGTATTGCACCGGGCGTC |
| | GTTGACACGCAGATGCAGAAAGATATTAGGGAAACATTGGGTCCTCA |
| | GGGCATGACACCCAAGGCTCTCGAGAGGTTTACTCAATTGTACAAG |
| | ACTTCGTCACTGCTGGACCCAAAGGTGCCTGCGGCGGTACTAGCGCA |
| | ACTCGTCCTGAAAGGTATTCCCGACTCTTTGAACGGTCAATATCTCCG |
| | CTACAACGATGAGCGACTGGGGCCGGTGCAGGGCTAG (SEQ ID |
| | NO: 27) |
| | MGKVILITGASRGIGLQLVKTVIEEDDECIVYGVARTEAGLQSLQREYGA |
| | DKFVYRVLDITDRSRMEALVEEIRQKHGKLDGIVANAGMLEPVKSISQS |
| | NSEHDIKQWERLFDVNFFSIVSLVALCLPLLKSSPFVGNIVFVSSGASVKP |
| | YNGWSAYGCSKAALNHFAMDIASEEPSDKVRAVCIAPGVVDTQMQKDI |
| | RETLGPQGMTPKALERFTQLYKTSSLLDPKVPAAVLAQLVLKGIPDSLN |
| | GQYLRYNDERLGPVQG (SEQ ID NO: 28) |
| YPL061W | ATGACTAAGCTACACTTTGACACTGCTGAACCAGTCAAGATCACACT |
| (ALD6) | TCCAAATGGTTTGACATACGAGCAACCAACCGGTCTATTCATTAACA |
| (Chr 16) | ACAAGTTTATGAAAGCTCAAGACGGTAAGACCTATCCCGTCGAAGAT |
| | CCTTCCACTGAAAACACCGTTTGTGAGGTCTCTTCTGCCACCACTGAA |
| | GATGTTGAATATGCTATCGAATGTGCCGACCGTGCTTTCCACGACACT |
| | GAATGGGCTACCCAAGACCCAAGAGAAAGAGGCCGTCTACTAAGTA |
| | AGTTGGCTGACGAATTGGAAAGCCAAATTGACTTGGTTTCTTCCATTG |
| | AAGCTTTGGACAATGGTAAAACTTTGGCCTTAGCCCGTGGGGATGTT |
| | ACCATTGCAATCAACTGTCTAAGAGATGCTGCTGCCTATGCCGACAA |
| | AGTCAACGGTAGAACAATCAACACCGGTGACGGCTACATGAACTTCA |
| | CCACCTTAGAGCCAATCGGTGTCTGTGGTCAAATTATTCCATGGAACT |
| | TTCCAATAATGATGTTGGCTTGGAAGATCGCCCCAGCATTGGCCATG |
| | GGTAACGTCTGTATCTTGAAACCCGCTGCTGTCACACCTTTAAATGCC |
| | CTATACTTTGCTTCTTTATGTAAGAAGGTTGGTATTCCAGCTGGTGTC |
| | GTCAACATCGTTCCAGGTCCTGGTAGAACTGTTGGTGCTGCTTTGACC |
| | AACGACCCAAGAATCAGAAAGCTGGCTTTTACCGGTTCTACAGAAGT |
| | CGGTAAGAGTGTTGCTGTCGACTCTTCTGAATCTAACTTGAAGAAAAT |
| | CACTTTGGAACTAGGTGGTAAGTCCGCCCATTTGGTCTTTGACGATGC |
| | TAACATTAAGAAGACTTTACCAAATCTAGTAAACGGTATTTTCAAGA |
| | ACGCTGGTCAAATTTGTTCCTCTGGTTCTAGAATTTACGTTCAAGAAG |
| | GTATTTACGACGAACTATTGGCTGCTTTCAAGGCTTACTTGGAAACCG |
| | AAATCAAAGTTGGTAATCCATTTGACAAGGCTAACTTCCAAGGTGCT |
| | ATCACTAACCGTCAACAATTCGACACAATTATGAACTACATCGATAT |
| | CGGTAAGAAAGAAGGCGCCAAGATCTTAACTGGTGGCGAAAAAGTT |
| | GGTGACAAGGGTTACTTCATCAGACCAACCGTTTTCTACGATGTTAAT |
| | GAAGACATGAGAATTGTTAAGGAAGAAATTTTTGGACCAGTTGTCAC |
| | TGTCGCAAAGTTCAAGACTTTAGAAGAAGGTGTCGAAATGGCTAACA |
| | GCTCTGAATTCGGTCTAGGTTCTGGTATCGAAACAGAATCTTTGAGCA |
| | CAGGTTTGAAGGTGGCCAAGATGTTGAAGGCCGGTACCGTCTGGATC |
| | AACACATACAACGATTTTGACTCCAGAGTTCCATTCGGTGGTGTTAAG |
| | CAATCTGGTTACGGTAGAGAAATGGGTGAAGAAGTCTACCATGCATA |
| | CACTGAAGTAAAAGCTGTCAGAATTAAGTTGTAA (SEQ ID NO: 29) |
| | MTKLHFDTAEPVKITLPNGLTYEQPTGLFINNKFMKAQDGKTYPVEDPS |
| | TENTVCEVSSATTEDVEYAIECADRAFHDTEWATQDPRERGRLLSKLAD |
| | ELESQIDLVSSIEALDNGKTLALARGDVTIAINCLRDAAAYADKVNGRTI |
| | NTGDGYMNFTTLEPIGVCGQIIPWNFPIMMLAWKIAPALAMGNVCILKP |
| | AAVTPLNALYFASLCKKVGIPAGVVNIVPGPGRTVGAALTNDPRIRKLAF |
| | TGSTEVGKSVAVDSSESNLKKITLELGGKSAHLVFDDANIKKTLPNLVNG |
| | IFKNAGQICSSGSRIYVQEGIYDELLAAFKAYLETEIKVGNPFDKANFQGA |
| | ITNRQQFDTIMNYIDIGKKEGAKILTGGEKVGDKGYFIRPTVFYDVNEDM |
| | RIVKEEIFGPVVTVAKFKTLEEGVEMANSSEFGLGSGIETESLSTGLKVAK |
| | MLKAGTVWINTYNDFDSRVPFGGVKQSGYGREMGEEVYHAYTEVKAV |
| | RIKL (SEQ ID NO: 30) |
| YPL088W | ATGGTTTTAGTTAAGCAGGTAAGACTCGGTAACTCAGGTCTTAAGAT |
| (Chr 16) | ATCACCGATAGTGATAGGATGTATGTCATACGGGTCCAAGAAATGGG |
| | CGGACTGGGTCATAGAGGACAAGACCCAAATTTTCAAGATTATGAAG |
| | CATTGTTACGATAAAGGTCTTCGTACTTTTGACACAGCAGATTTTTAT |
| | TCTAATGGTTTGAGTGAAAGAATAATTAAGGAGTTTCTGGAGTACTA |
| | CAGTATAAAGAGAGAAACGGTGGTGATTATGACCAAAATTTACTTCC |
| | CAGTTGATGAAACGCTTGATTTGCATCATAACTTCACTTTAAATGAAT |
| | TTGAAGAATTGGACTTGTCCAACCAGCGGGGTTTATCCAGAAAGCAT |
| | ATAATTGCTGGTGTCGAGAACTCTGTGAAAAGACTGGGCACATATAT |
| | AGACCTTTTACAAATTCACAGATTAGATCATGAAACGCCAATGAAAG |
| | AGATCATGAAGGCATTGAATGATGTTGTTGAAGCGGGCCACGTTAGA |
| | TACATTGGGGCTTCGAGTATGTTGGCAACTGAATTTGCAGAACTGCA |
| | GTTCACAGCCGATAAATATGGCTGGTTTCAGTTCATTTCTTCGCAGTC |
| | TTACTACAATTTGCTCTATCGTGAAGATGAACGCGAATTGATTCCTTT |
| | TGCCAAAAGACACAATATTGGTTTACTTCCATGGTCTCCTAACGCACG |
| | AGGCATGTTGACTCGTCCTCTGAACCAAAGCACGGACAGGATTAAGA |
| | GTGATCCAACTTTCAAGTCGTTACATTTGGATAATCTCGAAGAAGAA |
| | CAAAAGGAAATTATAAATCGTGTGGAAAAGGTGTCGAAGGACAAAA |
| | AAGTCTCGATGGCTATGCTCTCCATTGCATGGGTTTTGCATAAAGGAT |
| | GTCACCCTATTGTGGGATTGAACACTACAGCAAGAGTAGACGAAGCG |
| | ATTGCCGCACTACAAGTAACTCTAACAGAAGAAGAGATAAAGTACCT |
| | CGAGGAGCCCTACAAACCCCAGAGGCAAAGATGTTAA (SEQ ID |
| | NO: 31) |
| | MVLVKQVRLGNSGLKISPIVIGCMSYGSKKWADWVIEDKTQIFKIMKHC |
| | YDKGLRTFDTADFYSNGLSERIIKEFLEYYSIKRETVVIMTKIYFPVDETL |
| | DLHHNFTLNEFEELDLSNQRGLSRKHIIAGVENSVKRLGTYIDLLQIHRLD |
| | HETPMKEIMKALNDVVEAGHVRYIGASSMLATEFAELQFTADKYGWFQ |
| | FISSQSYYNLLYREDERELIPFAKRHNIGLLPWSPNARGMLTRPLNQSTDR |
| | IKSDPTFKSLHLDNLEEEQKEIINRVEKVSKDKKVSMAMLSIAWVLHKG |
| | CHPIVGLNTTARVDEAIAALQVTLTEEEIKYLEEPYKPQRQRC (SEQ ID |
| | NO: 32) |
| YCR105W | ATGCTTTACCCAGAAAAATTTCAGGGCATCGGTATTTCCAACGCAAA |
| (ADH7) | GGATTGGAAGCATCCTAAATTAGTGAGTTTTGACCCAAAACCCTTTG |
| (Chr 3) | GCGATCATGACGTTGATGTTGAAATTGAAGCCTGTGGTATCTGCGGA |
| | TCTGATTTTCATATAGCCGTTGGTAATTGGGGTCCAGTCCCAGAAAAT |
| | CAAATCCTTGGACATGAAATAATTGGCCGCGTGGTGAAGGTTGGATC |
| | CAAGTGCCACACTGGGGTAAAAATCGGTGACCGTGTTGGTGTTGGTG |
| | CCCAAGCCTTGGCGTGTTTTGAGTGTGAACGTTGCAAAAGTGACAAC |
| | GAGCAATACTGTACCAATGACCACGTTTTGACTATGTGGACTCCTTAC |
| | AAGGACGGCTACATTTCACAAGGAGGCTTTGCCTCCCACGTGAGGCT |
| | TCATGAACACTTTGCTATTCAAATACCAGAAAATATTCCAAGTCCGCT |
| | AGCCGCTCCATTATTGTGTGGTGGTATTACAGTTTTCTCTCCACTACT |
| | AAGAAATGGCTGTGGTCCAGGTAAGAGGGTAGGTATTGTTGGCATCG |
| | GTGGTATTGGGCATATGGGGATTCTGTTGGCTAAAGCTATGGGAGCC |
| | GAGGTTTATGCGTTTTCGCGAGGCCACTCCAAGCGGGAGGATTCTAT |
| | GAAACTCGGTGCTGATCACTATATTGCTATGTTGGAGGATAAAGGCT |
| | GGACAGAACAATACTCTAACGCTTTGGACCTTCTTGTCGTTTGCTCAT |
| | CATCTTTGTCGAAAGTTAATTTTGACAGTATCGTTAAGATTATGAAGA |
| | TTGGAGGCTCCATCGTTTCAATTGCTGCTCCTGAAGTTAATGAAAAGC |
| | TTGTTTTAAAACCGTTGGGCCTAATGGGAGTATCAATCTCAAGCAGTG |
| | CTATCGGATCTAGGAAGGAAATCGAACAACTATTGAAATTAGTTTCC |
| | GAAAAGAATGTCAAAATATGGGTGGAAAAACTTCCGATCAGCGAAG |
| | AAGGCGTCAGCCATGCCTTTACAAGGATGGAAAGCGGAGACGTCAA |
| | ATACAGATTTACTTTGGTCGATTATGATAAGAAATTCCATAAATAG |
| | (SEQ ID NO: 33) |
| | MLYPEKFQGIGISNAKDWKHPKLVSFDPKPFGDHDVDVEIEACGICGSDF |
| | HIAVGNWGPVPENQILGHEIIGRVVKVGSKCHTGVKIGDRVGVGAQALA |
| | CFECERCKSDNEQYCTNDHVLTMWTPYKDGYISQGGFASHVRLHEHFAI |
| | QIPENIPSPLAAPLLCGGITVFSPLLRNGCGPGKRVGIVGIGGIGHMGILLA |
| | KAMGAEVYAFSRGHSKREDSMKLGADHYIAMLEDKGWTEQYSNALDL |
| | LVVCSSSLSKVNFDSIVKIMKIGGSIVSIAAPEVNEKLVLKPLGLMGVSISS |
| | SAIGSRKEIEQLLKLVSEKNVKIWVEKLPISEEGVSHAFTRMESGDVKYR |
| | FTLVDYDKKFHK (SEQ ID NO: 34) |
| YDR541C | ATGTCTAATACAGTTCTAGTTTCTGGCGCTTCAGGTTTTATTGCCTTGC |
| (Chr 4) | ATATCCTGTCACAATTGTTAAAACAAGATTATAAGGTTATTGGAACTG |
| | TGAGATCCCATGAAAAAGAAGCAAAATTGCTAAGACAATTTCAACAT |
| | AACCCTAATTTAACTTTAGAAATTGTTCCGGACATTTCTCATCCAAAT |
| | GCTTTCGATAAGGTTCTGCAGAAACGTGGACGTGAGATTAGGTATGT |
| | TCTACACACGGCCTCTCCTTTTCATTATGATACTACCGAATATGAAAA |
| | AGACTTATTGATTCCCGCGTTAGAAGGTACAAAAAACATCCTAAATT |
| | CTATCAAGAAATATGCAGCAGACACTGTAGAGCGTGTTGTTGTGACT |
| | TCTTCTTGTACTGCTATTATAACCCTTGCAAAGATGGACGATCCCAGT |
| | GTGGTTTTTACAGAAGAGAGTTGGAACGAAGCAACCTGGGAAAGCTG |
| | TCAAATTGATGGGATAAATGCTTACTTTGCATCCAAGAAGTTTGCTGA |
| | AAAGGCTGCCTGGGAGTTCACAAAAGAGAATGAAGATCACATCAAA |
| | TTCAAACTAACAACAGTCAACCCTTCTCTTCTTTTTGGTCCTCAACTTT |
| | TCGATGAAGATGTGCATGGCCATTTGAATACTTCTTGCGAAATGATCA |
| | ATGGCCTAATTCATACCCCAGTAAATGCCAGTGTTCCTGATTTTCATT |
| | CCATTTTTATTGATGTAAGGGATGTGGCCCTAGCTCATCTGTATGCTT |
| | TCCAGAAGGAAAATACCGCGGGTAAAAGATTAGTGGTAACTAACGGT |
| | AAATTTGGAAACCAAGATATCCTGGATATTTTGAACGAAGATTTTCC |
| | ACAATTAAGAGGTCTCATTCCTTTGGGTAAGCCTGGCACAGGTGATC |
| | AAGTCATTGACCGCGGTTCAACTACAGATAATAGTGCAACGAGGAAA |
| | ATACTTGGCTTTGAGTTCAGAAGTTTACACGAAAGTGTCCATGATACT |
| | GCTGCCCAAATTTTGAAGAAGCAGAACAGATTATGA (SEQ ID NO: 35) |
| | MSNTVLVSGASGFIALHILSQLLKQDYKVIGTVRSHEKEAKLLRQFQHNP |
| | NLTLEIVPDISHPNAFDKVLQKRGREIRYVLHTASPFHYDTTEYEKDLLIP |
| | ALEGTKNILNSIKKYAADTVERVVVTSSCTAIITLAKMDDPSVVFTEESW |
| | NEATWESCQIDGINAYFASKKFAEKAAWEFTKENEDHIKFKLTTVNPSLL |
| | FGPQLFDEDVHGHLNTSCEMINGLIHTPVNASVPDFHSIFIDVRDVALAH |
| | LYAFQKENTAGKRLVVTNGKFGNQDILDILNEDFPQLRGLIPLGKPGTGD |
| | QVIDRGSTTDNSATRKILGFEFRSLHESVHDTAAQILKKQNRL (SEQ ID |
| | NO: 36) |
| YER081 | ATGACAAGCATTGACATTAACAACTTACAAAATACCTTTCAACAAGC |
| (SER3) | TATGAATATGAGCGGCTCCCCAGGCGCTGTTTGTACTTCACCTACGCA |
| (Chr 5) | ATCTTTCATGAATACCGTTCCACAGCGCTTGAATGCTGTAAAGCACCC |
| | AAAAATTTTGAAGCCTTTCTCAACGGGTGATATGAAGATTTTACTATT |
| | AGAAAACGTTAATCAAACTGCTATTACAATCTTCGAAGAGCAAGGTT |
| | ACCAAGTCGAATTCTATAAATCTTCATTGCCCGAGGAAGAGTTGATC |
| | GAAAAGATCAAGGACGTTCATGCTATTGGTATCAGATCAAAGACTAG |
| | ATTAACTTCAAATGTCTTACAACATGCGAAGAATCTGGTTTGTATTGG |
| | TTGTTTCTGTATCGGTACCAACCAAGTTGACTTAGACTACGCTACCAG |
| | CAGAGGTATTGCTGTTTTCAACTCGCCTTTCTCCAACTCAAGATCAGT |
| | AGCAGAATTGGTCATCGCTGAAATCATTAGTTTAGCAAGACAACTAG |
| | GTGATAGATCTATCGAATTACATACCGGTACATGGAATAAGGTTGCT |
| | GCTAGATGTTGGGAGGTAAGAGGAAAAACTCTTGGTATTATTGGGTA |
| | CGGTCACATTGGTTCCCAATTATCAGTTCTTGCAGAAGCTATGGGTTT |
| | GCATGTGTTGTACTACGATATTGTAACTATCATGGCCTTGGGTACTGC |
| | CAGACAAGTTTCTACATTAGATGAATTGTTGAATAAATCTGATTTTGT |
| | GACACTACATGTACCAGCTACTCCTGAAACTGAAAAAATGTTATCTG |
| | CCCCACAATTTGCTGCTATGAAGGATGGCGCTTATGTTATTAATGCTT |
| | CAAGAGGTACTGTCGTGGACATTCCATCTTTGATCCAAGCCGTGAAA |
| | GCCAACAAAATTGCAGGTGCTGCTTTGGATGTTTATCCACATGAACC |
| | AGCTAAGAACGGTGAAGGTTCATTTAACGATGAGCTAAATAGCTGGA |
| | CTTCTGAATTAGTTTCATTACCAAATATCATCTTGACACCACACATTG |
| | GTGGCTCTACCGAAGAAGCCCAAAGCTCAATCGGTATTGAAGTGGCT |
| | ACCGCATTGTCCAAATACATCAATGAAGGTAACTCTGTCGGTTCAGTC |
| | AACTTCCCAGAAGTGGCATTGAAATCATTGTCTTACGACCAAGAGAA |
| | CACTGTGCGTGTGTTATACATTCACCAAAATGTACCAGGTGTTTTGAA |
| | GACCGTCAATGATATTTTATCGAACCATAACATCGAAAAGCAATTTTC |
| | CGATTCAAATGGTGAAATTGCTTATTTAATGGCTGATATCTCTTCTGT |
| | TGACCAAAGCGATATTAAAGATATTTATGAACAACTAAATCAAACCT |
| | CTGCTAAGATCTCAATTAGATTGCTATATTAA (SEQ ID NO: 37) |
| | MTSIDINNLQNTFQQAMNMSGSPGAVCTSPTQSFMNTVPQRLNAVKHPK |
| | ILKPFSTGDMKILLLENVNQTAITIFEEQGYQVEFYKSSLPEEELIEKIKDV |
| | HAIGIRSKTRLTSNVLQHAKNLVCIGCFCIGTNQVDLDYATSRGIAVFNSP |
| | FSNSRSVAELVIAEIISLARQLGDRSIELHTGTWNKVAARCWEVRGKTLG |
| | IIGYGHIGSQLSVLAEAMGLHVLYYDIVTIMALGTARQVSTLDELLNKSD |
| | FVTLHVPATPETEKMLSAPQFAAMKDGAYVINASRGTVVDIPSLIQAVK |
| | ANKIAGAALDVYPHEPAKNGEGSFNDELNSWTSELVSLPNIILTPHIGGST |
| | EEAQSSIGIEVATALSKYINEGNSVGSVNFPEVALKSLSYDQENTVRVLYI |
| | HQNVPGVLKTVNDILSNHNIEKQFSDSNGEIAYLMADISSVDQSDIKDIYE |
| | QLNQTSAKISIRLLY(SEQ ID NO: 38) |
| YPL275W | ATGGTGGTCATCAATAAGCAATTAATGGTGAGTGGGATATTGCCGGC |
| (FDH2) | GTGGCTAAAAAATGAGTATGATCTGGAAGACAAAATAATTTCAACGG |
| (Chr 16) | TAGGTGCCGGTAGAATTGGATATAGGGTTCTGGAAAGATTGGTCGCA |
| | TTTAATCCGAAGAAGTTACTGTACTACGACTACCAGGAACTACCTGC |
| | GGAAGCAATCAATAGATTGAACGAGGCCAGCAAGCTTTTCAATGGCA |
| | GAGGTGATATTGTTCAGAGAGTAGAGAAATTGGAGGATATGGTTGCT |
| | CAGTCAGATGTTGTTACCATCAACTGTCCATTGCACAAGGACTCAAG |
| | GGGTTTATTCAATAAAAAGCTTATTTCCCACATGAAAGATGGTGCAT |
| | ACTTGGTGAATACCGCTAGAGGTGCTATTTGTGTCGCAGAAGATGTT |
| | GCCGAGGCAGTCAAGTCTGGTAAATTGGCTGGCTATGGTGGTGATGT |
| | CTGGGATAAGCAACCAGCACCAAAAGACCATCCCTGGAGGACTATGG |
| | ACAATAAGGACCACGTGGGAAACGCAATGACTGTTCATATCAGTGGC |
| | ACATCTCTGCATGCTCAAAAGAGGTACGCTCAGGGAGTAAAGAACAT |
| | CCTAAATAGTTACTTTTCCAAAAAGTTTGATTACCGTCCACAGGATAT |
| | TATTGTGCAGAATGGTTCTTATGCCACCAGAGCTTATGGACAGAAGA |
| | AATAA (SEQ ID NO: 39) |
| | MVVINKQLMVSGILPAWLKNEYDLEDKIISTVGAGRIGYRVLERLVAFN |
| | PKKLLYYDYQELPAEAINRLNEASKLFNGRGDIVQRVEKLEDMVAQSDV |
| | VTINCPLHKDSRGLFNKKLISHMKDGAYLVNTARGAICVAEDVAEAVKS |
| | GKLAGYGGDVWDKQPAPKDHPWRTMDNKDHVGNAMTVHISGTSLHA |
| | QKRYAQGVKNILNSYFSKKFDYRPQDIIVQNGSYATRAYGQKK (SEQ ID |
| | NO: 40) |
| YBR006W | ATGACTTTGAGTAAGTATTCTAAACCAACTCTAAACGACCCTAATTTA |
| (UGA5) | TTCAGAGAATCTGGTTATATTGACGGAAAATGGGTTAAGGGCACTGA |
| (Chr2) | CGAAGTTTTTGAGGTGGTAGACCCTGCTTCCGGCGAAATCATAGCAA |
| | GAGTTCCCGAACAACCAGTCTCCGTGGTTGAGGAAGCGATTGATGTT |
| | GCCTATGAAACTTTCAAGACGTACAAGAATACAACACCAAGAGAGA |
| | GGGCAAAGTGGCTCAGAAACATGTATAACTTAATGCTTGAAAATTTG |
| | GATGATCTGGCAACCATCATTACTTTAGAAAATGGTAAAGCTCTAGG |
| | GGAAGCTAAAGGAGAAATCAAATACGCGGCTTCGTATTTTGAGTGGT |
| | ACGCCGAGGAAGCACCCCGTTTATATGGTGCTACTATTCAACCCTTGA |
| | ACCCTCACAACAGAGTATTCACAATTAGGCAACCTGTTGGTGTATGC |
| | GGTATAATTTGTCCATGGAATTTTCCGAGCGCCATGATCACGAGAAA |
| | GGCCGCCGCTGCTTTAGCTGTGGGCTGCACAGTAGTCATCAAGCCAG |
| | ACTCTCAAACGCCGCTATCTGCTTTAGCAATGGCATATTTGGCTGAAA |
| | AGGCAGGCTTTCCCAAGGGTTCGTTTAATGTTATTCTTTCACATGCCA |
| | ACACACCAAAGCTTGGTAAAACATTATGTGAATCACCAAAAGTCAAG |
| | AAAGTTACTTTTACTGGTTCTACAAACGTCGGTAAAATCTTGATGAAA |
| | CAATCTTCTTCTACTTTGAAGAAACTGTCTTTTGAGCTGGGTGGTAAC |
| | GCCCCTTTCATAGTCTTTGAGGATGCCGATTTGGATCAAGCCTTGGAA |
| | CAAGCCATGGCTTGTAAATTTAGGGGTTTGGGTCAAACATGTGTGTG |
| | CGCAAATAGACTTTACGTTCACTCATCCATAATTGATAAATTTGCGAA |
| | ATTACTCGCGGAGAGGGTCAAAAAATTCGTAATTGGCCATGGTTTGG |
| | ACCCAAAAACTACACATGGTTGTGTCATTAACTCCAGCGCTATTGAA |
| | AAAGTTGAAAGACATAAACAGGATGCCATTGATAAGGGAGCAAAAG |
| | TTGTGCTTGAAGGTGGACGTTTAACTGAGTTAGGTCCTAACTTTTATG |
| | CTCCAGTAATTTTGTCACACGTTCCCTCAACAGCTATTGTTTCCAAGG |
| | AGGAGACTTTTGGTCCATTATGTCCAATCTTTTCTTTTGATACTATGG |
| | AAGAAGTTGTCGGATATGCTAATGATACTGAGTTTGGTTTAGCAGCA |
| | TATGTCTTTTCTAAAAATGTCAACACTTTATACACTGTGTCTGAAGCT |
| | TTGGAAACTGGTATGGTTTCATGTAATACAGGTGTTTTTTCGGATTGT |
| | TCTATACCATTTGGTGGTGTTAAAGAGTCAGGATTTGGAAGAGAAGG |
| | TTCGCTATATGGTATTGAAGATTACACTGTTTTGAAGACCATCACAAT |
| | TGGGAATTTGCCAAACAGCATTTAA (SEQ ID NO: 134) |
| | MTLSKYSKPTLNDPNLFRESGYIDGKWVKGTDEVFEVVDPASGEIIARVP |
| | EQPVSVVEEAIDVAYETFKTYKNTTPRERAKWLRNMYNLMLENLDDLA |
| | TIITLENGKALGEAKGEIKYAASYFEWYAEEAPRLYGATIQPLNPHNRVF |
| | TIRQPVGVCGIICPWNFPSAMITRKAAAALAVGCTVVIKPDSQTPLSALA |
| | MAYLAEKAGFPKGSFNVILSHANTPKLGKTLCESPKVKKVTFTGSTNVG |
| | KILMKQSSSTLKKLSFELGGNAPFIVFEDADLDQALEQAMACKFRGLGQ |
| | TCVCANRLYVHSSIIDKFAKLLAERVKKFVIGHGLDPKTTHGCVINSSAIE |
| | KVERHKQDAIDKGAKVVLEGGRLTELGPNFYAPVILSHVPSTAIVSKEET |
| | FGPLCPIFSFDTMEEVVGYANDTEFGLAAYVFSKNVNTLYTVSEALETG |
| | MVSCNTGVFSDCSIPFGGVKESGFGREGSLYGIEDYTVLKTITIGNLPNSI |
| | (SEQ ID NO: 135) |
| YOL059W | ATGCTTGCTGTCAGAAGATTAACAAGATACACATTCCTTAAGCGAAC |
| (Chr15) | GCATCCGGTGTTATATACTCGTCGTGCATATAAAATTTTGCCTTCAAG |
| | ATCTACTTTCCTAAGAAGATCATTATTACAAACACAACTGCACTCAAA |
| | GATGACTGCTCATACTAATATCAAACAGCACAAACACTGTCATGAGG |
| | ACCATCCTATCAGAAGATCGGACTCTGCCGTGTCAATTGTACATTTGA |
| | AACGTGCGCCCTTCAAGGTTACAGTGATTGGTTCTGGTAACTGGGGG |
| | ACCACCATCGCCAAAGTCATTGCGGAAAACACAGAATTGCATTCCCA |
| | TATCTTCGAGCCAGAGGTGAGAATGTGGGTTTTTGATGAAAAGATCG |
| | GCGACGAAAATCTGACGGATATCATAAATACAAGACACCAGAACGTT |
| | AAATATCTACCCAATATTGACCTGCCCCATAATCTAGTGGCCGATCCT |
| | GATCTTTTACACTCCATCAAGGGTGCTGACATCCTTGTTTTCAACATC |
| | CCTCATCAATTTTTACCAAACATAGTCAAACAATTGCAAGGCCACGT |
| | GGCCCCTCATGTAAGGGCCATCTCGTGTCTAAAAGGGTTCGAGTTGG |
| | GCTCCAAGGGTGTGCAATTGCTATCCTCCTATGTTACTGATGAGTTAG |
| | GAATCCAATGTGGCGCACTATCTGGTGCAAACTTGGCACCGGAAGTG |
| | GCCAAGGAGCATTGGTCCGAAACCACCGTGGCTTACCAACTACCAAA |
| | GGATTATCAAGGTGATGGCAAGGATGTAGATCATAAGATTTTGAAAT |
| | TGCTGTTCCACAGACCTTACTTCCACGTCAATGTCATCGATGATGTTG |
| | CTGGTATATCCATTGCCGGTGCCTTGAAGAACGTCGTGGCACTTGCAT |
| | GTGGTTTCGTAGAAGGTATGGGATGGGGTAACAATGCCTCCGCAGCC |
| | ATTCAAAGGCTGGGTTTAGGTGAAATTATCAAGTTCGGTAGAATGTTT |
| | TTCCCAGAATCCAAAGTCGAGACCTACTATCAAGAATCCGCTGGTGT |
| | TGCAGATCTGATCACCACCTGCTCAGGCGGTAGAAACGTCAAGGTTG |
| | CCACATACATGGCCAAGACCGGTAAGTCAGCCTTGGAAGCAGAAAA |
| | GGAATTGCTTAACGGTCAATCCGCCCAAGGGATAATCACATGCAGAG |
| | AAGTTCACGAGTGGCTACAAACATGTGAGTTGACCCAAGAATTCCCA |
| | TTATTCGAGGCAGTCTACCAGATAGTCTACAACAACGTCCGCATGGA |
| | AGACCTACCGGAGATGATTGAAGAGCTAGACATCGATGACGAATAG |
| | (SEQ ID NO: 136) |
| | MLAVRRLTRYTFLKRTHPVLYTRRAYKILPSRSTFLRRSLLQTQLHSKMT |
| | AHTNIKQHKHCHEDHPIRRSDSAVSIVHLKRAPFKVTVIGSGNWGTTIAK |
| | VIAENTELHSHIFEPEVRMWVFDEKIGDENLTDIINTRHQNVKYLPNIDLP |
| | HNLVADPDLLHSIKGADILVFNIPHQFLPNIVKQLQGHVAPHVRAISCLK |
| | GFELGSKGVQLLSSYVTDELGIQCGALSGANLAPEVAKEHWSETTVAYQ |
| | LPKDYQGDGKDVDHKILKLLFHRPYFHVNVIDDVAGISIAGALKNVVAL |
| | ACGFVEGMGWGNNASAAIQRLGLGEIIKFGRMFFPESKVETYYQESAGV |
| | ADLITTCSGGRNVKVATYMAKTGKSALEAEKELLNGQSAQGIITCREVH |
| | EWLQTCELTQEFPLFEAVYQIVYNNVRMEDLPEMIEELDIDDE (SEQ ID |
| | NO: 137) |

### Example 3

### Construction of S. cerevisiae Strain PNY2211

PNY2211 was constructed in several steps from S. cerevisiae strain PNY1507 (Example 12) as described in the following paragraphs. First the strain was modified to contain a phosophoketolase gene. Next, an acetolactate synthase gene (alsS) was added to the strain, using an integration vector targeted to sequence adjacent to the phosphoketolase gene. Finally, homologous recombination was used to remove the phosphoketolase gene and integration vector sequences, resulting in a scarless insertion of alsS in the intergenic region between pdc1Δ::ilvD (described in Example 11) and the native TRX1 gene of chromosome XII. The resulting genotype of PNY2211 is MATa ura3Δ::loxP his3Δ pdc6Δ pdc1Δ::P[PDC1]-DHAD|ilvD_Sm-PDC1t-P[FBA1]-ALS|alsS_Bs-CYC1t pdc5Δ::P[PDC5]-ADH| sadB_Ax-PDC5t gpd2Δ::loxP fra2Δ adh1Δ::UAS(PGK1)P[FBA1]-kivD_L1(y)-ADH1t.

A phosphoketolase gene cassette was introduced into PNY1507 (Example 12) by homologous recombination. The integration construct was generated as follows. The plasmid pRS423::CUP1-alsS+FBA-budA (previously described in US2009/0305363, which is herein incorporated by reference in its entirety) was digested with NotI and XmaI to remove the 1.8 kb FBA-budA sequence, and the vector was religated after treatment with Klenow fragment. Next, the CUP1 promoter was replaced with a TEF1 promoter variant (M4 variant previously described by Nevoigt et al. Appl. Environ. Microbial. 72: 5266-5273 (2006), which is herein incorporated by reference in its entirety)) via DNA synthesis and vector construction service from DNA2.0 (Menlo Park, Calif.). The resulting plasmid, pRS423::TEF(M4)-alsS was cut with StuI and MluI (removes 1.6 kb portion containing part of the alsS gene and CYC1 termintor), combined with the 4 kb PCR product generated from pRS426::GPD-xpk1+ADH-eutD (SEQ ID NO:249) with primers N1176 (SEQ ID NO:12) and N1177 (SEQ ID NO:13) and an 0.8 kb PCR product DNA generated from yeast genomic DNA (ENO1 promoter region) with primers N822 (SEQ ID NO:7) and N1178 (SEQ ID NO:14) and transformed into S. cerevisiae strain BY4741 (ATCC #201388); gap repair cloning methodology, see Ma et al. Gene 58:201-216 (1987). Transformants were obtained by plating cells on synthetic complete medium without histidine. Proper assembly of the expected plasmid (pRS423::TEF(M4)-xpk1+ENO1-eutD, SEQ ID NO:1) was confirmed by PCR (primers N821 (SEQ ID NO:6) and N1115 (SEQ ID NO:11)) and by restriction digest (BglI). Two clones were subsequently sequenced. The 3.1 kb TEF(M4)-xpk1 gene was isolated by digestion with SacI and NotI and cloned into the pUC19-URA3::ilvD-TRX1 vector (Clone A, cut with AflII). Cloning fragments were treated with Klenow fragment to generate blunt ends for ligation. Ligation reactions were transformed into E. coli Stbl3 cells, selecting for ampicillin resistance. Insertion of TEF(M4)-xpk1 was confirmed by PCR (primers N1110 (SEQ ID NO:9) and N1114 (SEQ ID NO:10)). The vector was linearized with AflII and treated with Klenow fragment. The 1.8 kb KpnI-HincII geneticin resistance cassette described in vector was cloned by ligation after Klenow fragment treatment. Ligation reactions were transformed into E. coli Stbl3 cells, selecting for ampicillin resistance. Insertion of the geneticin cassette was confirmed by PCR (primers N160SeqF5 (SEQ ID NO:4) and BK468 (SEQ ID NO:3)). The plasmid sequence is provided as SEQ ID NO:2 (pUC19-URA3::pdc1::TEF(M4)-xpk1::kan).

The resulting integration cassette (pdc1::TEF(M4)-xpk1::KanMX::TRX1) was isolated (AscI and NaeI digestion generated a 5.3 kb band that was gel purified) and transformed into PNY1507 using the Zymo Research Frozen-EZ Yeast Transformation Kit (Cat. No. T2001). Transformants were selected by plating on YPE plus 50 μg/ml G418. Integration at the expected locus was confirmed by PCR (primers N886 (SEQ ID NO:8) and N1214 (SEQ ID NO:15)). Next, plasmid pRS423::GAL1p-Cre (SEQ ID NO:123), encoding Cre recombinase, was used to remove the loxP-flanked KanMX cassette. Proper removal of the cassette was confirmed by PCR (primers oBP512 (SEQ ID NO:22) and N160SeqF5 (SEQ ID NO:4)). Finally, the alsS integration plasmid described in Example 9 (pUC19-kan::pdc1::FBA-alsS::TRX1, clone A) was transformed into this strain using the included geneticin selection marker. Two integrants were tested for acetolactate synthase activity by transformation with plasmids pYZ090ΔalsS (SEQ ID NO:248) and pBP915 (SEQ ID NO:84) transformed using Protocol #2 in Amberg, Burke and Strathern “Methods in Yeast Genetics” (2005)), and evaluation of growth and isobutanol production in glucose-containing media (methods for growth and isobutanol measurement are as follows: All strains were grown in synthetic complete medium, minus histidine and uracil containing 0.3% glucose and 0.3% ethanol as carbon sources (10 mL medium in 125 mL vented Erlenmeyer flasks (VWR Cat. No. 89095-260). After overnight incubation (30° C., 250 rpm in an Innova®40 New Brunswick Scientific Shaker), cultures were diluted back to 0.2 OD (Eppendorf BioPhotometer measurement) in synthetic complete medium containing 2% glucose and 0.05% ethanol (20 ml medium in 125 mL tightly-capped Erlenmeyer flasks (VWR Cat. No. 89095-260)). After 48 hours incubation (30° C., 250 rpm in an Innova®40 New Brunswick Scientific Shaker), culture supernatants (collected using Spin-X centrifuge tube filter units, Costar Cat. No. 8169) were analyzed by HPLC per methods described in U.S. Appl. Pub. No. 20070092957). One of the two clones was positive and was named PNY2218.

PNY2218 was treated with Cre recombinase, and the resulting clones were screened for loss of the xpk1 gene and pUC19 integration vector sequences by PCR (primers N886 (SEQ ID NO:8) and N160SeqR5 (SEQ ID NO:5)). This left only the alsS gene integrated in the pdc1-TRX1 intergenic region after recombination the DNA upstream of xpk1 and the homologous DNA introduced during insertion of the integration vector (a “scarless” insertion since vector, marker gene and loxP sequences are lost). Although this recombination could have occurred at any point, the vector integration appeared to be stable even without geneticin selection, and the recombination event was only observed after introduction of the Cre recombinase. One clone was designated PNY2211.

### Example 4

### YMR226c Deletion from S. cerevisiae Strain PNY2211 (Construction of PNY2248)

The gene YMR226c was deleted from S. cerevisiae strain PNY2211 (described in Example 3) by homologous recombination using a PCR amplified linear KanMX4-based deletion cassette available in S. cerevisiae strain BY4743 ymr226cΔ::KanMX4 (ATCC 4020812). Forward and reverse PCR primers N1237 (SEQ ID NO:16) and N1238 (SEQ ID NO:17), amplified a 2,051 bp ymr226cΔ::KanMX4 deletion cassette from chromosome XIII. The PCR product contained upstream and downstream sequences of 253 and 217 bp, respectively, flanking the ymr226cΔ::KanMX4 deletion cassette, that are 100% homologous to the sequences flanking the native YMR226c locus in strain PNY2211. Recombination and genetic exchange occur at the flanking homologous sequences effectively deleting the YMR226c gene and integrating the ymr226cΔ::KanMX4 deletion cassette.

Approximately 2.0 μg of the PCR amplified product was transformed into strain PNY2211 made competent using the lithium-acetate method previously described in Methods in Yeast Genetics (Cold Spring Harbor Laboratory Press, Cold Spring Harbor, N.Y., pp. 201-202 (2005)), and the transformation mix was plated on YPE plus geneticin (50 μg/mL) and incubated at 30° C. for selection of cells with an integrated ymr226cΔ::KanMX4 cassette. Transformants were screened for ymr226cΔ::KanMX4 by PCR, with a 5′ outward facing KanMX4 deletion cassette-specific internal primer N1240 (SEQ ID NO:19) paired with a flanking inward facing chromosome-specific primer N1239 (SEQ ID NO:18) and a 3′ outward-facing KanMX4 deletion cassette-specific primer N1241 (SEQ ID NO:20) paired with a flanking inward-facing chromosome-specific primer N1242 (SEQ ID NO:21). Positive PNY2211 ymr226cΔ::KanMX4 clones were obtained, one of which was designated PNY2248.

### Example 5

### Production of Isobutanol with Decreased DHMB Yield in YMR226c Knock-Out

PNY2211 ymr226cΔ::KanMX4 transformants and a non-deletion control (PNY2211 with native YMR226c) were tested for butanol production in glucose medium by first introducing the isobutanol pathway-containing plasmids pYZ090ΔalsS (SEQ ID NO:248, described in Example 9) and pBP915 (SEQ ID NO:84, described in Example 9) simultaneously by the Quick and Dirty lithium acetate transformation method described in Methods in Yeast Genetics (Cold Spring Harbor Laboratory Press, Cold Spring Harbor, N.Y. (2005)). Plasmid selection was based on histidine and uracil auxotrophy on selection plates containing ethanol (synthetic complete medium with 1.0% ethanol-his-ura). After three to five days, several transformants showing the most robust growth were adapted to glucose medium by patching onto SD 2.0% glucose+0.05% ethanol-his-ura and incubated 48 to 72 hours at 300° C. Three streaks showing the most robust growth were used to inoculate a 10 mL seed culture in SD 0.2% glucose+0.2% ethanol-his-ura in 125 mL vented flasks and grown at 30° C., 250 rpm for approximately 24 hours. Cells were then subcultured into synthetic complete medium with 2% glucose+0.05% ethanol-his-ura in 125 ml tightly-capped flasks and incubated 48 hours at 30° C. Culture supernatants collected after inoculation and after 48 hours incubation were analyzed by HPLC to determine production of isobutanol and by LC/MS to quantify DHMB. Controls strains were observed to produce DHMB at a molar yield of 0.03 to 0.07 mole per mole glucose. A peak corresponding to DHMB was not observed in culture supernatants of the ymr226cΔ strains, one of which was designated PNY2249.

### Example 6

### Inhibition of KARI by DHMB

### Enzymatic Production of (S)-Acetolactate

(S)-acetolactate was used as a starting material for DHMB synthesis. (S)-acetolactate was made enzymatically, as follows. An E. coli TOP10 strain (Invitrogen, Carlsbad, Calif.) modified to express Klebsiella BudB (previously described in U.S. Pat. No. 7,851,188, which is herein incorporated by reference in its entirety; see Example 9 of that patent) under IPTG control was used as a source of enzyme. It was grown in 200-1000 ml culture volumes. For example, 200 ml was grown in Luria Broth (Mediatech, Manassas, Va.) containing 0.1 mg/ml Ampicillin (Sigma, St. Louis, Mo.) in a 0.5 L conical flask, which was shaken at 250 rpm at 37° C. At OD 600 ˜0.4, isopropylthiogalactoside (Sigma, St. Louis, Mo.) was added to 0.4 mM, and growth was continued for 2 hours before the cells were collected by centrifugation, yielding ˜1 g wet weight cells. Likewise, partial purifications were conducted at scales from ˜0.5 to 5 g wet cells. For example, ˜0.5 g cells were suspended in 2.5 ml buffer containing 25 mM Na-MES pH 6, broken by sonication at 0° C., and clarified by centrifugation. Crude extract was supplemented with 0.1 mM thiamin pyrophosphate, 10 mM MgCl₂, and 1 mM EDTA (all from Sigma, St. Louis, Mo.). Next, 0.07 ml of 10% w/v aqueous streptomycin sulfate (Sigma, St. Louis, Mo.) was added and the sample was heated in a 56° C. water bath for 20 min. It was clarified by centrifugation, and ammonium sulfate was added to 50% of saturation. The mixture was centrifuged, and the pellet was brought up in 0.5 ml 25 mM Na-MES, pH 6.2, and used without further characterization. Acetolactate syntheses were also conducted at various scales. A large preparation was conducted as follows: 5.5 g sodium pyruvate was dissolved in 25 mM Na-MES, pH 6.2, to ˜45 ml and supplemented with 10 mM MgCl₂, 1 mM thiamin pyrophosphate, 1 mM EDTA (all from (Sigma, St. Louis, Mo.), 25 mM sodium acetate (Fisher Scientific, Fair Lawn N.J.), and 0.25 ml of a BudB preparation. The mixture was stirred under a pH meter at room temperature. As the reaction proceeded, CO₂ was evolved, and the pH rose. Pyruvic acid (Alfa, Ward Hill, Mass.) was added slowly via peristaltic pump to keep the pH between 6 and 7. As the pH rises, the enzyme reaction slows, but if it is allowed to fall below 6, decarboxylation of acetolactic acid becomes a problem. When the reaction was complete, the mixture was stored at −80° C.

Synthesis of DHMB

DHMB was synthesized chemically from (S)-acetolactate. Three ml of a crude acetolactate preparation at ˜0.8 M at pH ˜8 was treated with 1.2 equiv NaBH₄ (Aldrich Chemical Co, Milwaukee, Wis.). The reaction was allowed to sit at room temperature overnight before being divided in two and desalted in two portions on a 60 cm×1 cm diameter column of Biogel P-2 (Bio-Rad, Hercules, Calif.) using water as the mobile phase. The fractions containing mixed DHMBs were concentrated by rotary evaporation and adjusted to pH 2.2 with sulfuric acid.

The diastereomers of DHMB were separated using an HPLC system (consisting of an LKB 2249 pump and gradient controller (LKB, now a division of General Electric, Chalfont St Giles, UK) and a Hewlett-Packard (now Agilent, Santa Clara, Calif.) 1040A UV/vis detector) with a Waters Atlantis T3 (5 um, 4.6×150 mm) run at room temperature in 0.2% aqueous formic acid, pH 2.5, at a flow rate of 0.3 mL/min, with UV detection at 215 nm. “Fast” DHMB was eluted at 8.1 min and “slow” DHMB was eluted at 13.7 min. DHIV was not present. The pooled fractions were taken nearly to dryness, and coevaporated with toluene to remove residual formic acid. The residue was then dissolved in water and made basic with triethylamine (Fisher, Fair Lawn, N.J.).

### Concentration Determination and Absolute Structure of DHMB

The concentration of purified DHMB solutions was determined as follows. The concentration was estimated based on the mmol acetolactate used in the NaBH₄ reduction. To portions of the DHMBs, a known quantity of sodium benzoate (made by dissolving solid benzoic acid (ACS grade, Fisher Scientific, Fair Lawn, N.J.) in aqueous NaOH)) was added to give two-component mixtures in (approximately) equimolar amounts. A similar sample of DHIV was also prepared from the solid sodium salt obtained via custom synthesis (Albany Molecular Research, Albany N.Y.). The samples were coevaporated several times with D₂O (Aldrich, Milwaukee, Wis.) and redissolved in D₂O. Integrated proton NMR spectra were obtained and used to determine the mole ratio of DHIV or DHMB to benzoate. Comparison of the NMR spectra of the DHMBs with the literature spectra for the free acids in CDCl₃ (Kaneko et al., Phytochemistry 39: 115-120 (1995)) showed that fast DHMB was the erythro isomer. Since enzymatically synthesized acetolactate has the (S) configuration at C-2, the fast DHMB has the 2S, 3S configuration. Slow DHMB has the threo 2S, 3R configuration.

Dilutions of the NMR samples were also analyzed by LC/MS using separately prepared benzoic acid solutions as standards. Benzoic acid, DHIV, and the two isomers of DHMB were separated and quantified by LC/MS on a Waters (Milford, Mass.) AcquityTQD system, using an Atlantis T3 (part #186003539) column, as described above. Benzoic acid was detected at m/z=121 (negative ESI), and emerged at 2.05 min. The concentration of benzoate in the mixtures was within experimental uncertainty of the expected value. The experiment also showed that either isomer of DHMB had ˜80% of the sensitivity of DHIV in LC/MS (i.e., MS peak area observed/nmol injected) throughout the response range of the instrument. Thus, if a DHIV standard is used to quantify DHMB found in cell extracts or in enzymatic reactions, the apparent DHMB concentrations need to be multiplied by 1.25.

Measuring Inhibition of KARI by DHMB

Purified KARI encoded by genes either from Lactococcus lactis (SEQ ID NO: 262), a derivative of Pseudomonas fluorescens KARI known as JEA1 (U.S. Patent Application No. 2010/0197519), or a derivative of Anaerostipes caccae KARI known as K9D3 (SEQ ID NO:258), were tested for their sensitivity to DHMB inhibition in spectrophotometric assays in a Shimadzu (Kyoto, Japan) UV160U instrument with a TCC240A temperature control unit, set at 30° C. The buffer was 0.1 M K⁺ Hepes, pH 6.8, containing 10 mM MgCl2 and 1 mM EDTA. NADPH was present at 0.2 mM, and racemic acetolactate was present at either 3 mM or 0.725 mM (S) isomer. The rate of NADPH oxidation in the presence and absence of either fast or slow DHMB was measured. Vmax for each sample was calculated from the observed rate and the known acetolactate Km using the Michaelis-Menten equation. A volumetric Ki was estimated for each measurement in the presence of DHMB using the Michaelis-Menten equation as modified for competitive inhibition vs. acetolactate (the Km term in the MM equation is multiplied by (1+[I]/Ki), and the equation is solved for Ki. The results were converted to mM upon completion of the NMR experiment and are shown in Table 5.

TABLE 5
KI Values for KARI Inhibition by DHMB Isomers

| Strain | Fast DHMB | Slow DHMB |
|---|---|---|
| JEA1 | 0.23 mM | 0.23 mM |
| K9D3 | 0.3 mM | 0.2 mM |
| L. lactis | 2.8 mM | 2.3 mM |

### Example 7

### Inhibition of DHAD by DHMB

Purified dihydroxyacid dehydratase (DHAD) from Staphococcus mittans was tested for inhibition of conversion of dihydroxyisovalerate (DHIV) to 2-ketoisovalerate (2-KIV) by DHMB by using a modification of a colorimetric assay as described by Szamosi et al., Plant Phys. 101: 999-1004 (1993). The assay took place in a 2 mL Eppendorf tube placed in a heating block maintained at 30° C. The assay mixture had a final volume of 0.8 mL containing 100 mM Hepes-KOH buffer, pH 6.8, 10 mM MgCl₂, 0.5-10 mM DHIV, 0-40 mM DHMB, and 18 μg DHAD. The assay was initiated by adding a 10× concentrated stock of substrate. Samples were removed (0.35 mL) at times 0.1 and 30 minutes, and the reaction was stopped by mixing into 0.35 mL 0.1 N HCl with 0.05% 2,4-dinitrophenylhydrazine (Aldrich) in a second Eppendorf tube. After incubating 30 minutes at room temperature, 0.35 mL of 4N NaOH was added to the mixture, mixed, and centrifuged at 15,000×G for 2 minutes in a centrifuge (Beckman-Coulter Microfuge 18). The absorbance of the solution at 540 nm was then measured in a 1 cm pathlength cuvette using a Cary 300 Bio UV-Vis spectrophometer (Varian). Based on a standard curve using authentic 2-KIV (Fluka), 1 OD absorbance at 540 nm is produced by 0.28 mM 2-KIV. The rate of 2-KIV formation was measured in the presence and absence of either fast or slow DHMB. Both forms of DHMB behaved liked competitive inhibitors of DHIV. Their inhibition constants (Ki) were calculated from the Michaelis-Menten equation for simple competitive inhibition: v=S*Vmax/(S+Km*(1+I/Ki)), where v is the measured rate of 2-KIV formation, S is the initial concentration of DHIV, Vmax is the maximum rate calculated from the observed rate at 10 mM DHIV and no DHMB, Km is a previously measured constant of 0.5 mM, and I is the concentration of DHMB. The fast and slow isomers of DHMB had calculated inhibition constants of 7 mM and 5 mM, respectively.

### Example 8

### Identification of YMR226C Homologs

Homologs of the YMR226C gene of Saccharomyces cerevisiae were sought by

BLAST searches of the GenBank non-redundant nucleotide database (http://blast.ncbi.nlm.nih.gov/Blast.cgi), the Fungal Genomes BLAST Search Tool at the Saccharomyces Genome Database (http://www.yeastgenome.org/cgi-bin/blast-fungal.pl), and the BLAST Tool of the Genolevures Project (http://genolevures.org/blast.html#). Unique sequences from 18 yeast species showing high sequence identity to YMR226C were identified, and the complete ORF for these genes was recovered from the accessioned record in the associated database. The polypeptide sequences encoded by these ORFs were determined by the Translation feature of Vector NTI (Invitrogen, Carlsbad Calif.). The polynucleotide and polypeptide sequences are shown below in Table 6. The yeast species, nucleotide database accession number, and DNA and protein sequences are given in the Table. The S. kluyveri sequence is in the Genolevures database under the accession number given; the others are in GenBank. The percent identities between the sequences are shown in Table 7.

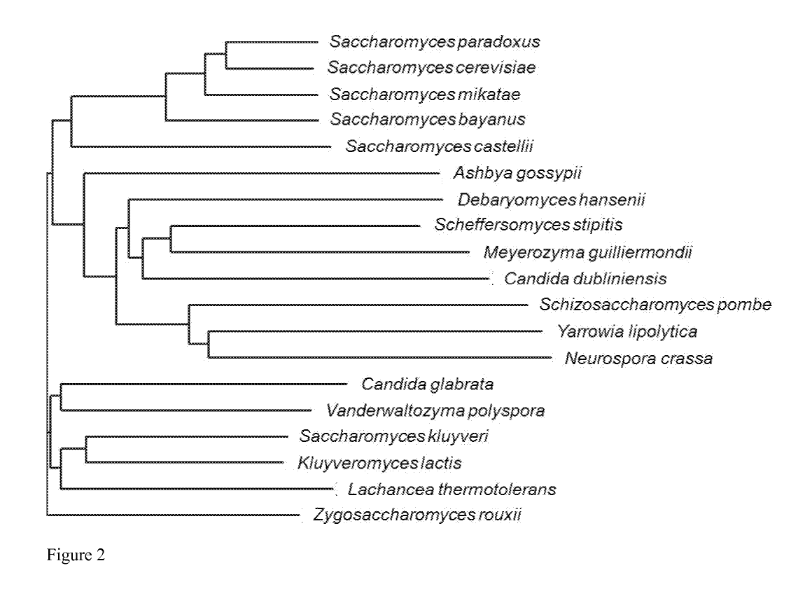

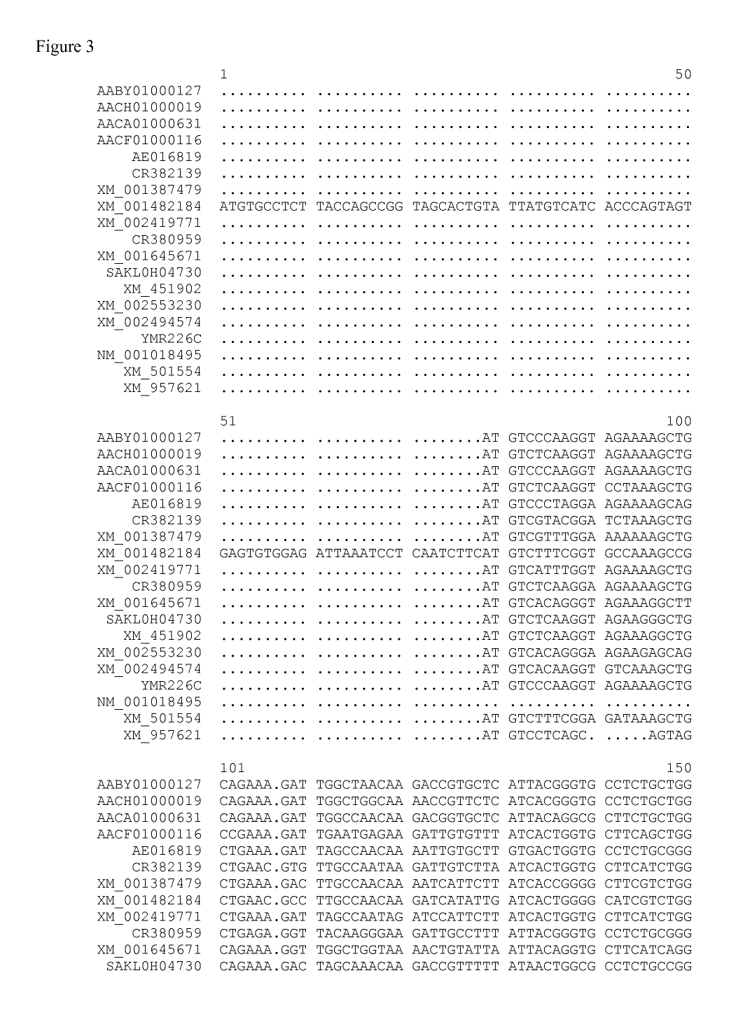

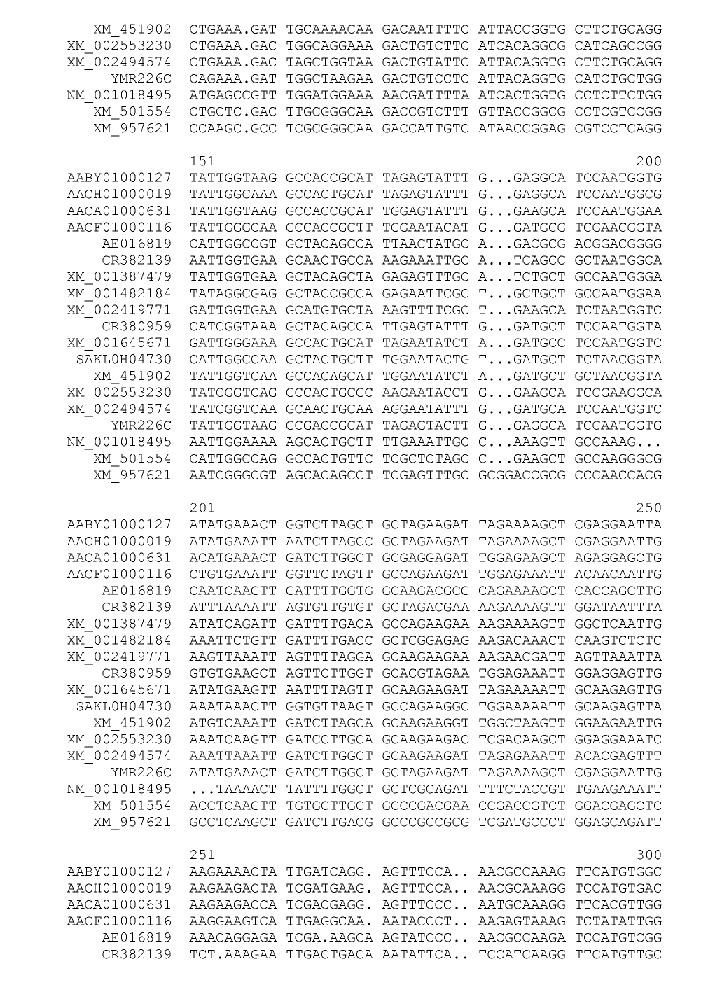

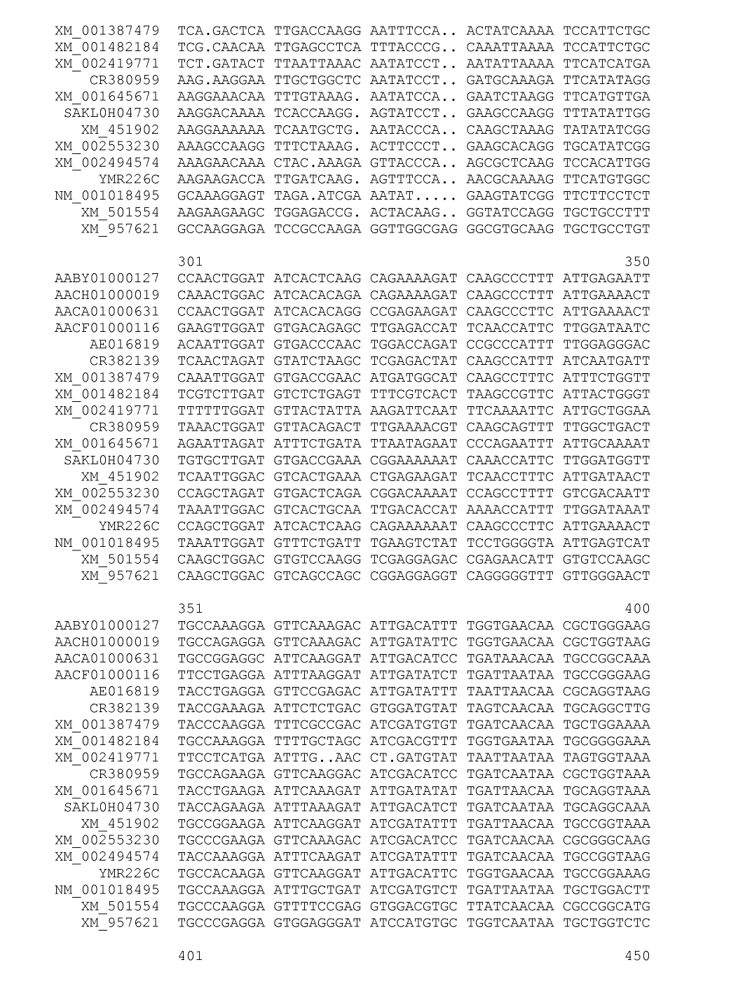

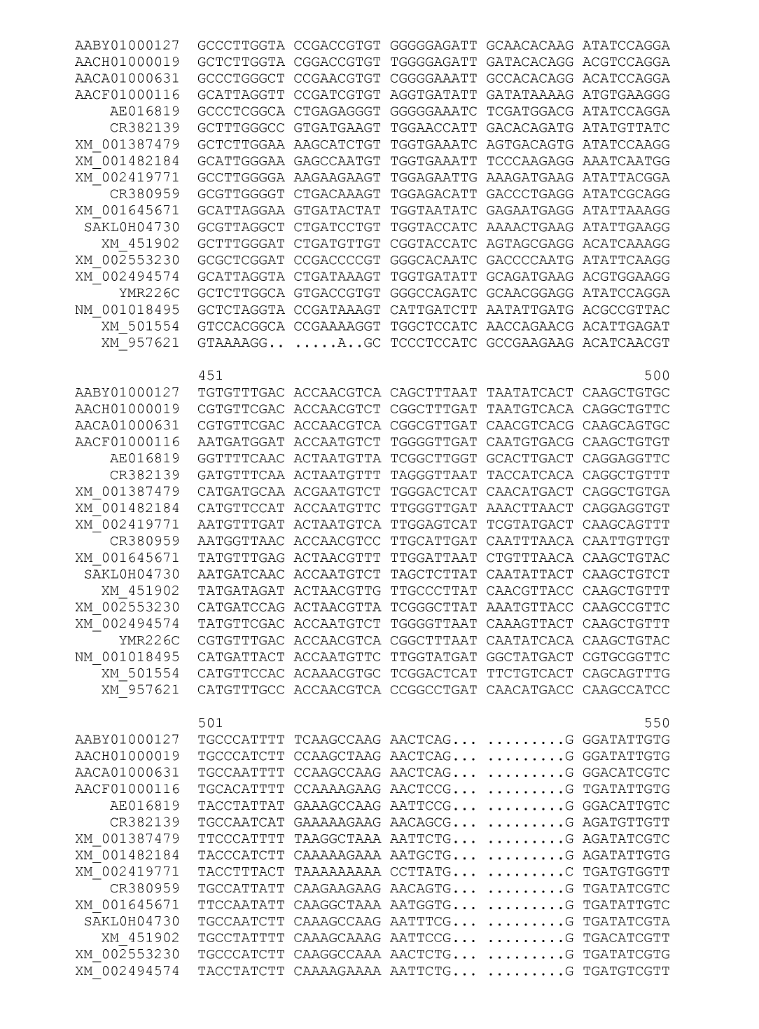

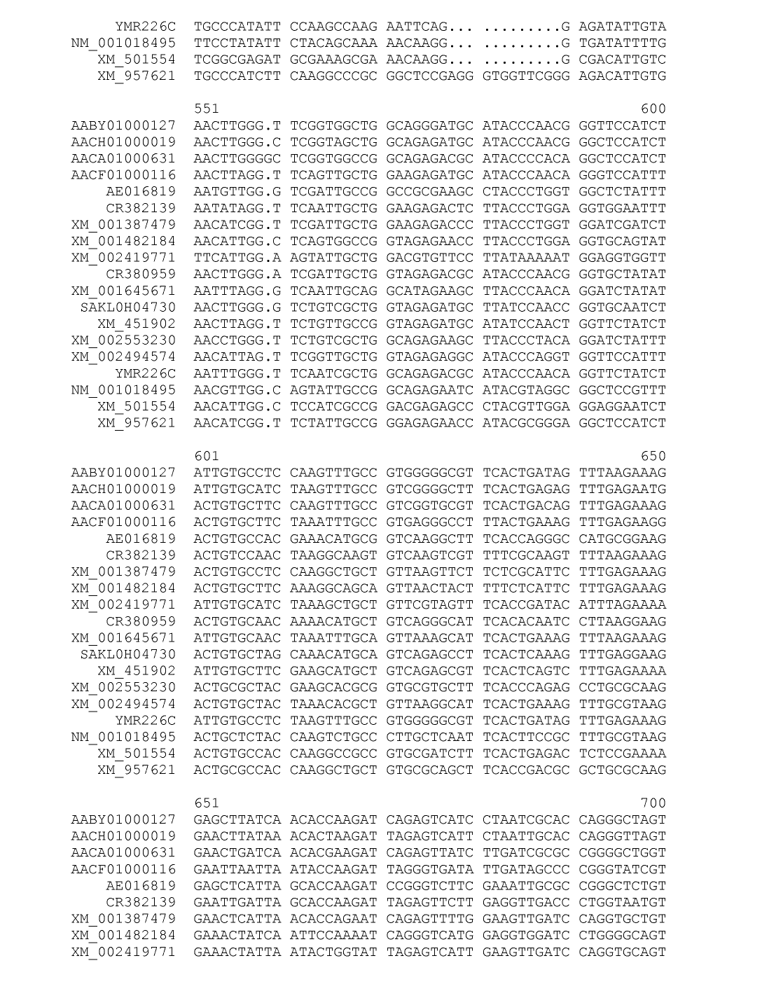

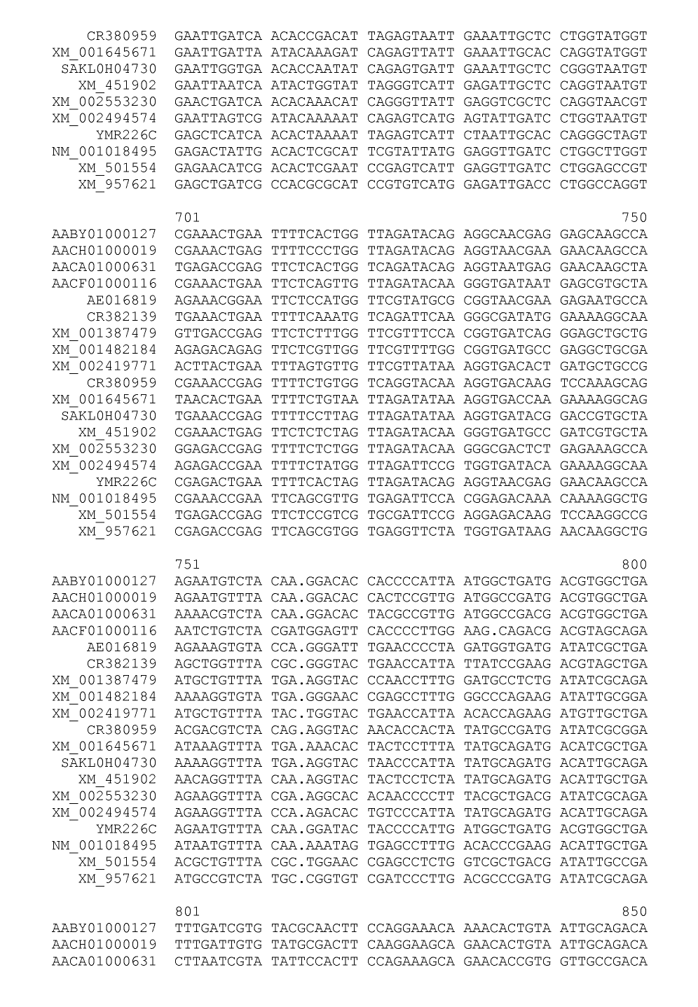

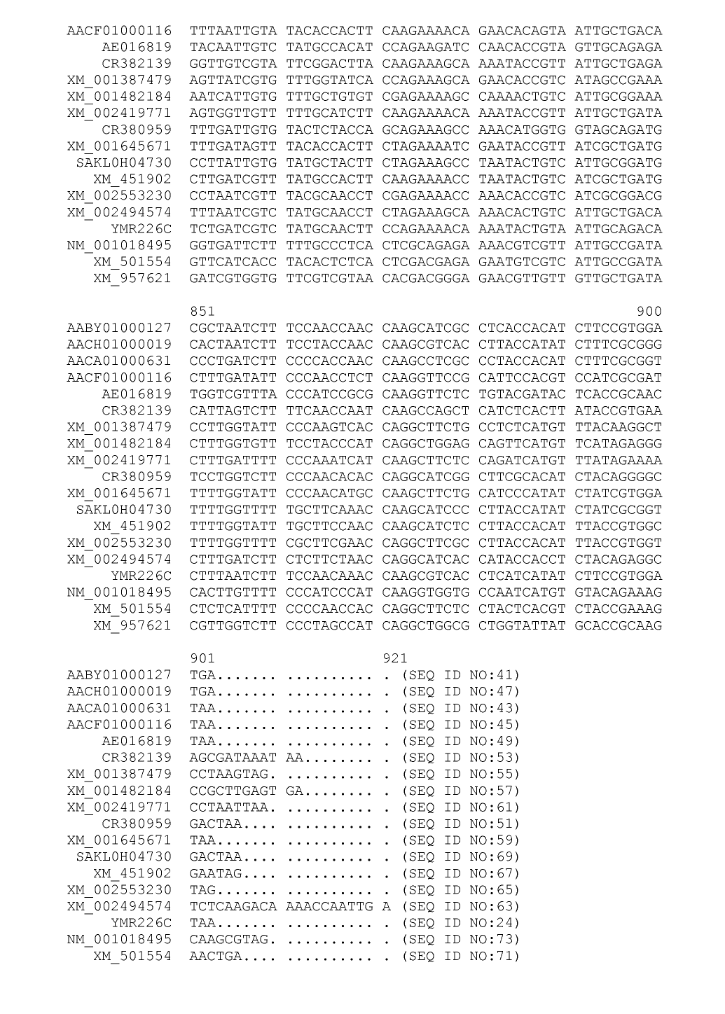

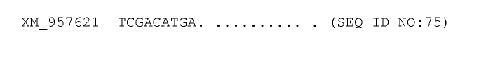

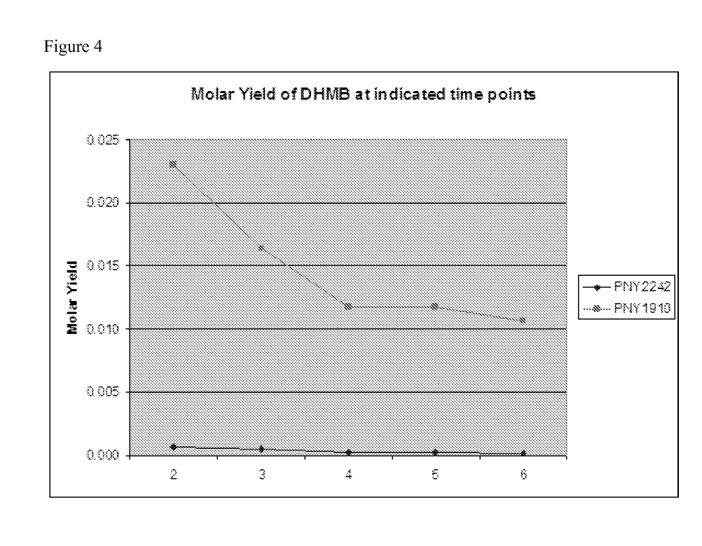

The 18 ORFs were aligned using AlignX (Vector NTI; the gene encoding a putative NADP+-dependent dehydrogenase from Neurospora crassa (XM_{—}957621, identified in the GenBank BLAST search using the YMR226C nucleotide sequence) was used as an outgroup. The resulting phylogenetic tree is shown in FIG. 2, and a sequence alignment is shown in FIG. 3.

The sequence identity of these homologs to YMR226C ranges from a minimum of 55% (Yarrowia hpolytica and Schizosaccharomyces pombe) to a maximum of 90% (S. paradoxus). A BLAST search also revealed a cDNA from S. pastorianus (accession number CJ997537) with 92% sequence identity over 484 base pairs, but since this species is a hybrid between S. bayanus (whose YMR226C homolog shows 82% identity to the S. cerevisiae sequence), and because only a partial ORF sequence was available, this sequence was not included in the comparison. When the YMR226C sequence from the canonical laboratory strain S288C was compared with the sequences from 12 other strains of S. cerevisiae, only 4 single-nucleotide polymorphisms are found (sequence identity 99.5%), indicating that this is a highly-conserved gene in that species.

TABLE 6
YMR226C Yeast Homologs

| | | |
|---|---|---|
| Species | Accession # | Sequence |

| | | |
|---|---|---|
| Saccharomyces | AABY01000127 | ATGTCCCAAGGTAGAAAAGCTGCAGAAAGATTGGCTAACAAGACCGTGCT |
| paradoxus | | CATTACGGGTGCCTCTGCTGGTATTGGTAAGGCCACCGCATTAGAGTATTT |
| | | GGAGGCATCCAATGGTGATATGAAACTGGTCTTAGCTGCTAGAAGATTAGA |
| | | AAAGCTCGAGGAATTAAAGAAAACTATTGATCAGGAGTTTCCAAACGCCA |
| | | AAGTTCATGTGGCCCAACTGGATATCACTCAAGCAGAAAAGATCAAGCCCT |
| | | TTATTGAGAATTTGCCAAAGGAGTTCAAAGACATTGACATTTTGGTGAACA |
| | | ACGCTGGGAAGGCCCTTGGTACCGACCGTGTGGGGGAGATTGCAACACAA |
| | | GATATCCAGGATGTGTTTGACACCAACGTCACAGCTTTAATTAATATCACT |
| | | CAAGCTGTGCTGCCCATTTTTCAAGCCAAGAACTCAGGGGATATTGTGAAC |
| | | TTGGGTTCGGTGGCTGGCAGGGATGCATACCCAACGGGTTCCATCTATTGT |
| | | GCCTCCAAGTTTGCCGTGGGGGCGTTCACTGATAGTTTAAGAAAGGAGCTT |
| | | ATCAACACCAAGATCAGAGTCATCCTAATCGCACCAGGGCTAGTCGAAACT |
| | | GAATTTTCACTGGTTAGATACAGAGGCAACGAGGAGCAAGCCAAGAATGT |
| | | CTACAAGGACACCACCCCATTAATGGCTGATGACGTGGCTGATTTGATCGT |
| | | GTACGCAACTTCCAGGAAACAAAACACTGTAATTGCAGACACGCTAATCTT |
| | | TCCAACCAACCAAGCATCGCCTCACCACATCTTCCGTGGATGA (SEQ ID |
| | | NO: 41) |
| | | MSQGRKAAERLANKTVLITGASAGIGKATALEYLEASNGDMKLVLAARRLEK |
| | | LEELKKTIDQEFPNAKVHVAQLDITQAEKIKPFIENLPKEFKDIDILVNNAGKAL |
| | | GTDRVGEIATQDIQDVFDTNVTALINITQAVLPIFQAKNSGDIVNLGSVAGRDA |
| | | YPTGSIYCASKFAVGAFTDSLRKELINTKIRVILIAPGLVETEFSLVRYRGNEEQ |
| | | AKNVYKDTTPLMADDVADLIVYATSRKQNTVIADTLIFPTNQASPHHIFRG* |
| | | (SEQ ID NO: 42) |
| Saccharomyces | AACA01000631 | ATGTCCCAAGGTAGAAAAGCTGCAGAAAGATTGGCCAACAAGACGGTGCT |
| bayanus | | CATTACAGGCGCTTCTGCTGGTATTGGTAAGGCCACCGCATTGGAGTATTT |
| | | GGAAGCATCCAATGGAAACATGAAACTGATCTTGGCTGCGAGGAGATTGG |
| | | AGAAGCTAGAGGAGCTGAAGAAGACCATCGACGAGGAGTTTCCCAATGCA |
| | | AAGGTTCACGTTGGCCAACTGGATATCACACAGGCCGAGAAGATCAAGCC |
| | | CTTCATTGAAAACTTGCCGGAGGCATTCAAGGATATTGACATCCTGATAAA |
| | | CAATGCCGGCAAAGCCCTGGGCTCCGAACGTGTCGGGGAAATTGCCACAC |
| | | AGGACATCCAGGACGTGTTCGACACCAACGTCACGGCGTTGATCAACGTCA |
| | | CGCAAGCAGTGCTGCCAATTTTCCAAGCCAAGAACTCAGGGGACATCGTCA |
| | | ACTTGGGGCTCGGTGGCCGGCAGAGACGCATACCCCACAGGCTCCATCTAC |
| | | TGTGCTTCCAAGTTTGCCGTCGGTGCGTTCACTGACAGTTTGAGAAAGGAA |
| | | CTGATCAACACGAAGATCAGAGTTATCTTGATCGCGCCGGGGCTGGTTGAG |
| | | ACCGAGTTCTCACTGGTCAGATACAGAGGTAATGAGGAACAAGCTAAAAA |
| | | CGTCTACAAGGACACTACGCCGTTGATGGCCGACGACGTGGCTGACTTAAT |
| | | CGTATATTCCACTTCCAGAAAGCAGAACACCGTGGTTGCCGACACCCTGAT |
| | | CTTCCCCACCAACCAAGCCTCGCCCTACCACATCTTTCGCGGTTAA (SEQ ID |
| | | NO: 43) |
| | | MSQGRKAAERLANKTVLITGASAGIGKATALEYLEASNGNMKLILAARRLEKL |
| | | EELKKTIDEEFPNAKVHVGQLDITQAEKIKPFIENLPEAFKDIDILINNAGKALGS |
| | | ERVGEIATQDIQDVFDTNVTALINVTQAVLPIFQAKNSGDIVNLGLGGRQRRIP |
| | | HRLHLLCFQVCRRCVH*QFEKGTDQHEDQSYLDRAGAG*DRVLTGQIQR**GT |
| | | S*KRLQGHYAVDGRRRG*LNRIFHFQKAEHRGCRHPDLPHQPSLALPHLSRL* |
| | | (SEQ ID NO: 44) |
| | | The sequence came from a comparative |
| | | genomics study using “draft” genome |
| | | sequences with 7-fold coverage |
| | | (Kellis et al, Nature 423: 241-254 (2003)). |
| Saccharomyces | AACF01000116 | ATGTCTCAAGGTCCTAAAGCTGCCGAAAGATTGAATGAGAAGATTGTGTTT |
| castellii | | ATCACTGGTGCTTCAGCTGGTATTGGGCAAGCCACCGCTTTGGAATACATG |
| | | GATGCGTCGAACGGTACTGTGAAATTGGTTCTAGTTGCCAGAAGATTGGAG |
| | | AAATTACAACAATTGAAGGAAGTCATTGAGGCAAAATACCCTAAGAGTAA |
| | | AGTCTATATTGGGAAGTTGGATGTGACAGAGCTTGAGACCATTCAACCATT |
| | | CTTGGATAATCTTCCTGAGGAATTTAAGGATATTGATATCTTGATTAATAAT |
| | | GCCGGGAAGGCATTAGGTTCCGATCGTGTAGGTGATATTGATATAAAAGAT |
| | | GTGAAGGGAATGATGGATACCAATGTCTTGGGGTTGATCAATGTGACGCAA |
| | | GCTGTGTTGCACATTTTCCAAAAGAAGAACTCCGGTGATATTGTGAACTTA |
| | | GGTTCAGTTGCTGGAAGAGATGCATACCCAACAGGGTCCATTTACTGTGCT |
| | | TCTAAATTTGCCGTGAGGGCCTTTACTGAAAGTTTGAGAAGGGAATTAATT |
| | | AATACCAAGATTAGGGTGATATTGATAGCCCCGGGTATCGTCGAAACTGAA |
| | | TTCTCAGTTGTTAGATACAAGGGTGATAATGAGCGTGCTAAATCTGTCTAC |
| | | GATGGAGTTCACCCCTTGGAAGCAGACGACGTAGCAGATTTAATTGTATAC |
| | | ACCACTTCAAGAAAACAGAACACAGTAATTGCTGACACTTTGATATTCCCA |
| | | ACCTCTCAAGGTTCCGCATTCCACGTCCATCGCGATTAA (SEQ ID NO: 45) |
| | | MSQGPKAAERLNEKIVFITGASAGIGQATALEYMDASNGTVKLVLVARRLEKL |
| | | QQLKEVIEAKYPKSKVYIGKLDVTELETIQPFLDNLPEEFKDIDILINNAGKALG |
| | | SDRVGDIDIKDVKGMMDTNVLGLINVTQAVLHIFQKKNSGDIVNLGSVAGRD |
| | | AYPTGSIYCASKFAVRAFTESLRRELINTKIRVILIAPGIVETEFSVVRYKGDNER |
| | | AKSVYDGVHPLEADDVADLIVYTTSRKQNTVIADTLIFPTSQGSAFHVHRD* |
| | | (SEQ ID NO: 46) |
| Saccharomyces | AACH01000019 | ATGTCTCAAGGTAGAAAAGCTGCAGAAAGATTGGCTGGCAAAACCGTTCTC |
| mikatae | | ATCACGGGTGCCTCTGCTGGTATTGGCAAAGCCACTGCATTAGAGTATTTG |
| | | GAGGCATCCAATGGCGATATGAAATTAATCTTAGCCGCTAGAAGATTAGAA |
| | | AAGCTCGAGGAATTGAAGAAGACTATCGATGAAGAGTTTCCAAACGCAAA |
| | | GGTCCATGTGACCAAACTGGACATCACACAGACAGAAAAGATCAAGCCCT |
| | | TTATTGAAAACTTGCCAGAGGAGTTCAAAGACATTGATATTCTGGTGAACA |
| | | ACGCTGGTAAGGCTCTTGGTACGGACCGTGTTGGGGAGATTGATACACAGG |
| | | ACGTCCAGGACGTGTTCGACACCAACGTCTCGGCTTTGATTAATGTCACAC |
| | | AGGCTGTTCTGCCCATCTTCCAAGCTAAGAACTCAGGGGATATTGTGAACT |
| | | TGGGCTCGGTAGCTGGCAGAGATGCATACCCAACGGGCTCCATCTATTGTG |
| | | CATCTAAGTTTGCCGTCGGGGCTTTCACTGAGAGTTTGAGAATGGAACTTA |
| | | TAAACACTAAGATTAGAGTCATTCTAATTGCACCAGGGTTAGTCGAAACTG |
| | | AGTTTTCCCTGGTTAGATACAGAGGTAACGAAGAACAAGCCAAGAATGTTT |
| | | ACAAGGACACCACTCCGTTGATGGCCGATGACGTGGCTGATTTGATTGTGT |
| | | ATGCGACTTCAAGGAAGCAGAACACTGTAATTGCAGACACACTAATCTTTC |
| | | CTACCAACCAAGCGTCACCTTACCATATCTTTCGCGGGTGA (SEQ ID NO: 47) |
| | | MSQGRKAAERLAGKTVLITGASAGIGKATALEYLEASNGDMKLILAARRLEKL |
| | | EELKKTIDEEFPNAKVHVTKLDITQTEKIKPFIENLPEEFKDIDILVNNAGKALG |
| | | TDRVGEIDTQDVQDVFDTNVSALINVTQAVLPIFQAKNSGDIVNLGSVAGRDA |
| | | YPTGSIYCASKFAVGAFTESLRMELINTKIRVILIAPGLVETEFSLVRYRGNEEQ |
| | | AKNVYKDTTPLMADDVADLIVYATSRKQNTVIADTLIFPTNQASPYHIFRG* |
| | | (SEQ ID NO: 48) |
| Ashbya | AE016819 | ATGTCCCTAGGAAGAAAAGCAGCTGAAAGATTAGCCAACAAAATTGTGCT |
| gossypii | | TGTGACTGGTGCCTCTGCGGGCATTGGCCGTGCTACAGCCATTAACTATGC |
| | | AGACGCGACGGACGGGGCAATCAAGTTGATTTTGGTGGCAAGACGCGCAG |
| | | AAAAGCTCACCAGCTTGAAACAGGAGATCGAAAGCAAGTATCCCAACGCC |
| | | AAGATCCATGTCGGACAATTGGATGTGACCCAACTGGACCAGATCCGCCCA |
| | | TTTTTGGAGGGACTACCTGAGGAGTTCCGAGACATTGATATTTTAATTAAC |
| | | AACGCAGGTAAGGCCCTCGGCACTGAGAGGGTGGGGGAAATCTCGATGGA |
| | | CGATATCCAGGAGGTTTTCAACACTAATGTTATCGGCTTGGTGCACTTGACT |
| | | CAGGAGGTTCTACCTATTATGAAAGCCAAGAATTCCGGGGACATTGTCAAT |
| | | GTTGGGTCGATTGCCGGCCGCGAAGCCTACCCTGGTGGCTCTATTTACTGT |
| | | GCCACGAAACATGCGGTCAAGGCTTTCACCAGGGCCATGCGGAAGGAGCT |
| | | CATTAGCACCAAGATCCGGGTCTTCGAAATTGCGCCGGGCTCTGTAGAAAC |
| | | GGAATTCTCCATGGTTCGTATGCGCGGTAACGAAGAGAATGCCAAGAAAG |
| | | TGTACCAGGGATTTGAACCCCTAGATGGTGATGATATCGCTGATACAATTG |
| | | TCTATGCCACATCCAGAAGATCCAACACCGTAGTTGCAGAGATGGTCGTTT |
| | | ACCCATCCGCGCAAGGTTCTCTGTACGATACTCACCGCAACTAA (SEQ ID |
| | | NO: 49) |
| | | MSLGRKAAERLANKIVLVTGASAGIGRATAINYADATDGAIKLILVARRAEKL |
| | | TSLKQEIESKYPNAKIHVGQLDVTQLDQIRPFLEGLPEEFRDIDILINNAGKALG |
| | | TERVGEISMDDIQEVFNTNVIGLVHLTQEVLPIMKAKNSGDIVNVGSIAGREAY |
| | | PGGSIYCATKHAVKAFTRAMRKELISTKIRVFEIAPGSVETEFSMVRMRGNEEN |
| | | AKKVYQGFEPLDGDDIADTIVYATSRRSNTVVAEMVVYPSAQGSLYDTHRN* |
| | | (SEQ ID NO: 50) |
| Candida | CR380959 | ATGTCTCAAGGAAGAAAAGCTGCTGAGAGGTTACAAGGGAAGATTGCCTT |
| glabrata | | TATTACGGGTGCCTCTGCGGGCATCGGTAAAGCTACAGCCATTGAGTATTT |
| | | GGATGCTTCCAATGGTAGTGTGAAGCTAGTTCTTGGTGCACGTAGAATGGA |
| | | GAAATTGGAGGAGTTGAAGAAGGAATTGCTGGCTCAATATCCTGATGCAA |
| | | AGATTCATATAGGTAAACTGGATGTTACAGACTTTGAAAACGTCAAGCAGT |
| | | TTTTGGCTGACTTGCCAGAAGAGTTCAAGGACATCGACATCCTGATCAATA |
| | | ACGCTGGTAAAGCGTTGGGGTCTGACAAAGTTGGAGACATTGACCCTGAG |
| | | GATATCGCAGGAATGGTTAACACCAACGTCCTTGCATTGATCAATTTAACA |
| | | CAATTGTTGTTGCCATTATTCAAGAAGAAGAACAGTGGTGATATCGTCAAC |
| | | TTGGGATCGATTGCTGGTAGAGACGCATACCCAACGGGTGCTATATACTGT |
| | | GCAACAAAACATGCTGTCAGGGCATTCACACAATCCTTAAGGAAGGAATT |
| | | GATCAACACCGACATTAGAGTAATTGAAATTGCTCCTGGTATGGTCGAAAC |
| | | CGAGTTTTCTGTGGTCAGGTACAAAGGTGACAAGTCCAAAGCAGACGACGT |
| | | CTACAGAGGTACAACACCACTATATGCCGATGATATCGCGGATTTGATTGT |
| | | GTACTCTACCAGCAGAAAGCCAAACATGGTGGTAGCAGATGTCCTGGTCTT |
| | | CCCAACACACCAGGCATCGGCTTCGCACATCTACAGGGGCGACTAA (SEQ |
| | | ID NO: 51) |
| | | MSQGRKAAERLQGKIAFITGASAGIGKATAIEYLDASNGSVKLVLGARRMEKL |
| | | EELKKELLAQYPDAKIHIGKLDVTDFENVKQFLADLPEEFKDIDILINNAGKAL |
| | | GSDKVGDIDPEDIAGMVNTNVLALINLTQLLLPLFKKKNSGDIVNLGSIAGRDA |
| | | YPTGAIYCATKHAVRAFTQSLRKELINTDIRVIEIAPGMVETEFSVVRYKGDKS |
| | | KADDVYRGTTPLYADDIADLIVYSTSRKPNMVVADVLVFPTHQASASHIYRGD |
| | | * (SEQ ID NO: 52) |
| Debaryomyces | CR382139 | ATGTCGTACGGATCTAAAGCTGCTGAACGTGTTGCCAATAAGATTGTCTTA |
| hansenii | | ATCACTGGTGCTTCATCTGGAATTGGTGAAGCAACTGCCAAAGAAATTGCA |
| | | TCAGCCGCTAATGGCAATTTAAAATTAGTGTTGTGTGCTAGACGAAAAGAA |
| | | AAGTTGGATAATTTATCTAAAGAATTGACTGACAAATATTCATCCATCAAG |
| | | GTTCATGTTGCTCAACTAGATGTATCTAAGCTCGAGACTATCAAGCCATTTA |
| | | TCAATGATTTACCGAAAGAATTCTCTGACGTGGATGTATTAGTCAACAATG |
| | | CAGGCTTGGCTTTGGGCCGTGATGAAGTTGGAACCATTGACACAGATGATA |
| | | TGTTATCGATGTTTCAAACTAATGTTTTAGGGTTAATTACCATCACACAGGC |
| | | TGTTTTGCCAATCATGAAAAAGAAGAACAGCGGAGATGTTGTTAATATAGG |
| | | TTCAATTGCTGGAAGAGACTCTTACCCTGGAGGTGGAATTTACTGTCCAAC |
| | | TAAGGCAAGTGTCAAGTCGTTTTCGCAAGTTTTAAGAAAGGAATTGATTAG |
| | | CACCAAGATTAGAGTTCTTGAGGTTGACCCTGGTAATGTTGAAACTGAATT |
| | | TTCAAATGTCAGATTCAAGGGCGATATGGAAAAGGCAAAGCTGGTTTACGC |
| | | GGGTACTGAACCATTATTATCCGAAGACGTAGCTGAGGTTGTCGTATTCGG |
| | | ACTTACAAGAAAGCAAAATACCGTTATTGCTGAGACATTAGTCTTTTCAAC |
| | | CAATCAAGCCAGCTCATCTCACTTATACCGTGAAAGCGATAAATAA (SEQ |
| | | ID NO: 53) |
| | | MSYGSKAAERVANKIVLITGASSGIGEATAKEIASAANGNLKLVLCARRKEKL |
| | | DNLSKELTDKYSSIKVHVAQLDVSKLETIKPFINDLPKEFSDVDVLVNNAGLAL |
| | | GRDEVGTIDTDDMLSMFQTNVLGLITITQAVLPIMKKKNSGDVVNIGSIAGRDS |
| | | YPGGGIYCPTKASVKSFSQVLRKELISTKIRVLEVDPGNVETEFSNVRFKGDME |
| | | KAKLVYAGTEPLLSEDVAEVVVFGLTRKQNTVIAETLVFSTNQASSSHLYRES |
| | | DK* (SEQ ID NO: 54) |
| Scheffersomyces | XM_001387479 | ATGTCGTTTGGAAAAAAAGCTGCTGAAAGACTTGCCAACAAAATCATTCTT |
| stipitis | | ATCACCGGGGCTTCGTCTGGTATTGGTGAAGCTACAGCTAGAGAGTTTGCA |
| (formerly | | TCTGCTGCCAATGGGAATATCAGATTGATTTTGACAGCCAGAAGAAAAGAA |
| Pichia | | AAGTTGGCTCAATTGTCAGACTCATTGACCAAGGAATTTCCAACTATCAAA |
| stipitis) | | ATCCATTCTGCCAAATTGGATGTGACCGAACATGATGGCATCAAGCCTTTC |
| | | ATTTCTGGTTTACCCAAGGATTTCGCCGACATCGATGTGTTGATCAACAATG |
| | | CTGGAAAAGCTCTTGGAAAAGCATCTGTTGGTGAAATCAGTGACAGTGATA |
| | | TCCAAGGCATGATGCAAACGAATGTCTTGGGACTCATCAACATGACTCAGG |
| | | CTGTGATTCCCATTTTTAAGGCTAAAAATTCTGGAGATATCGTCAACATCG |
| | | GTTCGATTGCTGGAAGAGACCCTTACCCTGGTGGATCGATCTACTGTGCCT |
| | | CCAAGGCTGCTGTTAAGTTCTTCTCGCATTCTTTGAGAAAGGAACTCATTAA |
| | | CACCAGAATCAGAGTTTTGGAAGTTGATCCAGGTGCTGTGTTGACCGAGTT |
| | | CTCTTTGGTTCGTTTCCACGGTGATCAGGGAGCTGCTGATGCTGTTTATGAA |
| | | GGTACCCAACCTTTGGATGCCTCTGATATCGCAGAAGTTATCGTGTTTGGTA |
| | | TCACCAGAAAGCAGAACACCGTCATAGCCGAAACCTTGGTATTCCCAAGTC |
| | | ACCAGGCTTCTGCCTCTCATGTTTACAAGGCTCCTAAGTAG (SEQ ID NO: 55) |
| | | MSFGKKAAERLANKIILITGASSGIGEATAREFASAANGNIRLILTARRKEKLAQ |
| | | LSDSLTKEFPTIKIHSAKLDVTEHDGIKPFISGLPKDFADIDVLINNAGKALGKAS |
| | | VGEISDSDIQGMMQTNVLGLINMTQAVIPIFKAKNSGDIVNIGSIAGRDPYPGGS |
| | | IYCASKAAVKFFSHSLRKELINTRIRVLEVDPGAVLTEFSLVRFHGDQGAADAV |
| | | YEGTQPLDASDIAEVIVFGITRKQNTVIAETLVFPSHQASASHVYKAPK* (SEQ |
| | | ID NO: 56) |
| Meyerozyma | XM_001482184 | ATGTGCCTCTTACCAGCCGGTAGCACTGTATTATGTCATCACCCAGTAGTG |
| guilliermondii | | AGTGTGGAGATTAAATCCTCAATCTTCATGTCTTTCGGTGCCAAAGCCGCT |
| (formerly | | GAACGCCTTGCCAACAAGATCATATTGATCACTGGGGCATCGTCTGGTATA |
| Pichia | | GGCGAGGCTACCGCCAGAGAATTCGCTGCTGCTGCCAATGGAAAAATTCTG |
| guilliermondii) | | TTGATTTTGACCGCTCGGAGAGAAGACAAACTCAAGTCTCTCTCGCAACAA |
| | | TTGAGCCTCATTTACCCGCAAATTAAAATCCATTCTGCTCGTCTTGATGTCT |
| | | CTGAGTTTTCGTCACTTAAGCCGTTCATTACTGGGTTGCCAAAGGATTTTGC |
| | | TAGCATCGACGTTTTGGTGAATAATGCGGGGAAAGCATTGGGAAGAGCCA |
| | | ATGTTGGTGAAATTTCCCAAGAGGAAATCAATGGCATGTTCCATACCAATG |
| | | TTCTTGGGTTGATAAACTTAACTCAGGAGGTGTTACCCATCTTCAAAAAGA |
| | | AAAATGCTGGAGATATTGTGAACATTGGCTCAGTGGCCGGTAGAGAACCTT |
| | | ACCCTGGAGGTGCAGTATACTGTGCTTCAAAGGCAGCAGTTAACTACTTTT |
| | | CTCATTCTTTGAGAAAGGAAACTATCAATTCCAAAATCAGGGTCATGGAGG |
| | | TGGATCCTGGGGCAGTAGAGACAGAGTTCTCGTTGGTTCGTTTTGGCGGTG |
| | | ATGCCGAGGCTGCGAAAAAGGTGTATGAGGGAACCGAGCCTTTGGGCCCA |
| | | GAAGATATTGCGGAAATCATTGTGTTTGCTGTGTCGAGAAAAGCCAAAACT |
| | | GTCATTGCGGAAACTTTGGTGTTTCCTACCCATCAGGCTGGAGCAGTTCAT |
| | | GTTCATAGAGGGCCGCTTGAGTGA (SEQ ID NO: 57) |
| | | MCLLPAGSTVLCHHPVVSVEIKSSIFMSFGAKAAERLANKIILITGASSGIGEAT |
| | | AREFAAAANGKILLILTARREDKLKSLSQQLSLIYPQIKIHSARLDVSEFSSLKPF |
| | | ITGLPKDFASIDVLVNNAGKALGRANVGEISQEEINGMFHTNVLGLINLTQEVL |
| | | PIFKKKNAGDIVNIGSVAGREPYPGGAVYCASKAAVNYFSHSLRKETINSKIRV |
| | | MEVDPGAVETEFSLVRFGGDAEAAKKVYEGTEPLGPEDIAEIIVFAVSRKAKT |
| | | VIAETLVFPTHQAGAVHVHRGPLE* (SEQ ID NO: 58) |
| Vanderwaltozyma | XM_001645671 | ATGTCACAGGGTAGAAAGGCTTCAGAAAGGTTGGCTGGTAAAACTGTATTA |
| polyspora | | ATTACAGGTGCTTCATCAGGGATTGGGAAAGCCACTGCATTAGAATATCTA |
| (formerly | | GATGCCTCCAATGGTCATATGAAGTTAATTTTAGTTGCAAGAAGATTAGAA |
| Kluyveromyces | | AAATTGCAAGAGTTGAAGGAAACAATTTGTAAAGAATATCCAGAATCTAA |
| polysporus) | | GGTTCATGTTGAAGAATTAGATATTTCTGATATTAATAGAATCCCAGAATTT |
| | | ATTGCAAAATTACCTGAAGAATTCAAAGATATTGATATATTGATTAACAAT |
| | | GCAGGTAAAGCATTAGGAAGTGATACTATTGGTAATATCGAGAATGAGGA |
| | | TATTAAAGGTATGTTTGAGACTAACGTTTTTGGATTAATCTGTTTAACACAA |
| | | GCTGTACTTCCAATATTCAAGGCTAAAAATGGTGGTGATATTGTCAATTTA |
| | | GGGTCAATTGCAGGCATAGAAGCTTACCCAACAGGATCTATATATTGTGCA |
| | | ACTAAATTTGCAGTTAAAGCATTCACTGAAAGTTTAAGAAAGGAATTGATT |
| | | AATACAAAGATCAGAGTTATTGAAATTGCACCAGGTATGGTTAACACTGAA |
| | | TTTTCTGTAATTAGATATAAAGGTGACCAAGAAAAGGCAGATAAAGTTTAT |
| | | GAAAACACTACTCCTTTATATGCAGATGACATCGCTGATTTGATAGTTTAC |
| | | ACCACTTCTAGAAAATCGAATACCGTTATCGCTGATGTTTTGGTATTCCCAA |
| | | CATGCCAAGCTTCTGCATCCCATATCTATCGTGGATAA (SEQ ID NO: 59) |
| | | MSQGRKASERLAGKTVLITGASSGIGKATALEYLDASNGHMKLILVARRLEKL |
| | | QELKETICKEYPESKVHVEELDISDINRIPEFIAKLPEEFKDIDILINNAGKALGSD |
| | | TIGNIENEDIKGMFETNVFGLICLTQAVLPIFKAKNGGDIVNLGSIAGIEAYPTGS |
| | | IYCATKFAVKAFTESLRKELINTKIRVIEIAPGMVNTEFSVIRYKGDQEKADKV |
| | | YENTTPLYADDIADLIVYTTSRKSNTVIADVLVFPTCQASASHIYRG* (SEQ ID |
| | | NO: 60) |
| Candida | XM_002419771 | ATGTCATTTGGTAGAAAAGCTGCTGAAAGATTAGCCAATAGATCCATTCTT |
| dubliniensis | | ATCACTGGTGCTTCATCTGGGATTGGTGAAGCATGTGCTAAAGTTTTCGCTG |
| | | AAGCATCTAATGGTCAAGTTAAATTAGTTTTAGGAGCAAGAAGAAAAGAA |
| | | CGATTAGTTAAATTATCTGATACTTTAATTAAACAATATCCTAATATTAAAA |
| | | TTCATCATGATTTTTTGGATGTTACTATTAAAGATTCAATTTCAAAATTCAT |
| | | TGCTGGAATTCCTCATGAATTTGAACCTGATGTATTAATTAATAATAGTGGT |
| | | AAAGCCTTGGGGAAAGAAGAAGTTGGAGAATTGAAAGATGAAGATATTAC |
| | | GGAAATGTTTGATACTAATGTCATTGGAGTCATTCGTATGACTCAAGCAGT |
| | | TTTACCTTTACTTAAAAAAAAACCTTATGCTGATGTGGTTTTCATTGGAAGT |
| | | ATTGCTGGACGTGTTCCTTATAAAAATGGAGGTGGTTATTGTGCATCTAAA |
| | | GCTGCTGTTCGTAGTTTCACCGATACATTTAGAAAAGAAACTATTAATACT |
| | | GGTATTAGAGTCATTGAAGTTGATCCAGGTGCAGTACTTACTGAATTTAGT |
| | | GTTGTTCGTTATAAAGGTGACACTGATGCTGCCGATGCTGTTTATACTGGTA |
| | | CTGAACCATTAACACCAGAAGATGTTGCTGAAGTGGTTGTTTTTGCATCTTC |
| | | AAGAAAACAAAATACCGTTATTGCTGATACTTTGATTTTCCCAAATCATCA |
| | | AGCTTCTCCAGATCATGTTTATAGAAAACCTAATTAA (SEQ ID NO: 61) |
| | | MSFGRKAAERLANRSILITGASSGIGEACAKVFAEASNGQVKLVLGARRKERL |
| | | VKLSDTLIKQYPNIKIHHDFLDVTIKDSISKFIAGIPHEFEPDVLINNSGKALGKE |
| | | EVGELKDEDITEMFDTNVIGVIRMTQAVLPLLKKKPYADVVFIGSIAGRVPYKN |
| | | GGGYCASKAAVRSFTDTFRKETINTGIRVIEVDPGAVLTEFSVVRYKGDTDAA |
| | | DAVYTGTEPLTPEDVAEVVVFASSRKQNTVIADTLIFPNHQASPDHVYRKPN* |
| | | (SEQ ID NO: 62) |
| Zygosaccharomyces | XM_002494574 | ATGTCACAAGGTGTCAAAGCTGCTGAAAGACTAGCTGGTAAGACTGTATTC |
| rouxii | | ATTACAGGTGCTTCTGCAGGTATCGGTCAAGCAACTGCAAAGGAATATTTG |
| | | GATGCATCCAATGGTCAAATTAAATTGATCTTGGCTGCAAGAAGATTAGAG |
| | | AAATTACACGAGTTTAAAGAACAAACTACAAAGAGTTACCCAAGCGCTCA |
| | | AGTCCACATTGGTAAATTGGACGTCACTGCAATTGACACCATAAAACCATT |
| | | TTTGGATAAATTACCAAAGGAATTTCAAGATATCGATATTTTGATCAACAA |
| | | TGCCGGTAAGGCATTAGGTACTGATAAAGTTGGTGATATTGCAGATGAAGA |
| | | CGTGGAAGGTATGTTCGACACCAATGTCTTGGGGTTAATCAAAGTTACTCA |
| | | AGCTGTTTTACCTATCTTCAAAAGAAAAAATTCTGGTGATGTCGTTAACATT |
| | | AGTTCGGTTGCTGGTAGAGAGGCATACCCAGGTGGTTCCATTTACTGTGCT |
| | | ACTAAACACGCTGTTAAGGCATTCACTGAAAGTTTGCGTAAGGAATTAGTC |
| | | GATACAAAAATCAGAGTCATGAGTATTGATCCTGGTAATGTAGAGACCGA |
| | | ATTTTCTATGGTTAGATTCCGTGGTGATACAGAAAAGGCAAAGAAGGTTTA |
| | | CCAAGACACTGTCCCATTATATGCAGATGACATTGCAGATTTAATCGTCTA |
| | | TGCAACCTCTAGAAAGCAAAACACTGTCATTGCTGACACTTTGATCTTCTCT |
| | | TCTAACCAGGCATCACCATACCACCTCTACAGAGGCTCTCAAGACAAAACC |
| | | AATTGA (SEQ ID NO: 63) |
| | | MSQGVKAAERLAGKTVFITGASAGIGQATAKEYLDASNGQIKLILAARRLEKL |
| | | HEFKEQTTKSYPSAQVHIGKLDVTAIDTIKPFLDKLPKEFQDIDILINNAGKALG |
| | | TDKVGDIADEDVEGMFDTNVLGLIKVTQAVLPIFKRKNSGDVVNISSVAGREA |
| | | YPGGSIYCATKHAVKAFTESLRKELVDTKIRVMSIDPGNVETEFSMVRFRGDTE |
| | | KAKKVYQDTVPLYADDIADLIVYATSRKQNTVIADTLIFSSNQASPYHLYRGS |
| | | QDKTN* (SEQ ID NO: 64) |
| Lachancea | XM_002553230 | ATGTCACAGGGAAGAAGAGCAGCTGAAAGACTGGCAGGAAAGACTGTCTT |
| thermotolerans | | CATCACAGGCGCATCAGCCGGTATCGGTCAGGCCACTGCGCAAGAATACCT |
| (formerly | | GGAAGCATCCGAAGGCAAAATCAAGTTGATCCTTGCAGCAAGAAGACTCG |
| Kluyveromyces | | ACAAGCTGGAGGAAATCAAAGCCAAGGTTTCTAAAGACTTCCCTGAAGCA |
| thermotolerans) | | CAGGTGCATATCGGCCAGCTAGATGTGACTCAGACGGACAAAATCCAGCCT |
| | | TTTGTCGACAATTTGCCCGAAGAGTTCAAAGACATCGACATCCTGATCAAC |
| | | AACGCGGGCAAGGCGCTCGGATCCGACCCCGTGGGCACAATCGACCCCAA |
| | | TGATATTCAAGGCATGATCCAGACTAACGTTATCGGGCTTATAAATGTTAC |
| | | CCAAGCCGTTCTGCCCATCTTCAAGGCCAAAAACTCTGGTGATATCGTGAA |
| | | CCTGGGTTCTGTCGCTGGCAGAGAAGCTTACCCTACAGGATCTATTTACTG |
| | | CGCTACGAAGCACGCGGTGCGTGCTTTCACCCAGAGCCTGCGCAAGGAACT |
| | | GATCAACACAAACATCAGGGTTATTGAGGTCGCTCCAGGTAACGTGGAGA |
| | | CCGAGTTTTCTCTGGTTAGATACAAGGGCGACTCTGAGAAAGCCAAGAAGG |
| | | TTTACGAAGGCACACAACCCCTTTACGCTGACGATATCGCAGACCTAATCG |
| | | TTTACGCAACCTCGAGAAAACCAAACACCGTCATCGCGGACGTTTTGGTTT |
| | | TCGCTTCGAACCAGGCTTCGCCTTACCACATTTACCGTGGTTAG (SEQ ID |
| | | NO: 65) |
| | | MSQGRRAAERLAGKTVFITGASAGIGQATAQEYLEASEGKIKLILAARRLDKL |
| | | EEIKAKVSKDFPEAQVHIGQLDVTQTDKIQPFVDNLPEEFKDIDILINNAGKALG |
| | | SDPVGTIDPNDIQGMIQTNVIGLINVTQAVLPIFKAKNSGDIVNLGSVAGREAYP |
| | | TGSIYCATKHAVRAFTQSLRKELINTNIRVIEVAPGNVETEFSLVRYKGDSEKA |
| | | KKVYEGTQPLYADDIADLIVYATSRKPNTVIADVLVFASNQASPYHIYRG* |
| | | (SEQ ID NO: 66) |
| Kluyveromyces | XM_451902 | ATGTCTCAAGGTAGAAAGGCTGCTGAAAGATTGCAAAACAAGACAATTTTC |
| lactis | | ATTACCGGTGCTTCTGCAGGTATTGGTCAAGCCACAGCATTGGAATATCTA |
| | | GATGCTGCTAACGGTAATGTCAAATTGATCTTAGCAGCAAGAAGGTTGGCT |
| | | AAGTTGGAAGAATTGAAGGAAAAAATCAATGCTGAATACCCACAAGCTAA |
| | | AGTATATATCGGTCAATTGGACGTCACTGAAACTGAGAAGATTCAACCTTT |
| | | CATTGATAACTTGCCGGAAGAATTCAAGGATATCGATATTTTGATTAACAA |
| | | TGCCGGTAAAGCTTTGGGATCTGATGTTGTCGGTACCATCAGTAGCGAGGA |
| | | CATCAAAGGTATGATAGATACTAACGTTGTTGCCCTTATCAACGTTACCCA |
| | | AGCTGTTTTGCCTATTTTCAAAGCAAAGAATTCCGGTGACATCGTTAACTTA |
| | | GGTTCTGTTGCCGGTAGAGATGCATATCCAACTGGTTCTATCTATTGTGCTT |
| | | CGAAGCATGCTGTCAGAGCGTTCACTCAGTCTTTGAGAAAAGAATTAATCA |
| | | ATACTGGTATTAGGGTCATTGAGATTGCTCCAGGTAATGTCGAAACTGAGT |
| | | TCTCTCTAGTTAGATACAAGGGTGATGCCGATCGTGCTAAACAGGTTTACA |
| | | AAGGTACTACTCCTCTATATGCAGATGACATTGCTGACTTGATCGTTTATGC |
| | | CACTTCAAGAAAACCTAATACTGTCATCGCTGATGTTTTGGTATTTGCTTCC |
| | | AACCAAGCATCTCCTTACCACATTTACCGTGGCGAATAG (SEQ ID NO: 67) |
| | | MSQGRKAAERLQNKTIFITGASAGIGQATALEYLDAANGNVKLILAARRLAKL |
| | | EELKEKINAEYPQAKVYIGQLDVTETEKIQPFIDNLPEEFKDIDILINNAGKALGS |
| | | DVVGTISSEDIKGMIDTNVVALINVTQAVLPIFKAKNSGDIVNLGSVAGRDAYP |
| | | TGSIYCASKHAVRAFTQSLRKELINTGIRVIEIAPGNVETEFSLVRYKGDADRAK |
| | | QVYKGTTPLYADDIADLIVYATSRKPNTVIADVLVFASNQASPYHIYRGE* |
| | | (SEQ ID NO: 68) |
| Saccharomyces | SAKL0H04730 | ATGTCTCAAGGTAGAAGGGCTGCAGAAAGACTAGCAAACAAGACCGTTTT |
| kluyveri | | TATAACTGGCGCCTCTGCCGGCATTGGCCAAGCTACTGCTTTGGAATACTG |
| | | TGATGCTTCTAACGGTAAAATAAACTTGGTGTTAAGTGCCAGAAGGCTGGA |
| | | AAAATTGCAAGAGTTAAAGGACAAAATCACCAAGGAGTATCCTGAAGCCA |
| | | AGGTTTATATTGGTGTGCTTGATGTGACCGAAACGGAAAAAATCAAACCAT |
| | | TCTTGGATGGTTTACCAGAAGAATTTAAAGATATTGACATCTTGATCAATA |
| | | ATGCAGGCAAAGCGTTAGGCTCTGATCCTGTTGGTACCATCAAAACTGAAG |
| | | ATATTGAAGGAATGATCAACACCAATGTCTTAGCTCTTATCAATATTACTC |
| | | AAGCTGTCTTGCCAATCTTCAAAGCCAAGAATTTCGGTGATATCGTAAACT |
| | | TGGGGTCTGTCGCTGGTAGAGATGCTTATCCAACCGGTGCAATCTACTGTG |
| | | CTAGCAAACATGCAGTCAGAGCCTTCACTCAAAGTTTGAGGAAGGAATTGG |
| | | TGAACACCAATATCAGAGTGATTGAAATTGCTCCGGGTAATGTTGAAACCG |
| | | AGTTTTCCTTAGTTAGATATAAAGGTGATACGGACCGTGCTAAAAAGGTTT |
| | | ATGAAGGTACTAACCCATTATATGCAGATGACATTGCAGACCTTATTGTGT |
| | | ATGCTACTTCTAGAAAGCCTAATACTGTCATTGCGGATGTTTTGGTTTTTGC |
| | | TTCAAACCAAGCATCCCCTTACCATATCTATCGCGGTGACTAA (SEQ ID |
| | | NO: 69) |
| | | MSQGRRAAERLANKTVFITGASAGIGQATALEYCDASNGKINLVLSARRLEKL |
| | | QELKDKITKEYPEAKVYIGVLDVTETEKIKPFLDGLPEEFKDIDILINNAGKALG |
| | | SDPVGTIKTEDIEGMINTNVLALINITQAVLPIFKAKNFGDIVNLGSVAGRDAYP |
| | | TGAIYCASKHAVRAFTQSLRKELVNTNIRVIEIAPGNVETEFSLVRYKGDTDRA |
| | | KKVYEGTNPLYADDIADLIVYATSRKPNTVIADVLVFASNQASPYHIYRGD* |
| | | (SEQ ID NO: 70) |
| Yarrowia | XM_501554 | ATGTCTTTCGGAGATAAAGCTGCTGCTCGACTTGCGGGCAAGACCGTCTTT |
| lipolytica | | GTTACCGGCGCCTCGTCCGGCATTGGCCAGGCCACTGTTCTCGCTCTAGCC |
| | | GAAGCTGCCAAGGGCGACCTCAAGTTTGTGCTTGCTGCCCGACGAACCGAC |
| | | CGTCTGGACGAGCTCAAGAAGAAGCTGGAGACCGACTACAAGGGTATCCA |
| | | GGTGCTGCCTTTCAAGCTGGACGTGTCCAAGGTCGAGGAGACCGAGAACAT |
| | | TGTGTCCAAGCTGCCCAAGGAGTTTTCCGAGGTGGACGTGCTTATCAACAA |
| | | CGCCGGCATGGTCCACGGCACCGAAAAGGTTGGCTCCATCAACCAGAACG |
| | | ACATTGAGATCATGTTCCACACAAACGTGCTCGGACTCATTTCTGTCACTCA |
| | | GCAGTTTGTCGGCGAGATGCGAAAGCGAAACAAGGGCGACATTGTCAACA |
| | | TTGGCTCCATCGCCGGACGAGAGCCCTACGTTGGAGGAGGAATCTACTGTG |
| | | CCACCAAGGCCGCCGTGCGATCTTTCACTGAGACTCTCCGAAAAGAGAACA |
| | | TCGACACTCGAATCCGAGTCATTGAGGTTGATCCTGGAGCCGTTGAGACCG |
| | | AGTTCTCCGTCGTGCGATTCCGAGGAGACAAGTCCAAGGCCGACGCTGTTT |
| | | ACGCTGGAACCGAGCCTCTGGTCGCTGACGATATTGCCGAGTTCATCACCT |
| | | ACACTCTCACTCGACGAGAGAATGTCGTCATTGCCGATACTCTCATTTTCCC |
| | | CAACCACCAGGCTTCTCCTACTCACGTCTACCGAAAGAACTGA (SEQ ID |
| | | NO: 71) |
| | | MSFGDKAAARLAGKTVFVTGASSGIGQATVLALAEAAKGDLKFVLAARRTDR |
| | | LDELKKKLETDYKGIQVLPFKLDVSKVEETENIVSKLPKEFSEVDVLINNAGMV |
| | | HGTEKVGSINQNDIEIMFHTNVLGLISVTQQFVGEMRKRNKGDIVNIGSIAGRE |
| | | PYVGGGIYCATKAAVRSFTETLRKENIDTRIRVIEVDPGAVETEFSVVRFRGDK |
| | | SKADAVYAGTEPLVADDIAEFITYTLTRRENVVIADTLIFPNHQASPTHVYRKN |
| | | * (SEQ ID NO: 72) |
| Schizosaccharomyces | NM_001018495 | ATGAGCCGTTTGGATGGAAAAACGATTTTAATCACTGGTGCCTCTTCTGGA |
| pombe | | ATTGGAAAAAGCACTGCTTTTGAAATTGCCAAAGTTGCCAAAGTAAAACTT |
| | | ATTTTGGCTGCTCGCAGATTTTCTACCGTTGAAGAAATTGCAAAGGAGTTA |
| | | GAATCGAAATATGAAGTATCGGTTCTTCCTCTTAAATTGGATGTTTCTGATT |
| | | TGAAGTCTATTCCTGGGGTAATTGAGTCATTGCCAAAGGAATTTGCTGATA |
| | | TCGATGTCTTGATTAATAATGCTGGACTTGCTCTAGGTACCGATAAAGTCAT |
| | | TGATCTTAATATTGATGACGCCGTTACCATGATTACTACCAATGTTCTTGGT |
| | | ATGATGGCTATGACTCGTGCGGTTCTTCCTATATTCTACAGCAAAAACAAG |
| | | GGTGATATTTTGAACGTTGGCAGTATTGCCGGCAGAGAATCATACGTAGGC |
| | | GGCTCCGTTTACTGCTCTACCAAGTCTGCCCTTGCTCAATTCACTTCCGCTT |
| | | TGCGTAAGGAGACTATTGACACTCGCATTCGTATTATGGAGGTTGATCCTG |
| | | GCTTGGTCGAAACCGAATTCAGCGTTGTGAGATTCCACGGAGACAAACAA |
| | | AAGGCTGATAATGTTTACAAAAATAGTGAGCCTTTGACACCCGAAGACATT |
| | | GCTGAGGTGATTCTTTTTGCCCTCACTCGCAGAGAAAACGTCGTTATTGCCG |
| | | ATACACTTGTTTTCCCATCCCATCAAGGTGGTGCCAATCATGTGTACAGAA |
| | | AGCAAGCGTAG (SEQ ID NO: 73) |
| | | MSRLDGKTILITGASSGIGKSTAFEIAKVAKVKLILAARRFSTVEEIAKELESKY |
| | | EVSVLPLKLDVSDLKSIPGVIESLPKEFADIDVLINNAGLALGTDKVIDLNIDDA |
| | | VTMITTNVLGMMAMTRAVLPIFYSKNKGDILNVGSIAGRESYVGGSVYCSTKS |
| | | ALAQFTSALRKETIDTRIRIMEVDPGLVETEFSVVRFHGDKQKADNVYKNSEPL |
| | | TPEDIAEVILFALTRRENVVIADTLVFPSHQGGANHVYRKQA* (SEQ ID NO: 74) |

TABLE 7
YMR226C Homolog Percent Identity

| Species | Sm | Sb | Sca | Ag | Dh | Ss | Mg | Cd | Cg | Vp | Sk | Kl | Lt | Zr | Sce | Sp | Yl | Nc |
|---|---|---|---|---|---|---|---|---|---|---|---|---|---|---|---|---|---|---|
| Saccharomyces paradoxus (“Spa”) | 88 | 82 | 70 | 64 | 62 | 62 | 58 | 57 | 67 | 68 | 68 | 69 | 68 | 68 | 90 | 55 | 55 | 56 |
| Saccharomyces mikatae (“Sm”) | | 82 | 70 | 64 | 60 | 62 | 58 | 56 | 67 | 69 | 68 | 70 | 68 | 69 | 86 | 57 | 56 | 57 |
| Saccharomyces bayanus (“Sb”) | | | 71 | 63 | 59 | 62 | 58 | 53 | 67 | 66 | 68 | 70 | 69 | 67 | 82 | 56 | 56 | 58 |
| Saccharomyces castellii (“Sca”) | | | | 60 | 62 | 61 | 60 | 59 | 65 | 69 | 69 | 71 | 64 | 70 | 69 | 57 | 53 | 54 |
| Ashbya gossypii (“Ag”) | | | | | 56 | 60 | 57 | 54 | 59 | 61 | 62 | 62 | 62 | 62 | 63 | 54 | 55 | 55 |
| Debaryomyces hansenii (“Dh”) | | | | | | 64 | 62 | 61 | 61 | 63 | 62 | 61 | 59 | 63 | 62 | 57 | 57 | 53 |
| Scheffersomyces stipitis (“Ss”) | | | | | | | 68 | 64 | 61 | 62 | 62 | 64 | 62 | 63 | 62 | 56 | 58 | 58 |
| Meyerozyma guilliermondii (“Mg”) | | | | | | | | 60 | 57 | 58 | 60 | 60 | 59 | 62 | 59 | 57 | 57 | 56 |
| Candida dubliniensis (“Cd”) | | | | | | | | | 57 | 62 | 59 | 60 | 54 | 60 | 58 | 57 | 53 | 49 |
| Candida glabrata (“Cg”) | | | | | | | | | | 69 | 70 | 68 | 67 | 67 | 66 | 55 | 56 | 55 |
| Vanderwaltozyma polyspora (“Vp”) | | | | | | | | | | | 71 | 72 | 67 | 70 | 71 | 58 | 52 | 51 |
| Saccharomyces kluyveri (“Sk”) | | | | | | | | | | | | 77 | 71 | 72 | 69 | 53 | 54 | 54 |
| Kluyveromyces lactis (“Kl”) | | | | | | | | | | | | | 71 | 72 | 71 | 56 | 52 | 54 |
| Lachancea thermotolerans (“Lt”) | | | | | | | | | | | | | | 69 | 69 | 53 | 60 | 58 |
| Zygosaccharomyces rouxii (“Zr”) | | | | | | | | | | | | | | | 69 | 58 | 55 | 55 |
| Saccharomyces cerevisiae (“Sce”) | | | | | | | | | | | | | | | | 55 | 55 | 56 |
| Schizosaccharomyces pombe (“Spo”) | | | | | | | | | | | | | | | | | 58 | 60 |
| Yarrowia lipolytica (“Yl”) | | | | | | | | | | | | | | | | | | 61 |
| Neurospora crassa (“Nc”) | | | | | | | | | | | | | | | | | | |

### Example 9

### Construction of PNY2204 and PNY1910

The purpose of this example is to describe construction of a vector to enable integration of a gene encoding acetolactate synthase into the naturally occurring intergenic region between the PDC1 and TRX1 coding sequences in Chromosome XII. Strains resulting from use of this vector are also described.

Construction of Integration Vector pUC19-kan::pdc1::FBA-alsS::TRX1

The FBA-alsS-CYCt cassette was constructed by moving the 1.7 kb BbvCI/PacI fragment from pRS426::GPD::alsS::CYC (described in U.S. Pat. No. 7,851,188, which is herein incorporated by reference in its entirety) to pRS426::FBA::ILV5::CYC (described in U.S. Pat. No. 7,851,188, which is herein incorporated by reference in its entirety), which had been previously digested with BbvCI/PacI to release the ILV5 gene. Ligation reactions were transformed into E. coli TOP10 cells and transformants were screened by PCR using primers N98SeqF1 (SEQ ID NO:243) and N99SeqR2 (SEQ ID NO:244). The FBA-alsS-CYCt cassette was isolated from the vector using BglII and NotI for cloning into pUC19-URA3::ilvD-TRX1 at the AflII site (Klenow fragment was used to make ends compatible for ligation). Transformants containing the alsS cassette in both orientations in the vector were obtained and confirmed by PCR using primers N98SeqF4 (SEQ ID NO:245) and N1111 (SEQ ID NO:250) for configuration “A” and N98SeqF4 (SEQ ID NO:245) and N1110 (SEQ ID NO:9) for configuration “B”. A geneticin selectable version of the “A” configuration vector was then made by removing the URA3 gene (1.2 kb NotI/NaeI fragment) and adding a geneticin cassette. Klenow fragment was used to make all ends compatible for ligation, and transformants were screened by PCR to select a clone with the geneticin resistance gene in the same orientation as the previous URA3 marker using primers BK468 (SEQ ID NO:3) and N160SeqF5 (SEQ ID NO:4). The resulting clone was called pUC19-kan::pdc1::FBA-alsS::TRX1 (clone A) (SEQ ID NO:246).

Construction of alsS Integrant Strains

The pUC19-kan::pdc1::FBA-alsS integration vector described above was linearized with PmeI and transformed into PNY1507 (Example 12). PmeI cuts the vector within the cloned pdc1-TRX1 intergenic region and thus leads to targeted integration at that location (Rodney Rothstein, Methods in Enzymology, 1991, volume 194, pp. 281-301). Transformants were selected on YPE plus 50 μg/ml G418. Patched transformants were screened by PCR for the integration event using primers N160SeqF5 (SEQ ID NO:4) and oBP512 (SEQ ID NO:22). Two transformants were tested indirectly for acetolactate synthase function by evaluating the strains ability to make isobutanol. To do this, additional isobutanol pathway genes were supplied on E. coli-yeast shuttle vectors (pYZ090ΔalsS and pBP915). One clone was designated as PNY2205. The plasmid-free parent strain was designated PNY2204 (MATa ura3Δ::loxP his3Δ pdc6Δ pdc1Δ::P[PDC1]-DHAD|ilvD_Sm-PDC1t-pUC19-loxP-kanMX-loxP-P[FBA1]-ALS|alsS_Bs-CYC1t pdc5Δ::P[PDC5]-ADH|sadB_Ax-PDC5t gpd2Δ::loxP fra2Δ adh1Δ::UAS(PGK1)P[FBA1]-kivD_L1(y)-ADH1t).

Isobutanol Pathway Plasmids (pBP915ΔalsS, pBP915, and pLH702)

pYZ090 (SEQ ID NO:203,) was digested with SpeI and NotI to remove most of the CUP1 promoter and all of the alsS coding sequence and CYC terminator. The vector was then self-ligated after treatment with Klenow fragment and transformed into E. coli Stbl3 cells, selecting for ampicillin resistance. Removal of the DNA region was confirmed for two independent clones by DNA sequencing across the ligation junction by PCR using primer N191 (SEQ ID NO:247). The resulting plasmid was named pYZ090ΔalsS (SEQ ID NO:248).

The pLH468 plasmid was constructed for expression of DHAD, KivD and HADH in yeast. pBP915 was constructed from pLH468 (SEQ ID NO:204) by deleting the kivD gene and 957 base pairs of the TDH3 promoter upstream of kivD. pLH468 was digested with SwaI and the large fragment (12896 bp) was purified on an agarose gel followed by a Gel Extraction kit (Qiagen; Valencia, Calif.). The isolated fragment of DNA was self-ligated with T4 DNA ligase and used to transform electrocompetent TOP10 Escherichia coli (Invitrogen; Carlsbad, Calif.). Plasmids from transformants were isolated and checked for the proper deletion by restriction analysis with the SwaI restriction enzyme. Isolates were also sequenced across the deletion site with primers oBP556 (SEQ ID NO:238) and oBP561 (SEQ ID NO:239). A clone with the proper deletion was designated pBP915 (pLH468ΔkivD) (SEQ ID NO:84).

pYZ090 was constructed to contain a chimeric gene having the coding region of the alsS gene from Bacillus subtilis (nt position 457-2172) expressed from the yeast CUP1 promoter (nt 2-449) and followed by the CYC1 terminator (nt 2181-2430) for expression of ALS, and a chimeric gene having the coding region of the ilvC gene from Lactococcus lactis (nt 3634-4656) expressed from the yeast ILV5 promoter (2433-3626) and followed by the ILV5 terminator (nt 4682-5304) for expression of KARI.

Construction of Plasmid pLH702

Plasmid pLH702 was constructed in a series of steps from pYZ090 (SEQ ID NO:203) as described in the following paragraphs. This plasmid expresses KARI variant K9D3 (described in Example 6) from the yeast ILV5 promoter.

pYZ058 (pHR81-P_{CUP1}-A1sS-P_{ILV5}-yeast KARI) was derived from pYZ090 (pHR81-P_{CUP1}-A1sS-P_{ILV5}-lactis KARI; SEQ ID NO: 203). pYZ090 was cut with PmeI and SfiI enzymes, and ligated with a PCR product of yeast KARI. The PCR product was amplified from genomic DNA of Saccharomyces cerevisiae BY4741 (Research Genetics Inc.) strain using upper primer 5′-catcatcacagtttaaacagtatgttgaagcaaatcaacttcggtgg-3′ (SEQ ID NO:251) and lower primer 5′-ggacgggccctgcaggccttattggttttctggtctcaactttctgac-3′ (SEQ ID NO:252), and digested with PmeI and SfiI enzymes. pYZ058 was confirmed by sequencing.

pLH550 (pHR81-PCUP1-A1sS-PILV5-Pf5.KARI) was derived from pYZ058. The wild type Pf5.KARI gene was PCR amplified with OT1349 (5′-catcatcacagtttaaacagtatgaaagttttctacgataaagactgcgacc-3′; SEQ ID NO:253) and OT1318 (5′-gcacttgataggcctgcagggccttagttatggctttgtcgacgattttg-3′; SEQ ID NO:254), digested with PmeI and SfiI enzymes and ligated with pYZ058 vector cut with PmeI and SfiI. The vector generated, pLH550, was confirmed by sequencing.

pLH556 was derived from pLH550 by digesting the vector with SpeI and NotI enzymes, and ligating with a linker annealed from OT1383 (5′-ctagtcaccggtggc-3′, SEQ ID NO:255) and OT1384 (5′-ggccgccaccggtga-3′, SEQ ID NO:256) which contains overhang sequences for SpeI and NotI sites. This cloning step eliminates the alsS gene and a large fragment of the PCUP1 promoter, with 160 bp residual upstream sequence that is not functional. pLH556 was confirmed by sequencing.

pHR81::ILV5p-K9D3 (pLH702, SEQ ID NO: 132) was derived from pLH556. The K9D3 mutant KARI gene was excised from vector pBAD-K9D3 using PmeI and SfiI enzymes, and ligated with pLH556 at PmeI and SfiI sites, replacing the Pf5.KARI gene with the K9D3 gene. The constructed vector was confirmed by sequencing.

Strain PNY1910 was derived from PNY2204 after transformation with plasmids pLH702 and pBP915. The transformed cells were plated on synthetic complete medium without histidine or uracil (1% ethanol as carbon source). Yeast colonies from the transformation on SE-Ura-His plates appeared after 5-7 days. The colonies were patched onto fresh SE-Ura-His plates, incubate at 30° C. for 3 days. The patched cells were inoculated into 25 mL SEG-Ura, His media with 2% glucose and 0.2% ethanol, and grown semi-aerobically in 125 mL shake flask with lid closed for 2-3 days at 30° C., to 2-30D. The cells were centrifuged and re-suspended in 1 mL of the anaerobic media (SEG-Ura, His media (2% glucose, 0.1% ethanol, 10 mg/L ergosterol, 50 mM MES, pH 5.5, thiamine 30 mg/L, nicotinic acid 30 mg/L). A calculated amount of cells were transferred to 45 mL total volume of the anaerobic media for a starting OD=0.2 in a 60 mL serum vial, with the top rubber lid tightly closed with crimper. This step is done in the regular bio-hood in air. The serum vials were incubated at 30 C, 200 rpm for 2 days. At 48 h, the samples were removed for OD and HPLC analysis of glucose, isobutanol and pathway intermediates. In the initial phase of the 48 h incubation, the air present in the head space (˜15 mL) is consumed by the growing yeast cells. After the oxygen in the head space is consumed, the culture becomes anaerobic. Therefore this experiment includes switching condition from aerobic to oxygen limiting and anaerobic conditions. All the clones produced isobutanol under these conditions, and one was selected and named PNY1910.

### Example 10

### Construction of PNY2242

PNY1528 (hADH Integrations in PNY2211)

Deletions/integrations were created by homologous recombination with PCR products containing regions of homology upstream and downstream of the target region and the URA3 gene for selection of transformants. The URA3 gene was removed by homologous recombination to create a scarless deletion/integration.

The scarless deletion/integration procedure was adapted from Akada et al., Yeast, 23:399 (2006). The PCR cassette for each deletion/integration was made by combining four fragments, A-B-U-C, and the gene to be integrated by cloning the individual fragments into a plasmid prior to the entire cassette being amplified by PCR for the deletion/integration procedure. The gene to be integrated was included in the cassette between fragments A and B. The PCR cassette contained a selectable/counter-selectable marker, URA3 (Fragment U), consisting of the native CEN.PK 113-7D URA3 gene, along with the promoter (250 bp upstream of the URA3 gene) and terminator (150 bp downstream of the URA3 gene) regions. Fragments A and C (each approximately 100 to 500 bp long) corresponded to the sequence immediately upstream of the target region (Fragment A) and the 3′ sequence of the target region (Fragment C). Fragments A and C were used for integration of the cassette into the chromosome by homologous recombination. Fragment B (500 bp long) corresponded to the 500 bp immediately downstream of the target region and was used for excision of the URA3 marker and Fragment C from the chromosome by homologous recombination, as a direct repeat of the sequence corresponding to Fragment B was created upon integration of the cassette into the chromosome.

YPRCΔ15 Deletion and Horse Liver adh Integration

The YPRCΔ15 locus was deleted and replaced with the horse liver adh gene, codon optimized for expression in Saccharomyces cerevisiae, along with the PDC5 promoter region (538 bp) from Saccaromyces cerevisiae and the ADH1 terminator region (316 bp) from Saccaromyces cerevisiae. The scarless cassette for the YPRCΔ15 deletion-P[PDC5]-adh_HL(y)-ADH1t integration was first cloned into plasmid pUC19-URA3MCS (described in Example 11).

Fragments A-B-U-C were amplified using Phusion High Fidelity PCR Master Mix (New England BioLabs; Ipswich, Mass.) and CEN.PK 113-7D genomic DNA as template, prepared with a Gentra Puregene Yeast/Bact kit (Qiagen; Valencia, Calif.). YPRCΔ15 Fragment A was amplified from genomic DNA with primer oBP622 (SEQ ID NO:76), containing a KpnI restriction site, and primer oBP623 (SEQ ID NO:77), containing a 5′ tail with homology to the 5′ end of YPRCΔ15 Fragment B. YPRCΔ15 Fragment B was amplified from genomic DNA with primer oBP624 (SEQ ID NO:78), containing a 5′ tail with homology to the 3′ end of YPRCΔ15 Fragment A, and primer oBP625 (SEQ ID NO:79), containing a FseI restriction site. PCR products were purified with a PCR Purification kit (Qiagen). YPRCΔ15 Fragment A—YPRCΔ15 Fragment B was created by overlapping PCR by mixing the YPRCΔ15 Fragment A and YPRCΔ15 Fragment B PCR products and amplifying with primers oBP622 (SEQ ID NO:76) and oBP625 (SEQ ID NO:79). The resulting PCR product was digested with KpnI and FseI and ligated with T4 DNA ligase into the corresponding sites of pUC19-URA3MCS after digestion with the appropriate enzymes. YPRCΔ15 Fragment C was amplified from genomic DNA with primer oBP626 (SEQ ID NO:80), containing a NotI restriction site, and primer oBP627 (SEQ ID NO:81), containing a PacI restriction site. The YPRCΔ15 Fragment C PCR product was digested with NotI and Pad and ligated with T4 DNA ligase into the corresponding sites of the plasmid containing YPRCΔ15 Fragments AB. The PDC5 promoter region was amplified from CEN.PK 113-7D genomic DNA with primer HY21 (SEQ ID NO:82), containing an AscI restriction site, and primer HY24 (SEQ ID NO:83), containing a 5′ tail with homology to the 5′ end of adh_H1(y). adh_H1(y)-ADH1t was amplified from pBP915 (SEQ ID NO:84) with primers HY25 (SEQ ID NO: 85), containing a 5′ tail with homology to the 3′ end of P[PDC5], and HY4 (SEQ ID NO:86), containing a PmeI restriction site. PCR products were purified with a PCR Purification kit (Qiagen). P[PDC5]-adh_HL(y)-ADH1t was created by overlapping PCR by mixing the P[PDC5] and adh_HL(y)-ADH1t PCR products and amplifying with primers HY21 (SEQ ID NO:82) and HY4 (SEQ ID NO:86). The resulting PCR product was digested with AscI and PmeI and ligated with T4 DNA ligase into the corresponding sites of the plasmid containing YPRCΔ15 Fragments ABC. The entire integration cassette was amplified from the resulting plasmid with primers oBP622 (SEQ ID NO:76) and oBP627 (SEQ ID NO:81).

Competent cells of PNY2211 (Example 3) were made and transformed with the YPRCΔ15 deletion-P[PDC5]-adh_HL(y)-ADH1t integration cassette PCR product using a Frozen-EZ Yeast Transformation II kit (Zymo Research; Orange, Calif.). Transformation mixtures were plated on synthetic complete media lacking uracil supplemented with 1% ethanol at 30° C. Transformants were screened for by PCR with primers URA3-end F (SEQ ID NO:87) and oBP637 (SEQ ID NO:88). Correct transformants were grown in YPE (1% ethanol) and plated on synthetic complete medium supplemented with 1% EtOH and containing 5-fluoro-orotic acid (0.1%) at 30° C. to select for isolates that lost the URA3 marker. The deletion of YPRCΔ15 and integration of P[PDC5]-adh_HL(y)-ADH1t were confirmed by PCR with external primers oBP636 (SEQ ID NO:89) and oBP637 (SEQ ID NO:88) using genomic DNA prepared with a YeaStar Genomic DNA kit (Zymo Research). A correct isolate of the following genotype was selected for further modification: CEN.PK 113-7D MATa ura3Δ::loxP his3Δ pdc6Δ pdc1Δ::P[PDC1]-DHAD|ilvD_Sm-PDC1t-P[FBA1]-ALS|alsS_Bs-CYC1t pdc5Δ::P[PDC5]-ADH|sadB_Ax-PDC5t gpd2Δ::loxP fra2Δ adh1Δ::UAS(PGK1)P[FBA1]-kivD_L1(y)-ADH1t yprcΔ15Δ::P[PDC5]-ADH|adh_H1-ADH1t.

Horse Liver adh Integration at fra2Δ

The horse liver adh gene, codon optimized for expression in Saccharomyces cerevisiae, along with the PDC1 promoter region (870 bp) from Saccaromyces cerevisiae and the ADH1 terminator region (316 bp) from Saccaromyces cerevisiae, was integrated into the site of the fra2 deletion. The scarless cassette for the fra24-P[PDC1]-adh_HL(y)-ADH1t integration was first cloned into plasmid pUC19-URA3MCS.

Fragments A-B-U-C were amplified using Phusion High Fidelity PCR Master Mix (New England BioLabs; Ipswich, Mass.) and CEN.PK 113-7D genomic DNA as template, prepared with a Gentra Puregene Yeast/Bact kit (Qiagen; Valencia, Calif.). fra2A Fragment C was amplified from genomic DNA with primer oBP695 (SEQ ID NO:90), containing a NotI restriction site, and primer oBP696 (SEQ ID NO:91), containing a PacI restriction site. The fra2A Fragment C PCR product was digested with NotI and Pad and ligated with T4 DNA ligase into the corresponding sites of pUC19-URA3MCS. fra2A Fragment B was amplified from genomic DNA with primer oBP693 (SEQ ID NO:92), containing a PmeI restriction site, and primer oBP694 (SEQ ID NO:93), containing a FseI restriction site. The resulting PCR product was digested with PmeI and FseI and ligated with T4 DNA ligase into the corresponding sites of the plasmid containing fra2A fragment C after digestion with the appropriate enzymes. fra2A Fragment A was amplified from genomic DNA with primer oBP691 (SEQ ID NO:94), containing BamHI and AsiSI restriction sites, and primer oBP692 (SEQ ID NO:95), containing AscI and SwaI restriction sites. The fra2A fragment A PCR product was digested with BamHI and AscI and ligated with T4 DNA ligase into the corresponding sites of the plasmid containing fra2A fragments BC after digestion with the appropriate enzymes. The PDC1 promoter region was amplified from CEN.PK 113-7D genomic DNA with primer HY16 (SEQ ID NO:96), containing an AscI restriction site, and primer HY19 (SEQ ID NO:97), containing a 5′ tail with homology to the 5′ end of adh_H1(y). adh_H1(y)-ADH1t was amplified from pBP915 with primers HY20 (SEQ ID NO:98), containing a 5′ tail with homology to the 3′ end of P[PDC1], and HY4 (SEQ ID NO:86), containing PmeI restriction site. PCR products were purified with a PCR Purification kit (Qiagen). P[PDC1]-adh_HL(y)-ADH1t was created by overlapping PCR by mixing the P[PDC1] and adh_HL(y)-ADH1t PCR products and amplifying with primers HY16 (SEQ ID NO:96) and HY4 (SEQ ID NO:86). The resulting PCR product was digested with AscI and PmeI and ligated with T4 DNA ligase into the corresponding sites of the plasmid containing fra2Δ Fragments ABC. The entire integration cassette was amplified from the resulting plasmid with primers oBP691 (SEQ ID NO:94) and oBP696 (SEQ ID NO:91).

Competent cells of the PNY2211 variant with adh_H1(y) integrated at YPRCΔ15 were made and transformed with the fra2Δ-P[PDC1]-adh_HL(y)-ADH1t integration cassette PCR product using a Frozen-EZ Yeast Transformation II kit (Zymo Research). Transformation mixtures were plated on synthetic complete media lacking uracil supplemented with 1% ethanol at 30° C. Transformants were screened for by PCR with primers URA3-end F (SEQ ID NO:87) and oBP731 (SEQ ID NO:99). Correct transformants were grown in YPE (1% ethanol) and plated on synthetic complete medium supplemented with 1% EtOH and containing 5-fluoro-orotic acid (0.1%) at 30° C. to select for isolates that lost the URA3 marker. The integration of P[PDC1]-adh_HL(y)-ADH1t was confirmed by colony PCR with internal primer HY31 (SEQ ID NO:100) and external primer oBP731 (SEQ ID NO: 99) and PCR with external primers oBP730 (SEQ ID NO:101) and oBP731 (SEQ ID NO:99) using genomic DNA prepared with a YeaStar Genomic DNA kit (Zymo Research). A correct isolate of the following genotype was designated PNY1528: CEN.PK 113-7D MATa ura3Δ::loxP his3Δ pdc6Δ pdc1Δ::P[PDC1]-DHAD|ilvD_Sm-PDC1t-P[FBA1]-ALS|alsS_Bs-CYC1t pdc5Δ::P[PDC5]-ADH|sadB_Ax-PDC5t gpd2Δ::loxP fra2Δ::P[PDC1]-ADH|adh_H1-ADH1t adh1Δ::UAS(PGK1)P[FBA1]-kivD_L1(y)-ADH1t yprcΔ15Δ::P[PDC5]-ADH|adh_H1-ADH1t.

PNY2237 (YMRC226c Deletion)

The gene YMR226c was deleted from S. cerevisiae strain PNY1528 by homologous recombination using a PCR amplified 2.0 kb linear scarless deletion cassette. The cassette was constructed from spliced PCR amplified fragments comprised of the URA3 gene, along with its native promoter and terminator as a selectable marker, upstream and downstream homology sequences flanking the YMR226c gene chromosomal locus to promote integration of the deletion cassette and removal of the native intervening sequence and a repeat sequence to promote recombination and removal of the URA3 marker. Forward and reverse PCR primers (N1251 and N1252, SEQ ID NOs:102 and 103, respectively), amplified a 1,208 bp URA3 expression cassette originating from pLA33 (pUC19::loxP-URA3-loxP (SEQ ID NO:104)). Forward and reverse primers (N1253 and N1254, SEQ ID NOs:105 and 106, respectively), amplified a 250 bp downstream homology sequence with a 3′ URA3 overlap sequence tag from a genomic DNA preparation of S. cerevisiae strain PNY2211 (above). Forward and reverse PCR primers (N1255 and N1256, SEQ ID NOs:107 and 108, respectively) amplified a 250 bp repeat sequence with a 5′ URA3 overlap sequence tag from a genomic DNA preparation of S. cerevisiae strain PNY2211. Forward and reverse PCR primers (N1257 and N1258, SEQ ID NOs:109 and 110, respectively) amplified a 250 bp upstream homology sequence with a 5′ repeat overlap sequence tag from a genomic DNA preparation of S. cerevisiae strain PNY2211.

Approximately 1.5 μg of the PCR amplified cassette was transformed into strain PNY1528 (above) made competent using the ZYMO Research Frozen Yeast Transformation Kit and the transformation mix plated on SE 1.0%-uracil and incubated at 30° C. for selection of cells with an integrated ymr226cΔ::URA3 cassette. Transformants appearing after 72 to 96 hours are subsequently short-streaked on the same medium and incubated at 30° C. for 24 to 48 hours. The short-streaks are screened for ymr226cΔ::URA3 by PCR, with a 5′ outward facing URA3 deletion cassette-specific internal primer (N1249, SEQ ID NO:111) paired with a flanking inward facing chromosome-specific primer (N1239, SEQ ID NO:112) and a 3′ outward-facing URA3 deletion cassette-specific primer (N1250, SEQ ID NO:113) paired with a flanking inward-facing chromosome-specific primer (N1242, SEQ ID NO:114). A positive PNY1528 ymr226cΔ::URA3 PCR screen resulted in 5′ and 3′ PCR products of 598 and 726 bp, respectively.

Three positive PNY1528 ymr226cΔ::URA3 clones were picked and cultured overnight in a YPE 1% medium of which 100 μL was plated on YPE 1%+5-FOA for marker removal. Colonies appearing after 24 to 48 hours were PCR screened for marker loss with 5′ and 3′ chromosome-specific primers (N1239; SEQ ID NO:112 and N1242; SEQ ID NO:114). A positive PNY1528 ymr226cΔ markerless PCR screen resulted in a PCR product of 801 bp. Multiple clones were obtained. Clone 2.1 is officially PNY2237.

PNY2238 (YMRC226C and ALD6 Deletion)

A vector was designed to replace the ALD6 coding sequence with a Cre-lox recyclable URA3 selection marker. Sequences 5′ and 3′ of ALD6 were amplified by PCR (primer pairs N1179 and N1180 and N1181 and N1182, respectively; SEQ ID NOs:115, 116, 117, and 118. respectively). After cloning these fragments into TOPO vectors (Invitrogen Cat. No. K2875-J10) and sequencing (M13 forward and reverse primers, SEQ ID NOs:119 and 120, respectively), the 5′ and 3′ flanks were cloned into pLA33 (pUC19::loxP::URA3::loxP) (SEQ ID NO:104) at the EcoRI and SphI sites, respectively. Each ligation reaction was transformed into E. coli Stbl3 cells, which were incubated on LB Amp plates to select for transformants. Proper insertion of sequences was confirmed by PCR (primers M13 forward and N1180 and M13 reverse and N1181, respectively).

The vector described above was linearized with AhdI and transformed into PNY2237 using the standard lithium acetate method (except that incubation of cells with DNA was extended to 2.5 h). Transformants were obtained by plating on synthetic complete medium minus uracil that provided 1% ethanol as the carbon source. Patched transformants were screened by PCR to confirm the deletion/integration, using primers N1212 (SEQ ID NO:121) and N1180 (5′ end) (SEQ ID NO:116) and N1181 (SEQ ID NO:117) and N1213 (SEQ ID NO:122) (3′ end). A plasmid carrying Cre recombinase (pRS423::GAL1p-Cre=SEQ ID No:123) was transformed into the strain using histidine marker selection. Transformants were passaged on YPE supplemented with 0.5% galactose. Colonies were screened for resistance to 5-FOA (loss of URA3 marker) and for histidine auxotrophy (loss of the Cre plasmid). Proper removal of the URA3 gene via the flanking loxP sites was confirmed by PCR (primers N1262 and N1263, SEQ ID NOs:124 and 125, respectively). Additionally, primers internal to the ALD6 gene (N1230 and N1231; SEQ ID NOs:126 and 127, respectively) were used to insure that no merodiploids were present. Finally, ald6Δ:loxP clones were screened by PCR to confirm that a translocation between ura3Δ::loxP (N1228 and N1229, SEQ ID NOs:128 and 129, respectively) and gpd2Δ:loxP (N1223 and N1225, SEQ ID NOs:130 and 131, respectively) had not occurred. Three positive clones were identified from screening transformants of PNY2237. Clone E was selected (PNY2238) for further development.

PNY2242

Strain PNY2242 was derived from PNY2238 after transformation with plasmids pLH702 (Example 9) and pYZ067ΔkivDΔhADH (below). Transformation mixtures were plated on synthetic complete medium without histidine or uracil (1% ethanol as carbon source). Transformants were patched to the same medium containing, instead, 2% glucose and 0.05% ethanol as carbon sources. Three patches were tested for isobutanol production. All three performed similarly in terms of glucose consumption and isobutanol production. One clone was designated PNY2242 and was further characterized under fermentation conditions, as described herein below.

pYZ067 (SEQ ID NO:133) was constructed to contain the following chimeric genes: 1) the coding region of the ilvD gene from S. mutans UA159 with a C-terminal Lumio tag expressed from the yeast FBA1 promoter followed by the FBA1 terminator for expression of dihydroxy acid dehydratase, 2) the coding region for horse liver ADH expressed from the yeast GPM1 promoter followed by the ADH1 terminator for expression of alcohol dehydrogenase, and 3) the coding region of the KivD gene from Lactococcus lactis expressed from the yeast TDH3 promoter followed by the TDH3 terminator for expression of ketoisovalerate decarboxylase.

Plasmid pYZ067ΔkivDΔhADH was constructed from pYZ067 by deleting the promoter-gene-terminator cassettes for both kivD and adh. pYZ067 was digested with BamHI and SacI (New England BioLabs; Ipswich, Mass.) and the 7934 bp fragment was purified on an agarose gel followed by a Gel Extraction kit (Qiagen; Valencia, Calif.). The isolated fragment of DNA was treated with DNA Polymerase I, Large (Klenow) Fragment (New England BioLabs; Ipswich, Mass.) and then self-ligated with T4 DNA ligase and used to transform competent TOP10 Escherichia coli (Invitrogen; Carlsbad, Calif.). Plasmids from transformants were isolated and checked for the proper deletion by sequence analysis. A correct plasmid isolate was designated pYZ067ΔkivDΔhADH (SEQ ID NO:261).

### Example 11

### Construction of Saccharomyces cerevisiae Strain BP1064 (PNY1503)

The strain BP1064 was derived from CEN.PK 113-7D (CBS 8340; Centraalbureau voor Schimmelcultures (CBS) Fungal Biodiversity Centre, Netherlands) and contains deletions of the following genes: URA3, HIS3, PDC1, PDC5, PDC6, and GPD2.

Deletions, which completely removed the entire coding sequence, were created by homologous recombination with PCR fragments containing regions of homology upstream and downstream of the target gene and either a G418 resistance marker or URA3 gene for selection of transformants. The G418 resistance marker, flanked by loxP sites, was removed using Cre recombinase (pRS423::PGAL1-cre; SEQ ID NO: 123). The URA3 gene was removed by homologous recombination to create a scarless deletion, or if flanked by loxP sites was removed using Cre recombinase.

The scarless deletion procedure was adapted from Akada et al., Yeast, 23:399, 2006. In general, the PCR cassette for each scarless deletion was made by combining four fragments, A-B-U-C, by overlapping PCR. The PCR cassette contained a selectable/counter-selectable marker, URA3 (Fragment U), consisting of the native CEN.PK 113-7D URA3 gene, along with the promoter (250 bp upstream of the URA3 gene) and terminator (150 bp downstream of the URA3 gene). Fragments A and C, each 500 bp long, corresponded to the 500 bp immediately upstream of the target gene (Fragment A) and the 3′ 500 bp of the target gene (Fragment C). Fragments A and C were used for integration of the cassette into the chromosome by homologous recombination. Fragment B (500 bp long) corresponded to the 500 bp immediately downstream of the target gene and was used for excision of the URA3 marker and Fragment C from the chromosome by homologous recombination, as a direct repeat of the sequence corresponding to Fragment B was created upon integration of the cassette into the chromosome. Using the PCR product ABUC cassette, the URA3 marker was first integrated into and then excised from the chromosome by homologous recombination. The initial integration deleted the gene, excluding the 3′ 500 bp. Upon excision, the 3′ 500 bp region of the gene was also deleted. For integration of genes using this method, the gene to be integrated was included in the PCR cassette between fragments A and B.

### URA3 Deletion

To delete the endogenous URA3 coding region, a ura3::loxP-kanMX-loxP cassette was PCR-amplified from pLA54 template DNA (SEQ ID NO:199). pLA54 contains the K. lactis TEF1 promoter and kanMX marker, and is flanked by loxP sites to allow recombination with Cre recombinase and removal of the marker. PCR was done using Phusion DNA polymerase and primers BK505 and BK506 (SEQ ID NOs:260 and 138). The URA3 portion of each primer was derived from the 5′ region upstream of the URA3 promoter and 3′ region downstream of the coding region such that integration of the loxP-kanMX-loxP marker resulted in replacement of the URA3 coding region. The PCR product was transformed into CEN.PK 113-7D using standard genetic techniques (Methods in Yeast Genetics, 2005, Cold Spring Harbor Laboratory Press, Cold Spring Harbor, N.Y., pp. 201-202) and transformants were selected on YPD containing G418 (100 μg/ml) at 30° C. Transformants were screened to verify correct integration by PCR using primers LA468 and LA492 (SEQ ID NOs:139 and 140) and designated CEN.PK 113-7D Δura3::kanMX.

### HIS3 Deletion

The four fragments for the PCR cassette for the scarless HIS3 deletion were amplified using Phusion High Fidelity PCR Master Mix (New England BioLabs; Ipswich, Mass.) and CEN.PK 113-7D genomic DNA as template, prepared with a Gentra Puregene Yeast/Bact kit (Qiagen; Valencia, Calif.). HIS3 Fragment A was amplified with primer oBP452 (SEQ ID NO:147) and primer oBP453 (SEQ ID NO:148), containing a 5′ tail with homology to the 5′ end of HIS3 Fragment B. HIS3 Fragment B was amplified with primer oBP454 (SEQ ID NO:149), containing a 5′ tail with homology to the 3′ end of HIS3 Fragment A, and primer oBP455 (SEQ ID NO:150), containing a 5′ tail with homology to the 5′ end of HIS3 Fragment U. HIS3 Fragment U was amplified with primer oBP456 (SEQ ID NO:151), containing a 5′ tail with homology to the 3′ end of HIS3 Fragment B, and primer oBP457 (SEQ ID NO:152), containing a 5′ tail with homology to the 5′ end of HIS3 Fragment C. HIS3 Fragment C was amplified with primer oBP458 (SEQ ID NO:153), containing a 5′ tail with homology to the 3′ end of HIS3 Fragment U, and primer oBP459 (SEQ ID NO:154). PCR products were purified with a PCR Purification kit (Qiagen). HIS3 Fragment AB was created by overlapping PCR by mixing HIS3 Fragment A and HIS3 Fragment B and amplifying with primers oBP452 (SEQ ID NO:147) and oBP455 (SEQ ID NO:150). HIS3 Fragment UC was created by overlapping PCR by mixing HIS3 Fragment U and HIS3 Fragment C and amplifying with primers oBP456 (SEQ ID NO:151) and oBP459 (SEQ ID NO:154). The resulting PCR products were purified on an agarose gel followed by a Gel Extraction kit (Qiagen). The HIS3 ABUC cassette was created by overlapping PCR by mixing HIS3 Fragment AB and HIS3 Fragment UC and amplifying with primers oBP452 (SEQ ID NO:147) and oBP459 (SEQ ID NO:154). The PCR product was purified with a PCR Purification kit (Qiagen).

Competent cells of CEN.PK 113-7D Δura3::kanMX were made and transformed with the HIS3 ABUC PCR cassette using a Frozen-EZ Yeast Transformation II kit (Zymo Research; Orange, Calif.). Transformation mixtures were plated on synthetic complete media lacking uracil supplemented with 2% glucose at 30° C. Transformants with a his3 knockout were screened for by PCR with primers oBP460 (SEQ ID NO:155) and oBP461 (SEQ ID NO:156) using genomic DNA prepared with a Gentra Puregene Yeast/Bact kit (Qiagen). A correct transformant was selected as strain CEN.PK 113-7D Δura3::kanMX Δhis3::URA3.

KanMX Marker Removal from the Δura3 Site and URA3 Marker Removal from the Δhis3 Site

The KanMX marker was removed by transforming CEN.PK 113-7D Δura3::kanMX Δhis3::URA3 with pRS423::PGAL1-cre (SEQ ID NO: 123) using a Frozen-EZ Yeast Transformation II kit (Zymo Research) and plating on synthetic complete medium lacking histidine and uracil supplemented with 2% glucose at 30° C. Transformants were grown in YP supplemented with 1% galactose at 30° C. for ˜6 hours to induce the Cre recombinase and KanMX marker excision and plated onto YPD (2% glucose) plates at 30° C. for recovery. An isolate was grown overnight in YPD and plated on synthetic complete medium containing 5-fluoro-orotic acid (0.1%) at 30° C. to select for isolates that lost the URA3 marker. 5-FOA resistant isolates were grown in and plated on YPD for removal of the pRS423::PGAL1-cre plasmid. Isolates were checked for loss of the KanMX marker, URA3 marker, and pRS423::PGAL1-cre plasmid by assaying growth on YPD+G418 plates, synthetic complete medium lacking uracil plates, and synthetic complete medium lacking histidine plates. A correct isolate that was sensitive to G418 and auxotrophic for uracil and histidine was selected as strain CEN.PK 113-7D Δura3::loxP Δhis3 and designated as BP857. The deletions and marker removal were confirmed by PCR and sequencing with primers oBP450 (SEQ ID NO:157) and oBP451 (SEQ ID NO:158) for Δura3 and primers oBP460 (SEQ ID NO:155) and oBP461 (SEQ ID NO:156) for Δhis3 using genomic DNA prepared with a Gentra Puregene Yeast/Bact kit (Qiagen).

### PDC6 Deletion

The four fragments for the PCR cassette for the scarless PDC6 deletion were amplified using Phusion High Fidelity PCR Master Mix (New England BioLabs) and CEN.PK 113-7D genomic DNA as template, prepared with a Gentra Puregene Yeast/Bact kit (Qiagen). PDC6 Fragment A was amplified with primer oBP440 (SEQ ID NO:159) and primer oBP441 (SEQ ID NO:160), containing a 5′ tail with homology to the 5′ end of PDC6 Fragment B. PDC6 Fragment B was amplified with primer oBP442 (SEQ ID NO:161), containing a 5′ tail with homology to the 3″ end of PDC6 Fragment A, and primer oBP443 (SEQ ID NO:162), containing a 5′ tail with homology to the 5′ end of PDC6 Fragment U. PDC6 Fragment U was amplified with primer oBP444 (SEQ ID NO:163), containing a 5′ tail with homology to the 3′ end of PDC6 Fragment B, and primer oBP445 (SEQ ID NO:164), containing a 5′ tail with homology to the 5′ end of PDC6 Fragment C. PDC6 Fragment C was amplified with primer oBP446 (SEQ ID NO:165), containing a 5′ tail with homology to the 3′ end of PDC6 Fragment U, and primer oBP447 (SEQ ID NO:166). PCR products were purified with a PCR Purification kit (Qiagen). PDC6 Fragment AB was created by overlapping PCR by mixing PDC6 Fragment A and PDC6 Fragment B and amplifying with primers oBP440 (SEQ ID NO:159) and oBP443 (SEQ ID NO:162). PDC6 Fragment UC was created by overlapping PCR by mixing PDC6 Fragment U and PDC6 Fragment C and amplifying with primers oBP444 (SEQ ID NO:163) and oBP447 (SEQ ID NO:166). The resulting PCR products were purified on an agarose gel followed by a Gel Extraction kit (Qiagen). The PDC6 ABUC cassette was created by overlapping PCR by mixing PDC6 Fragment AB and PDC6 Fragment UC and amplifying with primers oBP440 (SEQ ID NO:159) and oBP447 (SEQ ID NO:166). The PCR product was purified with a PCR Purification kit (Qiagen).

Competent cells of CEN.PK 113-7D Δura3::loxP Δhis3 were made and transformed with the PDC6 ABUC PCR cassette using a Frozen-EZ Yeast Transformation II kit (Zymo Research). Transformation mixtures were plated on synthetic complete media lacking uracil supplemented with 2% glucose at 30° C. Transformants with a pdc6 knockout were screened for by PCR with primers oBP448 (SEQ ID NO:167) and oBP449 (SEQ ID NO:168) using genomic DNA prepared with a Gentra Puregene Yeast/Bact kit (Qiagen). A correct transformant was selected as strain CEN.PK 113-7D Δura3::loxP Δhis3 Δpdc6::URA3.

CEN.PK 113-7D Δura3::loxP Δhis3 Δpdc6::URA3 was grown overnight in YPD and plated on synthetic complete medium containing 5-fluoro-orotic acid (0.1%) at 30° C. to select for isolates that lost the URA3 marker. The deletion and marker removal were confirmed by PCR and sequencing with primers oBP448 (SEQ ID NO:167) and oBP449 (SEQ ID NO:168) using genomic DNA prepared with a Gentra Puregene Yeast/Bact kit (Qiagen). The absence of the PDC6 gene from the isolate was demonstrated by a negative PCR result using primers specific for the coding sequence of PDC6, oBP554 (SEQ ID NO:169) and oBP555 (SEQ ID NO:170). The correct isolate was selected as strain CEN.PK 113-7D Δura3::loxP Δhis3 Δpdc6 and designated as BP891.

PDC1 Deletion ilvDSm Integration

The PDC1 gene was deleted and replaced with the ilvD coding region from Streptococcus mutans ATCC #700610. The A fragment followed by the ilvD coding region from Streptococcus mutans for the PCR cassette for the PDC1 deletion-ilvDSm integration was amplified using Phusion High Fidelity PCR Master Mix (New England BioLabs) and NYLA83 genomic DNA as template, prepared with a Gentra Puregene Yeast/Bact kit (Qiagen). NYLA83 is a strain which carries the PDC1 deletion-ilvDSm integration described in U.S. Patent Application Publication No. 2009/0305363, which is herein incorporated by reference in its entirety. PDC1 Fragment A-ilvDSm (SEQ ID NO:206) was amplified with primer oBP513 (SEQ ID NO:171) and primer oBP515 (SEQ ID NO:172), containing a 5′ tail with homology to the 5′ end of PDC1 Fragment B. The B, U, and C fragments for the PCR cassette for the PDC1 deletion-ilvDSm integration were amplified using Phusion High Fidelity PCR Master Mix (New England BioLabs) and CEN.PK 113-7D genomic DNA as template, prepared with a Gentra Puregene Yeast/Bact kit (Qiagen). PDC1 Fragment B was amplified with primer oBP516 (SEQ ID NO:173) containing a 5′ tail with homology to the 3′ end of PDC1 Fragment A-ilvDSm, and primer oBP517 (SEQ ID NO:174), containing a 5′ tail with homology to the 5′ end of PDC1 Fragment U. PDC1 Fragment U was amplified with primer oBP518 (SEQ ID NO:175), containing a 5′ tail with homology to the 3′ end of PDC1 Fragment B, and primer oBP519 (SEQ ID NO:176), containing a 5′ tail with homology to the 5′ end of PDC1 Fragment C. PDC1 Fragment C was amplified with primer oBP520 (SEQ ID NO:177), containing a 5′ tail with homology to the 3′ end of PDC1 Fragment U, and primer oBP521 (SEQ ID NO:178). PCR products were purified with a PCR Purification kit (Qiagen). PDC1 Fragment A-ilvDSm-B was created by overlapping PCR by mixing PDC1 Fragment A-ilvDSm and PDC1 Fragment B and amplifying with primers oBP513 (SEQ ID NO:171) and oBP517 (SEQ ID NO:174). PDC1 Fragment UC was created by overlapping PCR by mixing PDC1 Fragment U and PDC1 Fragment C and amplifying with primers oBP518 (SEQ ID NO:175) and oBP521 (SEQ ID NO:178). The resulting PCR products were purified on an agarose gel followed by a Gel Extraction kit (Qiagen). The PDC1 A-ilvDSm-BUC cassette (SEQ ID NO:207) was created by overlapping PCR by mixing PDC1 Fragment A-ilvDSm-B and PDC1 Fragment UC and amplifying with primers oBP513 (SEQ ID NO:171) and oBP521 (SEQ ID NO:178). The PCR product was purified with a PCR Purification kit (Qiagen).

Competent cells of CEN.PK 113-7D Δura3::loxP Δhis3 Δpdc6 were made and transformed with the PDC1 A-ilvDSm-BUC PCR cassette using a Frozen-EZ Yeast Transformation II kit (Zymo Research). Transformation mixtures were plated on synthetic complete media lacking uracil supplemented with 2% glucose at 30° C. Transformants with a pdc1 knockout ilvDSm integration were screened for by PCR with primers oBP511 (SEQ ID NO:179) and oBP512 (SEQ ID NO:180) using genomic DNA prepared with a Gentra Puregene Yeast/Bact kit (Qiagen). The absence of the PDC1 gene from the isolate was demonstrated by a negative PCR result using primers specific for the coding sequence of PDC1, oBP550 (SEQ ID NO:181) and oBP551 (SEQ ID NO:182). A correct transformant was selected as strain CEN.PK 113-7D Δura3::loxP Δhis3 Δpdc6 Δpdc1::ilvDSm-URA3.

CEN.PK 113-7D Δura3::loxP Δhis3 Δpdc6 Δpdc1::ilvDSm-URA3 was grown overnight in YPD and plated on synthetic complete medium containing 5-fluoro-orotic acid (0.1%) at 30° C. to select for isolates that lost the URA3 marker. The deletion of PDC1, integration of ilvDSm, and marker removal were confirmed by PCR and sequencing with primers oBP511 (SEQ ID NO:179) and oBP512 (SEQ ID NO:180) using genomic DNA prepared with a Gentra Puregene Yeast/Bact kit (Qiagen). The correct isolate was selected as strain CEN.PK 113-7D Δura3::loxP Δhis3 Δpdc6 Δpdc1::ilvDSm and designated as BP907.

PDC5 Deletion sadB Integration

The PDC5 gene was deleted and replaced with the sadB coding region from Achromobacter xylosoxidans (the sadB gene is described in U.S. Patent Appl. No. 2009/0269823, which is herein incorporated by reference in its entirety). A segment of the PCR cassette for the PDC5 deletion-sadB integration was first cloned into plasmid pUC19-URA3MCS.

pUC19-URA3MCS is pUC19 based and contains the sequence of the URA3 gene from Saccharomyces cerevisiae situated within a multiple cloning site (MCS). pUC19 contains the pMB1 replicon and a gene coding for beta-lactamase for replication and selection in Escherichia coli. In addition to the coding sequence for URA3, the sequences from upstream and downstream of this gene were included for expression of the URA3 gene in yeast. The vector can be used for cloning purposes and can be used as a yeast integration vector.

The DNA encompassing the URA3 coding region along with 250 bp upstream and 150 bp downstream of the URA3 coding region from Saccharomyces cerevisiae CEN.PK 113-7D genomic DNA was amplified with primers oBP438 (SEQ ID NO:145), containing BamHI, AscI, PmeI, and FseI restriction sites, and oBP439 (SEQ ID NO:146), containing XbaI, PacI, and NotI restriction sites, using Phusion High-Fidelity PCR Master Mix (New England BioLabs). Genomic DNA was prepared using a Gentra Puregene Yeast/Bact kit (Qiagen). The PCR product and pUC19 (SEQ ID NO:205) were ligated with T4 DNA ligase after digestion with BamHI and XbaI to create vector pUC19-URA3MCS. The vector was confirmed by PCR and sequencing with primers oBP264 (SEQ ID NO:143) and oBP265 (SEQ ID NO:144).

The coding sequence of sadB and PDC5 Fragment B were cloned into pUC19-URA3MCS to create the sadB-BU portion of the PDC5 A-sadB-BUC PCR cassette. The coding sequence of sadB was amplified using pLH468-sadB (SEQ ID NO:201) as template with primer oBP530 (SEQ ID NO:183), containing an AscI restriction site, and primer oBP531 (SEQ ID NO:184), containing a 5′ tail with homology to the 5′ end of PDC5 Fragment B. PDC5 Fragment B was amplified with primer oBP532 (SEQ ID NO:185), containing a 5′ tail with homology to the 3′ end of sadB, and primer oBP533 (SEQ ID NO:186), containing a PmeI restriction site. PCR products were purified with a PCR Purification kit (Qiagen). sadB-PDC5 Fragment B was created by overlapping PCR by mixing the sadB and PDC5 Fragment B PCR products and amplifying with primers oBP530 (SEQ ID NO:183) and oBP533 (SEQ ID NO:186). The resulting PCR product was digested with AscI and PmeI and ligated with T4 DNA ligase into the corresponding sites of pUC19-URA3MCS after digestion with the appropriate enzymes. The resulting plasmid was used as a template for amplification of sadB-Fragment B-Fragment U using primers oBP536 (SEQ ID NO:187) and oBP546 (SEQ ID NO:188), containing a 5′ tail with homology to the 5′ end of PDC5 Fragment C. PDC5 Fragment C was amplified with primer oBP547 (SEQ ID NO:189) containing a 5′ tail with homology to the 3′ end of PDC5 sadB-Fragment B-Fragment U, and primer oBP539 (SEQ ID NO:190). PCR products were purified with a PCR Purification kit (Qiagen). PDC5 sadB-Fragment B-Fragment U-Fragment C was created by overlapping PCR by mixing PDC5 sadB-Fragment B-Fragment U and PDC5 Fragment C and amplifying with primers oBP536 (SEQ ID NO:187) and oBP539 (SEQ ID NO:190). The resulting PCR product was purified on an agarose gel followed by a Gel Extraction kit (Qiagen). The PDC5 A-sadB-BUC cassette (SEQ ID NO:208) was created by amplifying PDC5 sadB-Fragment B-Fragment U-Fragment C with primers oBP542 (SEQ ID NO:191), containing a 5′ tail with homology to the 50 nucleotides immediately upstream of the native PDC5 coding sequence, and oBP539 (SEQ ID NO:190). The PCR product was purified with a PCR Purification kit (Qiagen).

Competent cells of CEN.PK 113-7D Δura3::loxP Δhis3 Δpdc6 Δpdc1::ilvDSm were made and transformed with the PDC5 A-sadB-BUC PCR cassette using a Frozen-EZ Yeast Transformation II kit (Zymo Research). Transformation mixtures were plated on synthetic complete media lacking uracil supplemented with 1% ethanol (no glucose) at 30 C. Transformants with a pdc5 knockout sadB integration were screened for by PCR with primers oBP540 (SEQ ID NO:192) and oBP541 (SEQ ID NO:193) using genomic DNA prepared with a Gentra Puregene Yeast/Bact kit (Qiagen). The absence of the PDC5 gene from the isolate was demonstrated by a negative PCR result using primers specific for the coding sequence of PDC5, oBP552 (SEQ ID NO:194) and oBP553 (SEQ ID NO:195). A correct transformant was selected as strain CEN.PK 113-7D Δura3::loxP Δhis3 Δpdc6 Δpdc1::ilvDSm Δpdc5::sadB-URA3.

CEN.PK 113-7D Δura3::loxP Δhis3 Δpdc6 Δpdc1::ilvDSm Δpdc5::sadB-URA3 was grown overnight in YPE (1% ethanol) and plated on synthetic complete medium supplemented with ethanol (no glucose) and containing 5-fluoro-orotic acid (0.1%) at 30 C to select for isolates that lost the URA3 marker. The deletion of PDC5, integration of sadB, and marker removal were confirmed by PCR with primers oBP540 (SEQ ID NO:192) and oBP541 (SEQ ID NO:193) using genomic DNA prepared with a Gentra Puregene Yeast/Bact kit (Qiagen). The correct isolate was selected as strain CEN.PK 113-7D Δura3::loxP Δhis3 Δpdc6 Δpdc1::ilvDSm Δpdc5::sadB and designated as BP913.

### GPD2 Deletion

To delete the endogenous GPD2 coding region, a gpd2::loxP-URA3-loxP cassette (SEQ ID NO:209) was PCR-amplified using loxP-URA3-loxP PCR (SEQ ID NO:202) as template DNA. loxP-URA3-loxP contains the URA3 marker from (ATCC #77107) flanked by loxP recombinase sites. PCR was done using Phusion DNA polymerase and primers LA512 and LA513 (SEQ ID NOs:141 and 142). The GPD2 portion of each primer was derived from the 5′ region upstream of the GPD2 coding region and 3′ region downstream of the coding region such that integration of the loxP-URA3-loxP marker resulted in replacement of the GPD2 coding region. The PCR product was transformed into BP913 and transformants were selected on synthetic complete media lacking uracil supplemented with 1% ethanol (no glucose). Transformants were screened to verify correct integration by PCR using primers oBP582 and AA270 (SEQ ID NOs:196 and 197).

The URA3 marker was recycled by transformation with pRS423::PGAL1-cre (SEQ ID NO:123) and plating on synthetic complete media lacking histidine supplemented with 1% ethanol at 30 C. Transformants were streaked on synthetic complete medium supplemented with 1% ethanol and containing 5-fluoro-orotic acid (0.1%) and incubated at 30 C to select for isolates that lost the URA3 marker. 5-FOA resistant isolates were grown in YPE (1% ethanol) for removal of the pRS423::PGAL1-cre plasmid. The deletion and marker removal were confirmed by PCR with primers oBP582 (SEQ ID NO:196) and oBP591 (SEQ ID NO:198). The correct isolate was selected as strain CEN.PK 113-7D Δura3::loxP Δhis3 Δpdc6 Δpdc1::ilvDSm Δpdc5::sadB Δgpd2::loxP and designated as BP1064 (PNY1503).

### Example 12

### Construction of Saccharomyces cerevisiae strains BP1135 (PNY1505) and PNY1507

The purpose of this Example is to describe construction of Saccharomyces cerevisiae strains BP1135 and PNY1507. These strains were derived from PNY1503 (BP1064). BP1135 contains an additional deletion of the FRA2 gene. PNY1507 was derived from BP1135 with additional deletion of the ADH1 gene, with integration of the kivD gene from Lactococcus lactis, codon optimized for expression in Saccharomyces cerevisiae, into the ADH1 locus.

### FRA2 Deletion

The FRA2 deletion was designed to delete 250 nucleotides from the 3′ end of the coding sequence, leaving the first 113 nucleotides of the FRA2 coding sequence intact. An in-frame stop codon was present 7 nucleotides downstream of the deletion. The four fragments for the PCR cassette for the scarless FRA2 deletion were amplified using Phusion High Fidelity PCR Master Mix (New England BioLabs; Ipswich, Mass.) and CEN.PK 113-7D genomic DNA as template, prepared with a Gentra Puregene Yeast/Bact kit (Qiagen; Valencia, Calif.). FRA2 Fragment A was amplified with primer oBP594 (SEQ ID NO:210) and primer oBP595 (SEQ ID NO:211), containing a 5′ tail with homology to the 5′ end of FRA2 Fragment B. FRA2 Fragment B was amplified with primer oBP596 (SEQ ID NO:212), containing a 5′ tail with homology to the 3′ end of FRA2 Fragment A, and primer oBP597 (SEQ ID NO:213), containing a 5′ tail with homology to the 5′ end of FRA2 Fragment U. FRA2 Fragment U was amplified with primer oBP598 (SEQ ID NO:214), containing a 5′ tail with homology to the 3′ end of FRA2 Fragment B, and primer oBP599 (SEQ ID NO:215 containing a 5′ tail with homology to the 5′ end of FRA2 Fragment C. FRA2 Fragment C was amplified with primer oBP600 (SEQ ID NO:216), containing a 5′ tail with homology to the 3′ end of FRA2 Fragment U, and primer oBP601 (SEQ ID NO:217). PCR products were purified with a PCR Purification kit (Qiagen). FRA2 Fragment AB was created by overlapping PCR by mixing FRA2 Fragment A and FRA2 Fragment B and amplifying with primers oBP594 (SEQ ID NO:210) and oBP597 (SEQ ID NO:213). FRA2 Fragment UC was created by overlapping PCR by mixing FRA2 Fragment U and FRA2 Fragment C and amplifying with primers oBP598 (SEQ ID NO:214) and oBP601 (SEQ ID NO:217). The resulting PCR products were purified on an agarose gel followed by a Gel Extraction kit (Qiagen). The FRA2 ABUC cassette was created by overlapping PCR by mixing FRA2 Fragment AB and FRA2 Fragment UC and amplifying with primers oBP594 (SEQ ID NO:210) and oBP601 (SEQ ID NO:217). The PCR product was purified with a PCR Purification kit (Qiagen).

Competent cells of PNY1503 were made and transformed with the FRA2 ABUC PCR cassette using a Frozen-EZ Yeast Transformation II kit (Zymo Research; Orange, Calif.). Transformation mixtures were plated on synthetic complete media lacking uracil supplemented with 1% ethanol at 30° C. Transformants with a fra2 knockout were screened for by PCR with primers oBP602 (SEQ ID NO:218) and oBP603 (SEQ ID NO:219) using genomic DNA prepared with a Gentra Puregene Yeast/Bact kit (Qiagen). A correct transformant was grown in YPE (yeast extract, peptone, 1% ethanol) and plated on synthetic complete medium containing 5-fluoro-orotic acid (0.1%) at 30° C. to select for isolates that lost the URA3 marker. The deletion and marker removal were confirmed by PCR with primers oBP602 (SEQ ID NO:218) and oBP603 (SEQ ID NO:219) using genomic DNA prepared with a Gentra Puregene Yeast/Bact kit (Qiagen). The absence of the FRA2 gene from the isolate was demonstrated by a negative PCR result using primers specific for the deleted coding sequence of FRA2, oBP605 (SEQ ID NO:220) and oBP606 (SEQ ID NO:221). The correct isolate was selected as strain CEN.PK 113-7D MATa ura3Δ::loxP his3Δ pdc6Δ pdc1Δ::P[PDC1]-DHAD|ilvD_Sm-PDC1t pdc5Δ::P[PDC5]-ADH|sadB_Ax-PDC5t gpd2Δ::loxP fra2Δ and designated as PNY1505 (BP1135).

ADH1 Deletion and kivD L1(y) Integration

The ADH1 gene was deleted and replaced with the kivD coding region from Lactococcus lactis codon optimized for expression in Saccharomyces cerevisiae. The scarless cassette for the ADH1 deletion-kivD_L1(y) integration was first cloned into plasmid pUC19-URA3MCS.

The kivD coding region from Lactococcus lactis codon optimized for expression in Saccharomyces cerevisiae was amplified using pLH468 (SEQ ID NO:204) as template with primer oBP562 (SEQ ID NO:222), containing a PmeI restriction site, and primer oBP563 (SEQ ID NO:223), containing a 5′ tail with homology to the 5′ end of ADH1 Fragment B. ADH1 Fragment B was amplified from genomic DNA prepared as above with primer oBP564 (SEQ ID NO:224), containing a 5′ tail with homology to the 3′ end of kivD_L1(y), and primer oBP565 (SEQ ID NO:225), containing a FseI restriction site. PCR products were purified with a PCR Purification kit (Qiagen). kivD_L1(y)-ADH 1 Fragment B was created by overlapping PCR by mixing the kivD_L1(y) and ADH1 Fragment B PCR products and amplifying with primers oBP562 (SEQ ID NO:222) and oBP565 (SEQ ID NO:225). The resulting PCR product was digested with PmeI and FseI and ligated with T4 DNA ligase into the corresponding sites of pUC19-URA3MCS after digestion with the appropriate enzymes. ADH1 Fragment A was amplified from genomic DNA with primer oBP505 (SEQ ID NO:226), containing a Sad restriction site, and primer oBP506 (SEQ ID NO:227), containing an AscI restriction site. The ADH1 Fragment A PCR product was digested with Sad and AscI and ligated with T4 DNA ligase into the corresponding sites of the plasmid containing kivD_L1(y)-ADH1 Fragment B. ADH1 Fragment C was amplified from genomic DNA with primer oBP507 (SEQ ID NO:228), containing a PacI restriction site, and primer oBP508 (SEQ ID NO:229), containing a SalI restriction site. The ADH1 Fragment C PCR product was digested with PacI and SalI and ligated with T4 DNA ligase into the corresponding sites of the plasmid containing ADH1 Fragment A-kivD_L1(y)-ADH1 Fragment B. The hybrid promoter UAS(PGK1)-P_{FBA1} was amplified from vector pRS316-UAS(PGK1)-P_{FBA1}-GUS (SEQ ID NO:242) with primer oBP674 (SEQ ID NO:230), containing an AscI restriction site, and primer oBP675 (SEQ ID NO:231), containing a PmeI restriction site. The UAS(PGK1)-P_{FBA1} PCR product was digested with AscI and PmeI and ligated with T4 DNA ligase into the corresponding sites of the plasmid containing kivD_L1(y)-ADH1 Fragments ABC. The entire integration cassette was amplified from the resulting plasmid with primers oBP505 (SEQ ID NO:226) and oBP508 (SEQ ID NO:229) and purified with a PCR Purification kit (Qiagen).

Competent cells of PNY1505 were made and transformed with the ADH1-kivD_L1(y) PCR cassette constructed above using a Frozen-EZ Yeast Transformation II kit (Zymo Research). Transformation mixtures were plated on synthetic complete media lacking uracil supplemented with 1% ethanol at 30° C. Transformants were grown in YPE (1% ethanol) and plated on synthetic complete medium containing 5-fluoro-orotic acid (0.1%) at 30° C. to select for isolates that lost the URA3 marker. The deletion of ADH1 and integration of kivD_L1(y) were confirmed by PCR with external primers oBP495 (SEQ ID NO:232) and oBP496 (SEQ ID NO:233) and with kivD_L1(y) specific primer oBP562 (SEQ ID NO:222) and external primer oBP496 (SEQ ID NO:233) using genomic DNA prepared with a Gentra Puregene Yeast/Bact kit (Qiagen). The correct isolate was selected as strain CEN.PK 113-7D MATa ura3Δ::loxP his3Δ pdc6Δ pdc1Δ::P[PDC1]-DHAD|ilvD_Sm-PDC1tpdc5Δ::P[PDC5]-ADH|sadB_Ax-PDC5t gpd2Δ::loxP fra2Δ adh1Δ::UAS(PGK1)P[FBA1]-kivD_L1(y)-ADH1t and designated as PNY1507 (BP1201).

### Example 13

### Isobutanol Production-PNY1910 and PNY2242

### Methods:

### Preparation of Inoculum Medium

1 L of inoculum medium contained: 6.7 g, Yeast Nitrogen Base w/o amino acids (Difco 0919-15-3); 2.8 g, Yeast Synthetic Drop-out Medium Supplement Without Histidine, Leucine, Tryptophan and Uracil (Sigma Y2001); 20 mL of 1% (w/v) L-Leucine; 4 mL of 1% (w/v) L-Tryptophan; 3 g of ethanol; 10 g of glucose.

### Preparation of Defined Fermentation Medium

The volume of broth after inoculation was 800 mL, with the following final composition, per liter: 5 g ammonium sulfate, 2.8 g potassium phosphate monobasic, 1.9 g magnesium sulfate septahydrate, 0.2 mL antifoam (Sigma DF204), Yeast Synthetic Drop-out Medium Supplement without Histidine, Leucine, Tryptophan, and Uracil (Sigma Y2001), 16 mg L-leucine, 4 mg L-tryptophan, 6 mL of a vitamin mixture (in 1 L water, 50 mg biotin, 1 g Ca-pantothenate, 1 g nicotinic acid, 25 g myo-inositol, 1 g thiamine chloride hydrochloride, 1 g pyridoxol hydrochloride, 0.2 g p-aminobenzoic acid) 6 mL of a trace mineral solution (in 1 L water, 15 g EDTA, 4.5 g zinc sulfate heptahydrate, 0.8 g manganese chloride dehydrate, 0.3 g cobalt chloride hexahydrate, 0.3 g copper sulfate pentahydrate, 0.4 g disodium molybdenum dehydrate, 4.5 g calcium chloride dihydrate, 3 g iron sulfate heptahydrate, 1 g boric acid, 0.1 g potassium iodide), 30 mg thiamine HCl, 30 mg nicotinic acid. The pH was adjusted to 5.2 with 2N KOH and glucose added to 10 g/L.

### Preparation of Inoculum

A 125 mL shake flask was inoculated directly from a frozen vial by pipetting the whole vial culture (approx. 1 ml) into 10 mL of the inoculum medium. The flask was incubated at 260 rpm and 30° C. The strain was grown overnight until OD about 1.0. OD at λ=600 nm was determined in a Beckman spectrophotometer (Beckman, USA).

### Bioreactor Experimental Design

Fermentations were carried out in 1 L Biostat B DCU3 fermenters (Sartorius, USA) with a working volume on 0.8 L. Off-gas composition was monitored by a Prima DB mass spectrometer (Thermo Electron Corp., USA). The temperature was maintained at 30 C and pH controlled at 5.2 with 2N KOH throughout the entire fermentation. Directly after inoculation with 80 mL of the inoculum, dO was controlled by agitation at 30%, pH was controlled at 5.25, aeration was controlled at 0.2 L/min. Once OD of approximately 3 was reached, the gas was switched to N2 for anaerobic cultivation. Throughout the fermentation, glucose was maintained in excess (5-20 g/L) by manual additions of a 50% (w/w) solution.

### Methods for Analyzing Cultivation Experiments

OD at λ=600 nm was determined in a spectrophotometer by pipetting a well mixed broth sample into a cuvette (CS500 VWR International, Germany). If biomass concentration of the sample exceeded the linear absorption range of the spectrophotometer (typically OD values from 0.000 to 0.300), the sample was diluted with 0.9% NaCl solution to yield values in the linear range.

Measurements of glucose, isobutanol, and other fermentation by-products in the culture supernatant were carried out by HPLC, using a Bio-Rad Aminex HPX-87H column (Bio-Rad, USA), with refractive index (RI) and a diode array (210 nm) detectors. Chromatographic separation was achieved using 0.01 NH₂SO₄ as the mobile phase with a flow rate of 0.6 mL/min and a column temperature of 40° C. Isobutanol retention time is 32.2 minutes under these conditions. Isobutanol concentration in off-gas samples was determined by mass-spectrometer.

### Results

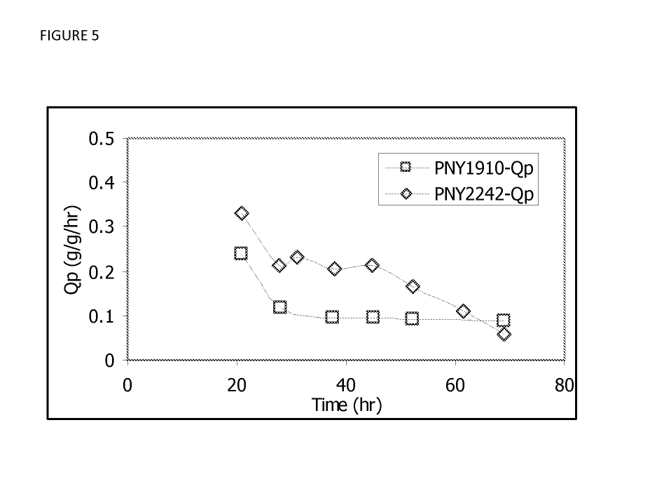

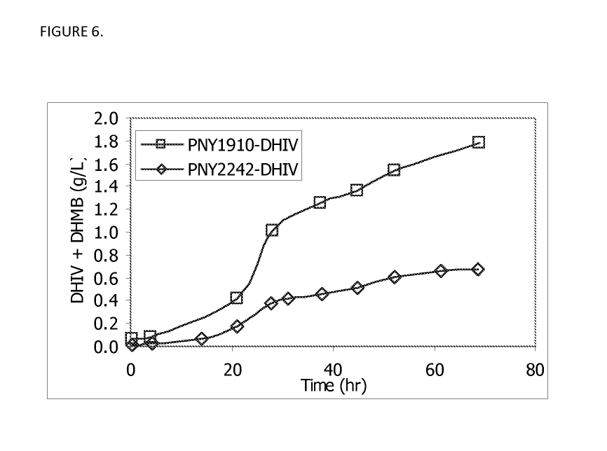

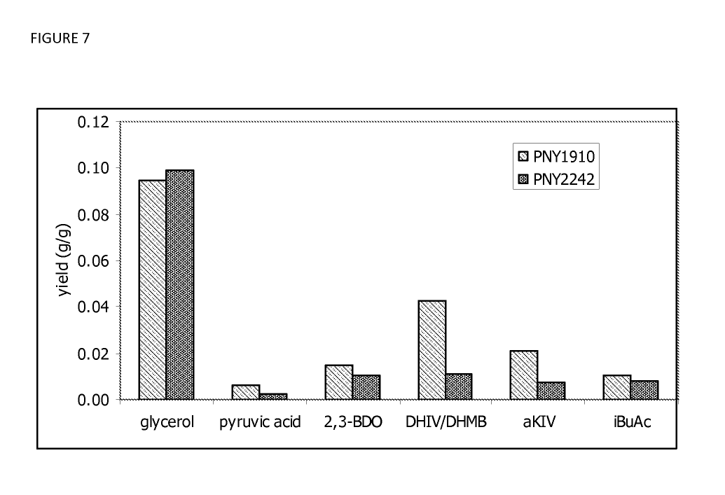

Maximal biomass concentration measured as optical density (OD), volumetric rate of isobutanol production, final isobutanol titer, and isobutanol yield on glucose are presented in the table below. The strain PNY2242 had higher titers and faster rates than the strain PNY1910 and produced isobutanol with higher specific rate and titer. The specific rates are shown in FIG. 5. Accumulation of the DHIV+DHMB in the culture supernatant was three times higher with PNY1910 compared to the PNY2242 strain (FIG. 6). Yield of glycerol, pyruvic acid, BDO, DHIV+DHMB*, αKIV, and isobutyric acid on glucose is shown in FIG. 7. *DHIV analyzed by HPLC method includes both DHIV and DHMB.

Table for Example 13

| | Max. | Rate | Titer | Yield |
|---|---|---|---|---|
| Strain | OD600 | (g/L/h) | (g/L) | (g/g) |
| PNY1910 | 5.0 | 0.16 | 10.9 | 0.25 |
| PNY2242 | 5.0 | 0.23 | 16.1 | 0.27 |

What is claimed is:

1. A recombinant yeast comprising a biosynthetic pathway capable of converting pyruvate to acetolactate, wherein said yeast produces less than 0.01 moles 2,3-dihydroxy-2-methyl butyrate (DHMB) per mole of sugar consumed.

2. A recombinant yeast comprising a biosynthetic pathway capable of converting pyruvate to acetolactate, wherein said yeast produces DHMB at a rate of less than about 1.0 mM/hour.

3. A recombinant yeast comprising a biosynthetic pathway capable of converting pyruvate to acetolactate, wherein said yeast produces an amount of 2,3-dihydroxy-3-isovalerate (DHIV) that is at least about 1.5 times the amount of DHMB produced.

4. A recombinant yeast comprising a heterologous biosynthetic pathway capable of converting pyruvate to acetolactate, wherein said yeast comprises reduced or eliminated acetolactate reductase activity.

5. The recombinant yeast of any one of claims 1-4, wherein the biosynthetic pathway is a butanol producing pathway.

6. The recombinant yeast of claim 5 comprising a recombinant ketol-acid reductoisomerase (KARI) enzyme.

7. The recombinant yeast of claim 6, wherein the KARI enzyme is capable of utilizing NADH.

8. The recombinant yeast of any one of claims 1-7, wherein the recombinant yeast is capable of producing a product under anaerobic conditions.

9. The recombinant yeast of any one of claims 1-8, wherein said yeast comprises at least one deletion, mutation, and/or substitution in an endogenous polynucleotide encoding a polypeptide having acetolactate reductase activity.

10. The recombinant yeast of any one of claims 1-9, wherein the recombinant yeast is free of an enzyme having acetolactate reductase activity.

11. The recombinant yeast of claim 9 or claim 10, wherein the polypeptide having acetolactate reductase activity comprises a polypeptide encoded by a polynucleotide selected from the group consisting of SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, and SEQ ID NO:73.

12. The recombinant yeast of any one of claims 9-11, wherein the polypeptide having acetolactate reductase activity is YMR226C.

13. The recombinant yeast of any one of claims 5-12, wherein the recombinant yeast comprises polynucleotides encoding polypeptides that catalyze the conversion of:
(a) pyruvate to acetolactate;
(b) acetolactate to 2,3-dihydroxyisovalerate;
(c) 2,3-dihydroxyisovalerate to 2-ketoisovalerate;
(d) 2-ketoisovalerate to isobutyraldehyde; and (e) isobutyraldehyde to isobutanol.

14. The recombinant yeast of claim 13, wherein the butanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

15. The recombinant yeast of any one of claims 1-14, wherein the recombinant yeast comprises at least one deletion, mutation, and/or substitution in an endogenous polynucleotide encoding a polypeptide having pyruvate decarboxylase activity.

16. The recombinant yeast of claim 15, wherein the polypeptide having pyruvate decarboxylate activity is selected from the group consisting of PDC1, PDC5, PDC6, and combinations thereof.

17. The recombinant yeast of any one of claims 1-16, wherein the yeast is free of an enzyme activity having pyruvate decarboxylase activity.

18. The recombinant yeast of any one of claims 5-17, wherein the butanol is isobutanol.

19. A method for the production of butanol comprising growing the recombinant yeast of any one of claims 5-18 under conditions whereby butanol is produced.

20. The method of claim 19, wherein the butanol is isobutanol.

21. A method for the production of butanol comprising:
(a) growing a recombinant yeast comprising a biosynthetic pathway capable of converting pyruvate to acetolactate under conditions whereby butanol is produced; and
(b) removing DHMB from the culture.

22. The method of claim 21, wherein the DHMB is removed by extraction into an organic phase.

23. The method of claim 22, wherein the DHMB is removed by reactive extraction.

24. The method of any one of claims 21-23, wherein the recombinant yeast comprises a recombinant ketol-acid reductoisomerase (KARI) enzyme.

25. The method of claim 24, wherein the KARI enzyme is capable of utilizing NADH.

26. The method of any one of claims 21-25, wherein the recombinant yeast comprises at least one deletion, mutation, and/or substitution in an endogenous polynucleotide encoding a polypeptide having pyruvate decarboxylase activity.

27. The method of any one of claims 21-26, wherein the recombinant yeast is free of an enzyme having pyruvate decarboxylase activity

28. The method of any one of claims 21-27, wherein the recombinant yeast comprises at least one deletion, mutation, and/or substitution in an endogenous polynucleotide encoding a polypeptide having acetolactate reductase activity.

29. The method of any one of claims 21-28, wherein the recombinant yeast is free of an enzyme having acetolactate reductase activity.

30. The method of claim 28 or claim 29, wherein the enzyme having acetolactate reductase activity comprises a polypeptide encoded by a polynucleotide selected from the group consisting of SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, and SEQ ID NO:73.

31. The recombinant yeast of any one of claims 28-30, wherein the polypeptide having acetolactate reductase activity is YMR226C.

32. The method of any one of claims 21-30, wherein the butanol is isobutanol.

33. The method of any one of claims 21-32, wherein the growing occurs in anaerobic conditions.

34. A composition produced by the method of any one of claims 21-33, wherein the composition comprises butanol and no more than about 0.5 mM DHMB.

35. A method of identifying a gene involved in DHMB production comprising:
(i) providing a collection of yeast strains comprising at least two or more gene deletions;
(ii) measuring the amount of DHMB produced by individual yeast strains;
(iii) selecting a yeast strain that produces no more than about 1.0 mM DHMB/hour; and
(iv) identifying the gene that is deleted in the selected yeast strain.

36. A method of identifying a gene involved in DHMB production comprising:
(i) providing a collection of yeast strains that over-express at least two or more genes;
(ii) measuring the amount of DHMB produced by individual yeast strains;
(iii) selecting a yeast strain that produces at least about 1.0 mM DHMB; and
(iv) identifying the gene that is over-expressed in the selected yeast strain.

37. The method of claim 35 or claim 36 further comprising creating a deletion, mutation, and/or substitution in the identified gene in a recombinant yeast comprising a biosynthetic pathway capable of converting pyruvate to acetolactate.

38. A recombinant yeast produced by the method of claim 37.

39. The recombinant yeast of claim 38, wherein the recombinant yeast comprises a recombinant ketol-acid reductoisomerase (KARI) enzyme.

40. The recombinant yeast of claim 39, wherein the KARI enzyme is capable of utilizing NADH.

41. The recombinant yeast of any one of claims 38-40, wherein the biosynthetic pathway is a butanol producing pathway.

42. The recombinant yeast of any one of claims 38-41, wherein the recombinant yeast comprises at least one deletion, mutation, and/or substitution in an endogenous polynucleotide encoding a polypeptide having pyruvate decarboxylase activity.

43. The recombinant yeast of any one of claims 38-42, wherein the yeast is free of an enzyme having pyruvate decarboxylase activity

44. The recombinant yeast of any one of claims 38-43, wherein the recombinant yeast is free of an enzyme having acetolactate reductase activity.

45. A method of producing butanol comprising growing the recombinant yeast of any one of claims 38-44 under conditions whereby butanol is produced.

46. The method of claim 45, wherein the butanol is isobutanol.

47. The method of claim 45 or claim 46, wherein the growing occurs in anaerobic conditions.

48. A composition comprising a i) recombinant yeast capable of producing butanol, ii) butanol, and iii) no more than about 0.5 mM DHMB.

49. The composition of claim 48, wherein the recombinant yeast comprises a butanol biosynthetic pathway.

50. The composition of claim 48 or claim 49, wherein the recombinant yeast comprises at least one deletion, mutation, and/or substitution in an endogenous polynucleotide encoding a polypeptide having pyruvate decarboxylase activity.

51. The composition of any one of claims 48-50, wherein the recombinant yeast comprises at least one deletion, mutation, and/or substitution in an endogenous polynucleotide encoding a polypeptide having acetolactate reductase activity.

52. The composition of claim 51, wherein the polypeptide having acetolactate reductase activity comprises a polypeptide encoded by a polynucleotide selected from the group consisting of SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, and SEQ ID NO:73.

53. The composition of claim 51 or 52, wherein the polypeptide having acetolactate reductase activity is YMR226C.

54. The composition of any one of claims 48-53, wherein the butanol is isobutanol.

55. A method for the production of butanol comprising:
(a) growing a recombinant yeast comprising a biosynthetic pathway capable of converting pyruvate to acetolactate under conditions whereby butanol is produced; and
(b) measuring DHIV concentration;
wherein steps a) and b) can be performed simultaneously or sequentially and in any order.

56. The method of claim 55, wherein the measuring comprises liquid chromatography-mass spectrometry.

57. A method for the production of butanol comprising:
(a) growing a recombinant yeast comprising a biosynthetic pathway capable of converting pyruvate to acetolactate under conditions whereby butanol is produced; and
(b) measuring DHMB concentration;
wherein steps a) and b) can be performed simultaneously or sequentially and in any order.

58. The method of claim 57, wherein the measuring comprises liquid chromatography-mass spectrometry.

59. A method for increasing ketol-acid reductoisomerase (KARI) activity comprising a) providing a composition comprising acetolactate, a KARI enzyme, and an acetolactate reductase enzyme and b) decreasing DHMB levels.

60. The method of claim 59, wherein said decreasing DHMB levels is achieved by decreasing acetolactate reductase enzyme activity.

61. The method of claim 59, wherein said decreasing DHMB levels is achieved by removing DHMB from the composition.

62. The method of any one of claims 59-61, wherein said acetolactate, said KARI enzyme, or said acetolactate reductase enzyme are present in a recombinant yeast.

63. The method of claim 62, wherein the recombinant yeast comprises a biosynthetic pathway capable of converting pyruvate to acetolactate.

64. A method for increasing dihydroxyacid dehydratase (DHAD) activity comprising a) providing a composition comprising dihydroxyisovalerate (DHIV) and a DHAD enzyme and b) decreasing DHMB levels.

65. The method of claim 64, wherein said decreasing DHMB levels is achieved by decreasing acetolactate reductase enzyme activity.

66. The method of claim 64, wherein said decreasing DHMB levels is achieved by removing DHMB from the composition.

67. The method of any one of claims 64-66, wherein said DHIV or said DHAD enzyme are present in a recombinant yeast.

68. The method of claim 67, wherein the recombinant yeast comprises a biosynthetic pathway capable of converting pyruvate to acetolactate.


SEQUENCE LISTING (262 sequences)

SEQ ID NO: 1 - DNA, 10934 bp, artificial sequence
  Synthetic construct
ggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcgcccgggccactagtcagatgccgcgggcacttgagcacctcatgcacagcaataacacaacacaatggttagtagcaacctgaattcggtcattgatgcatgcatgtgccgtgaagcgggacaaccagaaaagtcgtctataaatgccggcacgtgcgatcatcgtggcggggttttaagagtgcatatcacaaattgtcgcattaccgcggaaccgccagatattcattacttgacgcaaaagcgtttgaaataatgacgaaaaagaaggaagaaaaaaaaagaaaaataccgcttctaggcgggttatctactgatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtctcaaggtcaaaactgtatggaaaccttgtcacctcacttaattctagctagcctaccctgcaagtcaagaggtctccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaatcccttattccttctagctatttttcataaaaaaccaagcaactgcttatcaacacacaaacactaaatcaaagctgaggatggatttatttgagtcattagcacaaaaaattactggtaaagatcaaacaattgttttccctgaaggaactgaaccccgaattgtcggtgcggcagcgcgattagctgcagacggcttggttaagccgattgttttaggtgcaacggacaaagttcaggctgtggctaacgatttgaatgcggatttaacaggcgttcaagtccttgatcctgcgacatacccggctgaagataagcaagcaatgcttgatgccctcgttgaacggcggaaaggtaagaatacgccagaacaagcggctaaaatgctggaagatgaaaactactttggcacgatgctcgtttatatgggcaaagcggatgggatggtttcaggtgcaatccatccaactggtgatacggtacggccagcgttacaaattattaagaccaagcccggttcacaccgaatctcgggtgcatttatcatgcaaaagggtgaggaacgctacgtctttgctgactgtgccatcaatattgatcccgatgccgatacgttagcggaaattgccactcagagtgcggctactgctaaggtcttcgatattgacccgaaagttgcgatgctcagcttctcaactaagggttcggctaagggtgaaatggtcactaaagtgcaagaagcaacggccaaggcgcaagctgctgaaccggaattggctatcgatggtgaacttcaatttgacgcggccttcgttgaaaaagttggtttgcaaaaggctcctggttccaaagtagctggtcatgccaatgtctttgtatttccagagcttcagtctggtaatattggctataagattgcgcaacgatttggtcattttgaagcggtgggtcctgtcttgcaaggcctgaacaagccggtctccgacttgtcacgtggatgcagtgaagaagacgtttataaggttgcgattattacagcagcccaaggattagcttaattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtactagttctagagcggccgcccgcaaattaaagccttcgagcgtcccaaaaccttctcaagcaaggttttcagtataatgttacatgcgtacacgcgtttgtacagaaaaaaaagaaaaatttgaaatataaataacgttcttaatactaacataactattaaaaaaaataaatagggacctagacttcaggttgtctaactccttccttttcggttagagcggatgtgggaggagggcgtgaatgtaagcgtgacataactaattacatgattaattaattattttaaacccttccattgccaatcattaacttctggcaagtcagttccggcatcccggatataggcattgtgtttagcaagcatattatccatggattgaacgaaggccgcaccagtgttttccattgctggttgcgccgcaattgccgacttagctaagtcgaagcggtccatctggttcatgacccgtacgtcgaatggtgtggtaatatcaccattttcacggtaaccgtggacgtataagttatggttgtgacgatcaaagaagatgtcacgaactaagtcttcgtaaccgtggaaagcaaagaccactggtttgtccttagtaaagtaatggtcaaactcagcatctgacaagccccgcggatccttttcaggactacgtaacttcaagatgtcgaccacgttcacgaaacgaatcttcatctctgggaaactgtcgtgtagtaattggatggcagccaacgtttcaagcgttggttccgtcccagcagctgcaaagacaatgtctggttcgctaccttggtccgtacttgcccaatcaatgataccaagaccattgtcaactaattgcttagcttcttcaatgctgaaccattgttgacgtgggtgttttgacgtaaccacgtagttgatcttttcttggctccggaaaatgacgtcaccgacagctaataacgtgttggcatcggctggtaaatattcacgaatgtattctggtttcttttcggccaaatgagttaatgcacctggatcttggtgggtataaccattatggtcttgttggaatacagttgaagccgcgataatgttaagtgatgggtactttttacgccaatcaagttcattggctttacgtaaccacttgaagtgttgcgtcaacattgagtccacaacgcgtaggaaggcttcataactggcaaataacccatgacgtccagttaagacgtaaccttctaaccaaccttcagcttggtgttcagataactgagcatctaagacccggccagctggtgcttcatattggtcactatctggatgaatgtcttccatccattgacgattagtggtttcgaagacaccatataaacggttagacatggtttcatcaggtccgaacaaccggaagttatcaggatttttcttgatgacatcccgcaaatagtctgaccaaacgatcatatcttgcttaacattcgcgccttctttggacgtatcgaccgcataatcacggaagtttggtaagttcaaggctttcggatcgaccccaccattggtgattgggttagcagccatccgactgtccccagtaggaataatttctttaatatcatccttcaaagagccatcttcattgaagagttcttttggttgatatgattcgagccaatcaactaaagcatccgcatgttccatgtcattttgatcaacaggaatcggaatttgatgagcacggaatgaaccttcgatcttatcaccgtcccatgacttcggaccagtccagcccttaggtgcgcggaagacgatcattggccatactggcaatgttgcatcgttattttcgcgagcatgcttctggattgccttgatcttttcaacggcttcatccatggccttagctaaggctgggtgaaccttttcaggatcgtcaccttcaacgaagattggttcccaattcatgctttcgaagtattccttaatcttagcatcagaagtccgaccaaaaatcgttggattagaaatcttaaaaccatttaagttcaagattggtaaaacagccccgtcgttgattgggttaatgaacttcgttgattgccatgaagttgctaatggacccgtttcggattccccatcaccaacaacaaccgcggcgatttcgtcaggattgtcaagaattgccccaaccccgtgtgaaattgagtaaccaagttcgccaccttcgtggattgaaccgggtgtttcaggtgccgcatgggaagcaaccccacctgggaatgagaattgcttgaagagcttttgcatcccttcaacatcctgcgtaatttctggataaatatcggtgtaagtaccgtcaaggtaagagtttgaaaccatcacttgaccaccatgacctggaccttcaacgtagaacatcttcaaaccgtacttgttgatgacccggttaagatgagcatagataaagttttgaccggcaatcgtcccccagtgaccaattggatgaaccttaacgtcactggccttcaatggccgttgtaatagtggattatcttttaaataaagttgaccaactgataagtagttggcagcacgccagtacttatcaactttttgcaaatatgctggtgatgagtaatctgttgtcatcctcagctggaacttagattagattgctatgctttctctctaacgagcaagaagtaaaaaaagttgtaatagaacaagaaaaatgaaactgaagcttgagaaattgaagaccgtttattagcttaaatatcaatgggaggtcatcgaaagagaaaaaaatcaagaaagaaactctcaagaaaaagaaacgtgataaaaatttttattgcctctctcgacgaagagaaagaaacgaggcggtcccttttttcttttccaaacctttagtacgggtaattagcgacaccctagaggaagaaagaggggaaatttagtatgctgtgcttgggtgtcttgaagtggtacggcgatgcgcggagtccgagaaaatctggaagagtaaaaagggggtagaagcgttttgaagctatccgc

SEQ ID NO: 2 - DNA, 9220 bp, artificial sequence
  Synthetic construct
ccgcattgcggattacgtattctaatgttcagataacttcgtataatgtatgctatacgaagttatcgaacagagaaactaaatccacattaattgagagttctatctattagaaaatgcaaactccaactaaatgggaaaacagataacctcttttatttttttttaatgtttgatattcgagtctttttcttttgttaggtttatattcatcatttcaatgaataaaagaagcttcttattttggttgcaaagaatgaaaaaaaaggattttttcatacttctaaagcttcaattataaccaaaaattttataaatgaagagaaaaaatctagtagtatcaagttaaacttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatctcgagttttaatgttacttctcttgcagttagggaactataatgtaactcaaaataagattaaacaaactaaaataaaaagaagttatacagaaaaacccatataaaccagtactaatccataataataatacacaaaaaaactatcaaataaaaccagaaaacagattgaatagaaaaattttttcgatctccttttatattcaaaattcgatatatgaaaaagggaactctcagaaaatcaccaaatcaatttaattagatttttcttttccttctagcgttggaaagaaaaatttttctttttttttttagaaatgaaaaatttttgccgtaggaatcaccgtataaaccctgtataaacgctactctgttcacctgtgtaggctatgattgacccagtgttcattgttattgcgagagagcgggagaaaagaaccgatacaagagatccatgctggtatagttgtctgtccaacactttgatgaacttgtaggacgatgatgtgtatttagacgagtacgtgtgtgactattaagtagttatgatagagaggtttgtacggtgtgttctgtgtaattcgattgagaaaatggttatgaatccctagataacttcgtataatgtatgctatacgaagttatccagtgatgatacaacgagttagccaaggtgggggatcctctagagtcttaaggccgcccgcaaattaaagccttcgagcgtcccaaaaccttctcaagcaaggttttcagtataatgttacatgcgtacacgcgtttgtacagaaaaaaaagaaaaatttgaaatataaataacgttcttaatactaacataactattaaaaaaaataaatagggacctagacttcaggttgtctaactccttccttttcggttagagcggatgtgggaggagggcgtgaatgtaagcgtgacataactaattacatgattaattaattattttaaacccttccattgccaatcattaacttctggcaagtcagttccggcatcccggatataggcattgtgtttagcaagcatattatccatggattgaacgaaggccgcaccagtgttttccattgctggttgcgccgcaattgccgacttagctaagtcgaagcggtccatctggttcatgacccgtacgtcgaatggtgtggtaatatcaccattttcacggtaaccgtggacgtataagttatggttgtgacgatcaaagaagatgtcacgaactaagtcttcgtaaccgtggaaagcaaagaccactggtttgtccttagtaaagtaatggtcaaactcagcatctgacaagccccgcggatccttttcaggactacgtaacttcaagatgtcgaccacgttcacgaaacgaatcttcatctctgggaaactgtcgtgtagtaattggatggcagccaacgtttcaagcgttggttccgtcccagcagctgcaaagacaatgtctggttcgctaccttggtccgtacttgcccaatcaatgataccaagaccattgtcaactaattgcttagcttcttcaatgctgaaccattgttgacgtgggtgttttgacgtaaccacgtagttgatcttttcttggctccggaaaatgacgtcaccgacagctaataacgtgttggcatcggctggtaaatattcacgaatgtattctggtttcttttcggccaaatgagttaatgcacctggatcttggtgggtataaccattatggtcttgttggaatacagttgaagccgcgataatgttaagtgatgggtactttttacgccaatcaagttcattggctttacgtaaccacttgaagtgttgcgtcaacattgagtccacaacgcgtaggaaggcttcataactggcaaataacccatgacgtccagttaagacgtaaccttctaaccaaccttcagcttggtgttcagataactgagcatctaagacccggccagctggtgcttcatattggtcactatctggatgaatgtcttccatccattgacgattagtggtttcgaagacaccatataaacggttagacatggtttcatcaggtccgaacaaccggaagttatcaggatttttcttgatgacatcccgcaaatagtctgaccaaacgatcatatcttgcttaacattcgcgccttctttggacgtatcgaccgcataatcacggaagtttggtaagttcaaggctttcggatcgaccccaccattggtgattgggttagcagccatccgactgtccccagtaggaataatttctttaatatcatccttcaaagagccatcttcattgaagagttcttttggttgatatgattcgagccaatcaactaaagcatccgcatgttccatgtcattttgatcaacaggaatcggaatttgatgagcacggaatgaaccttcgatcttatcaccgtcccatgacttcggaccagtccagcccttaggtgcgcggaagacgatcattggccatactggcaatgttgcatcgttattttcgcgagcatgcttctggattgccttgatcttttcaacggcttcatccatggccttagctaaggctgggtgaaccttttcaggatcgtcaccttcaacgaagattggttcccaattcatgctttcgaagtattccttaatcttagcatcagaagtccgaccaaaaatcgttggattagaaatcttaaaaccatttaagttcaagattggtaaaacagccccgtcgttgattgggttaatgaacttcgttgattgccatgaagttgctaatggacccgtttcggattccccatcaccaacaacaaccgcggcgatttcgtcaggattgtcaagaattgccccaaccccgtgtgaaattgagtaaccaagttcgccaccttcgtggattgaaccgggtgtttcaggtgccgcatgggaagcaaccccacctgggaatgagaattgcttgaagagcttttgcatcccttcaacatcctgcgtaatttctggataaatatcggtgtaagtaccgtcaaggtaagagtttgaaaccatcacttgaccaccatgacctggaccttcaacgtagaacatcttcaaaccgtacttgttgatgacccggttaagatgagcatagataaagttttgaccggcaatcgtcccccagtgaccaattggatgaaccttaacgtcactggccttcaatggccgttgtaatagtggattatcttttaaataaagttgaccaactgataagtagttggcagcacgccagtacttatcaactttttgcaaatatgctggtgatgagtaatctgttgtcatcctcagctggaacttagattagattgctatgctttctctctaacgagcaagaagtaaaaaaagttgtaatagaacaagaaaaatgaaactgaagcttgagaaattgaagaccgtttattagcttaaatatcaatgggaggtcatcgaaagagaaaaaaatcaagaaagaaactctcaagaaaaagaaacgtgataaaaatttttattgcctctctcgacgaagagaaagaaacgaggcggtcccttttttcttttccaaacctttagtacgggtaattagcgacaccctagaggaagaaagaggggaaatttagtatgctgtgcttgggtgtcttgaagtggtacggcgatgcgcggagtccgagaaaatctggaagagtaaaaagggggtagaagcgttttgaagctatccgcggtggttaagcctaaccaggccaattcaacagactgtcggcaacttcttgtctggtctttccatggtaagtgacagtgcagtaataatatgaaccaatttatttttcgttacataaaaatgcttataaaactttaactaataattagagattaaatcgcaaacggccggccaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttcatggccttgcggccgcttaattaatctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatccggcgcgccgttttatttgtatcgaggtgtctagtcttctattacactaatgcagtttcagggttttggaaaccacactgtttaaacagtgttccttaatcaaggatacctctttttttttccttggttccactaattcatcggttttttttttggaagacatcttttccaacgaaaagaatatacatatcgtttaagagaaattctccaaatttgtaaagaagcggacccagacttaa

SEQ ID NO: 3 - DNA, 38 bp, artificial sequence
  BK468 primer
gcctcgagttttaatgttacttctcttgcagttaggga

SEQ ID NO: 4 - DNA, 22 bp, artificial sequence
  N160SeqF5 Primer
cctgaagtctaggtccctattt

SEQ ID NO: 5 - DNA, 19 bp, artificial sequence
  N160SeqR5 Primer
tgagcccgaaagagaggat

SEQ ID NO: 6 - DNA, 38 bp, artificial sequence
  N821 primer
cgcccgggccactagtcagatgccgcgggcacttgagc

SEQ ID NO: 7 - DNA, 44 bp, artificial sequence
  N822 primer
cgcctcagctttgatttagtgtttgtgtgttgataagcagttgc

SEQ ID NO: 8 - DNA, 20 bp, artificial sequence
  N886 primer
caatgattgttggtaaaggg

SEQ ID NO: 9 - DNA, 30 bp, artificial sequence
  N110 primer
gcgatttaatctctaattattagttaaagt

SEQ ID NO: 10 - DNA, 30 bp, artificial sequence
  N1114 primer
atatgctggtgatgagtaatctgttgtcat

SEQ ID NO: 11 - DNA, 30 bp, artificial sequence
  N1115 primer
tttttgtgctaatgactcaaataaatccat

SEQ ID NO: 12 - DNA, 62 bp, artificial sequence
  N1176 primer
gcatagcaatctaatctaagttccagctgaggatgacaacagattactcatcaccagcatat

SEQ ID NO: 13 - DNA, 62 bp, artificial sequence
  N1177 primer
atcaacacacaaacactaaatcaaagctgaggatggatttatttgagtcattagcacaaaaa

SEQ ID NO: 14 - DNA, 63 bp, artificial sequence
  N1178 primer
ggtatcgataagcttgatatcgaattcctgcgcccgggccactagtcagatgccgcgggcact

SEQ ID NO: 15 - DNA, 30 bp, artificial sequence
  N1214 primer
aaaaagggggtagaagcgttttgaagctat

SEQ ID NO: 16 - DNA, 24 bp, artificial sequence
  N1237 primer
gatgatgctatttggtgcagaggg

SEQ ID NO: 17 - DNA, 27 bp, artificial sequence
  N1238 primer
ccttactgaagtatttagttatcatgg

SEQ ID NO: 18 - DNA, 25 bp, artificial sequence
  N1239 primer
gacgaggataatgtgcatgaacggg

SEQ ID NO: 19 - DNA, 24 bp, artificial sequence
  N1240 primer
ggatgtatgggctaaatgtacggg

SEQ ID NO: 20 - DNA, 24 bp, artificial sequence
  N1241 primer
catccagtgtcgaaaacgagctcg

SEQ ID NO: 21 - DNA, 24 bp, artificial sequence
  N1242 primer
gtttcgttgcgttggtggaatggc

SEQ ID NO: 22 - DNA, 22 bp, artificial sequence
  oBP512 Primer
aaagttggcatagcggaaactt

SEQ ID NO: 23 - DNA, 804 bp, Saccharomyces cerevisiae
atgtcccaaggtagaaaagctgcagaaagattggctaagaagactgtcctcattacaggtgcatctgctggtattggtaaggcgaccgcattagagtacttggaggcatccaatggtgatatgaaactgatcttggctgctagaagattagaaaagctcgaggaattgaagaagaccattgatcaagagtttccaaacgcaaaagttcatgtggcccagctggatatcactcaagcagaaaaaatcaagcccttcattgaaaacttgccacaagagttcaaggatattgacattctggtgaacaatgccggaaaggctcttggcagtgaccgtgtgggccagatcgcaacggaggatatccaggacgtgtttgacaccaacgtcacggctttaatcaatatcacacaagctgtactgcccatattccaagccaagaattcaggagatattgtaaatttgggttcaatcgctggcagagacgcatacccaacaggttctatctattgtgcctctaagtttgccgtgggggcgttcactgatagtttgagaaaggagctcatcaacactaaaattagagtcattctaattgcaccagggctagtcgagactgaattttcactagttagatacagaggtaacgaggaacaagccaagaatgtttacaaggatactaccccattgatggctgatgacgtggctgatctgatcgtctatgcaacttccagaaaacaaaatactgtaattgcagacactttaatctttccaacaaaccaagcgtcacctcatcatatcttccgtggataa

SEQ ID NO: 24 - protein, 267 aa, Saccharomyces cerevisiae
MSQGRKAAERLAKKTVLITGASAGIGKATALEYLEASNGDMKLILAARRLEKLEELKKTIDQEFPNAKVHVAQLDITQAEKIKPFIENLPQEFKDIDILVNNAGKALGSDRVGQIATEDIQDVFDTNVTALINITQAVLPIFQAKNSGDIVNLGSIAGRDAYPTGSIYCASKFAVGAFTDSLRKELINTKIRVILIAPGLVETEFSLVRYRGNEEQAKNVYKDTTPLMADDVADLIVYATSRKQNTVIADTLIFPTNQASPHHIFRG

SEQ ID NO: 25 - DNA, 1410 bp, Saccharomyces cerevisiae
atgtcttattcagctgccgataatttacaagattcattccaacgtgccatgaacttttctggctctcctggtgcagtctcaacctcaccaactcagtcatttatgaacacactacctcgtcgtgtaagcattacaaagcaaccaaaggctttaaaacctttttctactggtgacatgaatattctactgttggaaaatgtcaatgcaactgcaatcaaaatcttcaaggatcagggttaccaagtagagttccacaagtcttctctacctgaggatgaattgattgaaaaaatcaaagacgtacacgctatcggtataagatccaaaactagattgactgaaaaaatactacagcatgccaggaatctagtttgtattggttgtttttgcataggtaccaatcaagtagacctaaaatatgccgctagtaaaggtattgctgttttcaattcgccattctccaattcaagatccgtagcagaattggtaattggtgagatcattagtttagcaagacaattaggtgatagatccattgaactgcatacaggtacatggaataaagtcgctgctaggtgttgggaagtaagaggaaaaactctcggtattattgggtatggtcacattggttcgcaattatcagttcttgcagaagctatgggcctgcatgtgctatactatgatatcgtgacaattatggccttaggtactgccagacaagtttctacattagatgaattgttgaataaatctgattttgtaacactacatgtaccagctactccagaaactgaaaaaatgttatctgctccacaattcgctgctatgaaggacggggcttatgttattaatgcctcaagaggtactgtcgtggacattccatctctgatccaagccgtcaaggccaacaaaattgcaggtgctgctttagatgtttatccacatgaaccagctaagaacggtgaaggttcatttaacgatgaacttaacagctggacttctgagttggtttcattaccaaatataatcctgacaccacatattggtggctctacagaagaagctcaaagttcaatcggtattgaggtggctactgcattgtccaaatacatcaatgaaggtaactctgtcggttctgtgaacttcccagaagtcagtttgaagtctttggactacgatcaagagaacacagtacgtgtcttgtatattcatcgtaacgttcctggtgttttgaagaccgttaatgatatcttatccgatcataatatcgagaaacagttttctgattctcacggcgagatcgcttatctaatggcagacatctcttctgttaatcaaagtgaaatcaaggatatatatgaaaagttgaaccaaacttctgccaaagtttccatcaggttattatactaa

SEQ ID NO: 26 - protein, 469 aa, Saccharomyces cerevisiae
MSYSAADNLQDSFQRAMNFSGSPGAVSTSPTQSFMNTLPRRVSITKQPKALKPFSTGDMNILLLENVNATAIKIFKDQGYQVEFHKSSLPEDELIEKIKDVHAIGIRSKTRLTEKILQHARNLVCIGCFCIGTNQVDLKYAASKGIAVFNSPFSNSRSVAELVIGEIISLARQLGDRSIELHTGTWNKVAARCWEVRGKTLGIIGYGHIGSQLSVLAEAMGLHVLYYDIVTIMALGTARQVSTLDELLNKSDFVTLHVPATPETEKMLSAPQFAAMKDGAYVINASRGTVVDIPSLIQAVKANKIAGAALDVYPHEPAKNGEGSFNDELNSWTSELVSLPNIILTPHIGGSTEEAQSSIGIEVATALSKYINEGNSVGSVNFPEVSLKSLDYDQENTVRVLYIHRNVPGVLKTVNDILSDHNIEKQFSDSHGEIAYLMADISSVNQSEIKDIYEKLNQTSAKVSIRLLY

SEQ ID NO: 27 - DNA, 792 bp, Saccharomyces cerevisiae
atgggcaaggttattttgattacaggtgcctcccgtgggattggcctgcaattggtgaaaactgttatcgaagaggacgatgaatgcatcgtctacggcgtagcaagaacggaagctggtctgcagtctttgcaaagagaatacggtgcagacaaatttgtctatcgtgtcctcgacatcacggacaggtctcgaatggaagcgttggtggaggaaatccggcaaaagcatggaaaactggacggtattgtcgcaaatgcggggatgctagaaccggtgaagtccatctcccagtccaactccgaacacgacatcaagcagtgggaacggctgttcgatgtgaactttttcagcattgtctctttggtggcactgtgtttacccctcttgaagagctcgccatttgtaggcaacattgtcttcgtcagctctggagccagtgtgaaaccatataacggatggtcggcgtacggctgctcgaaagccgcattaaaccactttgccatggacattgccagtgaagagcccagtgataaagtgcgtgccgtgtgtattgcaccgggcgtcgttgacacgcagatgcagaaagatattagggaaacattgggtcctcagggcatgacacccaaggctctcgagaggtttactcaattgtacaagacttcgtcactgctggacccaaaggtgcctgcggcggtactagcgcaactcgtcctgaaaggtattcccgactctttgaacggtcaatatctccgctacaacgatgagcgactggggccggtgcagggctag

SEQ ID NO: 28 - protein, 263 aa, Saccharomyces cerevisiae
MGKVILITGASRGIGLQLVKTVIEEDDECIVYGVARTEAGLQSLQREYGADKFVYRVLDITDRSRMEALVEEIRQKHGKLDGIVANAGMLEPVKSISQSNSEHDIKQWERLFDVNFFSIVSLVALCLPLLKSSPFVGNIVFVSSGASVKPYNGWSAYGCSKAALNHFAMDIASEEPSDKVRAVCIAPGVVDTQMQKDIRETLGPQGMTPKALERFTQLYKTSSLLDPKVPAAVLAQLVLKGIPDSLNGQYLRYNDERLGPVQG

SEQ ID NO: 29 - DNA, 1503 bp, Saccharomyces cerevisiae
atgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtgctttccacgacactgaatgggctacccaagacccaagagaaagaggccgtctactaagtaagttggctgacgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacagaagtcggtaagagtgttgctgtcgactcttctgaatctaacttgaagaaaatcactttggaactaggtggtaagtccgcccatttggtctttgacgatgctaacattaagaagactttaccaaatctagtaaacggtattttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaa

SEQ ID NO: 30 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 31 - DNA, 1029 bp, Saccharomyces cerevisiae
atggttttagttaagcaggtaagactcggtaactcaggtcttaagatatcaccgatagtgataggatgtatgtcatacgggtccaagaaatgggcggactgggtcatagaggacaagacccaaattttcaagattatgaagcattgttacgataaaggtcttcgtacttttgacacagcagatttttattctaatggtttgagtgaaagaataattaaggagtttctggagtactacagtataaagagagaaacggtggtgattatgaccaaaatttacttcccagttgatgaaacgcttgatttgcatcataacttcactttaaatgaatttgaagaattggacttgtccaaccagcggggtttatccagaaagcatataattgctggtgtcgagaactctgtgaaaagactgggcacatatatagaccttttacaaattcacagattagatcatgaaacgccaatgaaagagatcatgaaggcattgaatgatgttgttgaagcgggccacgttagatacattggggcttcgagtatgttggcaactgaatttgcagaactgcagttcacagccgataaatatggctggtttcagttcatttcttcgcagtcttactacaatttgctctatcgtgaagatgaacgcgaattgattccttttgccaaaagacacaatattggtttacttccatggtctcctaacgcacgaggcatgttgactcgtcctctgaaccaaagcacggacaggattaagagtgatccaactttcaagtcgttacatttggataatctcgaagaagaacaaaaggaaattataaatcgtgtggaaaaggtgtcgaaggacaaaaaagtctcgatggctatgctctccattgcatgggttttgcataaaggatgtcaccctattgtgggattgaacactacagcaagagtagacgaagcgattgccgcactacaagtaactctaacagaagaagagataaagtacctcgaggagccctacaaaccccagaggcaaagatgttaa

SEQ ID NO: 32 - protein, 342 aa, Saccharomyces cerevisiae
MVLVKQVRLGNSGLKISPIVIGCMSYGSKKWADWVIEDKTQIFKIMKHCYDKGLRTFDTADFYSNGLSERIIKEFLEYYSIKRETVVIMTKIYFPVDETLDLHHNFTLNEFEELDLSNQRGLSRKHIIAGVENSVKRLGTYIDLLQIHRLDHETPMKEIMKALNDVVEAGHVRYIGASSMLATEFAELQFTADKYGWFQFISSQSYYNLLYREDERELIPFAKRHNIGLLPWSPNARGMLTRPLNQSTDRIKSDPTFKSLHLDNLEEEQKEIINRVEKVSKDKKVSMAMLSIAWVLHKGCHPIVGLNTTARVDEAIAALQVTLTEEEIKYLEEPYKPQRQRC

SEQ ID NO: 33 - DNA, 1086 bp, Saccharomyces cerevisiae
atgctttacccagaaaaatttcagggcatcggtatttccaacgcaaaggattggaagcatcctaaattagtgagttttgacccaaaaccctttggcgatcatgacgttgatgttgaaattgaagcctgtggtatctgcggatctgattttcatatagccgttggtaattggggtccagtcccagaaaatcaaatccttggacatgaaataattggccgcgtggtgaaggttggatccaagtgccacactggggtaaaaatcggtgaccgtgttggtgttggtgcccaagccttggcgtgttttgagtgtgaacgttgcaaaagtgacaacgagcaatactgtaccaatgaccacgttttgactatgtggactccttacaaggacggctacatttcacaaggaggctttgcctcccacgtgaggcttcatgaacactttgctattcaaataccagaaaatattccaagtccgctagccgctccattattgtgtggtggtattacagttttctctccactactaagaaatggctgtggtccaggtaagagggtaggtattgttggcatcggtggtattgggcatatggggattctgttggctaaagctatgggagccgaggtttatgcgttttcgcgaggccactccaagcgggaggattctatgaaactcggtgctgatcactatattgctatgttggaggataaaggctggacagaacaatactctaacgctttggaccttcttgtcgtttgctcatcatctttgtcgaaagttaattttgacagtatcgttaagattatgaagattggaggctccatcgtttcaattgctgctcctgaagttaatgaaaagcttgttttaaaaccgttgggcctaatgggagtatcaatctcaagcagtgctatcggatctaggaaggaaatcgaacaactattgaaattagtttccgaaaagaatgtcaaaatatgggtggaaaaacttccgatcagcgaagaaggcgtcagccatgcctttacaaggatggaaagcggagacgtcaaatacagatttactttggtcgattatgataagaaattccataaatag

SEQ ID NO: 34 - protein, 361 aa, Saccharomyces cerevisiae
MLYPEKFQGIGISNAKDWKHPKLVSFDPKPFGDHDVDVEIEACGICGSDFHIAVGNWGPVPENQILGHEIIGRVVKVGSKCHTGVKIGDRVGVGAQALACFECERCKSDNEQYCTNDHVLTMWTPYKDGYISQGGFASHVRLHEHFAIQIPENIPSPLAAPLLCGGITVFSPLLRNGCGPGKRVGIVGIGGIGHMGILLAKAMGAEVYAFSRGHSKREDSMKLGADHYIAMLEDKGWTEQYSNALDLLVVCSSSLSKVNFDSIVKIMKIGGSIVSIAAPEVNEKLVLKPLGLMGVSISSSAIGSRKEIEQLLKLVSEKNVKIWVEKLPISEEGVSHAFTRMESGDVKYRFTLVDYDKKFHK

SEQ ID NO: 35 - DNA, 1035 bp, Saccharomyces cerevisiae
atgtctaatacagttctagtttctggcgcttcaggttttattgccttgcatatcctgtcacaattgttaaaacaagattataaggttattggaactgtgagatcccatgaaaaagaagcaaaattgctaagacaatttcaacataaccctaatttaactttagaaattgttccggacatttctcatccaaatgctttcgataaggttctgcagaaacgtggacgtgagattaggtatgttctacacacggcctctccttttcattatgatactaccgaatatgaaaaagacttattgattcccgcgttagaaggtacaaaaaacatcctaaattctatcaagaaatatgcagcagacactgtagagcgtgttgttgtgacttcttcttgtactgctattataacccttgcaaagatggacgatcccagtgtggtttttacagaagagagttggaacgaagcaacctgggaaagctgtcaaattgatgggataaatgcttactttgcatccaagaagtttgctgaaaaggctgcctgggagttcacaaaagagaatgaagatcacatcaaattcaaactaacaacagtcaacccttctcttctttttggtcctcaacttttcgatgaagatgtgcatggccatttgaatacttcttgcgaaatgatcaatggcctaattcataccccagtaaatgccagtgttcctgattttcattccatttttattgatgtaagggatgtggccctagctcatctgtatgctttccagaaggaaaataccgcgggtaaaagattagtggtaactaacggtaaatttggaaaccaagatatcctggatattttgaacgaagattttccacaattaagaggtctcattcctttgggtaagcctggcacaggtgatcaagtcattgaccgcggttcaactacagataatagtgcaacgaggaaaatacttggctttgagttcagaagtttacacgaaagtgtccatgatactgctgcccaaattttgaagaagcagaacagattatga

SEQ ID NO: 36 - protein, 344 aa, Saccharomyces cerevisiae
MSNTVLVSGASGFIALHILSQLLKQDYKVIGTVRSHEKEAKLLRQFQHNPNLTLEIVPDISHPNAFDKVLQKRGREIRYVLHTASPFHYDTTEYEKDLLIPALEGTKNILNSIKKYAADTVERVVVTSSCTAIITLAKMDDPSVVFTEESWNEATWESCQIDGINAYFASKKFAEKAAWEFTKENEDHIKFKLTTVNPSLLFGPQLFDEDVHGHLNTSCEMINGLIHTPVNASVPDFHSIFIDVRDVALAHLYAFQKENTAGKRLVVTNGKFGNQDILDILNEDFPQLRGLIPLGKPGTGDQVIDRGSTTDNSATRKILGFEFRSLHESVHDTAAQILKKQNRL

SEQ ID NO: 37 - DNA, 1410 bp, Saccharomyces cerevisiae
atgacaagcattgacattaacaacttacaaaatacctttcaacaagctatgaatatgagcggctccccaggcgctgtttgtacttcacctacgcaatctttcatgaataccgttccacagcgcttgaatgctgtaaagcacccaaaaattttgaagcctttctcaacgggtgatatgaagattttactattagaaaacgttaatcaaactgctattacaatcttcgaagagcaaggttaccaagtcgaattctataaatcttcattgcccgaggaagagttgatcgaaaagatcaaggacgttcatgctattggtatcagatcaaagactagattaacttcaaatgtcttacaacatgcgaagaatctggtttgtattggttgtttctgtatcggtaccaaccaagttgacttagactacgctaccagcagaggtattgctgttttcaactcgcctttctccaactcaagatcagtagcagaattggtcatcgctgaaatcattagtttagcaagacaactaggtgatagatctatcgaattacataccggtacatggaataaggttgctgctagatgttgggaggtaagaggaaaaactcttggtattattgggtacggtcacattggttcccaattatcagttcttgcagaagctatgggtttgcatgtgttgtactacgatattgtaactatcatggccttgggtactgccagacaagtttctacattagatgaattgttgaataaatctgattttgtgacactacatgtaccagctactcctgaaactgaaaaaatgttatctgccccacaatttgctgctatgaaggatggcgcttatgttattaatgcttcaagaggtactgtcgtggacattccatctttgatccaagccgtgaaagccaacaaaattgcaggtgctgctttggatgtttatccacatgaaccagctaagaacggtgaaggttcatttaacgatgagctaaatagctggacttctgaattagtttcattaccaaatatcatcttgacaccacacattggtggctctaccgaagaagcccaaagctcaatcggtattgaagtggctaccgcattgtccaaatacatcaatgaaggtaactctgtcggttcagtcaacttcccagaagtggcattgaaatcattgtcttacgaccaagagaacactgtgcgtgtgttatacattcaccaaaatgtaccaggtgttttgaagaccgtcaatgatattttatcgaaccataacatcgaaaagcaattttccgattcaaatggtgaaattgcttatttaatggctgatatctcttctgttgaccaaagcgatattaaagatatttatgaacaactaaatcaaacctctgctaagatctcaattagattgctatattaa

SEQ ID NO: 38 - protein, 469 aa, Saccharomyces cerevisiae
MTSIDINNLQNTFQQAMNMSGSPGAVCTSPTQSFMNTVPQRLNAVKHPKILKPFSTGDMKILLLENVNQTAITIFEEQGYQVEFYKSSLPEEELIEKIKDVHAIGIRSKTRLTSNVLQHAKNLVCIGCFCIGTNQVDLDYATSRGIAVFNSPFSNSRSVAELVIAEIISLARQLGDRSIELHTGTWNKVAARCWEVRGKTLGIIGYGHIGSQLSVLAEAMGLHVLYYDIVTIMALGTARQVSTLDELLNKSDFVTLHVPATPETEKMLSAPQFAAMKDGAYVINASRGTVVDIPSLIQAVKANKIAGAALDVYPHEPAKNGEGSFNDELNSWTSELVSLPNIILTPHIGGSTEEAQSSIGIEVATALSKYINEGNSVGSVNFPEVALKSLSYDQENTVRVLYIHQNVPGVLKTVNDILSNHNIEKQFSDSNGEIAYLMADISSVDQSDIKDIYEQLNQTSAKISIRLLY

SEQ ID NO: 39 - DNA, 711 bp, Saccharomyces cerevisiae
atggtggtcatcaataagcaattaatggtgagtgggatattgccggcgtggctaaaaaatgagtatgatctggaagacaaaataatttcaacggtaggtgccggtagaattggatatagggttctggaaagattggtcgcatttaatccgaagaagttactgtactacgactaccaggaactacctgcggaagcaatcaatagattgaacgaggccagcaagcttttcaatggcagaggtgatattgttcagagagtagagaaattggaggatatggttgctcagtcagatgttgttaccatcaactgtccattgcacaaggactcaaggggtttattcaataaaaagcttatttcccacatgaaagatggtgcatacttggtgaataccgctagaggtgctatttgtgtcgcagaagatgttgccgaggcagtcaagtctggtaaattggctggctatggtggtgatgtctgggataagcaaccagcaccaaaagaccatccctggaggactatggacaataaggaccacgtgggaaacgcaatgactgttcatatcagtggcacatctctgcatgctcaaaagaggtacgctcagggagtaaagaacatcctaaatagttacttttccaaaaagtttgattaccgtccacaggatattattgtgcagaatggttcttatgccaccagagcttatggacagaagaaataa

SEQ ID NO: 40 - protein, 236 aa, Saccharomyces cerevisiae
MVVINKQLMVSGILPAWLKNEYDLEDKIISTVGAGRIGYRVLERLVAFNPKKLLYYDYQELPAEAINRLNEASKLFNGRGDIVQRVEKLEDMVAQSDVVTINCPLHKDSRGLFNKKLISHMKDGAYLVNTARGAICVAEDVAEAVKSGKLAGYGGDVWDKQPAPKDHPWRTMDNKDHVGNAMTVHISGTSLHAQKRYAQGVKNILNSYFSKKFDYRPQDIIVQNGSYATRAYGQKK

SEQ ID NO: 41 - DNA, 804 bp, Saccharomyces paradoxus
atgtcccaaggtagaaaagctgcagaaagattggctaacaagaccgtgctcattacgggtgcctctgctggtattggtaaggccaccgcattagagtatttggaggcatccaatggtgatatgaaactggtcttagctgctagaagattagaaaagctcgaggaattaaagaaaactattgatcaggagtttccaaacgccaaagttcatgtggcccaactggatatcactcaagcagaaaagatcaagccctttattgagaatttgccaaaggagttcaaagacattgacattttggtgaacaacgctgggaaggcccttggtaccgaccgtgtgggggagattgcaacacaagatatccaggatgtgtttgacaccaacgtcacagctttaattaatatcactcaagctgtgctgcccatttttcaagccaagaactcaggggatattgtgaacttgggttcggtggctggcagggatgcatacccaacgggttccatctattgtgcctccaagtttgccgtgggggcgttcactgatagtttaagaaaggagcttatcaacaccaagatcagagtcatcctaatcgcaccagggctagtcgaaactgaattttcactggttagatacagaggcaacgaggagcaagccaagaatgtctacaaggacaccaccccattaatggctgatgacgtggctgatttgatcgtgtacgcaacttccaggaaacaaaacactgtaattgcagacacgctaatctttccaaccaaccaagcatcgcctcaccacatcttccgtggatga

SEQ ID NO: 42 - protein, 267 aa, Saccharomyces paradoxus
MSQGRKAAERLANKTVLITGASAGIGKATALEYLEASNGDMKLVLAARRLEKLEELKKTIDQEFPNAKVHVAQLDITQAEKIKPFIENLPKEFKDIDILVNNAGKALGTDRVGEIATQDIQDVFDTNVTALINITQAVLPIFQAKNSGDIVNLGSVAGRDAYPTGSIYCASKFAVGAFTDSLRKELINTKIRVILIAPGLVETEFSLVRYRGNEEQAKNVYKDTTPLMADDVADLIVYATSRKQNTVIADTLIFPTNQASPHHIFRG

SEQ ID NO: 43 - DNA, 805 bp, Saccharomyces bayanus
atgtcccaaggtagaaaagctgcagaaagattggccaacaagacggtgctcattacaggcgcttctgctggtattggtaaggccaccgcattggagtatttggaagcatccaatggaaacatgaaactgatcttggctgcgaggagattggagaagctagaggagctgaagaagaccatcgacgaggagtttcccaatgcaaaggttcacgttggccaactggatatcacacaggccgagaagatcaagcccttcattgaaaacttgccggaggcattcaaggatattgacatcctgataaacaatgccggcaaagccctgggctccgaacgtgtcggggaaattgccacacaggacatccaggacgtgttcgacaccaacgtcacggcgttgatcaacgtcacgcaagcagtgctgccaattttccaagccaagaactcaggggacatcgtcaacttggggctcggtggccggcagagacgcataccccacaggctccatctactgtgcttccaagtttgccgtcggtgcgttcactgacagtttgagaaaggaactgatcaacacgaagatcagagttatcttgatcgcgccggggctggttgagaccgagttctcactggtcagatacagaggtaatgaggaacaagctaaaaacgtctacaaggacactacgccgttgatggccgacgacgtggctgacttaatcgtatattccacttccagaaagcagaacaccgtggttgccgacaccctgatcttccccaccaaccaagcctcgccctaccacatctttcgcggttaa

SEQ ID NO: 44 - protein, 268 aa, Saccharomyces bayanus
  misc_feature (180)..(180): Xaa can be any naturally occurring amino acid
  misc_feature (202)..(202): Xaa can be any naturally occurring amino acid
  misc_feature (213)..(214): Xaa can be any naturally occurring amino acid
  misc_feature (218)..(218): Xaa can be any naturally occurring amino acid
  misc_feature (234)..(234): Xaa can be any naturally occurring amino acid
MSQGRKAAERLANKTVLITGASAGIGKATALEYLEASNGNMKLILAARRLEKLEELKKTIDEEFPNAKVHVGQLDITQAEKIKPFIENLPEAFKDIDILINNAGKALGSERVGEIATQDIQDVFDTNVTALINVTQAVLPIFQAKNSGDIVNLGLGGRQRRIPHRLHLLCFQVCRRCVHXQFEKGTDQHEDQSYLDRAGAGXDRVLTGQIQRXXGTSXKRLQGHYAVDGRRRGXLNRIFHFQKAEHRGCRHPDLPHQPSLALPHLSRL

SEQ ID NO: 45 - DNA, 804 bp, Saccharomyces castellii
atgtctcaaggtcctaaagctgccgaaagattgaatgagaagattgtgtttatcactggtgcttcagctggtattgggcaagccaccgctttggaatacatggatgcgtcgaacggtactgtgaaattggttctagttgccagaagattggagaaattacaacaattgaaggaagtcattgaggcaaaataccctaagagtaaagtctatattgggaagttggatgtgacagagcttgagaccattcaaccattcttggataatcttcctgaggaatttaaggatattgatatcttgattaataatgccgggaaggcattaggttccgatcgtgtaggtgatattgatataaaagatgtgaagggaatgatggataccaatgtcttggggttgatcaatgtgacgcaagctgtgttgcacattttccaaaagaagaactccggtgatattgtgaacttaggttcagttgctggaagagatgcatacccaacagggtccatttactgtgcttctaaatttgccgtgagggcctttactgaaagtttgagaagggaattaattaataccaagattagggtgatattgatagccccgggtatcgtcgaaactgaattctcagttgttagatacaagggtgataatgagcgtgctaaatctgtctacgatggagttcaccccttggaagcagacgacgtagcagatttaattgtatacaccacttcaagaaaacagaacacagtaattgctgacactttgatattcccaacctctcaaggttccgcattccacgtccatcgcgattaa

SEQ ID NO: 46 - protein, 267 aa, Saccharomyces castellii
MSQGPKAAERLNEKIVFITGASAGIGQATALEYMDASNGTVKLVLVARRLEKLQQLKEVIEAKYPKSKVYIGKLDVTELETIQPFLDNLPEEFKDIDILINNAGKALGSDRVGDIDIKDVKGMMDTNVLGLINVTQAVLHIFQKKNSGDIVNLGSVAGRDAYPTGSIYCASKFAVRAFTESLRRELINTKIRVILIAPGIVETEFSVVRYKGDNERAKSVYDGVHPLEADDVADLIVYTTSRKQNTVIADTLIFPTSQGSAFHVHRD

SEQ ID NO: 47 - DNA, 804 bp, Saccharomyces mikatae
atgtctcaaggtagaaaagctgcagaaagattggctggcaaaaccgttctcatcacgggtgcctctgctggtattggcaaagccactgcattagagtatttggaggcatccaatggcgatatgaaattaatcttagccgctagaagattagaaaagctcgaggaattgaagaagactatcgatgaagagtttccaaacgcaaaggtccatgtgaccaaactggacatcacacagacagaaaagatcaagccctttattgaaaacttgccagaggagttcaaagacattgatattctggtgaacaacgctggtaaggctcttggtacggaccgtgttggggagattgatacacaggacgtccaggacgtgttcgacaccaacgtctcggctttgattaatgtcacacaggctgttctgcccatcttccaagctaagaactcaggggatattgtgaacttgggctcggtagctggcagagatgcatacccaacgggctccatctattgtgcatctaagtttgccgtcggggctttcactgagagtttgagaatggaacttataaacactaagattagagtcattctaattgcaccagggttagtcgaaactgagttttccctggttagatacagaggtaacgaagaacaagccaagaatgtttacaaggacaccactccgttgatggccgatgacgtggctgatttgattgtgtatgcgacttcaaggaagcagaacactgtaattgcagacacactaatctttcctaccaaccaagcgtcaccttaccatatctttcgcgggtga

SEQ ID NO: 48 - protein, 267 aa, Saccharomyces mikatae
MSQGRKAAERLAGKTVLITGASAGIGKATALEYLEASNGDMKLILAARRLEKLEELKKTIDEEFPNAKVHVTKLDITQTEKIKPFIENLPEEFKDIDILVNNAGKALGTDRVGEIDTQDVQDVFDTNVSALINVTQAVLPIFQAKNSGDIVNLGSVAGRDAYPTGSIYCASKFAVGAFTESLRMELINTKIRVILIAPGLVETEFSLVRYRGNEEQAKNVYKDTTPLMADDVADLIVYATSRKQNTVIADTLIFPTNQASPYHIFRG

SEQ ID NO: 49 - DNA, 804 bp, Ashbya gossypii
atgtccctaggaagaaaagcagctgaaagattagccaacaaaattgtgcttgtgactggtgcctctgcgggcattggccgtgctacagccattaactatgcagacgcgacggacggggcaatcaagttgattttggtggcaagacgcgcagaaaagctcaccagcttgaaacaggagatcgaaagcaagtatcccaacgccaagatccatgtcggacaattggatgtgacccaactggaccagatccgcccatttttggagggactacctgaggagttccgagacattgatattttaattaacaacgcaggtaaggccctcggcactgagagggtgggggaaatctcgatggacgatatccaggaggttttcaacactaatgttatcggcttggtgcacttgactcaggaggttctacctattatgaaagccaagaattccggggacattgtcaatgttgggtcgattgccggccgcgaagcctaccctggtggctctatttactgtgccacgaaacatgcggtcaaggctttcaccagggccatgcggaaggagctcattagcaccaagatccgggtcttcgaaattgcgccgggctctgtagaaacggaattctccatggttcgtatgcgcggtaacgaagagaatgccaagaaagtgtaccagggatttgaacccctagatggtgatgatatcgctgatacaattgtctatgccacatccagaagatccaacaccgtagttgcagagatggtcgtttacccatccgcgcaaggttctctgtacgatactcaccgcaactaa

SEQ ID NO: 50 - protein, 267 aa, Ashbya gossypii
MSLGRKAAERLANKIVLVTGASAGIGRATAINYADATDGAIKLILVARRAEKLTSLKQEIESKYPNAKIHVGQLDVTQLDQIRPFLEGLPEEFRDIDILINNAGKALGTERVGEISMDDIQEVFNTNVIGLVHLTQEVLPIMKAKNSGDIVNVGSIAGREAYPGGSIYCATKHAVKAFTRAMRKELISTKIRVFEIAPGSVETEFSMVRMRGNEENAKKVYQGFEPLDGDDIADTIVYATSRRSNTVVAEMVVYPSAQGSLYDTHRN

SEQ ID NO: 51 - DNA, 807 bp, Candida glabrata
atgtctcaaggaagaaaagctgctgagaggttacaagggaagattgcctttattacgggtgcctctgcgggcatcggtaaagctacagccattgagtatttggatgcttccaatggtagtgtgaagctagttcttggtgcacgtagaatggagaaattggaggagttgaagaaggaattgctggctcaatatcctgatgcaaagattcatataggtaaactggatgttacagactttgaaaacgtcaagcagtttttggctgacttgccagaagagttcaaggacatcgacatcctgatcaataacgctggtaaagcgttggggtctgacaaagttggagacattgaccctgaggatatcgcaggaatggttaacaccaacgtccttgcattgatcaatttaacacaattgttgttgccattattcaagaagaagaacagtggtgatatcgtcaacttgggatcgattgctggtagagacgcatacccaacgggtgctatatactgtgcaacaaaacatgctgtcagggcattcacacaatccttaaggaaggaattgatcaacaccgacattagagtaattgaaattgctcctggtatggtcgaaaccgagttttctgtggtcaggtacaaaggtgacaagtccaaagcagacgacgtctacagaggtacaacaccactatatgccgatgatatcgcggatttgattgtgtactctaccagcagaaagccaaacatggtggtagcagatgtcctggtcttcccaacacaccaggcatcggcttcgcacatctacaggggcgactaa

SEQ ID NO: 52 - protein, 268 aa, Candida glabrata
MSQGRKAAERLQGKIAFITGASAGIGKATAIEYLDASNGSVKLVLGARRMEKLEELKKELLAQYPDAKIHIGKLDVTDFENVKQFLADLPEEFKDIDILINNAGKALGSDKVGDIDPEDIAGMVNTNVLALINLTQLLLPLFKKKNSGDIVNLGSIAGRDAYPTGAIYCATKHAVRAFTQSLRKELINTDIRVIEIAPGMVETEFSVVRYKGDKSKADDVYRGTTPLYADDIADLIVYSTSRKPNMVVADVLVFPTHQASASHIYRGD

SEQ ID NO: 53 - DNA, 813 bp, Debaryomyces hansenii
atgtcgtacggatctaaagctgctgaacgtgttgccaataagattgtcttaatcactggtgcttcatctggaattggtgaagcaactgccaaagaaattgcatcagccgctaatggcaatttaaaattagtgttgtgtgctagacgaaaagaaaagttggataatttatctaaagaattgactgacaaatattcatccatcaaggttcatgttgctcaactagatgtatctaagctcgagactatcaagccatttatcaatgatttaccgaaagaattctctgacgtggatgtattagtcaacaatgcaggcttggctttgggccgtgatgaagttggaaccattgacacagatgatatgttatcgatgtttcaaactaatgttttagggttaattaccatcacacaggctgttttgccaatcatgaaaaagaagaacagcggagatgttgttaatataggttcaattgctggaagagactcttaccctggaggtggaatttactgtccaactaaggcaagtgtcaagtcgttttcgcaagttttaagaaaggaattgattagcaccaagattagagttcttgaggttgaccctggtaatgttgaaactgaattttcaaatgtcagattcaagggcgatatggaaaaggcaaagctggtttacgcgggtactgaaccattattatccgaagacgtagctgaggttgtcgtattcggacttacaagaaagcaaaataccgttattgctgagacattagtcttttcaaccaatcaagccagctcatctcacttataccgtgaaagcgataaataa

SEQ ID NO: 54 - protein, 270 aa, Debaryomyces hansenii
MSYGSKAAERVANKIVLITGASSGIGEATAKEIASAANGNLKLVLCARRKEKLDNLSKELTDKYSSIKVHVAQLDVSKLETIKPFINDLPKEFSDVDVLVNNAGLALGRDEVGTIDTDDMLSMFQTNVLGLITITQAVLPIMKKKNSGDVVNIGSIAGRDSYPGGGIYCPTKASVKSFSQVLRKELISTKIRVLEVDPGNVETEFSNVRFKGDMEKAKLVYAGTEPLLSEDVAEVVVFGLTRKQNTVIAETLVFSTNQASSSHLYRESDK

SEQ ID NO: 55 - DNA, 810 bp, Scheffersomyces stipitis
atgtcgtttggaaaaaaagctgctgaaagacttgccaacaaaatcattcttatcaccggggcttcgtctggtattggtgaagctacagctagagagtttgcatctgctgccaatgggaatatcagattgattttgacagccagaagaaaagaaaagttggctcaattgtcagactcattgaccaaggaatttccaactatcaaaatccattctgccaaattggatgtgaccgaacatgatggcatcaagcctttcatttctggtttacccaaggatttcgccgacatcgatgtgttgatcaacaatgctggaaaagctcttggaaaagcatctgttggtgaaatcagtgacagtgatatccaaggcatgatgcaaacgaatgtcttgggactcatcaacatgactcaggctgtgattcccatttttaaggctaaaaattctggagatatcgtcaacatcggttcgattgctggaagagacccttaccctggtggatcgatctactgtgcctccaaggctgctgttaagttcttctcgcattctttgagaaaggaactcattaacaccagaatcagagttttggaagttgatccaggtgctgtgttgaccgagttctctttggttcgtttccacggtgatcagggagctgctgatgctgtttatgaaggtacccaacctttggatgcctctgatatcgcagaagttatcgtgtttggtatcaccagaaagcagaacaccgtcatagccgaaaccttggtattcccaagtcaccaggcttctgcctctcatgtttacaaggctcctaagtag

SEQ ID NO: 56 - protein, 269 aa, Scheffersomyces stipitis
MSFGKKAAERLANKIILITGASSGIGEATAREFASAANGNIRLILTARRKEKLAQLSDSLTKEFPTIKIHSAKLDVTEHDGIKPFISGLPKDFADIDVLINNAGKALGKASVGEISDSDIQGMMQTNVLGLINMTQAVIPIFKAKNSGDIVNIGSIAGRDPYPGGSIYCASKAAVKFFSHSLRKELINTRIRVLEVDPGAVLTEFSLVRFHGDQGAADAVYEGTQPLDASDIAEVIVFGITRKQNTVIAETLVFPSHQASASHVYKAPK

SEQ ID NO: 57 - DNA, 891 bp, Meyerozyma guilliermondii
atgtgcctcttaccagccggtagcactgtattatgtcatcacccagtagtgagtgtggagattaaatcctcaatcttcatgtctttcggtgccaaagccgctgaacgccttgccaacaagatcatattgatcactggggcatcgtctggtataggcgaggctaccgccagagaattcgctgctgctgccaatggaaaaattctgttgattttgaccgctcggagagaagacaaactcaagtctctctcgcaacaattgagcctcatttacccgcaaattaaaatccattctgctcgtcttgatgtctctgagttttcgtcacttaagccgttcattactgggttgccaaaggattttgctagcatcgacgttttggtgaataatgcggggaaagcattgggaagagccaatgttggtgaaatttcccaagaggaaatcaatggcatgttccataccaatgttcttgggttgataaacttaactcaggaggtgttacccatcttcaaaaagaaaaatgctggagatattgtgaacattggctcagtggccggtagagaaccttaccctggaggtgcagtatactgtgcttcaaaggcagcagttaactacttttctcattctttgagaaaggaaactatcaattccaaaatcagggtcatggaggtggatcctggggcagtagagacagagttctcgttggttcgttttggcggtgatgccgaggctgcgaaaaaggtgtatgagggaaccgagcctttgggcccagaagatattgcggaaatcattgtgtttgctgtgtcgagaaaagccaaaactgtcattgcggaaactttggtgtttcctacccatcaggctggagcagttcatgttcatagagggccgcttgagtga

SEQ ID NO: 58 - protein, 296 aa, Meyerozyma guilliermondii
MCLLPAGSTVLCHHPVVSVEIKSSIFMSFGAKAAERLANKIILITGASSGIGEATAREFAAAANGKILLILTARREDKLKSLSQQLSLIYPQIKIHSARLDVSEFSSLKPFITGLPKDFASIDVLVNNAGKALGRANVGEISQEEINGMFHTNVLGLINLTQEVLPIFKKKNAGDIVNIGSVAGREPYPGGAVYCASKAAVNYFSHSLRKETINSKIRVMEVDPGAVETEFSLVRFGGDAEAAKKVYEGTEPLGPEDIAEIIVFAVSRKAKTVIAETLVFPTHQAGAVHVHRGPLE

SEQ ID NO: 59 - DNA, 804 bp, Vanderwaltozyma polyspora
atgtcacagggtagaaaggcttcagaaaggttggctggtaaaactgtattaattacaggtgcttcatcagggattgggaaagccactgcattagaatatctagatgcctccaatggtcatatgaagttaattttagttgcaagaagattagaaaaattgcaagagttgaaggaaacaatttgtaaagaatatccagaatctaaggttcatgttgaagaattagatatttctgatattaatagaatcccagaatttattgcaaaattacctgaagaattcaaagatattgatatattgattaacaatgcaggtaaagcattaggaagtgatactattggtaatatcgagaatgaggatattaaaggtatgtttgagactaacgtttttggattaatctgtttaacacaagctgtacttccaatattcaaggctaaaaatggtggtgatattgtcaatttagggtcaattgcaggcatagaagcttacccaacaggatctatatattgtgcaactaaatttgcagttaaagcattcactgaaagtttaagaaaggaattgattaatacaaagatcagagttattgaaattgcaccaggtatggttaacactgaattttctgtaattagatataaaggtgaccaagaaaaggcagataaagtttatgaaaacactactcctttatatgcagatgacatcgctgatttgatagtttacaccacttctagaaaatcgaataccgttatcgctgatgttttggtattcccaacatgccaagcttctgcatcccatatctatcgtggataa

SEQ ID NO: 60 - protein, 267 aa, Vanderwaltozyma polyspora
MSQGRKASERLAGKTVLITGASSGIGKATALEYLDASNGHMKLILVARRLEKLQELKETICKEYPESKVHVEELDISDINRIPEFIAKLPEEFKDIDILINNAGKALGSDTIGNIENEDIKGMFETNVFGLICLTQAVLPIFKAKNGGDIVNLGSIAGIEAYPTGSIYCATKFAVKAFTESLRKELINTKIRVIEIAPGMVNTEFSVIRYKGDQEKADKVYENTTPLYADDIADLIVYTTSRKSNTVIADVLVFPTCQASASHIYRG

SEQ ID NO: 61 - DNA, 807 bp, Candida dubliniensis
atgtcatttggtagaaaagctgctgaaagattagccaatagatccattcttatcactggtgcttcatctgggattggtgaagcatgtgctaaagttttcgctgaagcatctaatggtcaagttaaattagttttaggagcaagaagaaaagaacgattagttaaattatctgatactttaattaaacaatatcctaatattaaaattcatcatgattttttggatgttactattaaagattcaatttcaaaattcattgctggaattcctcatgaatttgaacctgatgtattaattaataatagtggtaaagccttggggaaagaagaagttggagaattgaaagatgaagatattacggaaatgtttgatactaatgtcattggagtcattcgtatgactcaagcagttttacctttacttaaaaaaaaaccttatgctgatgtggttttcattggaagtattgctggacgtgttccttataaaaatggaggtggttattgtgcatctaaagctgctgttcgtagtttcaccgatacatttagaaaagaaactattaatactggtattagagtcattgaagttgatccaggtgcagtacttactgaatttagtgttgttcgttataaaggtgacactgatgctgccgatgctgtttatactggtactgaaccattaacaccagaagatgttgctgaagtggttgtttttgcatcttcaagaaaacaaaataccgttattgctgatactttgattttcccaaatcatcaagcttctccagatcatgtttatagaaaacctaattaa

SEQ ID NO: 62 - protein, 268 aa, Candida dubliniensis
MSFGRKAAERLANRSILITGASSGIGEACAKVFAEASNGQVKLVLGARRKERLVKLSDTLIKQYPNIKIHHDFLDVTIKDSISKFIAGIPHEFEPDVLINNSGKALGKEEVGELKDEDITEMFDTNVIGVIRMTQAVLPLLKKKPYADVVFIGSIAGRVPYKNGGGYCASKAAVRSFTDTFRKETINTGIRVIEVDPGAVLTEFSVVRYKGDTDAADAVYTGTEPLTPEDVAEVVVFASSRKQNTVIADTLIFPNHQASPDHVYRKPN

SEQ ID NO: 63 - DNA, 822 bp, Zygosaccharomyces rouxii
atgtcacaaggtgtcaaagctgctgaaagactagctggtaagactgtattcattacaggtgcttctgcaggtatcggtcaagcaactgcaaaggaatatttggatgcatccaatggtcaaattaaattgatcttggctgcaagaagattagagaaattacacgagtttaaagaacaaactacaaagagttacccaagcgctcaagtccacattggtaaattggacgtcactgcaattgacaccataaaaccatttttggataaattaccaaaggaatttcaagatatcgatattttgatcaacaatgccggtaaggcattaggtactgataaagttggtgatattgcagatgaagacgtggaaggtatgttcgacaccaatgtcttggggttaatcaaagttactcaagctgttttacctatcttcaaaagaaaaaattctggtgatgtcgttaacattagttcggttgctggtagagaggcatacccaggtggttccatttactgtgctactaaacacgctgttaaggcattcactgaaagtttgcgtaaggaattagtcgatacaaaaatcagagtcatgagtattgatcctggtaatgtagagaccgaattttctatggttagattccgtggtgatacagaaaaggcaaagaaggtttaccaagacactgtcccattatatgcagatgacattgcagatttaatcgtctatgcaacctctagaaagcaaaacactgtcattgctgacactttgatcttctcttctaaccaggcatcaccataccacctctacagaggctctcaagacaaaaccaattga

SEQ ID NO: 64 - protein, 273 aa, Zygosaccharomyces rouxii
MSQGVKAAERLAGKTVFITGASAGIGQATAKEYLDASNGQIKLILAARRLEKLHEFKEQTTKSYPSAQVHIGKLDVTAIDTIKPFLDKLPKEFQDIDILINNAGKALGTDKVGDIADEDVEGMFDTNVLGLIKVTQAVLPIFKRKNSGDVVNISSVAGREAYPGGSIYCATKHAVKAFTESLRKELVDTKIRVMSIDPGNVETEFSMVRFRGDTEKAKKVYQDTVPLYADDIADLIVYATSRKQNTVIADTLIFSSNQASPYHLYRGSQDKTN

SEQ ID NO: 65 - DNA, 804 bp, Lachancea thermotolerans
atgtcacagggaagaagagcagctgaaagactggcaggaaagactgtcttcatcacaggcgcatcagccggtatcggtcaggccactgcgcaagaatacctggaagcatccgaaggcaaaatcaagttgatccttgcagcaagaagactcgacaagctggaggaaatcaaagccaaggtttctaaagacttccctgaagcacaggtgcatatcggccagctagatgtgactcagacggacaaaatccagccttttgtcgacaatttgcccgaagagttcaaagacatcgacatcctgatcaacaacgcgggcaaggcgctcggatccgaccccgtgggcacaatcgaccccaatgatattcaaggcatgatccagactaacgttatcgggcttataaatgttacccaagccgttctgcccatcttcaaggccaaaaactctggtgatatcgtgaacctgggttctgtcgctggcagagaagcttaccctacaggatctatttactgcgctacgaagcacgcggtgcgtgctttcacccagagcctgcgcaaggaactgatcaacacaaacatcagggttattgaggtcgctccaggtaacgtggagaccgagttttctctggttagatacaagggcgactctgagaaagccaagaaggtttacgaaggcacacaacccctttacgctgacgatatcgcagacctaatcgtttacgcaacctcgagaaaaccaaacaccgtcatcgcggacgttttggttttcgcttcgaaccaggcttcgccttaccacatttaccgtggttag

SEQ ID NO: 66 - protein, 267 aa, Lachancea thermotolerans
MSQGRRAAERLAGKTVFITGASAGIGQATAQEYLEASEGKIKLILAARRLDKLEEIKAKVSKDFPEAQVHIGQLDVTQTDKIQPFVDNLPEEFKDIDILINNAGKALGSDPVGTIDPNDIQGMIQTNVIGLINVTQAVLPIFKAKNSGDIVNLGSVAGREAYPTGSIYCATKHAVRAFTQSLRKELINTNIRVIEVAPGNVETEFSLVRYKGDSEKAKKVYEGTQPLYADDIADLIVYATSRKPNTVIADVLVFASNQASPYHIYRG

SEQ ID NO: 67 - DNA, 807 bp, Kluyveromyces lactis
atgtctcaaggtagaaaggctgctgaaagattgcaaaacaagacaattttcattaccggtgcttctgcaggtattggtcaagccacagcattggaatatctagatgctgctaacggtaatgtcaaattgatcttagcagcaagaaggttggctaagttggaagaattgaaggaaaaaatcaatgctgaatacccacaagctaaagtatatatcggtcaattggacgtcactgaaactgagaagattcaacctttcattgataacttgccggaagaattcaaggatatcgatattttgattaacaatgccggtaaagctttgggatctgatgttgtcggtaccatcagtagcgaggacatcaaaggtatgatagatactaacgttgttgcccttatcaacgttacccaagctgttttgcctattttcaaagcaaagaattccggtgacatcgttaacttaggttctgttgccggtagagatgcatatccaactggttctatctattgtgcttcgaagcatgctgtcagagcgttcactcagtctttgagaaaagaattaatcaatactggtattagggtcattgagattgctccaggtaatgtcgaaactgagttctctctagttagatacaagggtgatgccgatcgtgctaaacaggtttacaaaggtactactcctctatatgcagatgacattgctgacttgatcgtttatgccacttcaagaaaacctaatactgtcatcgctgatgttttggtatttgcttccaaccaagcatctccttaccacatttaccgtggcgaatag

SEQ ID NO: 68 - protein, 268 aa, Kluyveromyces lactis
MSQGRKAAERLQNKTIFITGASAGIGQATALEYLDAANGNVKLILAARRLAKLEELKEKINAEYPQAKVYIGQLDVTETEKIQPFIDNLPEEFKDIDILINNAGKALGSDVVGTISSEDIKGMIDTNVVALINVTQAVLPIFKAKNSGDIVNLGSVAGRDAYPTGSIYCASKHAVRAFTQSLRKELINTGIRVIEIAPGNVETEFSLVRYKGDADRAKQVYKGTTPLYADDIADLIVYATSRKPNTVIADVLVFASNQASPYHIYRGE

SEQ ID NO: 69 - DNA, 807 bp, Saccharomyces kluyveri
atgtctcaaggtagaagggctgcagaaagactagcaaacaagaccgtttttataactggcgcctctgccggcattggccaagctactgctttggaatactgtgatgcttctaacggtaaaataaacttggtgttaagtgccagaaggctggaaaaattgcaagagttaaaggacaaaatcaccaaggagtatcctgaagccaaggtttatattggtgtgcttgatgtgaccgaaacggaaaaaatcaaaccattcttggatggtttaccagaagaatttaaagatattgacatcttgatcaataatgcaggcaaagcgttaggctctgatcctgttggtaccatcaaaactgaagatattgaaggaatgatcaacaccaatgtcttagctcttatcaatattactcaagctgtcttgccaatcttcaaagccaagaatttcggtgatatcgtaaacttggggtctgtcgctggtagagatgcttatccaaccggtgcaatctactgtgctagcaaacatgcagtcagagccttcactcaaagtttgaggaaggaattggtgaacaccaatatcagagtgattgaaattgctccgggtaatgttgaaaccgagttttccttagttagatataaaggtgatacggaccgtgctaaaaaggtttatgaaggtactaacccattatatgcagatgacattgcagaccttattgtgtatgctacttctagaaagcctaatactgtcattgcggatgttttggtttttgcttcaaaccaagcatccccttaccatatctatcgcggtgactaa

SEQ ID NO: 70 - protein, 268 aa, Saccharomyces kluyveri
MSQGRRAAERLANKTVFITGASAGIGQATALEYCDASNGKINLVLSARRLEKLQELKDKITKEYPEAKVYIGVLDVTETEKIKPFLDGLPEEFKDIDILINNAGKALGSDPVGTIKTEDIEGMINTNVLALINITQAVLPIFKAKNFGDIVNLGSVAGRDAYPTGAIYCASKHAVRAFTQSLRKELVNTNIRVIEIAPGNVETEFSLVRYKGDTDRAKKVYEGTNPLYADDIADLIVYATSRKPNTVIADVLVFASNQASPYHIYRGD

SEQ ID NO: 71 - DNA, 807 bp, Yarrowia lipolytica
atgtctttcggagataaagctgctgctcgacttgcgggcaagaccgtctttgttaccggcgcctcgtccggcattggccaggccactgttctcgctctagccgaagctgccaagggcgacctcaagtttgtgcttgctgcccgacgaaccgaccgtctggacgagctcaagaagaagctggagaccgactacaagggtatccaggtgctgcctttcaagctggacgtgtccaaggtcgaggagaccgagaacattgtgtccaagctgcccaaggagttttccgaggtggacgtgcttatcaacaacgccggcatggtccacggcaccgaaaaggttggctccatcaaccagaacgacattgagatcatgttccacacaaacgtgctcggactcatttctgtcactcagcagtttgtcggcgagatgcgaaagcgaaacaagggcgacattgtcaacattggctccatcgccggacgagagccctacgttggaggaggaatctactgtgccaccaaggccgccgtgcgatctttcactgagactctccgaaaagagaacatcgacactcgaatccgagtcattgaggttgatcctggagccgttgagaccgagttctccgtcgtgcgattccgaggagacaagtccaaggccgacgctgtttacgctggaaccgagcctctggtcgctgacgatattgccgagttcatcacctacactctcactcgacgagagaatgtcgtcattgccgatactctcattttccccaaccaccaggcttctcctactcacgtctaccgaaagaactga

SEQ ID NO: 72 - protein, 268 aa, Yarrowia lipolytica
MSFGDKAAARLAGKTVFVTGASSGIGQATVLALAEAAKGDLKFVLAARRTDRLDELKKKLETDYKGIQVLPFKLDVSKVEETENIVSKLPKEFSEVDVLINNAGMVHGTEKVGSINQNDIEIMFHTNVLGLISVTQQFVGEMRKRNKGDIVNIGSIAGREPYVGGGIYCATKAAVRSFTETLRKENIDTRIRVIEVDPGAVETEFSVVRFRGDKSKADAVYAGTEPLVADDIAEFITYTLTRRENVVIADTLIFPNHQASPTHVYRKN

SEQ ID NO: 73 - DNA, 780 bp, Schizosaccharomyces pombe
atgagccgtttggatggaaaaacgattttaatcactggtgcctcttctggaattggaaaaagcactgcttttgaaattgccaaagttgccaaagtaaaacttattttggctgctcgcagattttctaccgttgaagaaattgcaaaggagttagaatcgaaatatgaagtatcggttcttcctcttaaattggatgtttctgatttgaagtctattcctggggtaattgagtcattgccaaaggaatttgctgatatcgatgtcttgattaataatgctggacttgctctaggtaccgataaagtcattgatcttaatattgatgacgccgttaccatgattactaccaatgttcttggtatgatggctatgactcgtgcggttcttcctatattctacagcaaaaacaagggtgatattttgaacgttggcagtattgccggcagagaatcatacgtaggcggctccgtttactgctctaccaagtctgcccttgctcaattcacttccgctttgcgtaaggagactattgacactcgcattcgtattatggaggttgatcctggcttggtcgaaaccgaattcagcgttgtgagattccacggagacaaacaaaaggctgataatgtttacaaaaatagtgagcctttgacacccgaagacattgctgaggtgattctttttgccctcactcgcagagaaaacgtcgttattgccgatacacttgttttcccatcccatcaaggtggtgccaatcatgtgtacagaaagcaagcgtag

SEQ ID NO: 74 - protein, 259 aa, Schizosaccharomyces pombe
MSRLDGKTILITGASSGIGKSTAFEIAKVAKVKLILAARRFSTVEEIAKELESKYEVSVLPLKLDVSDLKSIPGVIESLPKEFADIDVLINNAGLALGTDKVIDLNIDDAVTMITTNVLGMMAMTRAVLPIFYSKNKGDILNVGSIAGRESYVGGSVYCSTKSALAQFTSALRKETIDTRIRIMEVDPGLVETEFSVVRFHGDKQKADNVYKNSEPLTPEDIAEVILFALTRRENVVIADTLVFPSHQGGANHVYRKQA

SEQ ID NO: 75 - skipped

SEQ ID NO: 76 - DNA, 26 bp, artificial sequence
  Primer OBP622
ggatatagcagttgttgtacactagc

SEQ ID NO: 77 - DNA, 49 bp, artificial sequence
  Primer OBP623
ccattgtttaaacggcgcgccggatcctttgcgaaaccctatgctctgt

SEQ ID NO: 78 - DNA, 49 bp, artificial sequence
  Primer OBP624
gcaaaggatccggcgcgccgtttaaacaatggaaggtcgggatgagcat

SEQ ID NO: 79 - DNA, 34 bp, artificial sequence
  Primer OBP625
aattggccggcctacgtaacattctgtcaaccaa

SEQ ID NO: 80 - DNA, 34 bp, artificial sequence
  Primer OBP626
aattgcggccgcttcatatatgacgtaataaaat

SEQ ID NO: 81 - DNA, 34 bp, artificial sequence
  Primer OBP627
aattttaattaattttttttcttggaatcagtac

SEQ ID NO: 82 - DNA, 40 bp, artificial sequence
  Primer HY21tt
ttaaggcgcgcctatttgtaatacgtatacgaattccttc

SEQ ID NO: 83 - DNA, 56 bp, artificial sequence
  Primer HY24ac
acttaataactttaccggctgttgacattttgttcttcttgttattgtattgtgtt

SEQ ID NO: 84 - DNA, 12896 bp, artificial sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggcgcgccactggtagagagcgactttgtatgccccaattgcgaaacccgcgatatccttctcgattctttagtacccgaccaggacaaggaaaaggaggtcgaaacgtttttgaagaaacaagaggaactacacggaagctctaaagatggcaaccagccagaaactaagaaaatgaagttgatggatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccacccgggccccccctcgaggtcgacggtatcgataagcttgatatcgaattcctgcagcccgggggatccactagttctagagcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaatcaaaaagttaaaattgtacgaatagattcaccacttcttaacaaatcaaacccttcattgattttctcgaatggcaatacatgtgtaattaaaggatcaagagcaaacttcttcgccataaagtcggcaacaagttttggaacactatccttgctcttaaaaccgccaaatatagctcccttccatgtacgaccgcttagcaacagcataggattcatcgacaaattttgtgaatcaggaggaacacctacgatcacactgactccatatgcctcttgacagcaggacaacgcagttaccatagtatcaagacggcctataacttcaaaagagaaatcaactccaccgtttgacatttcagtaaggacttcttgtattggtttcttataatcttgagggttaacacattcagtagccccgacctccttagcttttgcaaatttgtccttattgatgtctacacctataatcctcgctgcgcctgcagctttacaccccataataacgcttagtcctactcctcctaaaccgaatactgcacaagtcgaaccctgtgtaacctttgcaactttaactgcggaaccgtaaccggtggaaaatccgcaccctatcaagcaaactttttccagtggtgaagctgcatcgattttagcgacagatatctcgtccaccactgtgtattgggaaaatgtagaagtaccaaggaaatggtgtataggtttccctctgcatgtaaatctgcttgtaccatcctgcatagtacctctaggcatagacaaatcatttttaaggcagaaattaccctcaggatgtttgcagactctacacttaccacattgaggagtgaacagtgggatcactttatcaccaggacgaacagtggtaacaccttcacctatggattcaacgattccggcagcctcgtgtcccgcgattactggcaaaggagtaactagagtgccactcaccacatggtcgtcggatctacagattccggtggcaaccatcttgattctaacctcgtgtgcttttggtggcgctacttctacttcttctatgctaaacggctttttctcttcccacaaaactgccgctttacacttaataactttaccggctgttgacatcctcagctagctattgtaatatgtgtgtttgtttggattattaagaagaataattacaaaaaaaattacaaaggaaggtaattacaacagaattaagaaaggacaagaaggaggaagagaatcagttcattatttcttctttgttatataacaaacccaagtagcgatttggccatacattaaaagttgagaaccaccctccctggcaacagccacaactcgttaccattgttcatcacgatcatgaaactcgctgtcagctgaaatttcacctcagtggatctctctttttattcttcatcgttccactaacctttttccatcagctggcagggaacggaaagtggaatcccatttagcgagcttcctcttttcttcaagaaaagacgaagcttgtgtgtgggtgcgcgcgctagtatctttccacattaagaaatataccataaaggttacttagacatcactatggctatatatatatatatatatatatgtaacttagcaccatcgcgcgtgcatcactgcatgtgttaaccgaaaagtttggcgaacacttcaccgacacggtcatttagatctgtcgtctgcattgcacgtcccttagccttaaatcctaggcgggagcattctcgtgtaattgtgcagcctgcgtagcaactcaacatagcgtagtctacccagtttttcaagggtttatcgttagaagattctcccttttcttcctgctcacaaatcttaaagtcatacattgcacgactaaatgcaagcatgcggatcccccgggctgcaggaattcgatatcaagcttatcgataccgtcgactggccattaatctttcccatattagatttcgccaagccatgaaagttcaagaaaggtctttagacgaattacccttcatttctcaaactggcgtcaagggatcctggtatggttttatcgttttatttctggttcttatagcatcgttttggacttctctgttcccattaggcggttcaggagccagcgcagaatcattctttgaaggatacttatcctttccaattttgattgtctgttacgttggacataaactgtatactagaaattggactttgatggtgaaactagaagatatggatcttgataccggcagaaaacaagtagatttgactcttcgtagggaagaaatgaggattgagcgagaaacattagcaaaaagatccttcgtaacaagatttttacatttctggtgttgaagggaaagatatgagctatacagcggaatttccatatcactcagattttgttatctaattttttccttcccacgtccgcgggaatctgtgtatattactgcatctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatcttggctctcttgggctcaaggtgacaaggtcctcgaaaatagggcgcgccccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 85 - DNA, 56 bp, artificial sequence
  Primer HY25
aacacaatacaataacaagaagaacaaaatgtcaacagccggtaaagttattaagt

SEQ ID NO: 86 - DNA, 40 bp, artificial sequence
  Primer HY4
ggaagtttaaacaccacaggtgttgtcctctgaggacata

SEQ ID NO: 87 - DNA, 30 bp, artificial sequence
  Primer URA3 end F
gcatatttgagaagatgcggccagcaaaac

SEQ ID NO: 88 - DNA, 22 bp, artificial sequence
  Primer oBP637
tttttgcacagttaaactaccc

SEQ ID NO: 89 - DNA, 22 bp, artificial sequence
  Primer oBP636
catttttttccctctaagaagc

SEQ ID NO: 90 - DNA, 34 bp, artificial sequence
  Primer oBP695
aattgcggccgcatgacaggtgaaagaattgaaa

SEQ ID NO: 91 - DNA, 34 bp, artificial sequence
  Primer oBP696
aattttaattaaacgggcatcttatagtgtcgtt

SEQ ID NO: 92 - DNA, 34 bp, artificial sequence
  Primer oBP693
aattgtttaaacaaaggatgatattgttctatta

SEQ ID NO: 93 - DNA, 33 bp, artificial sequence
  Primer oBP694
aattggccggccgcaacgacgacaatgccaaac

SEQ ID NO: 94 - DNA, 40 bp, artificial sequence
  Primer oBP691
aattggatccgcgatcgcgacgttctctccgttgttcaaa

SEQ ID NO: 95 - DNA, 41 bp, artificial sequence
  Primer oBP692
aattggcgcgccatttaaatatatatgtatatatataacac

SEQ ID NO: 96 - DNA, 40 bp, artificial sequence
  Primer HY16
ttaaggcgcgccccgcacgccgaaatgcatgcaagtaacc

SEQ ID NO: 97 - DNA, 56 bp, artificial sequence
  Primer HY19
acttaataactttaccggctgttgacattttgattgatttgactgtgttattttgc

SEQ ID NO: 98 - DNA, 56 bp, artificial sequence
  Primer HY20
gcaaaataacacagtcaaatcaatcaaaatgtcaacagccggtaaagttattaagt

SEQ ID NO: 99 - DNA, 22 bp, artificial sequence
  Primer oBP731
tgttcccacaatctattaccta

SEQ ID NO: 100 - DNA, 34 bp, artificial sequence
  Primer HY31
gccgactttatggcgaagaagtttgctcttgatc

SEQ ID NO: 101 - DNA, 22 bp, artificial sequence
  Primer OBP730
ttgctccaaagagatgtcttta

SEQ ID NO: 102 - DNA, 23 bp, artificial sequence
  Primer N1251
ccaatgtggctgtggtttcaggg

SEQ ID NO: 103 - DNA, 26 bp, artificial sequence
  Primer N1252
gcaaggccatgaagctttttctttcc

SEQ ID NO: 104 - DNA, 4236 bp, artificial sequence
  Synthetic construct
gatccgcattgcggattacgtattctaatgttcagataacttcgtatagcatacattatacgaagttatgcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtatagcatacattatacgaagttatccagtgatgatacaacgagttagccaaggtgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagag

SEQ ID NO: 105 - DNA, 62 bp, artificial sequence
  Primer N1253
ggaaagaaaaagcttcatggccttgcgctcatcaacactaaaattagagtcattctaattgc

SEQ ID NO: 106 - DNA, 29 bp, artificial sequence
  Primer N1254
ttatccacggaagatatgatgaggtgacg

SEQ ID NO: 107 - DNA, 64 bp, artificial sequence
  Primer N1255
ggaccctgaaaccacagccacattggtaccttccttgccaaagggattaaacctgacaattacc

SEQ ID NO: 108 - DNA, 29 bp, artificial sequence
  Primer N1256
acctagctaaactaagtaaatctgtatac

SEQ ID NO: 109 - DNA, 29 bp, artificial sequence
  Primer N1257
gatgatgctatttggtgcagagggtgatg

SEQ ID NO: 110 - DNA, 60 bp, artificial sequence
  Primer N1258
cagatttacttagtttagctaggtgtatcaaaatacgttctcaatgttctatttcccgcc

SEQ ID NO: 111 - DNA, 28 bp, artificial sequence
  Primer N1249
gctttatggaccctgaaaccacagccac

SEQ ID NO: 112 - DNA, 25 bp, artificial sequence
  Primer N1239
gacgaggataatgtgcatgaacggg

SEQ ID NO: 113 - DNA, 27 bp, artificial sequence
  Primer N1250
ggaaagaaaaagcttcatggccttgcg

SEQ ID NO: 114 - DNA, 24 bp, artificial sequence
  Primer N1242
gtttcgttgcgttggtggaatggc

SEQ ID NO: 115 - DNA, 38 bp, artificial sequence
  Primer N1179
gcgaattctgatctgatgcgctttgcatatctcatatt

SEQ ID NO: 116 - DNA, 38 bp, artificial sequence
  Primer N1180
gcggtaccgccgtttcggcttggatacgccatatgcaa

SEQ ID NO: 117 - DNA, 38 bp, artificial sequence
  Primer N1181
gccgatcgtgtaccaacctgcatttctttccgtcatat

SEQ ID NO: 118 - DNA, 38 bp, artificial sequence
  Primer N1182
gcgcatgcgtgtaccttttgattggaattggttcgcag

SEQ ID NO: 119 - DNA, 17 bp, artificial sequence
  M13 forward primer
gtaaaacgacggccagt

SEQ ID NO: 120 - DNA, 22 bp, artificial sequence
  M13 reverse primer
cacacaggaaacagctatgacc

SEQ ID NO: 121 - DNA, 29 bp, artificial sequence
  Primer
ttctatgttaaattttaacgatgtagaca

SEQ ID NO: 122 - DNA, 30 bp, artificial sequence
  Primer N1213
atggcgaaatggcagtactcgggggaatgc

SEQ ID NO: 123 - DNA, 7463 bp, artificial sequence
  Synthetic construct
ccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaatgtccaatttactgcccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttatgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtccgccaccgcggtggagct

SEQ ID NO: 124 - DNA, 24 bp, artificial sequence
  Primer N1262
cacgtaagggcatgatagaattgg

SEQ ID NO: 125 - DNA, 26 bp, artificial sequence
  Primer N1263
ggatatagcagttgttgtacactagc

SEQ ID NO: 126 - DNA, 25 bp, artificial sequence
  Primer N1230
gtaagaaggttggtattccagctgg

SEQ ID NO: 127 - DNA, 26 bp, artificial sequence
  Primer N1231
ctgtttcgataccagaacctagaccg

SEQ ID NO: 128 - DNA, 27 bp, artificial sequence
  Primer N1228
caacggttcatcatctcatggatctgc

SEQ ID NO: 129 - DNA, 26 bp, artificial sequence
  Primer N1229
gttacttggttctggcgaggtattgg

SEQ ID NO: 130 - DNA, 24 bp, artificial sequence
  Primer N1223
caaggaattaccatcaccgtcacc

SEQ ID NO: 131 - DNA, 27 bp, artificial sequence
  Primer N1225
cttgtgtataatgataaattggttggg

SEQ ID NO: 132 - DNA, 9612 bp, artificial sequence
  Synthetic construct
aaacagtatggaagaatgtaagatggctaagatttactaccaagaagactgtaacttgtccttgttggatggtaagactatcgccgttatcggttacggttctcaaggtcacgctcatgccctgaatgctaaggaatccggttgtaacgttatcattggtttatacgaaggtgctaaggattggaaaagagctgaagaacaaggtttcgaagtctacaccgctgctgaagctgctaagaaggctgacatcattatgatcttgatcaacgatgaaaagcaggctaccatgtacaaaaacgacatcgaaccaaacttggaagccggtaacatgttgatgttcgctcacggtttcaacatccatttcggttgtattgttccaccaaaggacgttgatgtcactatgatcgctccaaagggtccaggtcacaccgttagatccgaatacgaagaaggtaaaggtgtcccatgcttggttgctgtcgaacaagacgctactggcaaggctttggatatggctttggcctacgctttagccatcggtggtgctagagccggtgtcttggaaactaccttcagaaccgaaactgaaaccgacttgttcggtgaacaagctgttttatgtggtggtgtctgcgctttgatgcaggccggttttgaaaccttggttgaagccggttacgacccaagaaacgcttacttcgaatgtatccacgaaatgaagttgatcgttgacttgatctaccaatctggtttctccggtatgcgttactctatctccaacactgctgaatacggtgactacattaccggtccaaagatcattactgaagataccaagaaggctatgaagaagattttgtctgacattcaagatggtacctttgccaaggacttcttggttgacatgtctgatgctggttcccaggtccacttcaaggctatgagaaagttggcctccgaacacccagctgaagttgtcggtgaagaaattagatccttgtactcctggtccgacgaagacaagttgattaacaactgatattttcctctggccctgcaggcctatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctccgcggtgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatctcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagtcaccggtggcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgttt

SEQ ID NO: 133 - DNA, 13114 bp, artificial sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtggccggcttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaacacgtgagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgaggatccactagttctagagcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaatcaaaaagttaaaattgtacgaatagattcaccacttcttaacaaatcaaacccttcattgattttctcgaatggcaatacatgtgtaattaaaggatcaagagcaaacttcttcgccataaagtcggcaacaagttttggaacactatccttgctcttaaaaccgccaaatatagctcccttccatgtacgaccgcttagcaacagcataggattcatcgacaaattttgtgaatcaggaggaacacctacgatcacactgactccatatgcctcttgacagcaggacaacgcagttaccatagtatcaagacggcctataacttcaaaagagaaatcaactccaccgtttgacatttcagtaaggacttcttgtattggtttcttataatcttgagggttaacacattcagtagccccgacctccttagcttttgcaaatttgtccttattgatgtctacacctataatcctcgctgcgcctgcagctttacaccccataataacgcttagtcctactcctcctaaaccgaatactgcacaagtcgaaccctgtgtaacctttgcaactttaactgcggaaccgtaaccggtggaaaatccgcaccctatcaagcaaactttttccagtggtgaagctgcatcgattttagcgacagatatctcgtccaccactgtgtattgggaaaatgtagaagtaccaaggaaatggtgtataggtttccctctgcatgtaaatctgcttgtaccatcctgcatagtacctctaggcatagacaaatcatttttaaggcagaaattaccctcaggatgtttgcagactctacacttaccacattgaggagtgaacagtgggatcactttatcaccaggacgaacagtggtaacaccttcacctatggattcaacgattccggcagcctcgtgtcccgcgattactggcaaaggagtaactagagtgccactcaccacatggtcgtcggatctacagattccggtggcaaccatcttgattctaacctcgtgtgcttttggtggcgctacttctacttcttctatgctaaacggctttttctcttcccacaaaactgccgctttacacttaataactttaccggctgttgacatcctcagctagctattgtaatatgtgtgtttgtttggattattaagaagaataattacaaaaaaaattacaaaggaaggtaattacaacagaattaagaaaggacaagaaggaggaagagaatcagttcattatttcttctttgttatataacaaacccaagtagcgatttggccatacattaaaagttgagaaccaccctccctggcaacagccacaactcgttaccattgttcatcacgatcatgaaactcgccgtcagctgaaatttcacctcagtggatctctctttttattcttcatcgttccactaacctttttccatcagctggcagggaacggaaagtggaatcccatttagcgagcttcctcttttcttcaagaaaagacgaagcttgtgtgtgggtgcgcgcgctagtatctttccacattaagaaatataccataaaggttacttagacatcactatggctatatatatatatatatatatatgtaacttagcaccatcgcgcgtgcatcactgcatgtgttaaccgaaaagtttggcgaacacttcaccgacacggtcatttagatctgtcgtctgcattgcacgtcccttagccttaaatcctaggcgggagcattctcgtgtaattgtgcagcctgcgtagcaactcaacatagcgtagtctacccagtttttcaagggtttatcgttagaagattctcccttttcttcctgctcacaaatcttaaagtcatacattgcacgactaaatgcaagcgacgtcagggaaagatatgagctatacagcggaatttccatatcactcagattttgttatctaattttttccttcccacgtccgcgggaatctgtgtatattactgcatctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatgcaagatttaaagtaaattcacggccctgcaggcctcagctcttgttttgttctgcaaataacttacccatctttttcaaaactttaggtgcaccctcctttgctagaataagttctatccaatacatcctatttggatctgcttgagcttctttcatcacggatacgaattcattttctgttctcacaattttggacacaactctgtcttccgttgccccgaaactttctggcagttttgagtaattccacataggaatgtcattataactctggttcggaccatgaatttccctctcaaccgtgtaaccatcgttattaatgataaagcagattgggtttatcttctctctaatggctagtcctaattcttggacagtcagttgcaatgatccatctccgataaacaataaatgtctagattctttatctgcaatttggctgcctagagctgcggggaaagtgtatcctatagatccccacaagggttgaccaataaaatgtgatttcgatttcagaaatatagatgaggcaccgaagaaagaagtgccttgttcagccacgatcgtctcattactttgggtcaaattttcgacagcttgccacagtctatcttgtgacaacagcgcgttagaaggtacaaaatcttcttgctttttatctatgtacttgcctttatattcaatttcggacaagtcaagaagagatgatatcagggattcgaagtcgaaattttggattctttcgttgaaaattttaccttcatcgatattcaaggaaatcattttattttcattaagatggtgagtaaatgcacccgtactagaatcggtaagctttacacccaacataagaataaaatcagcagattccacaaattccttcaagtttggctctgacagagtaccgttgtaaatccccaaaaatgagggcaatgcttcatcaacagatgatttaccaaagttcaaagtagtaataggtaacttagtctttgaaataaactgagtaacagtcttctctaggccgaacgatataatttcatggcctgtgattacaattggtttcttggcattcttcagactttcctgtattttgttcagaatctcttgatcagatgtattcgacgtggaattttccttcttaagaggcaaggatggtttttcagccttagcggcagctacatctacaggtaaattgatgtaaaccggctttctttcctttagtaaggcagacaacactctatcaatttcaacagttgcattctcggctgtcaataaagtcctggcagcagtaaccggttcgtgcatcttcataaagtgcttgaaatcaccatcagccaacgtatggtgaacaaacttaccttcgttctgcactttcgaggtaggagatcccacgatctcaacaacaggcaggttctcagcataggagcccgctaagccattaactgcggataattcgccaacaccaaatgtagtcaagaatgccgcagcctttttcgttcttgcgtacccgtcggccatataggaggcatttaactcattagcatttcccacccatttcatatctttgtgtgaaataatttgatctagaaattgcaaattgtagtcacctggtactccgaatatttcttctatacctaattcgtgtaatctgtccaacagatagtcacctactgtatacatgtttaaactttgtttactagtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 134 - DNA, 1494 bp, Saccharomyces cerevisiae
atgactttgagtaagtattctaaaccaactctaaacgaccctaatttattcagagaatctggttatattgacggaaaatgggttaagggcactgacgaagtttttgaggtggtagaccctgcttccggcgaaatcatagcaagagttcccgaacaaccagtctccgtggttgaggaagcgattgatgttgcctatgaaactttcaagacgtacaagaatacaacaccaagagagagggcaaagtggctcagaaacatgtataacttaatgcttgaaaatttggatgatctggcaaccatcattactttagaaaatggtaaagctctaggggaagctaaaggagaaatcaaatacgcggcttcgtattttgagtggtacgccgaggaagcaccccgtttatatggtgctactattcaacccttgaaccctcacaacagagtattcacaattaggcaacctgttggtgtatgcggtataatttgtccatggaattttccgagcgccatgatcacgagaaaggccgccgctgctttagctgtgggctgcacagtagtcatcaagccagactctcaaacgccgctatctgctttagcaatggcatatttggctgaaaaggcaggctttcccaagggttcgtttaatgttattctttcacatgccaacacaccaaagcttggtaaaacattatgtgaatcaccaaaagtcaagaaagttacttttactggttctacaaacgtcggtaaaatcttgatgaaacaatcttcttctactttgaagaaactgtcttttgagctgggtggtaacgcccctttcatagtctttgaggatgccgatttggatcaagccttggaacaagccatggcttgtaaatttaggggtttgggtcaaacatgtgtgtgcgcaaatagactttacgttcactcatccataattgataaatttgcgaaattactcgcggagagggtcaaaaaattcgtaattggccatggtttggacccaaaaactacacatggttgtgtcattaactccagcgctattgaaaaagttgaaagacataaacaggatgccattgataagggagcaaaagttgtgcttgaaggtggacgtttaactgagttaggtcctaacttttatgctccagtaattttgtcacacgttccctcaacagctattgtttccaaggaggagacttttggtccattatgtccaatcttttcttttgatactatggaagaagttgtcggatatgctaatgatactgagtttggtttagcagcatatgtcttttctaaaaatgtcaacactttatacactgtgtctgaagctttggaaactggtatggtttcatgtaatacaggtgttttttcggattgttctataccatttggtggtgttaaagagtcaggatttggaagagaaggttcgctatatggtattgaagattacactgttttgaagaccatcacaattgggaatttgccaaacagcatttaa

SEQ ID NO: 135 - protein, 497 aa, Saccharomyces cerevisiae
MTLSKYSKPTLNDPNLFRESGYIDGKWVKGTDEVFEVVDPASGEIIARVPEQPVSVVEEAIDVAYETFKTYKNTTPRERAKWLRNMYNLMLENLDDLATIITLENGKALGEAKGEIKYAASYFEWYAEEAPRLYGATIQPLNPHNRVFTIRQPVGVCGIICPWNFPSAMITRKAAAALAVGCTVVIKPDSQTPLSALAMAYLAEKAGFPKGSFNVILSHANTPKLGKTLCESPKVKKVTFTGSTNVGKILMKQSSSTLKKLSFELGGNAPFIVFEDADLDQALEQAMACKFRGLGQTCVCANRLYVHSSIIDKFAKLLAERVKKFVIGHGLDPKTTHGCVINSSAIEKVERHKQDAIDKGAKVVLEGGRLTELGPNFYAPVILSHVPSTAIVSKEETFGPLCPIFSFDTMEEVVGYANDTEFGLAAYVFSKNVNTLYTVSEALETGMVSCNTGVFSDCSIPFGGVKESGFGREGSLYGIEDYTVLKTITIGNLPNSI

SEQ ID NO: 136 - DNA, 1323 bp, Saccharomyces cerevisiae
atgcttgctgtcagaagattaacaagatacacattccttaagcgaacgcatccggtgttatatactcgtcgtgcatataaaattttgccttcaagatctactttcctaagaagatcattattacaaacacaactgcactcaaagatgactgctcatactaatatcaaacagcacaaacactgtcatgaggaccatcctatcagaagatcggactctgccgtgtcaattgtacatttgaaacgtgcgcccttcaaggttacagtgattggttctggtaactgggggaccaccatcgccaaagtcattgcggaaaacacagaattgcattcccatatcttcgagccagaggtgagaatgtgggtttttgatgaaaagatcggcgacgaaaatctgacggatatcataaatacaagacaccagaacgttaaatatctacccaatattgacctgccccataatctagtggccgatcctgatcttttacactccatcaagggtgctgacatccttgttttcaacatccctcatcaatttttaccaaacatagtcaaacaattgcaaggccacgtggcccctcatgtaagggccatctcgtgtctaaaagggttcgagttgggctccaagggtgtgcaattgctatcctcctatgttactgatgagttaggaatccaatgtggcgcactatctggtgcaaacttggcaccggaagtggccaaggagcattggtccgaaaccaccgtggcttaccaactaccaaaggattatcaaggtgatggcaaggatgtagatcataagattttgaaattgctgttccacagaccttacttccacgtcaatgtcatcgatgatgttgctggtatatccattgccggtgccttgaagaacgtcgtggcacttgcatgtggtttcgtagaaggtatgggatggggtaacaatgcctccgcagccattcaaaggctgggtttaggtgaaattatcaagttcggtagaatgtttttcccagaatccaaagtcgagacctactatcaagaatccgctggtgttgcagatctgatcaccacctgctcaggcggtagaaacgtcaaggttgccacatacatggccaagaccggtaagtcagccttggaagcagaaaaggaattgcttaacggtcaatccgcccaagggataatcacatgcagagaagttcacgagtggctacaaacatgtgagttgacccaagaattcccattattcgaggcagtctaccagatagtctacaacaacgtccgcatggaagacctaccggagatgattgaagagctagacatcgatgacgaatag

SEQ ID NO: 137 - protein, 440 aa, Saccharomyces cerevisiae
MLAVRRLTRYTFLKRTHPVLYTRRAYKILPSRSTFLRRSLLQTQLHSKMTAHTNIKQHKHCHEDHPIRRSDSAVSIVHLKRAPFKVTVIGSGNWGTTIAKVIAENTELHSHIFEPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGIQCGALSGANLAPEVAKEHWSETTVAYQLPKDYQGDGKDVDHKILKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEFPLFEAVYQIVYNNVRMEDLPEMIEELDIDDE

SEQ ID NO: 138 - DNA, 81 bp, artificial sequence
  Primer BK506
gggtaataactgatataattaaattgaagctctaatttgtgagtttagtacaccttggctaactcgttgtatcatcactgg

SEQ ID NO: 139 - DNA, 38 bp, artificial sequence; identical to SEQ ID NO: 3
  Primer LA468

SEQ ID NO: 140 - DNA, 31 bp, artificial sequence
  Primer LA492
gctaaattcgagtgaaacacaggaagaccag

SEQ ID NO: 141 - DNA, 90 bp, artificial sequence
  Primer LA512
gtattttggtagattcaattctctttccctttccttttccttcgctccccttccttatcagcattgcggattacgtattctaatgttcag

SEQ ID NO: 142 - DNA, 90 bp, artificial sequence
  Primer LA513
ttggttgggggaaaaagaggcaacaggaaagatcagagggggagggggggggagagtgtcaccttggctaactcgttgtatcatcactgg

SEQ ID NO: 143 - DNA, 21 bp, artificial sequence
  Primer oBP264
tcggtgcgggcctcttcgcta

SEQ ID NO: 144 - DNA, 21 bp, artificial sequence
  Primer oBP265
aatgtgagttagctcactcat

SEQ ID NO: 145 - DNA, 57 bp, artificial sequence
  Primer oBP438
aattggatccggcgcgccgtttaaacggccggccaatgtggctgtggtttcagggtc

SEQ ID NO: 146 - DNA, 49 bp, artificial sequence
  Primer oBP439
aatttctagattaattaagcggccgcaaggccatgaagctttttctttc

SEQ ID NO: 147 - DNA, 24 bp, artificial sequence
  Primer oBP452
ttctcgacgtgggcctttttcttg

SEQ ID NO: 148 - DNA, 49 bp, artificial sequence
  Primer oBP453
tgcagctttaaataatcggtgtcactactttgccttcgtttatcttgcc

SEQ ID NO: 149 - DNA, 49 bp, artificial sequence
  Primer oBP454
gagcaggcaagataaacgaaggcaaagtagtgacaccgattatttaaag

SEQ ID NO: 150 - DNA, 49 bp, artificial sequence
  Primer oBP455
tatggaccctgaaaccacagccacattgtaaccaccacgacggttgttg

SEQ ID NO: 151 - DNA, 49 bp, artificial sequence
  Primer oBP456
tttagcaacaaccgtcgtggtggttacaatgtggctgtggtttcagggt

SEQ ID NO: 152 - DNA, 49 bp, artificial sequence
  Primer oBP457
ccagaaaccctatacctgtgtggacgtaaggccatgaagctttttcttt

SEQ ID NO: 153 - DNA, 49 bp, artificial sequence
  Primer oBP458
attggaaagaaaaagcttcatggccttacgtccacacaggtatagggtt

SEQ ID NO: 154 - DNA, 22 bp, artificial sequence
  Primer oBP459
cataagaacacctttggtggag

SEQ ID NO: 155 - DNA, 22 bp, artificial sequence
  Primer oBP460
aggattatcattcataagtttc

SEQ ID NO: 156 - DNA, 23 bp, artificial sequence
  Primer oBP461
ttcttggagctgggacatgtttg

SEQ ID NO: 157 - DNA, 22 bp, artificial sequence
  Primer oBP450
tgatgatatttcataaataatg

SEQ ID NO: 158 - DNA, 23 bp, artificial sequence
  Primer oBP451
atgcgtccatctttacagtcctg

SEQ ID NO: 159 - DNA, 22 bp, artificial sequence
  Primer oBP440
tacgtacggaccaatcgaagtg

SEQ ID NO: 160 - DNA, 49 bp, artificial sequence
  Primer oBP441
aattcgtttgagtacactactaatggctttgttggcaatatgtttttgc

SEQ ID NO: 161 - DNA, 49 bp, artificial sequence
  Primer oBP442
atatagcaaaaacatattgccaacaaagccattagtagtgtactcaaac

SEQ ID NO: 162 - DNA, 49 bp, artificial sequence
  Primer oBP443
tatggaccctgaaaccacagccacattcttgttatttataaaaagacac

SEQ ID NO: 163 - DNA, 49 bp, artificial sequence
  Primer oBP444
ctcccgtgtctttttataaataacaagaatgtggctgtggtttcagggt

SEQ ID NO: 164 - DNA, 49 bp, artificial sequence
  Primer oBP445
taccgtaggcgtccttaggaaagatagaaggccatgaagctttttcttt

SEQ ID NO: 165 - DNA, 49 bp, artificial sequence
  Primer oBP446
attggaaagaaaaagcttcatggccttctatctttcctaaggacgccta

SEQ ID NO: 166 - DNA, 21 bp, artificial sequence
  Primer oBP447
ttattgtttggcatttgtagc

SEQ ID NO: 167 - DNA, 22 bp, artificial sequence
  Primer oBP448
ccaagcatctcataaacctatg

SEQ ID NO: 168 - DNA, 22 bp, artificial sequence
  Primer oBP449
tgtgcagatgcagatgtgagac

SEQ ID NO: 169 - DNA, 17 bp, artificial sequence
  Primer oBP554
agttattgataccgtac

SEQ ID NO: 170 - DNA, 19 bp, artificial sequence
  Primer oBP555
cgagataccgtaggcgtcc

SEQ ID NO: 171 - DNA, 24 bp, artificial sequence
  Primer oBP513
ttatgtatgctcttctgacttttc

SEQ ID NO: 172 - DNA, 49 bp, artificial sequence
  Primer oBP515
aataattagagattaaatcgctcattttttgccagtttcttcaggcttc

SEQ ID NO: 173 - DNA, 49 bp, artificial sequence
  Primer oBP516
agcctgaagaaactggcaaaaaatgagcgatttaatctctaattattag

SEQ ID NO: 174 - DNA, 49 bp, artificial sequence
  Primer oBP517
tatggaccctgaaaccacagccacatttttcaatcattggagcaatcat

SEQ ID NO: 175 - DNA, 49 bp, artificial sequence
  Primer oBP518
taaaatgattgctccaatgattgaaaaatgtggctgtggtttcagggtc

SEQ ID NO: 176 - DNA, 49 bp, artificial sequence
  Primer oBP519
accgtaggtgttgtttgggaaagtggaaggccatgaagctttttctttc

SEQ ID NO: 177 - DNA, 49 bp, artificial sequence
  Primer oBP520
ttggaaagaaaaagcttcatggccttccactttcccaaacaacacctac

SEQ ID NO: 178 - DNA, 23 bp, artificial sequence
  Primer oBP521
ttattgcttagcgttggtagcag

SEQ ID NO: 179 - DNA, 21 bp, artificial sequence
  Primer oBP511
tttttggtggttccggcttcc

SEQ ID NO: 180 - DNA, 22 bp, artificial sequence
  Primer oBP512
aaagttggcatagcggaaactt

SEQ ID NO: 181 - DNA, 16 bp, artificial sequence
  Primer oBP550
gtcattgacaccatct

SEQ ID NO: 182 - DNA, 19 bp, artificial sequence
  Primer oBP551
agagataccgtaggtgttg

SEQ ID NO: 183 - DNA, 33 bp, artificial sequence
  Primer oBP530
aattggcgcgccatgaaagctctggtttatcac

SEQ ID NO: 184 - DNA, 49 bp, artificial sequence
  Primer oBP531
tgaatcatgagttttatgttaattagctcaggcagcgcctgcgttcgag

SEQ ID NO: 185 - DNA, 49 bp, artificial sequence
  Primer oBP532
atcctctcgaacgcaggcgctgcctgagctaattaacataaaactcatg

SEQ ID NO: 186 - DNA, 34 bp, artificial sequence
  Primer oBP533
aattgtttaaacaagtaaataaattaatcagcat

SEQ ID NO: 187 - DNA, 49 bp, artificial sequence
  Primer oBP536
acacaatacaataacaagaagaacaaaatgaaagctctggtttatcacg

SEQ ID NO: 188 - DNA, 49 bp, artificial sequence
  Primer oBP546
agcgtatacatctgttgggaaagtagaaggccatgaagctttttctttc

SEQ ID NO: 189 - DNA, 49 bp, artificial sequence
  Primer oBP547
ttggaaagaaaaagcttcatggccttctactttcccaacagatgtatac

SEQ ID NO: 190 - DNA, 22 bp, artificial sequence
  Primer oBP539
ttattgtttagcgttagtagcg

SEQ ID NO: 191 - DNA, 72 bp, artificial sequence
  Primer oBP542
cataatcaatctcaaagagaacaacacaatacaataacaagaagaacaaaatgaaagctctggtttatcacg

SEQ ID NO: 192 - DNA, 21 bp, artificial sequence
  Primer oBP540
taggcataatcaccgaagaag

SEQ ID NO: 193 - DNA, 21 bp, artificial sequence
  Primer oBP541
aaaatggtaagcagctgaaag

SEQ ID NO: 194 - DNA, 17 bp, artificial sequence
  Primer oBP552
agttgttagaactgttg

SEQ ID NO: 195 - DNA, 19 bp, artificial sequence
  Primer oBP553
gacgatagcgtatacatct

SEQ ID NO: 196 - DNA, 22 bp, artificial sequence
  Primer oBP582
cttagcctctagccatagccat

SEQ ID NO: 197 - DNA, 23 bp, artificial sequence
  Primer AA270
ttagttttgctggccgcatcttc

SEQ ID NO: 198 - DNA, 21 bp, artificial sequence
  Primer oBP591
cccattaatatactattgaga

SEQ ID NO: 199 - DNA, 4586 bp, artificial sequence
  Synthetic construct
gggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgcattgcggattacgtattctaatgttcagataacttcgtatagcatacattatacgaagttatctagggattcataaccattttctcaatcgaattacacagaacacaccgtacaaacctctctatcataactacttaatagtcacacacgtactcgtctaaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaactcgagatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgtttttttttgttttgttctcactgtatttacatttctatttagtatttagttattcatataatctataacttcgtatagcatacattatacgaagttatccagtgatgatacaacgagttagccaaggtg

SEQ ID NO: 200 - DNA, 1716 bp, Streptococcus mutans
atgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatga

SEQ ID NO: 201 - DNA, 15456 bp, artificial sequence
  Synthetic construct
aaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggcgcgccactggtagagagcgactttgtatgccccaattgcgaaacccgcgatatccttctcgattctttagtacccgaccaggacaaggaaaaggaggtcgaaacgtttttgaagaaacaagaggaactacacggaagctctaaagatggcaaccagccagaaactaagaaaatgaagttgatggatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccacccgggccccccctcgaggtcgacggtatcgataagcttgatatcgaattcctgcagcccgggggatccactagttctagagcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaatcaggcagcgcctgcgttcgagaggatgatcttcatcgccttctccttggcgccattgaggaatacctgataggcgtgctcgatctcggccagctcgaagcgatgggtaatcatcttcttcaacggaagcttgtcggtcgaggcgaccttcatcagcatgggcgtcgtgttcgtgttcaccagtcccgtggtgatcgtcaggttcttgatccagagcttctgaatctcgaagtcaaccttgacgccatgcacgccgacgttggcgatgtgcgcgccgggcttgacgatctcctggcagatgtcccaagtcgccggtatgcccaccgcctcgatcgcaacatcgactccctctgccgcaatcctatgcacggcttcgacaacgttctccgtgccggagttgatggtgtgcgttgccccgagctccttggcgagctggaggcgattctcgtccatgtcgatcacgatgatggtcgagggggagtagaactgggcggtcaacagtacggacatgccgacggggcccgcgccgacaatagccaccgcatcgcccggctggacattcccatactggacgccgatttcgtggccggtgggcaggatgtcgctcagcaggacggcgatttcgtcgtcaattgtctgggggatcttgtagaggctgttgtcggcatgcgggatgcggacgtattcggcctgcacgccatcgatcatgtaacccaggatccacccgccgtcgcggcaatgggagtaaagctgcttcttgcagtagtcgcacgagccgcaagaagtgacgcaggaaatcaggaccttgtcgcctttcttgaactgcgtgacactctcgcccacttcctcgatgacgcctaccccttcatggcccaggatgcgcccgtcggcgacctctggattcttgcctttgtagatgccgagatccgtgccgcagatcgtggtcttcaaaacccgtactactacatccgtgggcttttgaagggtgggcttgggcttgtcttcaagcgagatcttgtggtcaccgtgataaaccagagctttcatcctcagctattgtaatatgtgtgtttgtttggattattaagaagaataattacaaaaaaaattacaaaggaaggtaattacaacagaattaagaaaggacaagaaggaggaagagaatcagttcattatttcttctttgttatataacaaacccaagtagcgatttggccatacattaaaagttgagaaccaccctccctggcaacagccacaactcgttaccattgttcatcacgatcatgaaactcgctgtcagctgaaatttcacctcagtggatctctctttttattcttcatcgttccactaacctttttccatcagctggcagggaacggaaagtggaatcccatttagcgagcttcctcttttcttcaagaaaagacgaagcttgtgtgtgggtgcgcgcgctagtatctttccacattaagaaatataccataaaggttacttagacatcactatggctatatatatatatatatatatatatgtaacttagcaccatcgcgcgtgcatcactgcatgtgttaaccgaaaagtttggcgaacacttcaccgacacggtcatttagatctgtcgtctgcattgcacgtcccttagccttaaatcctaggcgggagcattctcgtgtaattgtgcagcctgcgtagcaactcaacatagcgtagtctacccagtttttcaagggtttatcgttagaagattctcccttttcttcctgctcacaaatcttaaagtcatacattgcacgactaaatgcaagcatgcggatcccccgggctgcaggaattcgatatcaagcttatcgataccgtcgactggccattaatctttcccatattagatttcgccaagccatgaaagttcaagaaaggtctttagacgaattacccttcatttctcaaactggcgtcaagggatcctggtatggttttatcgttttatttctggttcttatagcatcgttttggacttctctgttcccattaggcggttcaggagccagcgcagaatcattctttgaaggatacttatcctttccaattttgattgtctgttacgttggacataaactgtatactagaaattggactttgatggtgaaactagaagatatggatcttgataccggcagaaaacaagtagatttgactcttcgtagggaagaaatgaggattgagcgagaaacattagcaaaaagatccttcgtaacaagatttttacatttctggtgttgaagggaaagatatgagctatacagcggaatttccatatcactcagattttgttatctaattttttccttcccacgtccgcgggaatctgtgtatattactgcatctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatgcaagatttaaagtaaattcacggccctgcaggcctcagctcttgttttgttctgcaaataacttacccatctttttcaaaactttaggtgcaccctcctttgctagaataagttctatccaatacatcctatttggatctgcttgagcttctttcatcacggatacgaattcattttctgttctcacaattttggacacaactctgtcttccgttgccccgaaactttctggcagttttgagtaattccacataggaatgtcattataactctggttcggaccatgaatttccctctcaaccgtgtaaccatcgttattaatgataaagcagattgggtttatcttctctctaatggctagtcctaattcttggacagtcagttgcaatgatccatctccgataaacaataaatgtctagattctttatctgcaatttggctgcctagagctgcggggaaagtgtatcctatagatccccacaagggttgaccaataaaatgtgatttcgatttcagaaatatagatgaggcaccgaagaaagaagtgccttgttcagccacgatcgtctcattactttgggtcaaattttcgacagcttgccacagtctatcttgtgacaacagcgcgttagaaggtacaaaatcttcttgctttttatctatgtacttgcctttatattcaatttcggacaagtcaagaagagatgatatcagggattcgaagtcgaaattttggattctttcgttgaaaattttaccttcatcgatattcaaggaaatcattttattttcattaagatggtgagtaaatgcacccgtactagaatcggtaagctttacacccaacataagaataaaatcagcagattccacaaattccttcaagtttggctctgacagagtaccgttgtaaatccccaaaaatgagggcaatgcttcatcaacagatgatttaccaaagttcaaagtagtaataggtaacttagtctttgaaataaactgagtaacagtcttctctaggccgaacgatataatttcatggcctgtgattacaattggtttcttggcattcttcagactttcctgtattttgttcagaatctcttgatcagatgtattcgacgtggaattttccttcttaagaggcaaggatggtttttcagccttagcggcagctacatctacaggtaaattgatgtaaaccggctttctttcctttagtaaggcagacaacactctatcaatttcaacagttgcattctcggctgtcaataaagtcctggcagcagtaaccggttcgtgcatcttcataaagtgcttgaaatcaccatcagccaacgtatggtgaacaaacttaccttcgttctgcactttcgaggtaggagatcccacgatctcaacaacaggcaggttctcagcataggagcccgctaagccattaactgcggataattcgccaacaccaaatgtagtcaagaatgccgcagcctttttcgttcttgcgtacccgtcggccatataggaggcatttaactcattagcatttcccacccatttcatatctttgtgtgaaataatttgatctagaaattgcaaattgtagtcacctggtactccgaatatttcttctatacctaattcgtgtaatctgtccaacagatagtcacctactgtatacattttgtttactagtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgaaaaagcgtgttttttattcaaaatgattctaactcccttacgtaatcaaggaatctttttgccttggcctccgcgtcattaaacttcttgttgttgacgctaacattcaacgctagtatatattcgtttttttcaggtaagttcttttcaacgggtcttactgatgaggcagtcgcgtctgaacctgttaagaggtcaaatatgtcttcttgaccgtacgtgtcttgcatgttattagctttgggaatttgcatcaagtcataggaaaatttaaatcttggctctcttgggctcaaggtgacaaggtcctcgaaaatagggcgcgccccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtct

SEQ ID NO: 202 - DNA, 1559 bp, artificial sequence
  Synthetic construct
gcattgcggattacgtattctaatgttcagtaccgttcgtataatgtatgctatacgaagttatgcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccagtgatgatacaacgagttagccaaggtg

SEQ ID NO: 203 - DNA, 11856 bp, artificial sequence
  Synthetic construct
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcgctgaggatgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctagttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagctacgtcaagcaatgccattcacacaatctggtgatgacgatgcctttaaaatctatcagtaaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctccgcggtgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 204 - DNA, 15539 bp, artificial sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggcgcgccactggtagagagcgactttgtatgccccaattgcgaaacccgcgatatccttctcgattctttagtacccgaccaggacaaggaaaaggaggtcgaaacgtttttgaagaaacaagaggaactacacggaagctctaaagatggcaaccagccagaaactaagaaaatgaagttgatggatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccacccgggccccccctcgaggtcgacggtatcgataagcttgatatcgaattcctgcagcccgggggatccactagttctagagcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaatcaaaaagttaaaattgtacgaatagattcaccacttcttaacaaatcaaacccttcattgattttctcgaatggcaatacatgtgtaattaaaggatcaagagcaaacttcttcgccataaagtcggcaacaagttttggaacactatccttgctcttaaaaccgccaaatatagctcccttccatgtacgaccgcttagcaacagcataggattcatcgacaaattttgtgaatcaggaggaacacctacgatcacactgactccatatgcctcttgacagcaggacaacgcagttaccatagtatcaagacggcctataacttcaaaagagaaatcaactccaccgtttgacatttcagtaaggacttcttgtattggtttcttataatcttgagggttaacacattcagtagccccgacctccttagcttttgcaaatttgtccttattgatgtctacacctataatcctcgctgcgcctgcagctttacaccccataataacgcttagtcctactcctcctaaaccgaatactgcacaagtcgaaccctgtgtaacctttgcaactttaactgcggaaccgtaaccggtggaaaatccgcaccctatcaagcaaactttttccagtggtgaagctgcatcgattttagcgacagatatctcgtccaccactgtgtattgggaaaatgtagaagtaccaaggaaatggtgtataggtttccctctgcatgtaaatctgcttgtaccatcctgcatagtacctctaggcatagacaaatcatttttaaggcagaaattaccctcaggatgtttgcagactctacacttaccacattgaggagtgaacagtgggatcactttatcaccaggacgaacagtggtaacaccttcacctatggattcaacgattccggcagcctcgtgtcccgcgattactggcaaaggagtaactagagtgccactcaccacatggtcgtcggatctacagattccggtggcaaccatcttgattctaacctcgtgtgcttttggtggcgctacttctacttcttctatgctaaacggctttttctcttcccacaaaactgccgctttacacttaataactttaccggctgttgacatcctcagctagctattgtaatatgtgtgtttgtttggattattaagaagaataattacaaaaaaaattacaaaggaaggtaattacaacagaattaagaaaggacaagaaggaggaagagaatcagttcattatttcttctttgttatataacaaacccaagtagcgatttggccatacattaaaagttgagaaccaccctccctggcaacagccacaactcgttaccattgttcatcacgatcatgaaactcgctgtcagctgaaatttcacctcagtggatctctctttttattcttcatcgttccactaacctttttccatcagctggcagggaacggaaagtggaatcccatttagcgagcttcctcttttcttcaagaaaagacgaagcttgtgtgtgggtgcgcgcgctagtatctttccacattaagaaatataccataaaggttacttagacatcactatggctatatatatatatatatatatatgtaacttagcaccatcgcgcgtgcatcactgcatgtgttaaccgaaaagtttggcgaacacttcaccgacacggtcatttagatctgtcgtctgcattgcacgtcccttagccttaaatcctaggcgggagcattctcgtgtaattgtgcagcctgcgtagcaactcaacatagcgtagtctacccagtttttcaagggtttatcgttagaagattctcccttttcttcctgctcacaaatcttaaagtcatacattgcacgactaaatgcaagcatgcggatcccccgggctgcaggaattcgatatcaagcttatcgataccgtcgactggccattaatctttcccatattagatttcgccaagccatgaaagttcaagaaaggtctttagacgaattacccttcatttctcaaactggcgtcaagggatcctggtatggttttatcgttttatttctggttcttatagcatcgttttggacttctctgttcccattaggcggttcaggagccagcgcagaatcattctttgaaggatacttatcctttccaattttgattgtctgttacgttggacataaactgtatactagaaattggactttgatggtgaaactagaagatatggatcttgataccggcagaaaacaagtagatttgactcttcgtagggaagaaatgaggattgagcgagaaacattagcaaaaagatccttcgtaacaagatttttacatttctggtgttgaagggaaagatatgagctatacagcggaatttccatatcactcagattttgttatctaattttttccttcccacgtccgcgggaatctgtgtatattactgcatctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatgcaagatttaaagtaaattcacggccctgcaggcctcagctcttgttttgttctgcaaataacttacccatctttttcaaaactttaggtgcaccctcctttgctagaataagttctatccaatacatcctatttggatctgcttgagcttctttcatcacggatacgaattcattttctgttctcacaattttggacacaactctgtcttccgttgccccgaaactttctggcagttttgagtaattccacataggaatgtcattataactctggttcggaccatgaatttccctctcaaccgtgtaaccatcgttattaatgataaagcagattgggtttatcttctctctaatggctagtcctaattcttggacagtcagttgcaatgatccatctccgataaacaataaatgtctagattctttatctgcaatttggctgcctagagctgcggggaaagtgtatcctatagatccccacaagggttgaccaataaaatgtgatttcgatttcagaaatatagatgaggcaccgaagaaagaagtgccttgttcagccacgatcgtctcattactttgggtcaaattttcgacagcttgccacagtctatcttgtgacaacagcgcgttagaaggtacaaaatcttcttgctttttatctatgtacttgcctttatattcaatttcggacaagtcaagaagagatgatatcagggattcgaagtcgaaattttggattctttcgttgaaaattttaccttcatcgatattcaaggaaatcattttattttcattaagatggtgagtaaatgcacccgtactagaatcggtaagctttacacccaacataagaataaaatcagcagattccacaaattccttcaagtttggctctgacagagtaccgttgtaaatccccaaaaatgagggcaatgcttcatcaacagatgatttaccaaagttcaaagtagtaataggtaacttagtctttgaaataaactgagtaacagtcttctctaggccgaacgatataatttcatggcctgtgattacaattggtttcttggcattcttcagactttcctgtattttgttcagaatctcttgatcagatgtattcgacgtggaattttccttcttaagaggcaaggatggtttttcagccttagcggcagctacatctacaggtaaattgatgtaaaccggctttctttcctttagtaaggcagacaacactctatcaatttcaacagttgcattctcggctgtcaataaagtcctggcagcagtaaccggttcgtgcatcttcataaagtgcttgaaatcaccatcagccaacgtatggtgaacaaacttaccttcgttctgcactttcgaggtaggagatcccacgatctcaacaacaggcaggttctcagcataggagcccgctaagccattaactgcggataattcgccaacaccaaatgtagtcaagaatgccgcagcctttttcgttcttgcgtacccgtcggccatataggaggcatttaactcattagcatttcccacccatttcatatctttgtgtgaaataatttgatctagaaattgcaaattgtagtcacctggtactccgaatatttcttctatacctaattcgtgtaatctgtccaacagatagtcacctactgtatacattttgtttactagtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgaaaaagcgtgttttttattcaaaatgattctaactcccttacgtaatcaaggaatctttttgccttggcctccgcgtcattaaacttcttgttgttgacgctaacattcaacgctagtatatattcgtttttttcaggtaagttcttttcaacgggtcttactgatgaggcagtcgcgtctgaacctgttaagaggtcaaatatgtcttcttgaccgtacgtgtcttgcatgttattagctttgggaatttgcatcaagtcataggaaaatttaaatcttggctctcttgggctcaaggtgacaaggtcctcgaaaatagggcgcgccccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 205 - DNA, 2686 bp, artificial sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 206 - DNA, 2237 bp, Artificial sequence
  Synthetic construct
ttatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaaatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgagcgatttaatctctaattatt

SEQ ID NO: 207 - DNA, 4420 bp, artificial sequence
  Synthetic construct
ttatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaaatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatgtcttccaaaaaaaaaaccgatgaattagtggaaccaaggaaaaaaaaagaggtatccttgattaaggaacactgtttaaacagtgtggtttccaaaaccctgaaactgcattagtgtaatagaagactagacacctcgatacaaataatggttactcaattcaaaactgccagcgaattcgactctgcaattgctcaagacaagctagttgtcgtagatttctacgccacttggtgcggtccatgtaaaatgattgctccaatgattgaaaaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttcatggccttccactttcccaaacaacacctacggtatctctcaagtcttatggggttccattggtttcaccactggtgctaccttgggtgctgctttcgctgctgaagaaattgatccaaagaagagagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactatgaaacccacagagtcgctaccaccggtgaatgggacaagttgacccaagacaagtctttcaacgacaactctaagatcagaatgattgaaatcatgttgccagtcttcgatgctccacaaaacttggttgaacaagctaagttgactgctgctaccaacgctaagcaataa

SEQ ID NO: 208 - DNA, 3521 bp, artificial sequence
  Synthetic construct
aaggaaataaagcaaataacaataacaccattattttaattttttttctattactgtcgctaacacctgtatggttgcaaccaggtgagaatccttctgatgcatactttatgcgtttatgcgttttgcgccccttggaaaaaaattgattctcatcgtaaatgcatactacatgcgtttatgggaaaagcctccatatccaaaggtcgcgtttcttttagaaaaactaatacgtaaacctgcattaaggtaagattatatcagaaaatgtgttgcaagaaatgcattatgcaattttttgattatgacaatctctcgaaagaaatttcatatgatgagacttgaataatgcagcggcgcttgctaaaagaacttgtatataagagctgccattctcgatcaatatactgtagtaagtcctttcctctctttcttattacacttatttcacataatcaatctcaaagagaacaacacaatacaataacaagaagaacaaaatgaaagctctggtttatcacggtgaccacaagatctcgcttgaagacaagcccaagcccacccttcaaaagcccacggatgtagtagtacgggttttgaagaccacgatctgcggcacggatctcggcatctacaaaggcaagaatccagaggtcgccgacgggcgcatcctgggccatgaaggggtaggcgtcatcgaggaagtgggcgagagtgtcacgcagttcaagaaaggcgacaaggtcctgatttcctgcgtcacttcttgcggctcgtgcgactactgcaagaagcagctttactcccattgccgcgacggcgggtggatcctgggttacatgatcgatggcgtgcaggccgaatacgtccgcatcccgcatgccgacaacagcctctacaagatcccccagacaattgacgacgaaatcgccgtcctgctgagcgacatcctgcccaccggccacgaaatcggcgtccagtatgggaatgtccagccgggcgatgcggtggctattgtcggcgcgggccccgtcggcatgtccgtactgttgaccgcccagttctactccccctcgaccatcatcgtgatcgacatggacgagaatcgcctccagctcgccaaggagctcggggcaacgcacaccatcaactccggcacggagaacgttgtcgaagccgtgcataggattgcggcagagggagtcgatgttgcgatcgaggcggtgggcataccggcgacttgggacatctgccaggagatcgtcaagcccggcgcgcacatcgccaacgtcggcgtgcatggcgtcaaggttgacttcgagattcagaagctctggatcaagaacctgacgatcaccacgggactggtgaacacgaacacgacgcccatgctgatgaaggtcgcctcgaccgacaagcttccgttgaagaagatgattacccatcgcttcgagctggccgagatcgagcacgcctatcaggtattcctcaatggcgccaaggagaaggcgatgaagatcatcctctcgaacgcaggcgctgcctgagctaattaacataaaactcatgattcaacgtttgtgtatttttttacttttgaaggttatagatgtttaggtaaataattggcatagatatagttttagtataataaatttctgatttggtttaaaatatcaactattttttttcacatatgttcttgtaattacttttctgtcctgtcttccaggttaaagattagcttctaatattttaggtggtttattatttaattttatgctgattaatttatttacttgtttaaacggccggccaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttcatggccttctactttcccaacagatgtatacgctatcgtccaagtcttgtggggttccattggtttcacagtcggcgctctattgggtgctactatggccgctgaagaacttgatccaaagaagagagttattttattcattggtgacggttctctacaattgactgttcaagaaatctctaccatgattagatggggtttgaagccatacatttttgtcttgaataacaacggttacaccattgaaaaattgattcacggtcctcatgccgaatataatgaaattcaaggttgggaccacttggccttattgccaacttttggtgctagaaactacgaaacccacagagttgctaccactggtgaatgggaaaagttgactcaagacaaggacttccaagacaactctaagattagaatgattgaagttatgttgccagtctttgatgctccacaaaacttggttaaacaagctcaattgactgccgctactaacgctaaacaataa

SEQ ID NO: 209 - DNA, 1685 bp, artificial sequence
  Synthetic construct
gtattttggtagattcaattctctttccctttccttttccttcgctccccttccttatcagcattgcggattacgtattctaatgttcagataacttcgtatagcatacattatacgaagttatgcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtatagcatacattatacgaagttatccagtgatgatacaacgagttagccaaggtgacactctccccccccctccccctctgatctttcctgttgcctctttttcccccaaccaa

SEQ ID NO: 210 - DNA, 22 bp, artificial sequence
  Primer oBP594
agctgtctcgtgttgtgggttt

SEQ ID NO: 211 - DNA, 49 bp, artificial sequence
  Primer oBP595
cttaataatagaacaatatcatcctttacgggcatcttatagtgtcgtt

SEQ ID NO: 212 - DNA, 49 bp, artificial sequence
  Primer oBP596
gcgccaacgacactataagatgcccgtaaaggatgatattgttctatta

SEQ ID NO: 213 - DNA, 49 bp, artificial sequence
  Primer oBP597
tatggaccctgaaaccacagccacattgcaacgacgacaatgccaaacc

SEQ ID NO: 214 - DNA, 49 bp, artificial sequence
  Primer oBP598
tccttggtttggcattgtcgtcgttgcaatgtggctgtggtttcagggt

SEQ ID NO: 215 - DNA, 49 bp, artificial sequence
  Primer oBP599
atcctctcgcggagtccctgttcagtaaaggccatgaagctttttcttt

SEQ ID NO: 216 - DNA, 49 bp, artificial sequence
  Primer oBP600
attggaaagaaaaagcttcatggcctttactgaacagggactccgcgag

SEQ ID NO: 217 - DNA, 22 bp, artificial sequence
  Primer oBP601
tcataccacaatcttagaccat

SEQ ID NO: 218 - DNA, 21 bp, artificial sequence
  Primer oBP602
tgttcaaacccctaaccaacc

SEQ ID NO: 219 - DNA, 22 bp, artificial sequence
  Primer oBP603
tgttcccacaatctattaccta

SEQ ID NO: 220 - DNA, 21 bp, artificial sequence
  Primer oBP605
tactgaacagggactccgcga

SEQ ID NO: 221 - DNA, 21 bp, artificial sequence
  Primer oBP606
tcataccacaatcttagacca

SEQ ID NO: 222 - DNA, 34 bp, artificial sequence
  Primer oBP562
aattgtttaaacatgtatacagtaggtgactatc

SEQ ID NO: 223 - DNA, 49 bp, artificial sequence
  Primer oBP563
aatcataaatcataagaaattcgcttatcagctcttgttttgttctgca

SEQ ID NO: 224 - DNA, 49 bp, artificial sequence
  Primer oBP564
ttatttgcagaacaaaacaagagctgataagcgaatttcttatgattta

SEQ ID NO: 225 - DNA, 34 bp, artificial sequence
  Primer oBP565
aattggccggccaaaaaaagcatgcacgtataca

SEQ ID NO: 226 - DNA, 32 bp, artificial sequence
  Primer oBP505
aattgagctcactgtagccctagacttgatag

SEQ ID NO: 227 - DNA, 35 bp, artificial sequence
  Primer oBP506
aattggcgcgcctgtatatgagatagttgattgta

SEQ ID NO: 228 - DNA, 35 bp, artificial sequence
  Primer oBP507
aattttaattaagtctaggttctttggctgttcaa

SEQ ID NO: 229 - DNA, 33 bp, artificial sequence
  Primer oBP508
aattgtcgactttagaagtgtcaacaacgtatc

SEQ ID NO: 230 - DNA, 34 bp, artificial sequence
  Primer oBP674
aattggcgcgccaattaccgtcgctcgtgatttg

SEQ ID NO: 231 - DNA, 34 bp, artificial sequence
  Primer oBP675
aattgtttaaacttgaatatgtattacttggtta

SEQ ID NO: 232 - DNA, 22 bp, artificial sequence
  Primer oBP495
ggagatatacaatagaacagat

SEQ ID NO: 233 - DNA, 22 bp, artificial sequence
  Primer oBP496
tagcaatggggtttttttcagt

SEQ ID NO: 234 - DNA, 47 bp, artificial sequence
  primer T FBA1SalI
attctacgtacgtcgacgcctacttggcttcacatacgttgcatacg

SEQ ID NO: 235 - DNA, 49 bp, artificial sequence
  primer B FBA1Spel
gtatcaaatactagtttgaatatgtattacttggttatggttatatatg

SEQ ID NO: 236 - DNA, 43 bp, artificial sequence
  primer T U/PGK1KpnI
actacagatggtaccaattaccgtcgctcgtgatttgtttgca

SEQ ID NO: 237 - DNA, 45 bp, artificial sequence
  primer B U/PGK1SalI
agcatccttgtcgacaggaccttgttgtgtgacgaaattggaagc

SEQ ID NO: 238 - DNA, 22 bp, artificial sequence
  Primer oBP556
tattttcgaggaccttgtcacc

SEQ ID NO: 239 - DNA, 25 bp, artificial sequence
  Primer oBP561
tggccattaatctttcccatattag

SEQ ID NO: 240 - DNA, 725 bp, artificial sequence
  Synthetic construct
aattaccgtcgctcgtgatttgtttgcaaaaagaacaaaactgaaaaaacccagacacgctcgacttcctgtcttcctattgattgcagcttccaatttcgtcacacaacaaggtcctgtcgacgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaa

SEQ ID NO: 241 - DNA, 10494 bp, artificial sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgctagggattcataaccattttctcaatcgaattacacagaacacaccgtacaaacctctctatcataactacttaatagtcacacacgtactcgtctaaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaactcgagatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaactagtatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatactccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgattaattaactagagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtc

SEQ ID NO: 242 - DNA, 8366 bp, artificial sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctccaccgcggtggtttaacgtatagacttctaatatatttctccatacttggtattttttattcactttttttatacatatttggtttttttatacaatatcaaaaatcaataataattaatttaataacaagaatatgttgctgtgatgactcttaatatacaattgggaagcattcaaggattggtacgacgtttgctttctgataattaaacgcttggcgtttaatcttttttgccgattaataatattattattaccctcttatttattagcattgtcttccgtactaaaaggagtagggaaaaaggcgaagtagagtgacacgtccattacccagcgcttcataagaatccatcaggtatattaaattgtgatgggagaacaaaacaaattgtactatgatgtcgagaaattggtgaattctttgcaggaaagttttgacctggattgcgcgcaaagtgtatcacttttcaccagtaaatcaaggagcaatgaggcttggttggaagagctagagaataaattcaagttaaaagatgatgttgaacttgatgatgtggaaaatttaagagccgaaattgacatgaagttaaatatgttggaggataaagtaagctactatgaaagactttacaaagaactcgaagagttccagaatgaaataaaaattaaaacagttgtgaataacagaagacaatcgcgaactccaaaatgagctatcaaaaacgatagatcgattaggatgactttgaaatgactccgcagtggactggccgttaatttcaagcgtgagtaaaatagtgcatgacaaaagatgagctaggcttttgtaaaaatatcttacgttgtaaaattttagaaatcattatttccttcatatcattttgtcattgaccttcagaagaaaagagccgaccaataatataaataaataaataaaaataatattccattatttctaaacagattcaatactcattaaaaaactatatcaattaatttgaattaacgcggccgctctagttaattaatcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggagtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacgtaccatactagtttgaatatgtattacttggttatggttatatatgacaaaagaaaaagaagaacagaagaataacgcaaggaagaacaataactgaaattgatagagaagtattatgtctttgtctttttataataaatcaagtgcagaaatccgttagacaacatgagggataaaatttaacgtgggcgaagaagaaggaaaaaagtttttgtgagggcgtaattgaagcgatctgttgattgtagattttttttttttgaggagtcaaagtcagaagagaacagacaaatggtattaaccatccaatacttttttggagcaacgctaagctcatgcttttccattggttacgtgctcagttgttagatatggaaagagaggatgctcacggcagcgtgactccaattgagcccgaaagagaggatgccacgttttcccgacggctgctagaatggaaaaaggaaaaatagaagaatcccattcctatcattatttacgtaatgacccacacatttttgagattttcaactattacgtattacgataatcctgctgtcattatcattattatctatatcgacgtatgcaacgtatgtgaagccaagtaggcgtcgacaggaccttgttgtgtgacgaaattggaagctgcaatcaataggaagacaggaagtcgagcgtgtctgggttttttcagttttgttctttttgcaaacaaatcacgagcgacggtaattggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 243 - DNA, 20 bp, artificial sequence
  Primer N98 SEQF1
cgtgttagtcacatcaggac

SEQ ID NO: 244 - DNA, 22 bp, artificial sequence
  Primer N99 SEQR2
catcgactgcattacgcaactc

SEQ ID NO: 245 - DNA, 20 bp, artificial sequence
  Primer N98 SEQF4
ggtttctgtctctggtgacg

SEQ ID NO: 246 - DNA, 7589 bp, artificial sequence
  Synthetic construct
tatttgtatcgaggtgtctagtcttctattacactaatgcagtttcagggttttggaaaccacactgtttaaacagtgttccttaatcaaggatacctctttttttttccttggttccactaattcatcggttttttttttggaagacatcttttccaacgaaaagaatatacatatcgtttaagagaaattctccaaatttgtaaagaagcggacccagacttaaggccgcccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgattaattaactagagagctttcgttttcatgagttccccgaattctttcggaagcttgtcacttgctaaattaatgttatcactgtagtcaaccgggacatcgatgatgacaggaccttcagcgttcatgccttgacgcagaacatctgccagctggtctggtgattctacgcgcaagccagttgctccgaagctttccgcatatttcacgatatcgatatttccgaaatcgaccgcagatgtacggttatattttttcaattgctggaatgcaaccatgtcatatgtgctgtcgttccatacaatgtgtacaattggtgcttttagtcgaactgctgtctctaattccattgctgagaataagaaaccgccgtcaccagagacagaaaccactttttctcccggtttcaccaatgaagcgccgattgcccaaggaagcgcaacgccgagtgtttgcataccgttactgatcattaatgttaacggctcgtagctgcggaaataacgtgacatccaaatggcgtgcgaaccgatatcgcaagttactgtaacatgatcatcgactgcattacgcaactctttaacgatttcaagagggtgcgctctgtctgatttccaatctgcaggcacctgctcaccttcatgcatatattgttttaaatcagaaaggattttctgctcacgctctgcaaattccactttcacagcatcgtgttcgatatgattgatcgtggacggaatgtcaccgatcaattcaagatcaggctggtaagcatgatcaatgtcagcgataatctcgtctaaatggataattgtccggtctccattgatattccagaatttcggatcatattcaatcgggtcatagccgatcgtcagaacaacatctgcctgctctagcagtaaatcgccaggctggttgcggaacaaaccgatacggccaaaatattgatcctctaaatctctagaaagggtaccggcagcttgatatgtttcaacaaatggaagctgaacctttttcaaaagcttgcgaaccgctttaattgcttccggtcttccgcctttcatgccgaccaaaacgacaggaagttttgctgtttggatttttgctatggccgcactgattgcatcatctgctgcaggaccgagttttggcgctgcaacagcacgcacgtttttcgtatttgtgacttcattcacaacatcttgcggaaagctcacaaaagcggccccagcctgccctgctgacgctatcctaaatgcatttgtaacagcttccggtatattttttacatcttgaacttctacactgtattttgtaatcggctggaatagcgccgcattatccaaagattgatgtgtccgttttaaacgatctgcacggatcacgtttccagcaagcgcaacgacagggtctccttcagtgttcgctgtcagcaggcctgttgccaagttagaggcacccggtcctgatgtgactaacacgactcccggttttccagttaaacggccgactgcttgggccatgaatgctgcgttttgttcgtgccgggcaacgataatttcaggtcctttatcttgtaaagcgtcaaataccgcatcaatttttgcacctggaatgccaaatacatgtgtgacaccttgctccactaagcaatcaacaacaagctccgcccctctgtttttcacaagggatttttgttcttttgttgcttttgtcaacatcctcagctctagatttgaatatgtattacttggttatggttatatatgacaaaagaaaaagaagaacagaagaataacgcaaggaagaacaataactgaaattgatagagaagtattatgtctttgtctttttataataaatcaagtgcagaaatccgttagacaacatgagggataaaatttaacgtgggcgaagaagaaggaaaaaagtttttgtgagggcgtaattgaagcgatctgttgattgtagattttttttttttgaggagtcaaagtcagaagagaacagacaaatggtattaaccatccaatacttttttggagcaacgctaagctcatgcttttccattggttacgtgctcagttgttagatatggaaagagaggatgctcacggcagcgtgactccaattgagcccgaaagagaggatgccacgttttcccgacggctgctagaatggaaaaaggaaaaatagaagaatcccattcctatcattatttacgtaatgacccacacatttttgagattttcaactattacgtattacgataatcctgctgtcattatcattattatctatatcgacgtatgcaacgtatgtgaagccaagtaggcaattatttagtactgtcagtattgttattcatttcagatcttaagcctaaccaggccaattcaacagactgtcggcaacttcttgtctggtctttccatggtaagtgacagtgcagtaataatatgaaccaatttatttttcgttacataaaaatgcttataaaactttaactaataattagagattaaatcgcaaacggccgactctagaggatcccccaccttggctaactcgttgtatcatcactggataacttcgtatagcatacattatacgaagttatctagggattcataaccattttctcaatcgaattacacagaacacaccgtacaaacctctctatcataactacttaatagtcacacacgtactcgtctaaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaactcgagatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgataacttcgtatagcatacattatacgaagttatctgaacattagaatacgtaatccgcaatgcggggccgcttaattaatctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatccggcgcgccgttt

SEQ ID NO: 247 - DNA, 38 bp, artificial sequence
  Primer N191
atccgcggatagatctcccattaccgacatttgggcgc

SEQ ID NO: 248 - DNA, 9593 bp, artificial sequence
  Synthetic construct
ggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagctacgtcaagcaatgccattcacacaatctggtgatgacgatgcctttaaaatctatcagtaaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctccgcggtgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatctcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactag

SEQ ID NO: 249 - DNA, 11017 bp, artificial sequence
  Synthetic construct
gatcccccgggctgcaggaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggatagatctagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacgctgaggatgacaacagattactcatcaccagcatatttgcaaaaagttgataagtactggcgtgctgccaactacttatcagttggtcaactttatttaaaagataatccactattacaacggccattgaaggccagtgacgttaaggttcatccaattggtcactgggggacgattgccggtcaaaactttatctatgctcatcttaaccgggtcatcaacaagtacggtttgaagatgttctacgttgaaggtccaggtcatggtggtcaagtgatggtttcaaactcttaccttgacggtacttacaccgatatttatccagaaattacgcaggatgttgaagggatgcaaaagctcttcaagcaattctcattcccaggtggggttgcttcccatgcggcacctgaaacacccggttcaatccacgaaggtggcgaacttggttactcaatttcacacggggttggggcaattcttgacaatcctgacgaaatcgccgcggttgttgttggtgatggggaatccgaaacgggtccattagcaacttcatggcaatcaacgaagttcattaacccaatcaacgacggggctgttttaccaatcttgaacttaaatggttttaagatttctaatccaacgatttttggtcggacttctgatgctaagattaaggaatacttcgaaagcatgaattgggaaccaatcttcgttgaaggtgacgatcctgaaaaggttcacccagccttagctaaggccatggatgaagccgttgaaaagatcaaggcaatccagaagcatgctcgcgaaaataacgatgcaacattgccagtatggccaatgatcgtcttccgcgcacctaagggctggactggtccgaagtcatgggacggtgataagatcgaaggttcattccgtgctcatcaaattccgattcctgttgatcaaaatgacatggaacatgcggatgctttagttgattggctcgaatcatatcaaccaaaagaactcttcaatgaagatggctctttgaaggatgatattaaagaaattattcctactggggacagtcggatggctgctaacccaatcaccaatggtggggtcgatccgaaagccttgaacttaccaaacttccgtgattatgcggtcgatacgtccaaagaaggcgcgaatgttaagcaagatatgatcgtttggtcagactatttgcgggatgtcatcaagaaaaatcctgataacttccggttgttcggacctgatgaaaccatgtctaaccgtttatatggtgtcttcgaaaccactaatcgtcaatggatggaagacattcatccagatagtgaccaatatgaagcaccagctggccgggtcttagatgctcagttatctgaacaccaagctgaaggttggttagaaggttacgtcttaactggacgtcatgggttatttgccagttatgaagccttcctacgcgttgtggactcaatgttgacgcaacacttcaagtggttacgtaaagccaatgaacttgattggcgtaaaaagtacccatcacttaacattatcgcggcttcaactgtattccaacaagaccataatggttatacccaccaagatccaggtgcattaactcatttggccgaaaagaaaccagaatacattcgtgaatatttaccagccgatgccaacacgttattagctgtcggtgacgtcattttccggagccaagaaaagatcaactacgtggttacgtcaaaacacccacgtcaacaatggttcagcattgaagaagctaagcaattagttgacaatggtcttggtatcattgattgggcaagtacggaccaaggtagcgaaccagacattgtctttgcagctgctgggacggaaccaacgcttgaaacgttggctgccatccaattactacacgacagtttcccagagatgaagattcgtttcgtgaacgtggtcgacatcttgaagttacgtagtcctgaaaaggatccgcggggcttgtcagatgctgagtttgaccattactttactaaggacaaaccagtggtctttgctttccacggttacgaagacttagttcgtgacatcttctttgatcgtcacaaccataacttatacgtccacggttaccgtgaaaatggtgatattaccacaccattcgacgtacgggtcatgaaccagatggaccgcttcgacttagctaagtcggcaattgcggcgcaaccagcaatggaaaacactggtgcggccttcgttcaatccatggataatatgcttgctaaacacaatgcctatatccgggatgccggaactgacttgccagaagttaatgattggcaatggaagggtttaaaataattaattaatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcgggcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaattaagctaatccttgggctgctgtaataatcgcaaccttataaacgtcttcttcactgcatccacgtgacaagtcggagaccggcttgttcaggccttgcaagacaggacccaccgcttcaaaatgaccaaatcgttgcgcaatcttatagccaatattaccagactgaagctctggaaatacaaagacattggcatgaccagctactttggaaccaggagccttttgcaaaccaactttttcaacgaaggccgcgtcaaattgaagttcaccatcgatagccaattccggttcagcagcttgcgccttggccgttgcttcttgcactttagtgaccatttcacccttagccgaacccttagttgagaagctgagcatcgcaactttcgggtcaatatcgaagaccttagcagtagccgcactctgagtggcaatttccgctaacgtatcggcatcgggatcaatattgatggcacagtcagcaaagacgtagcgttcctcacccttttgcatgataaatgcacccgagattcggtgtgaaccgggcttggtcttaataatttgtaacgctggccgtaccgtatcaccagttggatggattgcacctgaaaccatcccatccgctttgcccatataaacgagcatcgtgccaaagtagttttcatcttccagcattttagccgcttgttctggcgtattcttacctttccgccgttcaacgagggcatcaagcattgcttgcttatcttcagccgggtatgtcgcaggatcaaggacttgaacgcctgttaaatccgcattcaaatcgttagccacagcctgaactttgtccgttgcacctaaaacaatcggcttaaccaagccgtctgcagctaatcgcgctgccgcaccgacaattcggggttcagttccttcagggaaaacaattgtttgatctttaccagtaattttttgtgctaatgactcaaataaatccatcctcagcgagatagttgattgtatgcttggtatagcttgaaatattgtgcagaaaaagaaacaaggaagaaagggaacgagaacaatgacgaggaaacaaaagattaataattgcaggtctatttatacttgatagcaagacagcaaacttttttttatttcaaattcaagtaactggaaggaaggccgtataccgttgctcattagagagtagtgtgcgtgaatgaaggaaggaaaaagtttcgtgtgcttcgagatacccctcatcagctctggaacaacgacatctgttggtgctgtctttgtcgttaattttttcctttagtgtcttccatcatttttttgtcattgcggatatggtgagacaacaacgggggagagagaaaagaaaaaaaaagaaaagaagttgcatgcgcctattattacttcaatagatggcaaatggaaaaagggtagtgaaacttcgatatgatgatggctatcaagtctagggctacagtattagttcgttatgtaccaccatcaatgaggcagtgtaattggtgtagtcttgtttagcccattatgtcttgtctggtatctgttctattgtatatctcccctccgccacctacatgttagggagaccaacgaaggtattataggaatcccgatgtatgggtttggttgccagaaaag

SEQ ID NO: 250 - DNA, 30 bp, artificial sequence
  primer N1111
tatttgtatcgaggtgtctagtcttctatt

SEQ ID NO: 251 - DNA, 47 bp, artificial sequence
  upper primer
catcatcacagtttaaacagtatgttgaagcaaatcaacttcggtgg

SEQ ID NO: 252 - DNA, 48 bp, artificial sequence
  lower primer
ggacgggccctgcaggccttattggttttctggtctcaactttctgac

SEQ ID NO: 253 - DNA, 52 bp, artificial sequence
  Primer OT1349
catcatcacagtttaaacagtatgaaagttttctacgataaagactgcgacc

SEQ ID NO: 254 - DNA, 51 bp, artificial sequence
  Primer OT1318
gcacttgataggcctgcagggccttagttcttggctttgtcgacgattttg

SEQ ID NO: 255 - DNA, 15 bp, artificial sequence
  Primer OT1383
ctagtcaccggtggc

SEQ ID NO: 256 - DNA, 15 bp, artificial sequence
  Primer OT1384
ggccgccaccggtga

SEQ ID NO: 257 - protein, 343 aa, artificial sequence
  Synthetic construct
MEECKMAKIYYQEDCNLSLLDGKTIAVIGYGSQGHAHALNAKESGCNVIIGLYEGSKSWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 258 - protein, 343 aa, artificial sequence
  KARI variant k9d3
MEECKMAKIYYQEDCNLSLLDGKTIAVIGYGSQGHAHALNAKESGCNVIIGLYEGAKDWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 259 - protein, 343 aa, artificial sequence
  Kari variant K9G9
MEECKMAKIYYQEDCNLSLLDGKTIAVIGYGSQGHAHALNAKESGCNVIIGLYEGAKEWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 260 - DNA, 80 bp, artificial sequence
  BK505 primer
ttccggtttctttgaaatttttttgattcggtaatctccgagcagaaggagcattgcggattacgtattctaatgttcag

SEQ ID NO: 261 - DNA, 7938 bp, artificial sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtggccggcttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaacacgtgagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgaggatcccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 262 - protein, 340 aa, Lactococcus lactis
MAVTMYYEDDVEVSALAGKQIAVIGYGSQGHAHAQNLRDSGHNVIIGVRHGKSFDKAKEDGFETFEVGEAVAKADVIMVLAPDELQQSIYEEDIKPNLKAGSALGFAHGFNIHFGYIKVPEDVDVFMVAPKAPGHLVRRTYTEGFGTPALFVSHQNASGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDEVKKNMKLVLADIQSGKFAQDFVDDFKAGRPKLIAYREAAKNLEIEKIGAELRQAMPFTQSGDDDAFKIYQ